(19) United States
(12) Patent Application Publication
(10) Pub. No.: US 2025/0034584 A1
(43) Pub. Date: Jan. 30, 2025

(54) GENETICALLY ENGINEERED LAND PLANTS THAT EXPRESS AN INCREASED SEED YIELD PROTEIN AND/OR AN INCREASED SEED YIELD RNA

(71) Applicant: YIELD10 BIOSCIENCE, INC., Woburn, MA (US)

(72) Inventors: Jihong TANG, West Roxbury, MA (US); Meghna MALIK, Saskatoon (CA); Nirmala SHARMA, Saskatoon (CA); Claire BURKITT, Saskatoon (CA); Yuanyuan JI, Saskatoon (CA); Madana M.R. AMBAVARAM, Andover, MA (US); Kieran RYAN, Sharon, MA (US); Oliver P. PEOPLES, Arlington, MA (US); Kristi D. SNELL, Belmont, MA (US); Frank Anthony SKRALY, Watertown, MA (US); Venkatesh BOLLINA, Saskatoon (CA)

(21) Appl. No.: 18/821,276

(22) Filed: Aug. 30, 2024

Related U.S. Application Data
(63) Continuation of application No. 17/273,159, filed on Mar. 3, 2021, now Pat. No. 12,084,672.
(60) Provisional application No. 62/834,766, filed on Apr. 16, 2019.
(60) Provisional application No. 62/744,932, filed on Oct. 12, 2018.
(60) Provisional application No. 62/726,653, filed on Sep. 4, 2018.

(51) Int. Cl. C12N 15/82; C07K 14/415

(52) U.S. Cl. CPC C12N 15/8262; C07K 14/415; C12N 15/8249

(57) ABSTRACT

A genetically engineered land plant that expresses a protein that has homology to a plant invertase inhibitor and/or a pectin methylesterase inhibitor and that increases seed yield with increased expression (“an ISY protein”) is disclosed. The plant comprises a modified gene for the ISY protein. The ISY protein comprises one or more of an ISY protein of Camelina sativa comprising SEQ ID NO: 2 or a fragment, Camelina sativa homolog, or ortholog thereof. The modified gene comprises a promoter and a nucleic acid sequence encoding the ISY protein. The promoter is non-cognate with respect to the nucleic acid sequence encoding the ISY protein. The modified gene is configured such that transcription of the nucleic acid sequence is initiated from the promoter and results in expression of the ISY protein. A genetically engineered land plant that expresses an RNA that increases seed yield with increased expression also is disclosed.

### CROSS REFERENCE TO RELATED APPLICATIONS

This application is a continuation of U.S. application Ser. No. 17/273,159, filed Mar. 3, 2021, which is a national stage application of International Appl. PCT/US2019/049281, filed Sep. 3, 2019, and which claims benefit of U.S. Provisional Application No. 62/726,653, filed Sep. 4, 2018, U.S. Provisional Application No. 62/744,932, filed Oct. 12, 2018, and U.S. Provisional Application No. 62/834,766, filed Apr. 16, 2019, all of which are hereby incorporated by reference.

### FIELD OF THE INVENTION

The present invention relates to genetically engineered land plants, and more particularly to genetically engineered land plants that express a protein that has homology to a plant invertase inhibitor and/or a pectin methylesterase inhibitor and that increases seed yield with increased expression (“an ISY protein”) and/or an RNA that increases seed yield with increased expression (“an ISY RNA”).

### BACKGROUND OF THE INVENTION

The world faces a major challenge in the next 30 years to meet the increased demands for food production to feed a growing global population, which is expected to reach 9 billion by the year 2050. Food output will need to be increased by up to 70% in view of the growing population. Increased demand for improved diet, concomitant land use changes for new living space and infrastructure, alternative uses for crops and changing weather patterns will add to the challenge.

Major agricultural crops include food crops, such as maize, wheat, oats, barley, soybean, millet, sorghum, pulses, bean, tomato, corn, rice, cassava, sugar beets, and potatoes, forage crop plants, such as hay, alfalfa, and silage corn, and oilseed crops, such as camelina, Brassica species (e.g. B. napus (canola), B. rapa, B. juncea, and B. carinata), crambe, soybean, sunflower, safflower, oil palm, flax, and cotton, among others. Productivity of these crops, and others, is limited by numerous factors, including for example relative inefficiency of photochemical conversion of light energy to fixed carbon during photosynthesis, as well as loss of fixed carbon by photorespiration and/or other essential metabolic pathways having enzymes catalyzing decarboxylation reactions. For seed (grain), tuber or fruit crops, the ratio of seed, tubers or fruit produced per unit plant biomass is also a major determinant of crop productivity. Crop productivity is also limited by the availability of water. Achieving step changes in crop yield requires new approaches.

Increasing seed yield in major crops can be viewed as a two-step carbon optimization problem, the first is improving photosynthetic carbon fixation and the second is optimizing the flow of fixed carbon to seed production versus vegetative biomass (roots, stems, leaves etc.). The ratio of harvested seed to vegetative plant biomass is also described as the harvest index. Increasing the harvest index of seed, fruit and tuber crops is an objective of this invention.

There are a number of examples of using genetic engineering to increase carbon capture in crops including the use of genes from other photosynthetic species such as algae. It has recently been shown by Schnell et al., WO2015/103074, that Camelina plants transformed to express CCP1 of the eukaryotic algal species Chlamydomonas reinhardtii have reduced transpiration rates, increased CO₂ assimilation rates and higher yield than control plants which do not express the CCP1 gene. Although these engineered plants had higher seed yield, the inventors also disclosed that the size of individual seeds from these plants was significantly reduced. A reasonable interpretation of these results is that the CCP1 protein increases carbon capture efficiency, resulting in increased availability of carbon for seed production. This in turn results in an increased number of seed bearing structures and seeds being produced, but the plant is either unable to provide sufficient fixed carbon to produce the large number of seeds at full size or the increased availability of fixed carbon in green tissue is signaling the plant to shut off the flow of carbon to seeds.

In WO2017/136668, to Yield10 Bioscience, a number of orthologs of CCP1 from algal species that share common protein sequence domains including mitochondrial membrane domains and transporter protein domains were shown to increase seed yield when expressed constitutively in Camelina plants.

In WO2018/156686, to Yield10 Bioscience, CCP1 and its orthologs from other algae are referred to as mitochondrial transporter proteins. The inventors tested the impact of expressing CCP1 or its algal orthologs using seed-specific promoters with the unexpected outcome that both seed yield and seed size increased. Again this implies that in the original CCP1 plants where CCP1 was constitutively expressed the plant was shutting off flow of carbon to seeds. These inventors also recognized the benefits of combining constitutive expression and seed-specific expression of CCP1 or any of its orthologs in the same plant as a means to further increase seed yield.

In WO2018/232232, to Yield10 Bioscience, genetically engineered plants that express a plant CCP1-like mitochondrial transporter protein are disclosed. The genetically engineered plants include a modified gene for the plant CCP1-like mitochondrial transporter protein. The modified gene includes a promoter and a nucleic acid sequence encoding the plant CCP1-like mitochondrial transporter protein. The promoter is non-cognate with respect to the nucleic acid sequence.

In WO2019/104278, to Yield10 Bioscience, genetically engineered plants that express a plant CCP1-like mitochondrial transporter protein and a LCID/E gene are disclosed.

The primary product of photosynthesis is sucrose (sugar), which is then transported throughout the plant and used in plants cells and tissues as a source of both fixed carbon and energy. Sucrose partitioning to seeds and other carbon sink tissues such as fruits or tubers is a key factor in determining yield. Plant invertases and their respective inhibitors have been shown to have a significant role in regulating the partitioning of sugar in plants. Invertases accomplish this through regulating phloem loading, unloading, and sucrose transport (Lammens et al. (2008), J Mol Biol, 377(2):378-385). Plant invertases are a class of proteins that hydrolyze sucrose into fructose and glucose. Plant invertases include cell wall invertases (CWIs) and vacuolar invertases. Cell wall invertases, located within the cell wall, play key roles in the unloading of sucrose from the apoplast to the sink tissues. Cell wall and vacuolar invertases are highly stable proteins due to the presence of glycans. As a result, their activity may be highly dependent on posttranslational regulation by their inhibitory proteins, termed plant invertase inhibitors, which include cell wall invertase inhibitors (CWIIs) and vacuolar invertase inhibitors. Thus, for example, cell wall invertases interact with cell wall invertase inhibitors, which post-transcriptionally regulate their activity.

The plant invertase inhibitors, including cell wall invertase inhibitors, are small peptides, with molecular masses (MW) ranging from 15 to 23 kDa, and may be localized to either the cell wall or vacuole. The cell wall invertase inhibitors are expressed in pollen development, early developing seeds, and senescing leaves, indicative of assimilate allocation being a limiting factor at these stages of development.

Quantitative trait loci analysis for fruit size in tomato (Lin5), and grain size in rice (GIF1) and maize (MN1) identified mutations in cell wall invertases that led to reduction in their activity in pedicel/fruit tissues (Wang et al. (2008), Nature Genetics, 40(11):1370-1374; Fridman et al. (2004), Science, 305(5691):1786-1789; Cheng et al. (1996), Plant Cell, 8(6):971-983). Fruit-specific suppression of the cell wall invertase inhibitor (CWII) in tomato or rice led to increases in net seed/grain weight of 22% and 10%, respectively (Wang et al. (2008), Nature Genetics, 40(11):1370-1374; Jin et al. (2009), Plant Cell, 21(7):2072-2089).

Thus, based on the teachings in the literature, two general approaches can be used to enhance assimilate flux into sink tissues to increase the yield of seeds, fruits or tubers: overexpression of CWI or repression of its inhibitor protein CWII. Methods using one or both of these general approaches for producing plants having an increased number of seeds and methods for producing plants having increased assimilate partitioning directed into fruits, seeds and/or other plant part (e.g., roots and/or tubers), and/or increased seed, root and/or tuber size, or any combination thereof and transgenic plants made by these methods were disclosed in Sederoff et al., WO2014209792.

“Transgenic plants,” “GMO crops,” and/or “biotech traits” are not widely accepted in some regions and countries and are subject to regulatory approval processes that are very time consuming and prohibitively expensive. The current regulatory framework for transgenic plants results in significant costs (˜$136 million per trait; McDougall, P. 2011, “The cost and time involved in the discovery, development, and authorization of a new plant biotechnology derived trait.” Crop Life International) and lengthy product development timelines that limit the number of technologies that are brought to market. This has severely impaired private investment and the adoption of innovation in this crucial sector. Recent advances in genome editing technologies provide an opportunity to precisely remove genes or edit control sequences to significantly improve plant productivity (Belhaj (2013), Plant Methods, 9:39; Khandagale & Nadaf (2016), Plant Biotechnol Rep, 10:327-343) and open the way to produce plants that may benefit from an expedited regulatory path, or possibly unregulated status.

These risks have severely impaired private investment and the adoption of innovation in this crucial sector. Recent changes in the regulations governing genetically modified crops by USDA-APHIS in the United States and new technologies such as genome editing have begun to change this situation. For example, a corn plant which has been genetically engineered to modify the activity of a corn gene using only DNA sequences from corn, technically described as cis-genic, not transgenic, may be classified as non-regulated provided the engineered corn plant contains no foreign DNA sequences. Advances in genome editing technologies provide an opportunity to precisely remove or insert DNA sequences in the plant genome of interest to inactivate specific plant genes or to alter their expression by modifying their promoter sequences to improve plant performance (Belhaj (2013), Plant Methods, 9:39; Khandagale & Nadaf (2016), Plant Biotechnol Rep, 10:327-343). Genetically engineered plants made using this approach contain no foreign DNA sequences and may also be categorized as non-regulated by USDA-APHIS. In both cases however, the regulatory status of the engineered plants are appropriately subject to the usual criteria for approval of any new plant variety.

Given the costs and challenges associated with obtaining regulatory approval and societal acceptance of transgenic crops there is a need to identify, where possible, plant genes, that can be genetically engineered to increase seed, fruit or tuber yield, particularly without relying on genes, control sequences, or proteins derived from non-plants to the extent possible. Herein we disclose novel plant genes and methods for identifying those novel plant genes which can be used to increase the yield of seed, fruits and tubers in major food crops. We also disclose methods of use of those novel plant genes, and fragments thereof, to increase the yield of seed, fruits, and/or tubers. We also disclose plants engineered to have higher expression of those genes having higher seed, fruit, and/or tuber yield.

### BRIEF SUMMARY OF THE INVENTION

A genetically engineered land plant that expresses a protein that has homology to a plant invertase inhibitor and/or a pectin methylesterase inhibitor and that increases seed yield with increased expression (“an ISY protein”) is disclosed. The genetically engineered land plant comprises a modified gene for the ISY protein. The ISY protein comprises one or more of (i) an ISY protein of Camelina sativa comprising SEQ ID NO: 2; (ii) a fragment of an ISY protein of Camelina sativa comprising SEQ ID NO: 2; (iii) a Camelina sativa homolog of an ISY protein of Camelina sativa comprising SEQ ID NO: 2; or (iv) an ortholog of an ISY protein of Camelina sativa comprising SEQ ID NO: 2. The modified gene comprises (i) a promoter and (ii) a nucleic acid sequence encoding the ISY protein. The promoter is non-cognate with respect to the nucleic acid sequence encoding the ISY protein. The modified gene is configured such that transcription of the nucleic acid sequence is initiated from the promoter and results in expression of the ISY protein.

In some examples, the ISY protein comprises the ISY protein of Camelina sativa comprising SEQ ID NO: 2. In some of these examples, the ISY protein of Camelina sativa comprising SEQ ID NO: 2 corresponds to one or more of ISY protein of Camelina sativa of SEQ ID NO: 2, ISY protein of Camelina sativa of SEQ ID NO: 5, modified ISY protein of Camelina sativa of SEQ ID NO: 138, or modified ISY protein of Camelina sativa of SEQ ID NO: 140.

In some examples, the ISY protein comprises the fragment of an ISY protein of Camelina sativa comprising SEQ ID NO: 2. In some of these examples, the fragment corresponds to 114 to 133 contiguous amino acids of SEQ ID NO: 2.

In some examples, the ISY protein comprises the Camelina sativa homolog of an ISY protein of Camelina sativa comprising SEQ ID NO: 2. In some of these examples, the Camelina sativa homolog corresponds to one or more Camelina sativa proteins of SEQ ID NOS: 21-40.

In some examples, the ISY protein comprises the ortholog of an ISY protein of Camelina sativa comprising SEQ ID NO: 2. In some of these examples, the ortholog corresponds to one or more (i) canola proteins of SEQ ID NOS: 69, 71, 73, and/or 75; (ii) soybean proteins of SEQ ID NOS: 89, 91, 93, and/or 95; or (iii) corn proteins of SEQ ID NOS: 99, 101, 103, 105, and/or 107.

In some examples, the ISY protein comprises (i) (a) a cysteine residue at position 40, (b) a leucine residue at position 59, (c) a cysteine residue at position 98, and (d) a cysteine residue at position 138, with numbering of positions relative to ISY protein of Camelina sativa of SEQ ID NO: 5, and (ii) an overall identity of at least 10% with ISY protein of Camelina sativa of SEQ ID NO: 2. In some of these examples, the ISY protein further comprises only between 1 to 14 amino acid residues N-terminal to the cysteine residue at position 40, with numbering of positions relative to ISY protein of Camelina sativa of SEQ ID NO: 5. Also in some of these examples, the ISY protein lacks an N-terminal signal peptide. Also in some of these examples, the ISY protein lacks a PKF motif.

In some examples, the ISY protein comprises (i) (a) a cysteine residue at position 25, (b) a cysteine residue at position 40, (c) a leucine residue at position 59, (d) a cysteine residue at position 98, and (e) a cysteine residue at position 138, with numbering of positions relative to ISY protein of Camelina sativa of SEQ ID NO: 5, and (ii) an overall identity of at least 10% with ISY protein of Camelina sativa of SEQ ID NO: 5. In some of these examples, the ISY protein lacks a PKF motif.

In some examples, the ISY protein is heterologous with respect to the genetically engineered land plant. In some examples, the ISY protein is homologous with respect to the genetically engineered land plant.

In some examples, the promoter is a constitutive promoter. In some examples, the promoter is a seed-specific promoter.

In some examples, the modified gene is integrated into genomic DNA of the genetically engineered land plant.

In some examples, the modified gene is stably expressed in the genetically engineered land plant.

In some examples, the genetically engineered land plant exhibits increased expression of the ISY protein in comparison to a reference land plant that does not include the modified gene.

In some examples, the genetically engineered land plant exhibits increased seed yield, fruit yield, and/or tuber yield in comparison to a reference land plant that does not include the modified gene.

In some examples, the genetically engineered land plant is a C3 plant. In some examples, the genetically engineered land plant is a C4 plant.

In some examples, the genetically engineered land plant is a food crop plant selected from the group consisting of maize, wheat, oat, barley, soybean, millet, sorghum, potato, sugar beet, pulse, chickpea, green pea, yellow pea, lentils, bean, tomato, and rice. In some examples, the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

A genetically engineered land plant that expresses an RNA that increases seed yield with increased expression (“an ISY RNA”) also is disclosed. The genetically engineered land plant comprises a modified gene for the ISY RNA. The ISY RNA comprises a contiguous sequence of codons encoding a protein that has homology to a plant invertase inhibitor and/or a pectin methylesterase inhibitor (“an encoded protein”). The encoded protein comprises one or more of (i) a protein of Camelina sativa comprising SEQ ID NO: 2; (ii) a fragment of a protein of Camelina sativa comprising SEQ ID NO: 2; (iii) a Camelina sativa homolog of a protein of Camelina sativa comprising SEQ ID NO: 2; or (iv) an ortholog of a protein of Camelina sativa comprising SEQ ID NO: 2. The modified gene comprises (i) a promoter and (ii) a nucleic acid sequence encoding the ISY RNA. The promoter is non-cognate with respect to the nucleic acid sequence encoding the ISY RNA. The modified gene is configured such that transcription of the nucleic acid sequence is initiated from the promoter and results in expression of the ISY RNA.

In some examples, the encoded protein comprises the protein of Camelina sativa comprising SEQ ID NO: 2.

In some examples, the encoded protein comprises the fragment of a protein of Camelina sativa comprising SEQ ID NO: 2, wherein the fragment corresponds to 114 to 133 contiguous amino acids of SEQ ID NO: 2.

In some examples, the encoded protein comprises the Camelina sativa homolog of a protein of Camelina sativa comprising SEQ ID NO: 2, wherein the Camelina sativa homolog corresponds to one or more Camelina sativa proteins of SEQ ID NOS: 21-40.

In some examples, the encoded protein comprises the ortholog of a protein of Camelina sativa comprising SEQ ID NO: 2, wherein the ortholog corresponds to one or more (i) canola proteins of SEQ ID NOS: 69, 71, 73, and/or 75; (ii) soybean proteins of SEQ ID NOS: 89, 91, 93, and/or 95; or (iii) corn proteins of SEQ ID NOS: 99, 101, 103, 105, and/or 107.

In some examples, the encoded protein comprises (i) (a) a cysteine residue at position 40, (b) a leucine residue at position 59, (c) a cysteine residue at position 98, and (d) a cysteine residue at position 138, with numbering of positions relative to the protein of Camelina sativa of SEQ ID NO: 5, and (ii) an overall identity of at least 10% with the protein of Camelina sativa of SEQ ID NO: 2.

In some examples, the encoded protein comprises (i) (a) a cysteine residue at position 25, (b) a cysteine residue at position 40, (c) a leucine residue at position 59, (d) a cysteine residue at position 98, and (e) a cysteine residue at position 138, with numbering of positions relative to the protein of Camelina sativa of SEQ ID NO: 5, and (ii) an overall identity of at least 10% with the protein of Camelina sativa of SEQ ID NO: 5.

In some examples, the encoded protein is heterologous with respect to the genetically engineered land plant.

In some examples, the encoded protein is homologous with respect to the genetically engineered land plant.

In some examples, the promoter is a constitutive promoter. In some examples, the promoter is a seed-specific promoter.

In some examples, the modified gene is integrated into genomic DNA of the genetically engineered land plant.

In some examples, the modified gene is stably expressed in the genetically engineered land plant.

In some examples, the genetically engineered land plant exhibits increased expression of the ISY RNA in comparison to a reference land plant that does not include the modified gene.

In some examples, the genetically engineered land plant exhibits increased seed yield, fruit yield, and/or tuber yield in comparison to a reference land plant that does not include the modified gene.

In some examples, the genetically engineered land plant is a food crop plant selected from the group consisting of maize, wheat, oat, barley, soybean, millet, sorghum, potato, sugar beet, pulse, chickpea, green pea, yellow pea, lentils, bean, tomato, and rice. In some examples, the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

In some examples, the nucleic acid sequence encoding the ISY RNA is at least 80% identical to SEQ ID NO: 1. In some of these examples, the nucleic acid sequence encoding the ISY RNA comprises SEQ ID NO: 1.

In some examples, expression of the ISY RNA results in expression of the encoded protein. In some examples, expression of the ISY RNA does not result in expression of the encoded protein.

### BRIEF DESCRIPTION OF THE DRAWINGS

FIG. 1A-B shows a multiple sequence alignment of three putative Camelina plant invertase inhibitor/pectin methylesterase inhibitor proteins, or increased seed yield (ISY) proteins, that incorporate CSA15G017550.1, with nine proteins identified as known plant invertase inhibitors according to CLUSTAL 0(1.2.4). The three ISY proteins differ in size with respect to the total number of amino acids due to different start codons. Sequences are as follows: Csa15g017550 protein with 134 amino acids (Csa15g017550_134aa; SEQ ID NO: 2); a 168 amino acid protein containing a different start codon using sequence upstream of Csa15g017550 (Csa15g017550_168aa; SEQ ID NO: 5); a 190 amino acid fragment containing sequence upstream of Csa15g017550 (Csa15g017550_190aa; SEQ ID NO: 4); Zea mays (XP_008668976.1) (SEQ ID NO: 8); Arabidopsis thaliana (AT5G64620) (SEQ ID NO: 9); Arabidopsis thaliana (AEE32232.1) (SEQ ID NO: 10); Solanum lycopersicum (Solyc12g099190) (SEQ ID NO: 11); Solanum lycopersicum (Solyc12g099200) (SEQ ID NO: 12); Beta vulgaris subsp. vulgaris (XP_010685378.1) (SEQ ID NO: 13); Nicotiana tabacum (AY145781.1) (SEQ ID NO: 14); Nicotiana tabacum (Y12805.1) (SEQ ID NO: 15); Solanum tuberosum cultivar Shepody (GU321341.1) (SEQ ID NO: 16). Csa15g017550.1 has been annotated as a putative plant invertase inhibitor/pectin methylesterase inhibitor. Sequences identified as known plant invertase inhibitors were described by Wan et al. (2018), Trends Plant Sci, 23:163-177, and Tang et al. (2017), J Exp Bot, 68:469-482, and are further described in TABLE 3.

FIG. 2 shows a multiple sequence alignment of two ISY proteins that incorporate Csa15g017550.1, with two cell wall invertase inhibitors from Camelina previously described in Sederoff et al., U.S. Pub. No. 2016/0138038, according to CLUSTAL 0(1.2.4). The three homeologs to each of the two cell wall invertase inhibitors (CWII1 and CWII2) are shown in the alignment. Sequences are as follows: the Csa15g017550 protein with 134 amino acids (Csa15g017550_134aa; SEQ ID NO: 2); a 168 amino acid protein containing a different start codon using sequence upstream of Csa15g017550 (Csa15g017550_168aa; SEQ ID NO: 5); Csa03g051630, cell wall invertase inhibitor protein 1 (CWII1) with 159 amino acids (SEQ ID NO: 77); CWII1 homeolog Csa14g051860, CWII1 with 155 amino acids (SEQ ID NO: 79); CWII1 homeolog Csa17g075360, CWII1 with 159 amino acids (SEQ ID NO: 81); CWII2 Csa02g074171, CWII2 with 180 amino acids (SEQ ID NO: 83); CWII2 homeolog Csa11g101740, CWII2 with 181 amino acids (SEQ ID NO: 85); CWII2 homeolog Csa18g038260, CWII2 with 181 amino acids (SEQ ID NO: 87). The PKF amino acid signature sequence for cell wall invertase inhibitors can be found in the CWII1 sequences (Csa03g051630, Csa14g051860, and Csa17g075360) from residues 110-112.

FIG. 3 shows a plasmid map of transformation vector pMBXO58 (SEQ ID NO: 19) expressing the CCP1 gene from the 35S constitutive promoter. Plasmid pMBXO58 contains a CaMV35S constitutive promoter operably linked to CCP1 from Chlamydomonas reinhardtii fused to a C-terminal myc tag operably linked to an OCS3 termination sequence. An expression cassette for the bar gene, driven by the mannopine synthase promoter, imparts transgenic plants resistance to the herbicide bialaphos.

FIG. 4 shows a plasmid map of transformation vector pMBXO84 (SEQ ID NO: 96). Plasmid pMBXO84 contains a seed-specific expression cassette, driven by the promoter from the soya bean oleosin isoform A gene, for expression of CCP1 from Chlamydomonas reinhardtii. An expression cassette for the bar gene, driven by the CaMV35S promoter, imparts transgenic plants resistance to the herbicide bialaphos.

FIG. 5 shows analysis of multiple open reading frames of the gene fragment in SEQ ID NO: 7 producing different size ISY proteins. “M” Predicted start codon according to Camelina Sativa Genome Browser Csa15g017550.1 that produces a 134 amino acid protein (SEQ ID NO: 2). “L,” Alternative start codon, encoded by TTG, that produces a 168 amino acid protein (SEQ ID NO: 5). This start codon was chosen based on alignments with other cell wall invertase inhibitors (FIG. 1). “T” first amino acid of 190 amino acid large amino acid sequence encompassing Csa15g017550. The binding sites of primers P675, P676, P677, and P678 used for PCR and RT-PCR (FIG. 7) experiments are shown.

FIG. 6 shows a Phobius-generated plot of a predicted N-terminal signal peptide for the 168 amino acid ISY protein (SEQ ID NO: 5). The Phobius plot shows the probability of predicted transmembrane domain (X), cytoplasmic domain (square), non-cytoplasmic domain (triangle), and signal peptide sequence (circle). Only a signal peptide and a non-cytoplasmic domain were predicted. The Y-axis corresponds to posterior label probability, plotted from 0 to 1 in increments of 0.2. The X-axis corresponds to amino acid residue number of the 168 amino acid ISY protein of SEQ ID NO: 5, plotted from 0 to 168 in increments of 20.

FIG. 7 shows experiments to verify the presence of the Csa15g017550 gene and RNA transcript in Camelina sativa germplasm 10CS0043 (abbreviated WT43). A) Map of 576 bp of DNA (SEQ ID NO: 7) in the region of the Csa15g017550 gene. The coding sequences for the 134 amino acid and 168 amino acid ISY proteins are shown. Primer binding sites for primers P675, P676, P677, and P678 for PCR and RT PCR experiments are shown. B) PCR experiments on genomic DNA from wild-type Camelina line WT43 showing the presence of the Csa15g017550 gene (SEQ ID NO: 1), as well as sequence upstream of the Csa15g017550 gene, in the genome. A PCR reaction with primer pair P675/P676 produced a band consistent with the expected 0.236 kb fragment. A PCR reaction with primer pair P677/P678 produced a band consistent with the expected 0.269 kb fragment. C) RT-PCR experiments with RNA from transgenic line M116 16-0365, expressing the CCP1 gene from the 35S constitutive promoter in vector pMBXO58 (SEQ ID NO: 19, FIG. 3), and wild-type control line WT43 line 16-0359. Increased expression of the putative plant invertase inhibitor is observed in leaves and siliques of CCP1 lines. Control experiments to probe the presence of transcripts of the actin gene were also performed. D) RT-PCR experiments with RNA from developing seeds transformed with vector pMBXO84 (SEQ ID 96; FIG. 4), expressing the CCP1 gene from the seed-specific oleosin promoter (lines ND79, ND11, ND04, and ND78), compared to the wild-type control line WT43. Control experiments to probe the presence of transcripts of the actin gene were also performed

FIG. 8 shows the syntenic analysis of Csa15g017550. The putative plant invertase inhibitor/pectin methylesterase inhibitor (Csa15g017550, white arrow) was found to be on subgenome G1 and is flanked by coding sequences for Csa15g017560 on the left and by Csa15g017530 on the right. The two homeologs of the left flanking gene Csa15g017560 on subgenomes G2 (Csa19g018770) and G3 (Csa01g015740) are shown. The sequence homeolog of the right flanking gene Csa15g017530 on subgenome 2 (Csa19g018760) and on subgenome 3 (Csa01g015720) are shown. No homeologs were identified for the putative plant invertase inhibitor/pectin methylesterase inhibitor Csa15g017550. On subgenome 2, there was no CDS annotated at the expected genomic position in the Camelina genome database. On subgenome G3, the gene Csa01g015730.1 is present with only 28% sequence identity to Csa15g017550. Figure is not drawn to scale.

FIG. 9 shows a map for pMBXS1168 (SEQ ID NO: 17), a transformation vector designed for Agrobacterium-mediated transformation of dicots, including Camelina. The vector contains the double enhanced CaMV35S constitutive promoter (2× 35S) operably linked to a gene (SEQ ID NO: 1) encoding the 134 amino acid protein for Csa15g017550 (SEQ ID NO: 2) operably linked to a 35S polyadenylation sequence. The visual marker DsRed2B is used to identify transgenic seeds.

FIG. 10 shows a map for pMBXS1169 (SEQ ID NO: 18), a transformation vector designed for Agrobacterium-mediated transformation of dicots, including Camelina. The vector contains the double enhanced CaMV35S constitutive promoter (2× 35S) operably linked to a gene (SEQ ID NO: 6) encoding the 168 amino acid ISY protein (SEQ ID NO: 5) operably linked to a 35S polyadenylation sequence. The visual marker DsRed2B is used to identify transgenic seeds.

FIG. 11 shows a comparison of seed yield and number of tertiary branches in Camelina lines transformed with transformation vectors pMBXS1168 and pMBXS1169. Lines are as follows: control line NJ02, wild-type Suneson transformed with pMBXO58 expressing CCP1; OX10, line of NJ02 transformed with pMBXS1168 (SEQ ID NO: 17) expressing the 134 amino acid Csa15g017550 of SEQ ID NO: 2; PA22, line of NJ02 transformed with pMBXS1169 (SEQ ID NO: 18) expressing the 168 amino acid ISY protein of SEQ ID NO: 5; WTSU, wild-type Suneson; line OY12, wild-type Suneson transformed with pMBXS1168; line OZ27, wild-type Suneson transformed with pMBXS1169. *, indicates statistically significant results according to the student's t-test (p<0.05).

FIG. 12A-E shows the results from a second growth experiment of T3 generation Camelina lines transformed with transformation vectors pMBXS1168 and pMBXS1169. A) Average T4 seed yield (in grams) of C3004 camelina lines compared to wild-type controls (WTSU), n=4 plants. B) Average number of tertiary branches per plant of C3004 camelina lines compared to wild-type controls, n=3-4 plants. C) Average number of siliques per plant of C3004 camelina lines compared to wild-type controls, n=3-4 plants. D) Average number of seeds per plant of C3004 camelina lines compared to wild-type controls, n=4 plants. E) Average 1000 seed weight of C3004 camelina lines compared to wild-type controls, n=4 plants. C3004 camelina lines of pMBXS1168 (OY02, OY04, OY12, OY15 and OY17) and pMBXS1169 (OZ12, OZ15, OZ19, OZ21, OZ26 and OZ27) and the corresponding wildtype (WTSU) were grown in a Conviron BDR16 Growth Chamber set at a 16 hour photoperiod and a 22° C./18° C. day/night temperature. With the lights on, a light reading of 450-650 μmol/m²/see light intensity was recorded 12 inches below the bulbs in the chamber. *=ANOVA & DMRT significant (p=0.05).

FIG. 13A-C shows the results from photosynthetic measurements of select Camelina sativa cv. Suneson C3004 transformants compared to their wild-type controls under field growth conditions. A) Photosynthesis, CO₂ fixation (pmol/m²/sec), n=4-10 plants. B) Effective quantum yield of Photosystem II (PSII), n=4-10 plants. C) Electron transfer rate (ETR) of PSII (pmol/m²/sec), n=4-10 plants. T4 generation seeds of C3004 Camelina sativa cv. Suneson lines transformed with pMBXS1168 (OY03, OY04, OY15 and OY17) and pMBXS1169 (OZ15, OZ19, OZ21, and OZ26) and the corresponding wildtype controls (WTSU) were planted in a field trial in Saskatoon, Canada during the 2019 growth season. Photosynthesis measurements were performed on T4 generation plants at a pre-bolting stage about 3.5 weeks after germination. Data analyzed by Student's t-tests. **, *=t-test significant at p≤0.01 and p≤0.05, respectively.

FIG. 14 shows a multiple sequence alignment of the Camelina ISY proteins with four orthologs from the Brassica napus ZS11 cultivar according to CLUSTAL 0(1.2.4). Sequences are as follows: Csa15g017550_134aa, ISY protein with 134 amino acids (SEQ ID NO: 2); Csa15g017550_168aa, ISY protein with 168 amino acids (SEQ ID NO: 5); XP_013675048, B. napus ortholog with 175 amino acids (SEQ ID NO: 75); XP_013675049, B. napus ortholog with 175 amino acids (SEQ ID NO: 71); XP_013714182, B. napus ortholog with 175 amino acids (SEQ ID NO: 69); and XP_013718725, B. napus ortholog with 175 amino acids (SEQ ID NO: 73).

FIG. 15 shows a multiple sequence alignment of the Camelina ISY proteins with four orthologs from the Glycine max “Williams 82” cultivar according to CLUSTAL 0(1.2.4). Sequences are as follows: Csa15g017550_134aa, ISY protein with 134 amino acids (SEQ ID NO: 2); Csa15g017550_168aa, ISY protein with 168 amino acids (SEQ ID NO: 5); XP_003550932.1, Glycine max ortholog with 183 amino acids (SEQ ID NO: 89); XP_006579665.1, Glycine max ortholog with 183 amino acids (SEQ ID NO: 91); NP_001235997, Glycine max ortholog with 187 amino acids (SEQ ID NO: 93); and XP_006604724, Glycine max ortholog with 183 amino acids (SEQ ID NO: 95).

FIG. 16A-B shows a multiple sequence alignment of the Camelina ISY proteins with five orthologs identified from the maize reference genome Zea mays B73 according to CLUSTAL 0(1.2.4). Sequences are as follows: Csa15g017550_134aa, ISY protein with 134 amino acids (SEQ ID NO: 2); Csa15g017550_168aa, ISY protein with 168 amino acids (SEQ ID NO: 5); NP_001143588, maize ortholog with 217 amino acids (SEQ ID NO: 99); NP_001148423, maize ortholog with 228 amino acids (SEQ ID NO: 101); NP_001149041, maize ortholog with 211 amino acids (SEQ ID NO: 103); XP_008655849, maize ortholog with 195 amino acids (SEQ ID NO: 105); XP_008668976, maize ortholog with 176 amino acids (SEQ ID NO: 107).

FIG. 17A-C shows a multiple sequence alignment of the Camelina ISY proteins with the crop orthologs from canola, soybean, and corn listed in TABLE 12. Sequences are as follows: Csa15g017550_134aa, ISY protein with 134 amino acids (SEQ ID NO: 2) from Camelina sativa; Csa15g017550_168aa, ISY protein with 168 amino acids (SEQ ID NO: 5) from Camelina sativa; XP_013675048, B. napus ortholog with 175 amino acids (SEQ ID NO: 75); XP_013675049, B. napus ortholog with 175 amino acids (SEQ ID NO: 71); XP_013714182, B. napus ortholog with 175 amino acids (SEQ ID NO: 69); XP_013718725, B. napus ortholog with 175 amino acids (SEQ ID NO: 73); XP_003550932.1, Glycine max ortholog with 183 amino acids (SEQ ID NO: 89); XP_006579665.1, Glycine max ortholog with 183 amino acids (SEQ ID NO: 91); NP_001235997, Glycine max ortholog with 187 amino acids (SEQ ID NO: 93); XP_006604724, Glycine max ortholog with 183 amino acids (SEQ ID NO: 95); NP_001143588, maize ortholog with 217 amino acids (SEQ ID NO: 99); NP_001148423, maize ortholog with 228 amino acids (SEQ ID NO: 101); NP_001149041, maize ortholog with 211 amino acids (SEQ ID NO: 103); XP_008655849, maize ortholog with 195 amino acids (SEQ ID NO: 105); and XP_008668976, maize ortholog with 176 amino acids (SEQ ID NO: 107).

FIG. 18 shows maps of DNA constructs for transformation of a canola ortholog of the Camelina ISY gene into canola. A) Transformation construct pMBXS1274 (SEQ ID NO: 193) contains a gene fragment (gene sequence XM_013819595.2, SEQ ID NO: 70), expressed from the double enhanced CaMV35S constitutive promoter (2× 35S), that encodes the 175 amino acid protein in SEQ ID NO: 71. B) Construct pMBXS1277 (SEQ ID NO: 196) contains the SEQ ID NO: 194 DNA fragment, expressed from the double enhanced CaMV35S constitutive promoter (2× 35S), to produce the 138 amino acid protein of SEQ ID NO: 195. The visual marker DsRed2B in both constructs is used to identify transgenic seeds.

FIG. 19 shows plasmid maps for 1) pMBXS1269 (SEQ ID NO: 197) and B) pMBXS1270 (SEQ ID NO: 198), vectors designed for Agrobacterium-mediated transformation of Canola with the 134 amino acid and 168 amino acid Camelina ISY genes, respectively. A) Transformation vector pMBXS1269 contains the double enhanced CaMV35S constitutive promoter (2× 35S) operably linked to a gene (SEQ ID NO: 1) encoding the 134 amino acid protein for Csa15g017550 (SEQ ID NO: 2) operably linked to a 35S polyadenylation sequence. B) Transformation vector pMBXS1270 contains the double enhanced CaMV35S constitutive promoter (2× 35S) operably linked to a gene (SEQ ID NO: 6) encoding the 168 amino acid Camelina ISY protein (SEQ ID NO: 5) operably linked to a 35S polyadenylation sequence. An expression cassette for the bar gene, driven by the double enhanced CaMV35S constitutive promoter (2× 35S), imparts transgenic plants resistance to the herbicide bialaphos in both vectors.

FIG. 20A-C shows maps of DNA for transformation into soybean. A) Plasmid pMBXO74 (SEQ ID NO: 128) contains a constitutive CaMV35S promoter for expression of CCP1 from Chlamydomonas reinhardtii in the pJAZZ (Lucigen) linear vector. The codon usage of the CCP1 gene is from the native Chlamydomonas reinhardtii gene. The 2.7 kb, Apa I-Swa I CaMV35S-CCP1-terminator fragment was excised and co-bombarded with a hygromycin cassette into soybean embryogenic cultures. B) DNA fragment pMBXO92 (SEQ ID NO: 129) contains a constitutive CaMV35S promoter for expression of CCP1 from Chlamydomonas reinhardtii. This CaMV35S promoter has a slightly different sequence than the one in pMBXO74. The CCP1 gene is codon optimized for soybean. The 2.2 kb, Hind III—Eco RI CaMV35S-CCP1—terminator fragment was co-bombarded with a hygromycin cassette into soybean embryogenic cultures. C) Plasmid pMBXO75 (SEQ ID NO: 130) contains a seed-specific expression cassette, driven by the promoter from the soya bean oleosin isoform A gene, for expression of CCP1 from Chlamydomonas reinhardtii in the pJAZZ (Lucigen) linear vector. The CCP1 gene is codon optimized for soybean. The 2.2 kb, SmaI Oleosin-CCP1-oleosin terminator fragment was excised and co-bombarded with a hygromycin cassette in soybean embryogenic cultures.

FIG. 21 shows a plasmid map of rice transformation vector pMBXS1091 (SEQ ID NO: 97). Plasmid pMBXS1091 contains an expression cassette for CCP1 from Chlamydomonas reinhardtii fused to a C-terminal myc tag. The expression of the ccp1-myc gene is controlled by the promoter from the rice beta-fructofuranosidase insoluble isoenzyme 1 (CIN1) gene (LOC_Os02g33110). An expression cassette for the hptI gene, driven by the CaMV35S promoter and including the hsp70 intron as well as an intron from the bean catalase-1 gene (CAT-1) imparts transgenic plants resistance to the herbicide hygromycin.

FIG. 22 details a strategy for promoter replacement in front of the native Csa15g017550 sequence using genome editing and a homologous directed repair mechanism. Guide #1 and Guide #2 are used to excise the promoter to be replaced (Promoter 1). A new promoter cassette (Promoter 2), flanked by sequences with homology to the upstream and downstream region of Promoter 1, is introduced and is inserted into the site previously occupied by Promoter 1 using the homologous directed repair mechanism.

FIG. 23 shows a phylogenetic tree (cladogram) of proteins encoded by differentially expressed genes in select gene ontology (GO) categories that were identified in RNA Sequencing data of leaves of a soybean line expressing the CCP1 gene from a constitutive promoter. The 168 amino acid Camelina ISY protein (SEQ ID NO: 5) was included in the alignment. The select GO categories are shown in TABLE 16. The proteins were grouped into five categories for further analysis and include: Group 1, XP_003526981.1 (SEQ ID NO: 141), XP_006589707.1 (SEQ ID NO: 142), XP_003535958.1 (SEQ ID NO: 143), and XP_003555714.4(SEQ ID NO: 144); Group 2, Camelina ISY protein (SEQ ID NO: 5), XP_003543834.1 (SEQ ID NO: 145), XP_006587735.1 (SEQ ID NO: 146), NP_001237215.2 (SEQ ID NO: 147), and NP_001235420.1 (SEQ ID NO: 148); Group 3, XP_003517432.1 (SEQ ID NO: 149), XP_003530773.1 (SEQ ID NO: 150), XP_003525240.1 (SEQ ID NO: 151); Group 4, XP_003531408.1 (SEQ ID NO: 152), XP_003526711.1 (SEQ ID NO: 153), XP_003553658.2 (SEQ ID NO: 154); Group 5, XP_025981966.1 (SEQ ID NO: 155), XP_003528739.1 (SEQ ID NO: 156).

FIG. 24A-B shows a multiple sequence alignment of the Group 1 proteins of the phylogenetic tree (cladogram) in FIG. 23 according to CLUSTAL 0(1.2.4). Group 1 proteins have GO terms of probable pectate lyases. Sequences are as follows: XP_003526981.1 (SEQ ID NO: 141), XP_006589707.1 (SEQ ID NO: 142), XP_003535958.1 (SEQ ID NO: 143), and XP_003555714.4 (SEQ ID NO: 144).

FIG. 25A-B shows a multiple sequence alignment of the Group 2 proteins of the phylogenetic tree in FIG. 23 according to CLUSTAL 0(1.2.4). Group 2 proteins include the Camelina ISY protein, and proteins that have GO terms of pectinesterases or cell wall/vacuolar inhibitors of fructosidase 1. Sequences are as follows: Camelina ISY protein (SEQ ID NO: 5), XP_003543834.1 (SEQ ID NO: 145), XP_006587735.1 (SEQ ID NO: 146), NP_001237215.2 (SEQ ID NO: 147), and NP_001235420.1 (SEQ ID NO: 148).

FIG. 26 shows a multiple sequence alignment of the Camelina ISY protein with the two cell wall/vacuolar inhibitor of fructosidase 1 proteins in Group 2 of the phylogenetic tree in FIG. 23 according to CLUSTAL 0(1.2.4). Sequences are as follows: Camelina ISY protein (SEQ ID NO: 5), NP_001237215.2 (SEQ ID NO: 147), and NP_001235420.1 (SEQ ID NO: 148).

FIG. 27 shows a multiple sequence alignment of the Group 3 proteins of the phylogenetic tree in FIG. 23 according to CLUSTAL 0(1.2.4). Group 3 proteins have GO terms of pectinesterase inhibitor or plant invertase/pectin methylesterase inhibitor superfamily protein. Sequences are as follows: XP_003517432.1 (SEQ ID NO: 149), XP_003530773.1 (SEQ ID NO: 150), XP_003525240.1 (SEQ ID NO: 151).

FIG. 28A-B shows a multiple sequence alignment of the Group 4 proteins of the phylogenetic tree in FIG. 23 according to CLUSTAL 0(1.2.4). Group 4 proteins have GO terms of probable pectinesterase/pectinesterase inhibitors. Sequences are as follows: XP_003531408.1 (SEQ ID NO: 152), XP_003526711.1 (SEQ ID NO: 153), XP_003553658.2 (SEQ ID NO: 154).

FIG. 29A-B shows a multiple sequence alignment of the Group 5 proteins of the phylogenetic tree in FIG. 23 according to CLUSTAL 0(1.2.4). Group 5 proteins have GO terms of pectinesterase/pectinesterase inhibitors. Sequences are as follows: XP_025981966.1 (SEQ ID NO: 155) and XP_003528739.1 (SEQ ID NO: 156).

FIG. 30 shows a phylogenetic tree (cladogram) of proteins encoded by differentially expressed genes in select gene ontology (GO) categories that were identified in RNA Sequencing data of leaves of soybean lines transformed with pMBXO74 (FIG. 20A) expressing the CCP1 gene from a 35S constitutive promoter (C1/A comparison in TABLE 18). The 168 amino acid Camelina ISY protein (SEQ ID NO: 5) was included in the alignment. The select GO categories are shown in TABLE 16. Genes that are downregulated are indicated in the figure with a triangle. Genes that are upregulated by more than 100 fold are indicated in the figure with a shaded circle.

FIG. 31 shows a phylogenetic tree (cladogram) of proteins encoded by differentially expressed genes in select gene ontology (GO) categories that were identified in RNA Sequencing data of maturing seeds of a soybean line transformed with pMBXO74 (FIG. 20A) expressing the CCP1 gene from a 35S constitutive promoter (F1/D comparison in TABLE 19). The 168 amino acid Camelina ISY protein (SEQ ID NO: 5) was included in the alignment. The select GO categories are shown in TABLE 16. Genes that are downregulated are indicated in the figure with a triangle. Genes that are upregulated by more than 100 fold are indicated in the figure with a shaded circle.

FIG. 32 shows a phylogenetic tree (cladogram) of proteins encoded by differentially expressed genes in select gene ontology (GO) categories that were identified in RNA Sequencing data of developing seeds of a soybean line transformed with pMBXO75 (FIG. 20C) expressing the CCP1 gene from a soybean oleosin seed specific promoter (P/L comparison in TABLE 19). The 168 amino acid Camelina ISY protein (SEQ ID NO: 5) was included in the alignment. The select GO categories are shown in TABLE 16. Genes that are downregulated are indicated in the figure with a triangle. Genes that are upregulated by more than 100 fold are indicated in the figure with a shaded circle.

FIG. 33 shows a phylogenetic tree (cladogram) of proteins encoded by differentially expressed genes in select gene ontology (GO) categories that were identified in RNA Sequencing data of maturing seeds of a soybean line transformed with pMBXO75 (FIG. 20C) expressing the CCP1 gene from a soybean oleosin seed specific promoter (K/H comparison in TABLE 19). The 168 amino acid Camelina ISY protein (SEQ ID NO: 5) was included in the alignment. The select GO categories are shown in TABLE 16. Genes that are downregulated are indicated in the figure with a triangle. Genes that are upregulated by more than 100 fold are indicated in the figure with a shaded circle.

### DETAILED DESCRIPTION OF THE INVENTION

A genetically engineered land plant that expresses a protein that has homology to a plant invertase inhibitor and/or a pectin methylesterase inhibitor and that increases seed yield with increased expression (“an ISY protein”) is disclosed. The genetically engineered land plant comprises a modified gene for the ISY protein. The ISY protein comprises one or more of (i) an ISY protein of Camelina sativa comprising SEQ ID NO: 2; (ii) a fragment of an ISY protein of Camelina sativa comprising SEQ ID NO: 2; (iii) a Camelina sativa homolog of an ISY protein of Camelina sativa comprising SEQ ID NO: 2; or (iv) an ortholog of an ISY protein of Camelina sativa comprising SEQ ID NO: 2. The modified gene comprises (i) a promoter and (ii) a nucleic acid sequence encoding the ISY protein. The promoter is non-cognate with respect to the nucleic acid sequence encoding the ISY protein. The modified gene is configured such that transcription of the nucleic acid sequence is initiated from the promoter and results in expression of the ISY protein.

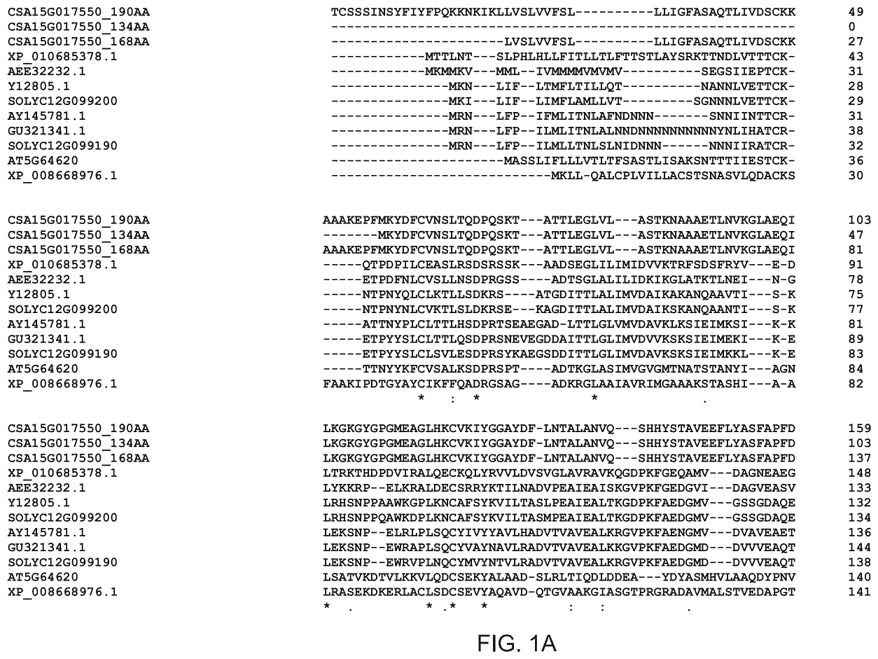

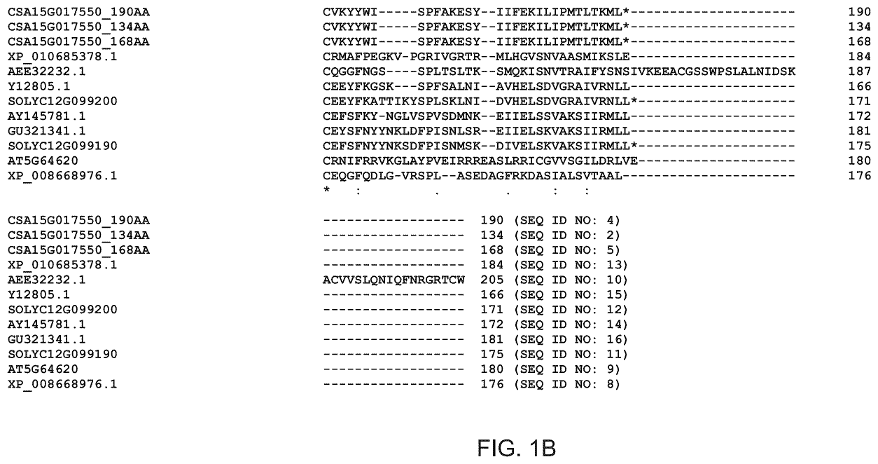

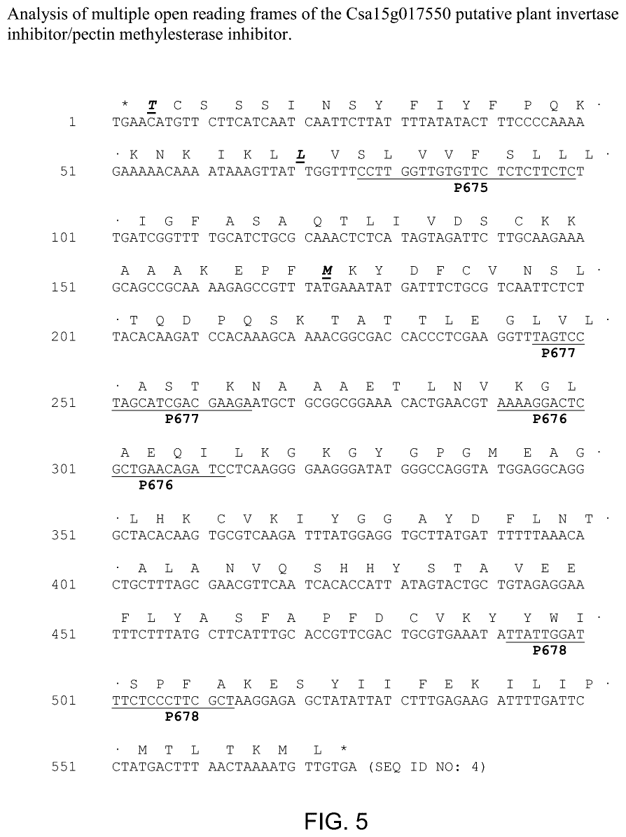

Constitutive expression of a mitochondrial metabolite transporter encoded by the algal gene CCP1 in Camelina was noted to not only increase carbon fixation and seed yield but also to result in smaller seed size (WO2015/103074, to the University of Massachusetts). Independently we have shown that similar changes in seed yield and seed size are obtained when CCP1 is expressed in canola and soybean. Gene expression analysis of the CCP1 high yield Camelina lines was performed using the RNASeq technology to determine the impacts of over-expressing the CCP1 gene on the expression of native Camelina genes. One gene in particular, Csa15g017550 (SEQ ID NO: 1) that encodes a 134 amino acid protein (SEQ ID NO: 2) was found to be highly up-regulated and is a putative plant invertase inhibitor and/or pectin methylesterase inhibitor (also termed “plant invertase inhibitor/pectin methylesterase inhibitor”). Analysis of the upstream region of the Csa15g017550 gene shows that there are several alternative start sites (FIG. 5). Using the methionine predicted start codon according to Camelina Sativa Genome Browser, Csa15g017550.1 produces a 134 amino acid protein (SEQ ID NO: 2). An alternative leucine start codon upstream, encoded by TTG, produces a 168 amino acid protein (SEQ ID NO: 5). This alternative start codon was chosen based on alignments with other cell wall invertase inhibitors (FIG. 1). The plant invertase inhibitor/pectin methylesterase inhibitor family of genes is very large and diverse with the majority of genes being assigned to this family based on regions of in most cases limited sequence homology. Very few individual genes have been characterized at the molecular level and so it is reasonable to conclude that for the vast majority of genes listed in this family their actual activity and function is unknown. Cell wall invertase inhibitor (CWII) genes for increasing seed yield have been studied in Camelina. PCT/US2014/043407 to North Carolina State University demonstrated that reduced expression of CWII genes in Camelina results in increased seed yield.

We hypothesized that expression of CCP1 in the high yielding plants had increased the level of fixed carbon and had increased seed numbers albeit with a smaller seed size. We hypothesized that the putative up-regulated plant invertase inhibitor/pectin methylesterase inhibitor gene homolog may be responsible for reducing the flow of carbon to the increased number of seeds resulting in smaller seed size. In order to test this hypothesis we overexpressed both the 134 amino acid (SEQ ID NO: 2) and 168 amino acid (SEQ ID NO: 5) plant invertase inhibitor/pectin methylesterase inhibitor ortholog proteins in Camelina using a constitutive promoter with the expectation that this would result in plants with reduced seed size and lower seed yield.

Surprisingly, we found that increased expression of this putative plant invertase inhibitor/pectin methylesterase inhibitor ortholog gene resulted in a large increase in branching, seed yield, and, in some plants, an increase in the average size of individual seeds.

Based on these results we re-investigated the homology with characterized plant invertase inhibitor and/or pectin methylesterase inhibitor genes and concluded that this gene is a novel gene with an as yet unknown function in Camelina. Furthermore, although Camelina is an allohexaploid with 3 copies of each chromosome, we found that Csa15g017550.1 is only present in a single copy. Examination of the region of the chromosome where Csa15g017550.1 is located on the other two chromosomes indicated that the Csa15g017550.1 is not present on the other two chromosomes. tBLASTn (search excludes Camelina sativa) and FASTA searches of other crop species to identify homologs of the Camelina Csa15g017550 did not reveal any genes encoding proteins with greater than 58% identity over the length of the whole protein. For these reasons we believe that the Camelina Csa15g017550 gene is very rare and of unknown function. To distinguish it from characterized plant invertase inhibitor and pectin methylesterase inhibitor genes we refer to this gene and similar genes with weak homology to plant invertase inhibitor and pectin methylesterase inhibitor genes in other crop species whose expression is increased by expression of CCP1 as the increased seed yield gene (“ISY gene”).

Accordingly, disclosed herein are genetically engineered land plants having increased expression of the ISY gene (SEQ ID NO: 1) encoding a 134 amino acid protein (SEQ ID NO: 2), and genetic constructs, materials and methods for making such plants. Such plants may have increased branching and/or higher seed, tuber or fruit yield and optionally increased seed, tuber or fruit size as compared to reference land plants that do not have increased expression of the ISY gene.

Also disclosed herein are genetic constructs such that the ISY gene is expressed from different translation start sites, such as the ISY gene with an alternative start codon (SEQ ID NO: 6) encoding a 168 amino acid protein (SEQ ID NO: 5), and plants expressing them which have increased branching and/or higher seed, tuber or fruit yield and optionally increased seed, tuber or fruit size as compared to the same plants which do not have increased expression of the ISY gene.

Also disclosed herein are methods for identifying ISY genes in any land plant. The methods comprise genetically engineering land plants to have increased expression of one or more mitochondrial metabolite transporter genes to produce plants having higher seed yield and optionally smaller seed size, then carrying out gene expression studies to identify native plant ISY genes whose expression is significantly increased in the plants having increased expression of the one or more mitochondrial metabolite transporter genes wherein the ISY genes are native plant genes whose expression is significantly increased and which have sequence homology to invertase inhibitor/pectin methylesterase inhibitor genes. We refer to these genes herein as ISY genes. An ISY gene can be, for example, a gene encoding a protein having 10-65% sequence identity to at least one protein encoded by a gene of the plant invertase inhibitor/pectin methylesterase inhibitor family of genes and whose expression is significantly increased, e.g. by at least 5%, 10%, 20%, or more, in plants engineered to have higher expression of one or more mitochondrial metabolite transporter genes.

Also disclosed herein are land plants genetically engineered to have increased expression of the ISY genes identified by the aforementioned genetic constructs, materials and methods having increased branching and/or higher seed, tuber or fruit yield and optionally increased seed, tuber or fruit size.

Also disclosed herein are genetically engineered land plants made using the materials and methods having increased expression of the ISY genes and increased expression of mitochondrial metabolite transporter genes which have higher seed, tuber or fruit yield and possibly increased seed, tuber or fruit size. Plants are engineered to have higher levels of expression of the identified ISY genes such that they have higher seed, fruit or tuber yield. Preferably the plants engineered to have higher expression of the ISY genes are the same species of plant as the ISY genes used to increase the expression of the ISY genes. For example the Camelina ISY gene preferably is expressed in Camelina, the canola ISY gene preferably is expressed in canola, the soy ISY gene preferably is expressed in soy, rice ISY preferably is expressed in rice, wheat ISY gene preferably is expressed in wheat, potato ISY gene preferably is expressed in potato, sugar beet ISY gene preferably is increased in sugar beet, tomato ISY gene preferably is increased in tomato, and so on. When the ISY gene expression is increased then the degree of branching, yield of seeds, fruit or tubers and optionally seed, fruit or tuber size is also increased.

The term “land plant” includes mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage.

Land plants encompass all annual and perennial monocotyledonous or dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Elaeis, Saccharum, Avena, Hordeum, Secale, Triticum, Sorghum, Picea, Populus, Camelina, Beta, Solanum, and Carthamus. Preferred plants are those from the following plant families: Amaranthaceae, Asteraceae, Brassicaceae, Carophyllaceae, Chenopodiaceae, Compositae, Cruciferae, Cucurbitaceae, Euphorbiaceae, Fabaceae, Labiatae, Leguminosae, Papilionoideae, Liliaceae, Linaceae, Malvaceae, Poaceae, Rosaceae, Rubiaceae, Saxifragaceae, Scrophulariaceae, Solanaceae, Sterculiaceae, Tetragoniaceae, Theaceae, and Umbelliferae.

The land plant can be a monocotyledonous plant or a dicotyledonous plant. Preferred dicotyledonous plants are selected in particular from the dicotyledonous crop plants such as, for example, Asteraceae such as sunflower, tagetes or calendula and others; Compositae, especially the genus Lactuca, very particularly the species sativa (lettuce) and others; Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others; Cucurbitaceae such as melon, pumpkin/squash or zucchini and others; Leguminosae, particularly the genus Glycine, very particularly the species max (soybean), soya, and alfalfa, pea, beans or peanut and others; Rubiaceae, preferably the subclass Lamiidae such as, for example Coffea arabica or Coffea liberica (coffee bush) and others; Solanaceae, particularly the genus Lycopersicon, very particularly the species esculentum (tomato), the genus Solanum, very particularly the species tuberosum (potato) and melongena (aubergine) and the genus Capsicum, very particularly the genus annuum (pepper) and tobacco or paprika and others; Sterculiaceae, preferably the subclass Dilleniidae such as, for example, Theobroma cacao (cacao bush) and others; Theaceae, preferably the subclass Dilleniidae such as, for example, Camellia sinensis or Thea sinensis (tea shrub) and others; Umbelliferae, particularly the genus Daucus (very particularly the species carota (carrot)) and Apium (very particularly the species graveolens dulce (celery)) and others; and linseed, cotton, hemp, flax, cucumber, spinach, carrot, sugar beet and the various tree, nut and grapevine species, in particular banana and kiwi fruit. Preferred monocotyledonous plants include maize, rice, wheat, sugarcane, sorghum, oats and barley.

Of particular interest are oilseed plants. In oilseed plants of interest the oil is accumulated in the seed and can account for greater than 10%, greater than 15%, greater than 18%, greater than 25%, greater than 35%, greater than 50% by weight of the weight of dry seed. Oil crops encompass by way of example: Borago officinalis (borage); Camelina (false flax); Brassica species such as B. campestris, B. napus, B. rapa, B. carinata (mustard, oilseed rape or turnip rape); Cannabis sativa (hemp); Carthamus tinctorius (safflower); Cocos nucifera (coconut); Crambe abyssinica (crambe); Cuphea species (Cuphea species yield fatty acids of medium chain length, in particular for industrial applications); Elaeis guinensis (African oil palm); Elaeis oleifera (American oil palm); Glycine max (soybean); Gossypium hirsutum (American cotton); Gossypium barbadense (Egyptian cotton); Gossypium herbaceum (Asian cotton); Helianthus annuus (sunflower); Jatropha curcas (jatropha); Linum usitatissimum (linseed or flax); Oenothera biennis (evening primrose); Olea europaea (olive); Oryza sativa (rice); Ricinus communis (castor); Sesamum indicum (sesame); Thlaspi caerulescens (pennycress); Triticum species (wheat); Zea mays (maize), and various nut species such as, for example, walnut or almond.

Camelina species, commonly known as false flax, are native to Mediterranean regions of Europe and Asia and seem to be particularly adapted to cold semiarid climate zones (steppes and prairies). The species Camelina sativa was historically cultivated as an oilseed crop to produce vegetable oil and animal feed. In addition to being useful as an industrial oilseed crop, Camelina is a very useful model system for developing new tools and genetically engineered approaches to enhancing the yield of crops in general and for enhancing the yield of seed and seed oil in particular. Demonstrated transgene improvements in Camelina obtained through genetic engineering of specific genes can then be deployed in other major food and feed crops including oilseed crops including Brassica species including B. napus (canola), B. rapa, B. juncea, B. carinata, crambe, soybean, sunflower, safflower, oil palm, flax, and cotton and starch crops including maize, grain sorghum, wheat, rice, oats, barley, potatoes and sucrose producing crops such as sugar beet. Sugarcane and pulses including peas, chickpeas, lentils and the like.

As will be apparent, the land plant can be a C3 photosynthesis plant, i.e. a plant in which RubisCO catalyzes carboxylation of ribulose-1,5-bisphosphate by use of CO₂ drawn directly from the atmosphere, such as for example, Camelina, canola, soybean, wheat, rice, oat, barley, sweet potato, potato, sugar beet among others. The land plant also can be a C4 plant, i.e. a plant in which RubisCO catalyzes carboxylation of ribulose-1,5-bisphosphate by use of CO₂ shuttled via malate or aspartate from mesophyll cells to bundle sheath cells, such as for example maize, millet, sorghum, sugarcane among others.

Accordingly, in some examples the genetically engineered land plant is a C3 plant. Also, in some examples the genetically engineered land plant is a C4 plant. Also, in some examples the genetically engineered land plant is a major food crop plant selected from the group consisting of maize, wheat, oat, barley, soybean, millet, sorghum, potato, sugar beet, pulse, chickpea, green pea, yellow pea, lentils, bean, tomato, and rice. In some of these examples, the genetically engineered land plant is maize. Also, in some examples the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

As noted, the genetically engineered land plant comprises a modified gene for the ISY protein. The ISY protein comprises one or more of (i) an ISY protein of Camelina sativa comprising SEQ ID NO: 2; (ii) a fragment of an ISY protein of Camelina sativa comprising SEQ ID NO: 2; (iii) a Camelina sativa homolog of an ISY protein of Camelina sativa comprising SEQ ID NO: 2; or (iv) an ortholog of an ISY protein of Camelina sativa comprising SEQ ID NO: 2.

The term “fragment,” as used herein in reference to a polynucleotide sequence or polypeptide sequence, means a contiguous part of the polynucleotide sequence or polypeptide sequence that is less than the entire polynucleotide sequence or polypeptide sequence.

The phrases “fragment of an ISY protein of Camelina sativa comprising SEQ ID NO: 2” and “fragment of a protein of Camelina sativa comprising SEQ ID NO: 2,” as used herein, means at least 30 contiguous amino acids of SEQ ID NO: 2. Such a fragment can be, for example, 30 or more contiguous amino acids of SEQ ID NO: 2. Such a fragment also can be, for example, 40 or more, 50 or more, 60 or more, 70 or more, 80 or more, 90 or more, or 100 or more contiguous amino acids of SEQ ID NO: 2. Such a fragment also can be, for example, 114 to 133 contiguous amino acids of SEQ ID NO: 2.

The term “ortholog,” as used herein, means a polynucleotide sequence or polypeptide sequence possessing a high degree of homology, i.e. sequence relatedness, to a subject sequence and being a functional equivalent of the subject sequence, wherein the sequence that is orthologous is from a species that is different than that of the subject sequence. Homology may be quantified by determining the degree of identity and/or similarity between the sequences being compared.

As used herein, “percent homology” of two polynucleotide sequences or of two polypeptide sequences is the percent identity over the length of the entire sequence determined using the ALIGNX alignment function of the Vector NTI software package (Vector NTI Advance, Version 11.5.3, ThermoFisher), which uses the Clustal W algorithm. Default parameters of the program were used.

The percentage of sequence identity between two polypeptides can also be determined by making a pairwise sequence alignment. This can be done using EMBOSS Needle Pairwise Sequence Alignment (PROTEIN) tool using default settings (matrix: BLOSUM62; gap open: 10; gap extend: 0.5; output format: pair; end gap penalty: false; end gap open: 10; end gap extend: 0.5) (website:ebi.ac.uk/Tools/psa/emboss_needle/). This also can be done using other pairwise sequence alignment tools that are analogous.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Many other polypeptides will meet the same criteria.

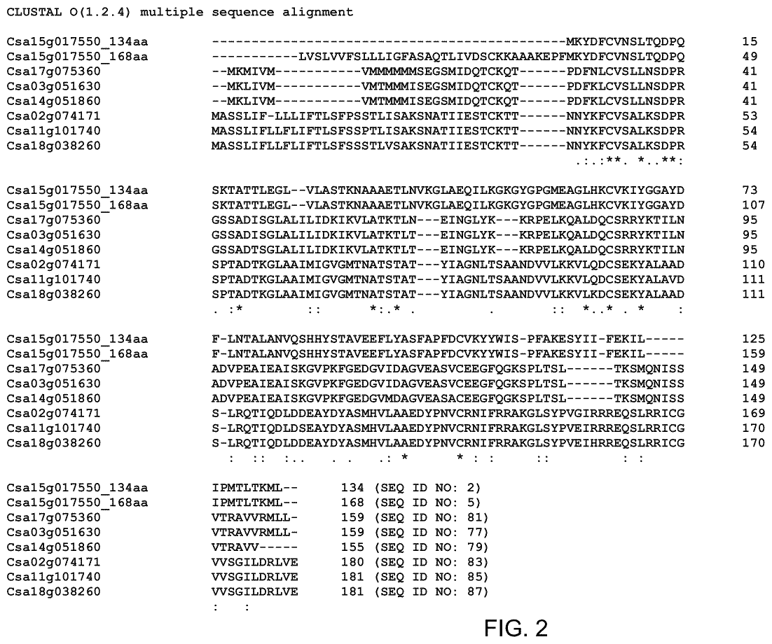

For reference, ISY genes as defined herein can have some homology to classical plant invertase inhibitor and/or pectin methylesterase inhibitor genes. Plant invertase inhibitor and pectin methylesterase inhibitor genes are well known in the art and comprise a large family of genes in most crop species. For example, BLAST searches using the Camelina genome database (website://www.camelinadb.ca/prairiegold/cgi-bin/blast.cgi) yielded 49 hits when using the 168 amino acid protein (SEQ ID NO: 5) and an additional 17 hits when using the 507-bp coding sequence (SEQ ID NO: 6) (TABLE 6). Keyword searches of the NCBI Entrez Gene database (website://www.ncbi.nlm.nih.gov/search/?ga_suggest=a&utm_expid=.fAeHyO5JTBGxnObh 2WlrCA.1&utm_referrer=https %3A %2F %2Fwww.ncbi.nlm.nih.gov %2FClass %2FMLACou rse %2FOriginal8 Hour %2FEntrez %2F), found a total of 324 genes in the Camelina genome. This included 51 annotated as invertase inhibitors, 200 annotated as pectinesterase/pectinesterase inhibitors, as well as 73 sequences that belong to an uncharacterized subfamily of plant invertase inhibitor/pectin methylesterase inhibitor domains. For the most part systematic studies on the expression and function of individual genes within the family are very limited with only a few genes having been studied in any detail. Examples of CWII genes in Camelina that have been studied include two cell wall invertase inhibitor genes described in Sederoff et al., U.S. Pub. No. 2016/0138038. Sederoff used RNAi gene silencing to reduce expression of these genes in Camelina and demonstrated an increase in seed yield. Three copies of each of these genes are present in the Camelina genome (Camelina is an allohexaploid). These include homeologs of cell wall invertase inhibitor 1 (CWII1) (SEQ ID NO: 76 (Csa03g051630), SEQ ID NO: 78 (Csa14g051860), and SEQ ID NO: 80 (Csa17g075360)) and homeologs of CWII2 (SEQ ID NO: 82 (Csa02g074171), SEQ ID NO: 84 (Csa11g101740), and SEQ ID NO: 86 (Csa18g038260)). A CLUSTAL alignment of the proteins encoded by these genes to the ISY protein is shown in FIG. 2 and their percent homology to the 168 (SEQ ID NO: 5) and 134 (SEQ ID NO: 2) amino acid Camelina ISY protein is shown in TABLE 7.

Accordingly, the ISY gene is derived from a plant or is a synthetic version of a plant ISY gene.

In some examples the source plant is a different type of plant than the genetically engineered land plant. In accordance with these examples, the ISY protein can be heterologous with respect to the genetically engineered land plant. By this it is meant that the particular ISY protein derived from the source plant is not normally encoded, expressed, or otherwise present in plants of the type from which the genetically engineered land plant is derived. This can be because plants of the type from which the genetically engineered land plant is derived do not normally encode, express, or otherwise include the particular ISY protein, and this can be so whether or not the plants normally express a different, endogenous ISY protein. The genetically engineered land plant expresses the particular ISY protein based on expressing the modified gene for the ISY protein. Accordingly, the modified gene can be used to accomplish modified expression of the ISY protein, and particularly increased expression of any endogenous ISY proteins.

Also in some examples the source plant is the same type of plant as the genetically engineered land plant. In accordance with these examples, the ISY protein can be homologous with respect to the genetically engineered land plant. By this it is meant that the particular ISY protein is normally encoded, and may normally be expressed, in plants of the type from which the genetically engineered land plant is derived. In accordance with these examples, the plant can be genetically engineered to include additional copies of a gene for the ISY protein and/or to express an endogenous copy of a gene for the ISY protein at higher levels and/or in a tissue-preferred manner based on modification and/or replacement of a promoter for the endogenous copy of the gene. Again, the genetically engineered land plant expresses the particular ISY protein based on comprising the modified gene for the ISY protein, resulting in modified expression of the ISY protein, and particularly increased expression of the ISY gene.

As discussed above, it is believed that increased expression of an ISY protein may enhance the flow of carbon from source tissue to sink tissue. The sink tissue may be seed, fruit or tuber.

Suitable ISY proteins are disclosed herein or can be identified using the materials and methods disclosed herein for example by increasing the expression of mitochondrial transporters in a plant and carrying out gene expression analysis followed by sequence homology searches of genes whose expression is increased. Such searches can be carried out, for example, by use of BLAST, e.g. tblastn, and databases including translated polynucleotides, whole genome shotgun sequences, and/or transcriptome assembly sequences, among other sequences and databases. Potential orthologs of ISY may be identified, for example, based on percentage of identity and/or percentage of similarity, with respect to polypeptide sequence, of individual sequences in the databases in comparison to Camelina sativa ISY proteins of SEQ ID NO: 2 and/or SEQ ID NO: 5. For example, potential orthologs of ISY may be identified based on percentage of identity of an individual sequence in a database of at least 25%, e.g. at least 30%, at least 35%, at least 40%, at least 45%, at least 50%, at least 55%, at least 60%, at least 65%, at least 70%, at least 75%, at least 80%, at least 90%, or at least 95%, wherein the individual sequence is derived from a land plant or a eukaryotic alga.

Following identification of an ISY gene, genetic engineering of a plant to express the ISY gene or a truncated version of it can be carried out by methods that are known in the art, as discussed in detail below.

The genetically engineered land plant can be a genetically engineered land plant that includes no heterologous proteins, e.g. wherein the ISY protein is homologous with respect to the genetically engineered land plant, or only one heterologous protein, e.g. wherein the only heterologous plant protein that the genetically engineered land plant comprises is the ISY protein.

Considering the ISY protein in more detail, again as noted the ISY protein comprises one or more of (i) an ISY protein of Camelina sativa comprising SEQ ID NO: 2; (ii) a fragment of an ISY protein of Camelina sativa comprising SEQ ID NO: 2; (iii) a Camelina sativa homolog of an ISY protein of Camelina sativa comprising SEQ ID NO: 2; or (iv) an ortholog of an ISY protein of Camelina sativa comprising SEQ ID NO: 2.

In some examples, the ISY protein comprises an ISY protein of Camelina sativa comprising SEQ ID NO: 2. In some of these examples, the ISY protein of Camelina sativa comprising SEQ ID NO: 2 corresponds to one or more of ISY protein of Camelina sativa of SEQ ID NO: 2, ISY protein of Camelina sativa of SEQ ID NO: 5, modified ISY protein of Camelina sativa of SEQ ID NO: 138, or modified ISY protein of Camelina sativa of SEQ ID NO: 140. The ISY protein of Camelina sativa of SEQ ID NO: 2 is the Camelina sativa ISY protein having 134 amino acids as described herein. Likewise, ISY protein of Camelina sativa of SEQ ID NO: 5 is the Camelina sativa ISY protein having 168 amino acids as described herein. The modified ISY protein of Camelina sativa of SEQ ID NO: 138 is a modified version of the ISY protein of SEQ ID NO: 5 in which the N-terminal leucine has been replaced with an N-terminal methionine. This modified ISY protein can be encoded, for example, by the nucleotide sequence of SEQ ID NO: 137, which is identical to SEQ ID NO: 6 except that the TTG alternative start codon has been replaced by an ATG start codon. The modified ISY protein of Camelina sativa of SEQ ID NO: 140 is a modified version of the ISY protein of SEQ ID NO: 5 in which the N-terminal leucine has been preceded with an N-terminal methionine. This modified ISY protein can be encoded, for example, by the nucleotide sequence of SEQ ID NO: 139, which is identical to SEQ ID NO: 6 except that the TTG alternative start codon has been preceded by an ATG start codon. The ISY protein of Camelina sativa comprising SEQ ID NO: 2 also can correspond to additional modified ISY proteins, for example additional modified ISY proteins similar to SEQ ID NO: 2, SEQ ID NO: 5, SEQ ID NO: 138, or SEQ ID NO: 140, but including one or more additional amino acid residues at the N-terminus and/or C-terminus.

In some examples, the ISY protein comprises a fragment of an ISY protein of Camelina sativa comprising SEQ ID NO: 2. In some of these examples, the fragment corresponds to 30 or more contiguous amino acids of SEQ ID NO: 2. In some of these examples, the fragment corresponds to 40 or more, 50 or more, 60 or more, 70 or more, 80 or more, 90 or more, or 100 or more contiguous amino acids of SEQ ID NO: 2. In some of these examples, the fragment corresponds to 114 to 133 contiguous amino acids of SEQ ID NO: 2. Thus, in some of these examples the fragment corresponds a protein that is identical to ISY protein of Camelina sativa of SEQ ID NO: 2, except that the fragment lacks 1 to 20 amino acids at the N-terminus of the fragment relative to SEQ ID NO: 2. Also in some examples, the fragment corresponds a protein that is identical to ISY protein of Camelina sativa of SEQ ID NO: 2, except that the fragment lacks 1 to 20 amino acids at the C-terminus of the fragment relative to SEQ ID NO: 2. Also in some examples, the fragment corresponds a protein that is identical to ISY protein of Camelina sativa of SEQ ID NO: 2, except that the fragment lacks 1 to 10 amino acids at the N-terminus of the fragment, and 1 to 10 amino acids at the C-terminus of the fragment, relative to SEQ ID NO: 2. In some examples, the fragment is as described, except that the fragment also includes a methionine residue at the N-terminus of the fragment. Based on results obtained for the 134 amino acid ISY protein of SEQ ID NO: 2, which lacks a signal peptide and a stretch of N-terminal amino acids relative to characterized invertase inhibitors, it is believed that increased expression of fragments of ISY proteins would be sufficient for increased seed yield, fruit yield, and/or tuber yield.

In some examples, the ISY protein comprises a Camelina sativa homolog of an ISY protein of Camelina sativa comprising SEQ ID NO: 2. In some of these examples, the Camelina sativa homolog corresponds to one or more Camelina sativa proteins of SEQ ID NOS: 21-40. These homologs are described below.

In some examples, the ISY protein comprises the ortholog of an ISY protein of Camelina sativa comprising SEQ ID NO: 2. In some of these examples, the ortholog corresponds to one or more (i) canola proteins of SEQ ID NOS: 69, 71, 73, and/or 75; (ii) soybean proteins of SEQ ID NOS: 89, 91, 93, and/or 95; or (iii) corn proteins of SEQ ID NOS: 99, 101, 103, 105, and/or 107. These orthologs are described below.

In some examples, the ISY protein comprises (i) (a) a cysteine residue at position 40, (b) a leucine residue at position 59, (c) a cysteine residue at position 98, and (d) a cysteine residue at position 138, with numbering of positions relative to ISY protein of Camelina sativa of SEQ ID NO: 5, and (ii) an overall identity of at least 10% with ISY protein of Camelina sativa of SEQ ID NO: 2. In some of these examples, the ISY protein further comprises only between 1 to 14 amino acid residues N-terminal to the cysteine residue at position 40, with numbering of positions relative to ISY protein of Camelina sativa of SEQ ID NO: 5. Also in some of these examples, the ISY protein lacks an N-terminal signal peptide. Also in some of these examples, the ISY protein lacks a PKF motif. Based on results obtained for the 134 amino acid ISY protein of SEQ ID NO: 2, which includes a cysteine residue at position 40, a leucine residue at position 59, a cysteine residue at position 98, and a cysteine residue at position 138, with numbering of positions relative to ISY protein of Camelina sativa of SEQ ID NO: 5, and which includes only between 1 to 14 amino acid residues N-terminal to the cysteine residue at position 40, namely 5 amino acid residues N-terminal to the cysteine residue at position 40, also with numbering of positions relative to ISY protein of Camelina sativa of SEQ ID NO: 5, and which also lacks an N-terminal signal peptide and a PKF motif, it is believed that increased expression of an ISY protein having these characteristics would be sufficient for increased seed yield, fruit yield, and/or tuber yield.

In some examples, the ISY protein comprises (i) (a) a cysteine residue at position 25, (b) a cysteine residue at position 40, (c) a leucine residue at position 59, (d) a cysteine residue at position 98, and (e) a cysteine residue at position 138, with numbering of positions relative to ISY protein of Camelina sativa of SEQ ID NO: 5, and (ii) an overall identity of at least 10% with ISY protein of Camelina sativa of SEQ ID NO: 5. In some of these examples, the ISY protein lacks a PKF motif. Based on results obtained for the 168 amino acid ISY protein of SEQ ID NO: 5, which includes a cysteine residue at position 25, a cysteine residue at position 40, a leucine residue at position 59, a cysteine residue at position 98, and a cysteine residue at position 138, with numbering of positions relative to ISY protein of Camelina sativa of SEQ ID NO: 5, and which lacks a PKF motif, it is believed that increased expression of an ISY protein having these characteristics also would be sufficient for increased seed yield, fruit yield, and/or tuber yield.

The modified gene for the ISY gene comprises (i) a promoter and (ii) a nucleic acid sequence encoding the ISY gene.

The promoter is non-cognate with respect to the nucleic acid sequence encoding the ISY gene. A promoter that is non-cognate with respect to a nucleic acid sequence means that the promoter is not naturally paired with the nucleic acid sequence in organisms from which the promoter and/or the nucleic acid sequence are derived. Instead, the promoter has been paired with the nucleic acid sequence based on use of recombinant DNA techniques to create a modified gene.

The modified gene for the ISY gene is configured such that transcription of the nucleic acid sequence encoding the ISY gene is initiated from the promoter and results in expression of the ISY gene. Accordingly, in the context of the modified gene, the promoter functions as a promoter of transcription of the nucleic acid sequence, and thus of expression of the ISY gene. In a preferred example, the expression of the ISY gene is higher in the genetically engineered land plant than in a corresponding plant that does not include the modified gene.

In some examples, the promoter is a constitutive promoter. In some examples, the promoter is a seed-specific promoter. In some examples, the modified gene is integrated into genomic DNA of the genetically engineered land plant. In some examples, the modified gene is stably expressed in the genetically engineered land plant. In some examples the nucleic acid sequence encodes a wild-type ISY gene. In some examples, the nucleic acid sequence encodes a variant, modified, mutant, or otherwise non-wild-type ISY gene. These exemplary characteristics, and others, of the promoter, the nucleic acid sequence, and the modified gene are discussed in detail below.

The genetically engineered land plant also can be a genetically engineered land plant that expresses nucleic acid sequences encoding ISY genes in both a seed-specific and/or a constitutive manner, wherein the nucleic acid sequences encoding the ISY genes may be the same or different nucleic acid sequences, e.g. from the same source plant or from different source plants. In some examples the genetically engineered land plant (i) expresses the ISY gene in a seed-specific manner, and (ii) expresses another ISY gene constitutively, the other ISY gene also corresponding to an ortholog of ISY derived from a source plant.

The genetically engineered land plant can exhibit increased expression of the ISY protein in comparison to a reference land plant that does not include the modified gene. The genetically engineered land plant also can exhibit increased seed yield, fruit yield, and/or tuber yield in comparison to a reference land plant that does not include the modified gene. For example, the genetically engineered land plant can have a seed yield that is at least 5% higher, at least 10% higher, at least 20% higher, at least 40% higher, at least 60% higher, or at least 80% higher, than for a corresponding reference land plant that does not comprise the modified gene. Also for example, the genetically engineered land plant can have a fruit yield that is at least 5% higher, at least 10% higher, at least 20% higher, at least 40% higher, at least 60% higher, or at least 80% higher, than for a corresponding reference land plant that does not comprise the modified gene. Also for example, the genetically engineered land plant can have a tuber yield that is at least 5% higher, at least 10% higher, at least 20% higher, at least 40% higher, at least 60% higher, or at least 80% higher, than for a corresponding reference land plant that does not comprise the modified gene.

As noted above, following identification of an ISY gene of a source plant, genetic engineering of a land plant to express the ISY gene can be carried out by methods that are known in the art, for example as follows.

DNA constructs useful in the methods described herein include transformation vectors capable of introducing transgenes or other modified nucleic acid sequences into plants. As used herein, “genetically engineered” refers to an organism in which a nucleic acid fragment containing a heterologous nucleotide sequence has been introduced, or in which the expression of a homologous gene has been modified, for example by genome editing. Transgenes in the genetically engineered organism are preferably stable and inheritable. Heterologous nucleic acid fragments may or may not be integrated into the host genome.

Several plant transformation vector options are available, including those described in Gene Transfer to Plants, 1995, Potrykus et al., eds., Springer-Verlag Berlin Heidelberg New York, Genetically engineered Plants: A Production System for Industrial and Pharmaceutical Proteins, 1996, Owen et al., eds., John Wiley & Sons Ltd. England, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB10 and hygromycin selection derivatives thereof. See, for example, U.S. Pat. No. 5,639,949.

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. See, for example, U.S. Pat. No. 5,639,949. Alternatively, DNA fragments containing the transgene and the necessary regulatory elements for expression of the transgene can be excised from a plasmid and delivered to the plant cell using microprojectile bombardment-mediated methods.

Zinc-finger nucleases (ZFNs) are also useful in that they allow double strand DNA cleavage at specific sites in plant chromosomes such that targeted gene insertion or deletion can be performed (Shukla et al., 2009, Nature 459: 437-441; Townsend et al., 2009, Nature 459: 442-445).

The CRISPR/Cas9 system (Sander, J. D. and Joung, J. K., Nature Biotechnology, published online Mar. 2, 2014; doi; 10.1038/nbt.2842) is particularly useful for editing plant genomes to modulate the expression of homologous genes encoding enzymes. All that is required to achieve a CRISPR/Cas edit is a Cas enzyme, or other CRISPR nuclease (Murugan et al. (2017), Mol Cell, 68:15), and a single guide RNA (sgRNA) as reviewed extensively by others (Belhag et al. (2015), Curr Opin Biotech, 32: 76; Khandagale & Nadaf (2016), Plant Biotechnol Rep, 10:327-343). Several examples of the use of this technology to edit the genomes of plants have now been reported (Belhaj et al. (2013), Plant Methods, 9:39; Zhang et al. (2016), Journal of Genetics and Genomics, 43: 251).

TALENs (transcriptional activator-like effector nucleases) or meganucleases can also be used for plant genome editing (Malzahn et al., Cell Biosci, 2017, 7:21).

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128), Methods for plant regeneration from protoplasts have also been described (Evans et al., in Handbook of Plant Cell Culture, Vol 1, (Macmillan Publishing Co., New York, 1983); Vasil, IK in Cell Culture and Somatic Cell Genetics (Academic, Orlando, 1984)).

Recombinase technologies which are useful for producing the disclosed genetically engineered land plants include the cre-lox, FLP/FRT and Gin systems. Methods by which these technologies can be used for the purpose described herein are described for example in (U.S. Pat. No. 5,527,695; Dale and Ow, 1991, Proc. Natl. Acad. Sci. USA 88: 10558-10562; Medberry et al., 1995, Nucleic Acids Res. 23: 485-490).

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques. See, for example, McCormick et al., 1986, Plant Cell Rep. 5: 81-84. These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain genetically engineered land plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Genetically engineered Res. 9: 471-476). Other plants that have successfully been transformed by in planta methods include rapeseed and radish (vacuum infiltration, Ian and Hong, 2001, Genetically engineered Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

The cells that have been transformed may be grown into plants in accordance with conventional techniques. See, for example, McCormick et al. Plant Cell Reports 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Genetically engineered land plants can be produced using conventional techniques to express any genes of interest in plants or plant cells (Methods in Molecular Biology, 2005, vol. 286, Genetically engineered Plants: Methods and Protocols, Pena L., ed., Humana Press, Inc. Totowa, NJ; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 1997, Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Seed-specific promoters can be used to target gene expression to seeds in particular. Seed-specific promoters include promoters that are expressed in various tissues within seeds and at various stages of development of seeds. Seed-specific promoters can be absolutely specific to seeds, such that the promoters are only expressed in seeds, or can be expressed preferentially in seeds, e.g. at rates that are higher by 2-fold, 5-fold, 10-fold, or more, in seeds relative to one or more other tissues of a plant, e.g. stems, leaves, and/or roots, among other tissues. Seed-specific promoters include, for example, seed-specific promoters of dicots and seed-specific promoters of monocots, among others. For dicots, seed-specific promoters include, but are not limited to, bean β-phaseolin, napin, β-conglycinin, soybean oleosin 1, Arabidopsis thaliana sucrose synthase, flax conlinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, and globulin 1.

Chemical-regulated promoters can be used to modulate the expression of a gene in a plant through the application of an exogenous chemical regulator.

Specific exemplary promoters useful for expression of genes in dicots and monocots are provided in TABLE 1 and TABLE 2, respectively.

TABLE 1
Promoters useful for expression of genes in dicots.

| | | Native organism | Gene ID* |
|---|---|---|---|
| Gene/Promoter | Expression | of promoter | (SEQ ID NO) |
| CaMV 35S | Constitutive | Cauliflower | (SEQ ID NO: 108) |
| | | mosaic virus | |
| Hsp70 | Constitutive | Glycine max | Glyma.02G093200 |
| | | | (SEQ ID NO: 41) |
| Chlorophyll A/B Binding | Constitutive | Glycine max | Glyma.08G082900 |
| Protein (Cab5) | | | (SEQ ID NO: 42) |
| Pyruvate phosphate | Constitutive | Glycine max | Glyma.06G252400 |
| dikinase (PPDK) | | | (SEQ ID NO: 43) |
| Actin | Constitutive | Glycine max | Glyma.19G147900 |
| | | | (SEQ ID NO: 44) |
| ADP-glucose pyrophos- | Seed-specific | Glycine max | Glyma.04G011900 |
| phorylase (AGPase) | | | (SEQ ID NO: 45) |
| Glutelin C (GluC) | Seed-specific | Glycine max | Glyma.03G163500 |
| | | | (SEQ ID NO: 46) |
| β-fructofuranosidase | Seed-specific | Glycine max | Glyma.17G227800 |
| insoluble isoenzyme 1 (CIN1) | | | (SEQ ID NO: 47) |
| MADS-Box | Cob-specific | Glycine max | Glyma.04G257100 |
| | | | (SEQ ID NO: 48) |
| Glycinin (subunit G1) | Seed-specific | Glycine max | Glyma.03G163500 |
| | | | (SEQ ID NO: 49) |
| oleosin isoform A | Seed-specific | Glycine max | Glyma.16G071800 |
| | | | (SEQ ID NO: 50) |
| Hsp70 | Constitutive | Brassica napus | BnaA09g05860D |
| Chlorophyll A/B Binding | Constitutive | Brassica napus | BnaA04g20150D |
| Protein (Cab5) | | | |
| Pyruvate phosphate | Constitutive | Brassica napus | BnaA01g18440D |
| dikinase (PPDK) | | | |
| Actin | Constitutive | Brassica napus | BnaA03g34950D |
| ADP-glucose pyrophos- | Seed-specific | Brassica napus | BnaA06g40730D |
| phorylase (AGPase) | | | |
| Glutelin C (GluC) | Seed-specific | Brassica napus | BnaA09g50780D |
| β-fructofuranosidase | Seed-specific | Brassica napus | BnaA04g05320D |
| insoluble isoenzyme 1 (CIN1) | | | |
| MADS-Box | Cob-specific | Brassica napus | BnaA05g02990D |
| Glycinin (subunit G1) | Seed-specific | Brassica napus | BnaA01g08350D |
| oleosin isoform A | Seed-specific | Brassica napus | BnaC06g12930D |
| 1.7S napin (napA) | Seed-specific | Brassica napus | BnaA01g17200D |

*Gene ID includes sequence information for coding regions as well as associated promoters. 5′ UTRs, and 3′ UTRs and are available at Phytozome (see JGI website phytozome.jgi.doe.gov/pz/portal.html).

TABLE 2
Promoters useful for expression of genes in monocots, including maize and rice.

| Gene/Promoter | Expression | Rice* | Maize* | Other |
|---|---|---|---|---|
| Hsp70 | Constitutive | LOC_Os05g38530 | GRMZM2G310431 | |
| | | (SEQ ID NO: 51) | (SEQ ID NO: 59) | |
| Chlorophyll A/B | Constitutive | LOC_Os01g41710 | AC207722.2_FG009 | |
| Binding Protein | | (SEQ ID NO: 52) | (SEQ ID NO: 60) | |
| (Cab5) | | | GRMZM2G351977 | |
| | | | (SEQ ID NO: 61) | |
| maize ubiquitin | Constitutive | | (SEQ ID NO: 109) | |
| promoter/maize | | | | |
| ubiquitin intron | | | | |
| (sequence listed in | | | | |
| Genbank KT962835) | | | | |
| maize ubiquitin | Constitutive | | (SEQ ID NO: 110) | |
| promoter/maize | | | | |
| ubiquitin intron | | | | |
| (maize promoter and | | | | |
| intron sequence with | | | | |
| 99% identity to | | | | |
| sequence in Genbank | | | | |
| KT985051.1) | | | | |
| CaMV 35S | Constitutive | — | — | Cauliflower |
| | | | | mosaic virus |
| | | | | (SEQ ID NO: 108) |
| Pyruvate phosphate | Constitutive | LOC_Os05g33570 | GRMZM2G306345 | |
| dikinase (PPDK) | | (SEQ ID NO: 53) | (SEQ ID NO: 62) | |
| Actin | Constitutive | LOC_Os03g50885 | GRMZM2G047055 | |
| | | (SEQ ID NO: 54) | (SEQ ID NO: 63) | |
| Hybrid cab5/hsp70 | Constitutive | N/A | SEQ ID NO: 64 | |
| intron promoter | | | | |
| ADP-glucose | Secd- | LOC_Os01g44220 | GRMZM2G429899 | |
| pyrophos-phorylase | specific | (SEQ ID NO: 55) | (SEQ ID NO: 65) | |
| (AGPase) | | | | |
| Glutelin C (GluC) | Seed- | LOC_Os02g25640 | N/A | |
| | specific | (SEQ ID NO: 56) | | |
| β-fructofuranosidase | Seed- | LOC_Os02g33110 | GRMZM2G139300 | |
| insoluble isoenzyme | specific | (SEQ ID NO: 57) | (SEQ ID NO: 66) | |
| 1 (CIN1) | | | | |
| MADS-Box | Cob-specific | LOC_Os12g10540 | GRMZM2G160687 | |
| | | (SEQ ID NO: 58) | (SEQ ID NO: 67) | |
| Maize TrpA | Seed- | | GRMZM5G841619 | |
| promoter | specific | | (SEQ ID NO: 111) | |

*Gene ID includes sequence information for coding regions as well as associated promoters. 5′ UTRs, and 3′ UTRs and are available at Phytozome (see JGI website phytozome.jgi.doe.gov/pz/portal.html).

Certain embodiments use genetically engineered land plants or plant cells having multi-gene expression constructs harboring more than one transgene and promoter. The promoters can be the same or different.

Any of the described promoters can be used to control the expression of one or more of genes, their homologs and/or orthologs as well as any other genes of interest in a defined spatiotemporal manner.

Nucleic acid sequences intended for expression in genetically engineered land plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra. The following is a description of various components of typical expression cassettes.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

The coding sequence of the selected gene may be genetically engineered by altering the coding sequence for optimal expression in the crop species of interest. Methods for modifying coding sequences to achieve optimal expression in a particular crop species are well known (Perlak et al., 1991, Proc. Natl. Acad. Sci. USA 88: 3324 and Koziel et al., 1993, Biotechnology 11: 194-200).

Individual plants within a population of genetically engineered land plants that express a recombinant gene(s) may have different levels of gene expression. The variable gene expression is due to multiple factors including multiple copies of the recombinant gene, chromatin effects, and gene suppression. Accordingly, a phenotype of the genetically engineered land plant may be measured as a percentage of individual plants within a population. The yield of a plant can be measured simply by weighing. The yield of seed from a plant can also be determined by weighing. The increase in seed weight from a plant can be due to a number of factors, including an increase in the number or size of the seed pods, an increase in the number of seed and/or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A genetically engineered land plant can be produced by selection of transformed seeds or by selection of transformed plant cells and subsequent regeneration.

In some embodiments, the genetically engineered land plants are grown (e.g., on soil) and harvested. In some embodiments, above ground tissue is harvested separately from below ground tissue. Suitable above ground tissues include shoots, stems, leaves, flowers, grain, and seed. Exemplary below ground tissues include roots and root hairs. In some embodiments, whole plants are harvested and the above ground tissue is subsequently separated from the below ground tissue.

Genetic constructs may encode a selectable marker to enable selection of transformation events. There are many methods that have been described for the selection of transformed plants (for review see (Miki et al., Journal of Biotechnology, 2004, 107, 193-232) and references incorporated within). Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of genetically engineered plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296). Improved versions of many of the fluorescent proteins have been made for various applications. It will be apparent to those skilled in the art how to use the improved versions of these proteins, including combinations, for selection of transformants.

The plants modified for enhanced yield may have stacked input traits that include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-dichlorophenoxyacetic acid (2,4-D) and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialaphos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162).

The genetically engineered land plant that expresses an ISY gene, as disclosed, can be further modified for further enhanced yield too.

For example, the genetically engineered land plant can express one or more mitochondrial transporter proteins that are also expressed as members of carbon-concentrating mechanisms of eukaryotic algae, as well as expressing an ISY gene. In some examples, the mitochondrial transporter protein is a CCP1 mitochondrial transporter protein. In some examples the mitochondrial transporter protein is expressed under the control of plant promoters which may be constitutive, tissue-specific, or seed-specific. Such genetically engineered land plants are expected to have further enhanced yield as compared to plants not expressing the mitochondrial transporter protein, the ISY gene, or both. For example, such genetically engineered land plants may have improved performance, such as increased CO₂ fixation rates, reduced transpiration, and/or increased biomass and/or seed yield.

Thus, in some examples the genetically engineered land plant expresses a CCP1 mitochondrial transporter protein. In these examples, the genetically engineered land plant comprises a modified gene for the CCP1 mitochondrial transporter protein. The CCP1 mitochondrial transporter protein comprises: (i) CCP1 of Chlamydomonas reinhardtii of SEQ ID NO: 112 or (ii) an ortholog of CCP1 as disclosed in PCT/US2018/037740 and PCT/US2017/016421 to Yield10 Bioscience. The ortholog of CCP1 can be, for example, an algal CCP1 ortholog, such as a CCP1 ortholog of Gonium pectorale (e.g. SEQ ID NO: 113 or SEQ ID NO: 114), Volvox carterif nagariensis (e.g. SEQ ID NO: 115), Ettlia oleoabundans (e.g. SEQ ID NO: 116), Chlorella sorokiniana (e.g. SEQ ID NO: 117). The ortholog of CCP1 also can be, for example, a plant CCP1 ortholog, such as a CCP1 ortholog of Erigeron breviscapus (e.g. SEQ ID NO: 118), Zea nicaraguensis (e.g. SEQ ID NO: 119), Poa pratensis (e.g. SEQ ID NO: 120), Cosmos bipinnatus (e.g. SEQ ID NO: 121), Glycine max (e.g. SEQ ID NO: 122), Zea mays (e.g. SEQ ID NO: 123), Oryza sativa (e.g. SEQ ID NO: 124), Triticum aestivum (e.g. SEQ ID NO: 125), Sorghum bicolor (e.g. SEQ ID NO: 126), or Solanum tuberosum (e.g. SEQ ID NO: 127).

The CCP1 mitochondrial transporter protein is localized to mitochondria of the genetically engineered land plant based on a mitochondrial targeting signal. The modified gene for the CCP1 mitochondrial transporter protein comprises (i) another promoter and (ii) a nucleic acid sequence encoding the CCP1 mitochondrial transporter protein. The other promoter is non-cognate with respect to the nucleic acid sequence. The modified gene for the CCP1 mitochondrial transporter protein is configured such that transcription of the nucleic acid sequence encoding the CCP1 mitochondrial transporter protein is initiated from the other promoter and results in expression of the CCP1 mitochondrial transporter protein.

A genetically engineered land plant that expresses an RNA that increases seed yield with increased expression (“an ISY RNA”) also is disclosed. The genetically engineered land plant comprises a modified gene for the ISY RNA. The ISY RNA comprises a contiguous sequence of codons encoding a protein that has homology to a plant invertase inhibitor and/or a pectin methylesterase inhibitor (“an encoded protein”). The encoded protein comprises one or more of (i) a protein of Camelina sativa comprising SEQ ID NO: 2; (ii) a fragment of a protein of Camelina sativa comprising SEQ ID NO: 2; (iii) a Camelina sativa homolog of a protein of Camelina sativa comprising SEQ ID NO: 2; or (iv) an ortholog of a protein of Camelina sativa comprising SEQ ID NO: 2. The modified gene comprises (i) a promoter and (ii) a nucleic acid sequence encoding the ISY RNA. The promoter is non-cognate with respect to the nucleic acid sequence encoding the ISY RNA. The modified gene is configured such that transcription of the nucleic acid sequence is initiated from the promoter and results in expression of the ISY RNA.

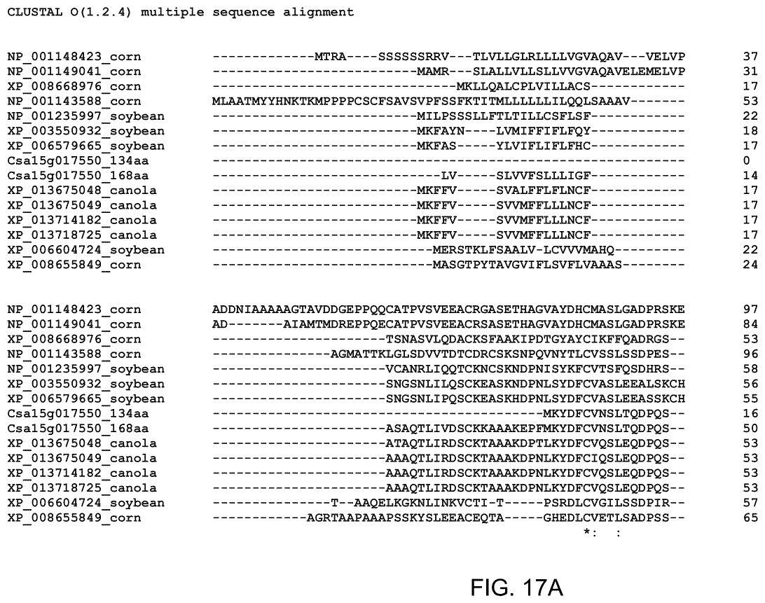

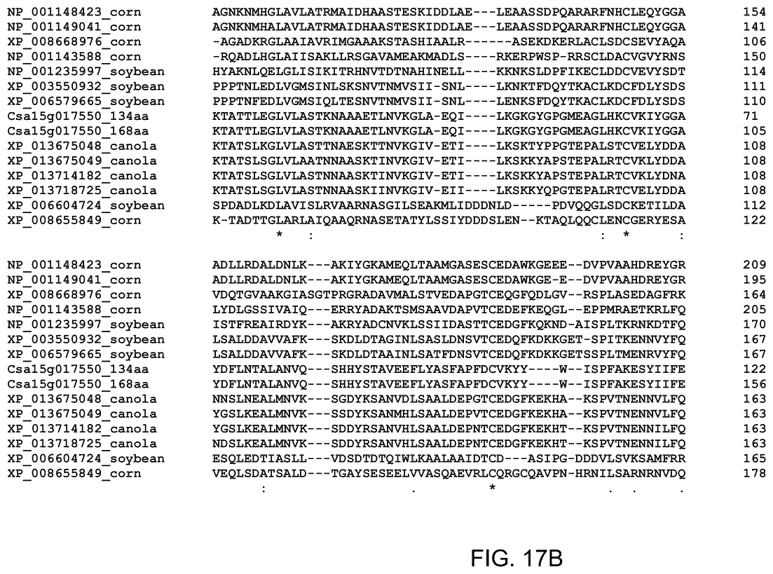

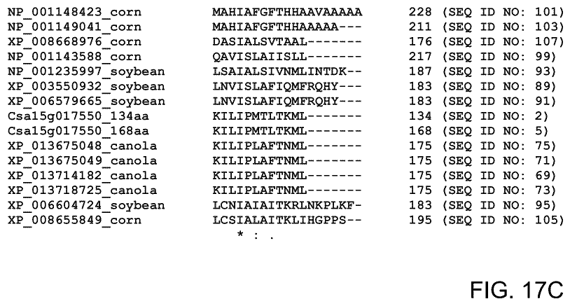

An ISY RNA expressed from an ISY gene may cause an increase in seed yield independent of ISY protein. In Camelina, the ISY gene is a weakly transcribed gene, with a coding potential score of 0.885 (website://cpc.gao-lab.org/programs/cpc.do, Kang Y-J et al, Nucleic Acids Research, 2017, Vol. 45, Web Server issue W12-16), and is only expressed at low levels in senescing leaves and early silique development (Kagale et al, Plant Journal (2016) 88, 879-894 supplementary data). Also, it is not entirely clear which codon or codons correspond to start codons for ISY protein in wild-type Camelina plants. These observations may reflect a role for ISY RNA as, for example, a long non-coding RNA (lncRNA) and/or as a bifunctional RNA (bifRNA). An lncRNA is an RNA, generally including more than 200 nucleotides, that can perform regulatory functions in a cell in its form as RNA, without necessarily coding a protein (Zhang et al., Plant Sciences, 2013, 4, 1038-1045; Hube and Francastel, Frontiers in Genetics, 2018, 9, Article 140). A bifRNA is an RNA that can perform regulatory functions in a cell in its form as RNA and that also codes a protein (Hube and Francastel, 2018). For comparison, a messenger RNA (mRNA) is an RNA that codes a protein, without apparently performing regulatory functions in a cell in its form as RNA. Regarding the ISY gene, it is possible, for example, that the ISY gene can be transcribed to produce an lncRNA that can cause an increase in seed yield based on a regulatory function, without translation of ISY protein from the ISY RNA. It also is possible, for example, that the ISY gene can be transcribed to produce a bifRNA that can cause an increase in seed yield based on both a regulatory function and translation of ISY protein from the ISY RNA. Translation of SEQ ID NO: 3, the genomic region that contains the coding regions for the 168 amino acid protein (DNA SEQ ID NO: 6, encoded protein SEQ ID NO: 5) and the 134 amino acid protein (DNA SEQ ID NO: 1, encoded protein SEQ ID NO: 2), shows that there are no apparent additional start codons for the gene upstream of the TTG start codon of the 168 amino acid protein (FIG. 5). The TTG start codon of SEQ ID NO: 6 would also be the expected start codon based on alignment for the best orthologs of the Camelina ISY gene in canola, soybean, and corn (FIG. 17A-B) as well as alignments to known plant invertase inhibitors (FIG. 1A-B). A weak start codon may be consistent with a role for the ISY gene in expressing an lncRNA and/or a bifRNA. lncRNAs have important functions in plant growth and development (Huang et al, 2017, Proceedings of the National Academy of Sciences, U.S.A., 2017, 114:E3149-E3158, website:www.pnas.org/cgi/doi/10.1073/pnas.1617483114; Zhang et al., 2013). bifRNAs may too. Thus, for example, ISY RNA may act as a transcript to enhance the pathways related to seed yield or regulate the genes that lead to increased plant seed yield, and this may be so independent of, or in conjunction with, expression of ISY protein.

In some examples, the genetically engineered land plant is a food crop plant selected from the group consisting of maize, wheat, oat, barley, soybean, millet, sorghum, potato, sugar beet, pulse, chickpea, green pea, yellow pea, lentils, bean, tomato, and rice. In some examples, the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

As noted, the ISY RNA comprises a contiguous sequence of codons encoding a protein that has homology to a plant invertase inhibitor and/or a pectin methylesterase inhibitor (“an encoded protein”). Also as noted, the encoded protein comprises one or more of (i) a protein of Camelina sativa comprising SEQ ID NO: 2; (ii) a fragment of a protein of Camelina sativa comprising SEQ ID NO: 2; (iii) a Camelina sativa homolog of a protein of Camelina sativa comprising SEQ ID NO: 2; or (iv) an ortholog of a protein of Camelina sativa comprising SEQ ID NO: 2.

In some examples, the protein of Camelina sativa comprising SEQ ID NO: 2 corresponds to one or more of ISY protein of Camelina sativa of SEQ ID NO: 2, ISY protein of Camelina sativa of SEQ ID NO: 5, modified ISY protein of Camelina sativa of SEQ ID NO: 138, or modified ISY protein of Camelina sativa of SEQ ID NO: 140, each as described above.

In some examples, the fragment of a protein of Camelina sativa comprising SEQ ID NO: 2 corresponds to 30 or more contiguous amino acids of SEQ ID NO: 2, as described above. In some of these examples, the fragment corresponds to 40 or more, 50 or more, 60 or more, 70 or more, 80 or more, 90 or more, or 100 or more contiguous amino acids of SEQ ID NO: 2, also as described above. In some of these examples, the fragment corresponds to 114 to 133 contiguous amino acids of SEQ ID NO: 2, also as described above.

In some examples, the Camelina sativa homolog of a protein of Camelina sativa comprising SEQ ID NO: 2 corresponds to one or more Camelina sativa proteins of SEQ ID NOS: 21-40.

In some examples, the ortholog of a protein of Camelina sativa comprising SEQ ID NO: 2 corresponds to one or more (i) canola proteins of SEQ ID NOS: 69, 71, 73, and/or 75; (ii) soybean proteins of SEQ ID NOS: 89, 91, 93, and/or 95; or (iii) corn proteins of SEQ ID NOS: 99, 101, 103, 105, and/or 107.

In some examples, the encoded protein comprises (i) (a) a cysteine residue at position 40, (b) a leucine residue at position 59, (c) a cysteine residue at position 98, and (d) a cysteine residue at position 138, with numbering of positions relative to the protein of Camelina sativa of SEQ ID NO: 5, and (ii) an overall identity of at least 10% with the protein of Camelina sativa of SEQ ID NO: 2.

In some examples, the encoded protein comprises (i) (a) a cysteine residue at position 25, (b) a cysteine residue at position 40, (c) a leucine residue at position 59, (d) a cysteine residue at position 98, and (e) a cysteine residue at position 138, with numbering of positions relative to the protein of Camelina sativa of SEQ ID NO: 5, and (ii) an overall identity of at least 10% with the protein of Camelina sativa of SEQ ID NO: 5.

In some examples, the encoded protein is heterologous with respect to the genetically engineered land plant. In some examples, the encoded protein is homologous with respect to the genetically engineered land plant.

As noted, the modified gene comprises (i) a promoter and (ii) a nucleic acid sequence encoding the ISY RNA. Also, the promoter is non-cognate with respect to the nucleic acid sequence encoding the ISY RNA. In addition, the modified gene is configured such that transcription of the nucleic acid sequence is initiated from the promoter and results in expression of the ISY RNA.

In some examples, the promoter is a constitutive promoter. In some examples, the promoter is a seed-specific promoter.

In some examples, the modified gene is integrated into genomic DNA of the genetically engineered land plant. In some examples, the modified gene is stably expressed in the genetically engineered land plant.

In some examples, the genetically engineered land plant exhibits increased expression of the ISY RNA in comparison to a reference land plant that does not include the modified gene. In some examples, the genetically engineered land plant exhibits increased seed yield, fruit yield, and/or tuber yield in comparison to a reference land plant that does not include the modified gene.

In some examples, the nucleic acid sequence encoding the ISY RNA is at least 80% identical to SEQ ID NO: 1. The nucleic acid sequence can be, for example, at least 85%, at least 90%, at least 95%, at least 96%, at least 97%, at least 98%, or at least 99% identical to SEQ ID NO: 1. In some examples, the nucleic acid sequence encoding the ISY RNA comprises SEQ ID NO: 1. In some examples, the nucleic acid sequence encoding the ISY RNA comprises SEQ ID NO: 6. In some examples, the nucleic acid sequence encoding the ISY RNA comprises SEQ ID NO: 7.

As noted above, an ISY RNA expressed from an ISY gene may cause an increase in seed yield independent of ISY protein. Thus, in some examples, expression of the ISY RNA results in expression of the encoded protein. In some of these examples, the ISY RNA is a bifRNA that causes an increase in seed yield based on a regulatory function and based on translation of the encoded protein from the ISY RNA. Also, in some examples expression of the ISY RNA does not result in expression of the encoded protein. In some of these examples, the ISY RNA is an lncRNA that causes an increase in seed yield based on a regulatory function, without translation of the encoded protein from the ISY RNA.

### EXAMPLES

### Example 1. Expression of CCP1 in Camelina Using a Constitutive Promoter, Impact on Seed Yield and Seed Size, Identification of Up-Regulated ISY Genes in Lines Having Higher Seed Yield and Optionally Reduced Seed Size

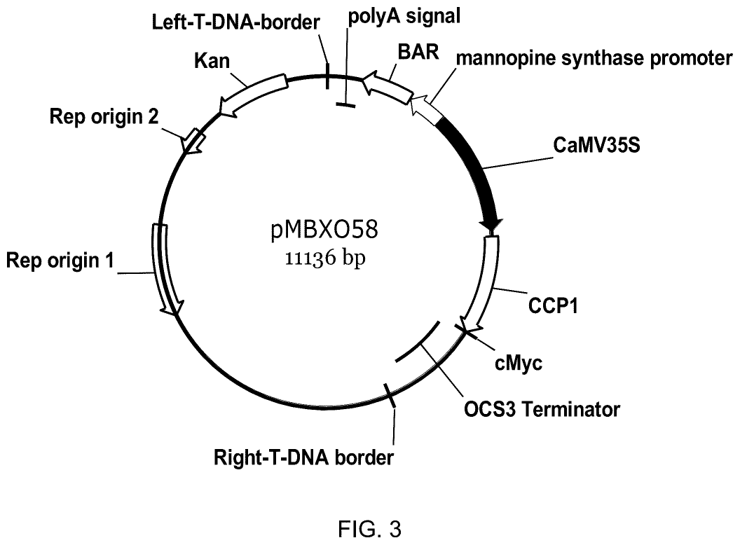

Schnell et al., WO 2015/103074, have reported that heterologous expression of CCP1 from Chlamydomonas reinhardtii in Camelina leads to significant improvements in levels of photosynthesis in the transgenic plants. For these experiments a vector equivalent to pMBXO58 (FIG. 3, SEQ ID NO: 19) was used.

Schnell et al., WO 2015/103074, also disclose that functional data suggest that Camelina plants transformed to express CCP1 of Chlamydomonas reinhardtii respond to higher CO₂ transport capacity by decreasing transpiration and gas exchange (i.e. closing stomata), and that in field studies involving comparison of plants of three transformants versus wild-type plants, two of the three transformants exhibited an increase in overall oil yield (lb/acre) of 43% and 76%, respectively. Schnell et al., WO 2015/103074, also reported a decrease in seed size in higher yielding Camelina lines expressing CCP1 constitutively.

The Csa15g017550 gene was identified from transcriptome analysis (RNASeq) of Camelina lines engineered to constitutively express the CCP1 gene from the 35S constitutive promoter using greenhouse grown samples (J. Zuber, RNAi Mediated Silencing of Cell Wall Invertase Inhibitors to Increase Sucrose Allocation to Sink Tissues in Transgenic Camelina sativa Engineered with a Carbon Concentrating Mechanism, thesis submitted to the Graduate School of The University of Massachusetts, Amherst, Master of Science, May 2015).

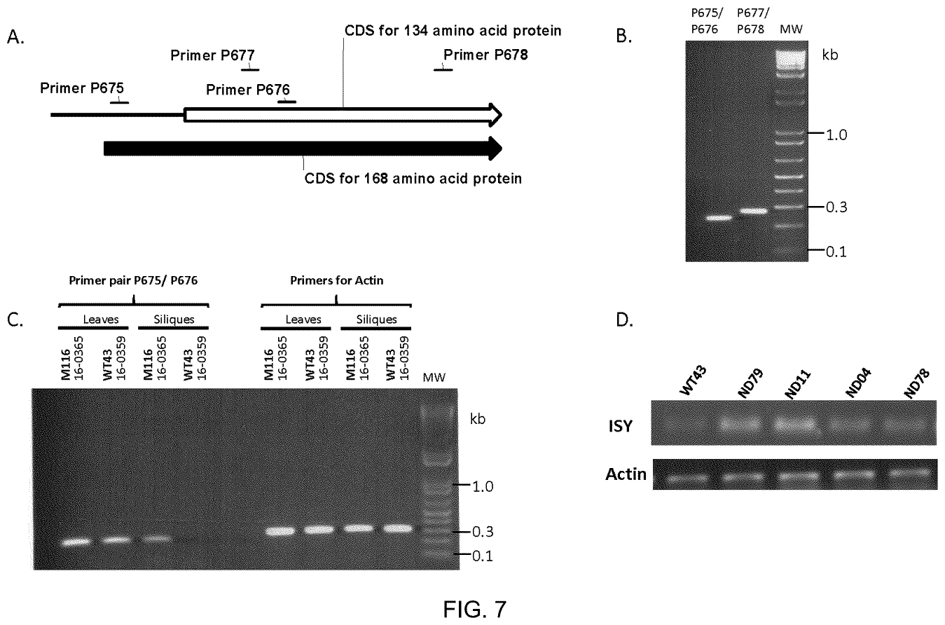

Subsequent analysis of the expression of the Csa15g017550 gene by RT-PCR in Camelina lines transformed with pMBXO58 (FIG. 3, SEQ ID NO: 19) at Yield10 Bioscience with primers shown in TABLE 4 also showed an increase in Csa15g017550 mRNA (FIG. 7C).

The Csa15g017550 gene has limited sequence homology to genes from the plant invertase inhibitor/pectin methylesterase inhibitor superfamily. Cell wall invertase inhibitor (CWII) genes have previously been shown in Camelina to be useful targets for down regulation using RNAi to increase seed yield in Camelina (Sederoff et al., U.S. Pub. No. 2016/0138038). Fruit-specific suppression of the cell wall invertase inhibitor (CWII) in tomato or rice led to increases in net seed/grain weight of 22% and 10%, respectively (Wang et al. (2008) Nat Genet, 40(11):1370-1374; Jin et al, Plant Cell, (2009) 21(7):2072-89).

### Example 2. Expression of CCP1 in Camelina Using a Seed-Specific Promoter and RT-PCR Analysis of the Impact on Expression of the Csa15g017550 Gene Identified in Example 1

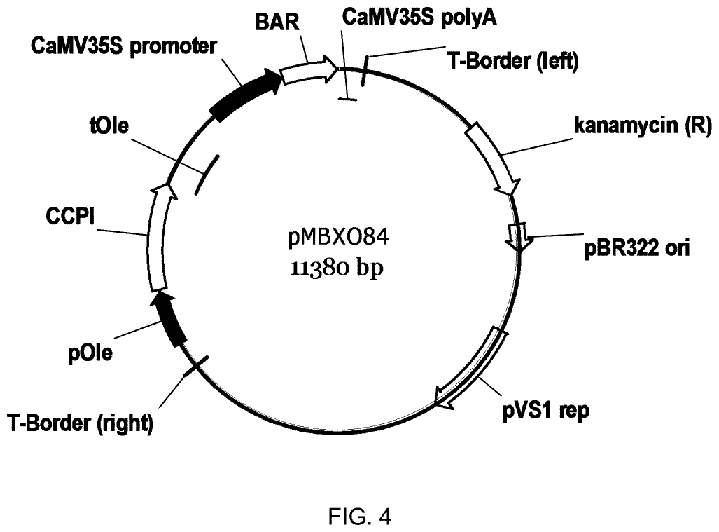

In co-pending Patent Application, PCT/US2018/019105 (WO2018/156686) to Yield10 Bioscience, a seed-specific expression construct for CCP1 was described (pMBXO84 (FIG. 4, SEQ ID NO: 96)). Transformation of pMBXO84 into Camelina resulted in higher seed yield and a normal seed size. RT-PCR analysis was performed on RNA isolated from these lines to compare the expression of the Csa15g017550 gene in wild-type and transgenic lines and a small amount of overexpression of the Csa15g017550 gene was observed (FIG. 7D) using primers shown in TABLE 4.

### Example 3. Novel Gene to Increase Seed Yield (Csa15g017550.1)

On the Camelina sativa genome browser (website://www.camelinadb.ca/), a 612 bp fragment for Csa15g017550.1 is provided that contains a 141 bp 5′ UTR, a 405 bp coding sequence (CDS; SEQ ID NO: 1), and a 66 bp 3′ UTR, such that the encoded protein is predicted to be 134 amino acids in length (SEQ ID NO: 2). A protein with a longer sequence (SEQ ID NO: 20) was annotated as a “putative invertase inhibitor” in GenBank (LOC104745594) (webiste://www.ncbi.nlm.nih.gov/nucleotide/727584402?report=genbank&log$=nucltop&bla st_rank=1&RID=YAF599RN015), however this record was removed from GenBank during standard genome annotation processing.

This protein is predicted to be related to the plant invertase inhibitor/pectin methylesterase inhibitor superfamily. An alignment of the predicted 134 amino acid protein (SEQ ID NO: 2) with other plant invertase inhibitors was performed. This alignment (TABLE 3, FIG. 1) showed that the 134 amino acid protein (SEQ ID NO: 2) was shorter than the other invertase inhibitors, which were 168 to 205 amino acids in length. SEQ ID NO: 2 is missing a stretch of amino acids at the N-terminus, often associated with a signal peptide, and only possesses three of the four conserved cysteine residues that are typically observed in plant invertase inhibitors (Wan et al (2018), Trends Plant Sci, 23:163-177; Tang at al. (2017), J Exp Bot, 68:469-482). In fact the gene was labeled a “partial mRNA” in Genbank under the accession XM_010466881.1 (this accession has recently been removed from Genbank due to standard genome annotation processing). A larger DNA fragment was downloaded from NCBI (LOC104745594; website://www.ncbi.nlm.nih.gov/gene/104745594) that contains additional sequence on the 5′ and 3′ ends. The record for LOC104745594 was later removed from NCBI because the model on which it was based was not predicted in a later annotation. A 3376 bp portion of this fragment (SEQ ID NO: 3) was searched for an alternative start site to produce a larger protein with a fourth conserved cysteine. A 576 bp DNA fragment (SEQ ID NO: 7) was identified within SEQ ID NO: 3 that encodes for a 190 amino acid open reading frame that contains an internal TTG (base pair 70; FIG. 5). This TTG encodes a Leucine at amino acid 23 (FIG. 5) that if used as an alternative start site would yield a 168 amino acid protein (SEQ ID NO: 5). SEQ ID NO: 5 contains the fourth conserved cysteine (FIG. 1A, FIG. 5).

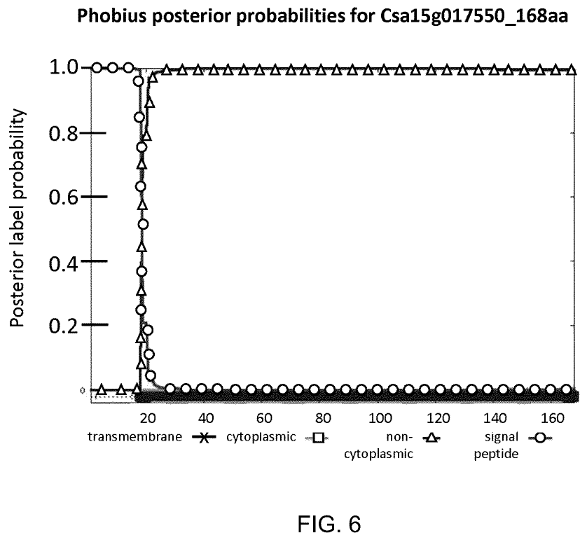

The Phobius transmembrane topology and signal peptide prediction program (website://phobius.sbc.su.se/) was used to probe the 134 amino acid protein (SEQ ID NO: 2) and the 168 amino acid protein (SEQ ID NO: 5) sequences for a signal peptide. A putative 17 amino acid signal peptide was identified on the 168 amino acid protein (SEQ ID NO: 5; FIG. 6) and the protein was predicted to be non-cytoplasmic. No signal peptide was identified on the 134 amino acid sequence (SEQ ID NO: 2).

The 134 amino acid protein (SEQ ID NO: 2) and the 168 amino acid protein (SEQ ID NO: 5) sequences were also analyzed using the TargetP 1.1 Server (website://www.cbs.dtu.dk/cgi-bin/webface2.fcgi?jobid=5B84A216000034F4085302D6&wait=20) which predicts chloroplast, mitochondrial, and signal peptides. This program also predicted the presence of a signal peptide involved in a secretory pathway for the 168 amino acid protein and a transit peptide length of 17 amino acids. For the 134 amino acid protein no signal peptide was predicted.

The 134 amino acid protein (SEQ ID NO: 2) and the 168 amino acid protein (SEQ ID NO: 5) were also analyzed using the Plant-mPloc software (website://www.csbio.sjtu.edu.cn/bioinf/plant-multi/) useful for predicting the subcellular localization of proteins. This program predicted the location of both the 134 and 168 amino acid proteins in the cell membrane.

Based on these analyses the function of this gene is unknown and so we have defined it herein as the increased seed yield gene (ISY).

TABLE 3
Plant invertase inhibitor sequences used for comparison to the novel Camelina plant ISY protein

| | | | | Size of | |
|---|---|---|---|---|---|
| | | | | encoded | |
| | SEQ ID of | | Function of | protein | Experimentally |
| Crop | Protein | Gene ID | protein | (amino acids) | verified |
| Camelina sativa | SEQ ID | Csa15g017550.1 | Putative plant | 134 | no |
| | NO: 2 | | invertase | | |
| | | | inhibitor/pectin | | |
| | | | methylesterase | | |
| | | | inhibitor | | |
| Camelina sativa | SEQ ID | Csa15g017550.1 | Putative plant | 190 | no |
| | NO: 4 | with additional | invertase | | |
| | | upstream region | inhibitor/pectin | | |
| | | (SEQ ID NO: 7) | methylesterase | | |
| | | | inhibitor | | |
| Camelina sativa | SEQ ID | Csa15g017550.1 | Putative plant | 168 | no |
| | NO: 5 | with alternative start | invertase | | |
| | | codon | inhibitor/pectin | | |
| | | (SEQ ID NO: 6) | methylesterase | | |
| | | | inhibitor | | |
| Zea mays | SEQ ID | XP_008668976.1 | Cell | 176 | yes² |
| | NO: 8 | | wall/vacuolar | | |
| | | | inhibitor of | | |
| | | | fructosidase 2 | | |
| Arabidopsis | SEQ ID | AT5G64620 | Cell | 180 | yes² |
| thaliana | NO: 9 | | wall/vacuolar | | |
| | | | inhibitor of | | |
| | | | fructosidase | | |
| Arabidopsis | SEQ ID | AEE32232.1 | Cell | 205 | |
| thaliana | NO: 10 | | wall/vacuolar | | |
| | | | inhibitor of | | |
| | | | fructosidase 1 | | |
| Solanum | SEQ ID | Solyc12g099190 | Invertase | 175 | yes² |
| lycopersicum¹ | NO: 11 | | inhibitor | | |
| Solanum | SEQ ID | Solyc12g099200 | Invertase | 171 | yes² |
| lycopersicum¹ | NO: 12 | | inhibitor | | |
| Beta vulgaris | SEQ ID | XP_010685378.1 | Predicted cell | 184 | yes³ |
| subsp. vulgaris | NO: 13 | | wall/vacuolar | | |
| | | | inhibitor of | | |
| | | | fructosidase 1 | | |
| Nicotiana | SEQ ID | AY145781.1 | Vacuolar | 172 | yes³ |
| tabacum | NO: 14 | | invertase | | |
| | | | inhibitor | | |
| Nicotiana | SEQ ID | Y12805.1 | Invertase | 166 | yes² |
| tabacum | NO: 15 | | inhibitor | | |
| Solanum | SEQ ID | GU321341.1 | Putative | 181 | yes² |
| tuberosum | NO: 16 | | invertase | | |
| cultivar | | | inhibitor | | |
| Shepody | | | (INVINH2A) | | |

¹Protein sequence obtained from website://solgenomics.net/search/locus.
²Protein listed as experimentally characterized in review by Wan et al. (2018), Trends Plant Sci, 23: 163-177.
³Protein listed as experimentally characterized in review by Tang et al. (2017), J ExpBot, 68: 469-482.

Most invertase inhibitors also contain a conserved three amino acid conserved motif of Proline-Lysine-Phenylalanine (PKF; Tang et al. (2017), J Exp Bot, 68:469-482). Neither the 134 amino acid protein (SEQ ID NO: 2) nor the 168 amino acid protein (SEQ ID NO: 5) contain the conserved PKF motif. The presence of the PKF motif can be found at position 132 through 134 of the Beta vulgaris predicted cell wall/vacuolar inhibitor of fructosidase 1 (Gene ID XP_010685378.1, SEQ ID NO: 13, FIG. 1).

Since Genbank removed the record for XM_010466881 (Csa15g017550.1), an effort was made to verify the presence of the gene in the genome. Genomic DNA was extracted from plants of Camelina sativa germplasm 10CS0043 (abbreviated WT43; germplasm obtained from Kevin Falk at Agriculture and Agri-Food Canada). PCR experiments were conducted using primer pair P675 and P676, as well as primer pair P677 and P678 (TABLE 4). The binding sites of these primers within SEQ ID NO: 7 (contains 576 bp DNA fragment that encodes for a 190 amino acid open reading frame that contains an internal TTG) is shown in FIG. 5 and FIG. 7. These PCR reactions demonstrated that the two overlapping regions of the gene were present in genomic DNA of WT43. A PCR reaction with primer pair P675/P676 produced a band consistent with the expected 0.236 kb fragment (FIG. 7B). A PCR reaction with primer pair P677/P678 produced a band consistent with the expected 0.269 kb fragment.

RNA transcript levels of the ISY gene were also tested in RNA isolated from the WT43 line (line 16-0359) and a transgenic line expressing CCP1 (MI16 line 16-0365). RNA was isolated from leaves of both lines just prior to bolting and from siliques collected 12 days after flowering. RT-PCR experiments with RNA from both the transgenic and control lines with primer pair P675/P676 (FIG. 5 and FIG. 7) showed that the transgenic line expressing the CCP1 gene contained more transcript for the ISY compared to the wild-type line in both leaves and siliques (FIG. 7).

TABLE 4
Primers used to verify the presence
of Csa15g017550.1 in genomic DNA and
RNA transcripts.

| | | Expected PCR |
|---|---|---|
| Primer | Sequence | or RT-PCR product |
| P675 | 5′-CCTTGGTTGTG | 0.236 kb fragment |
| | TTCTCTCTTCT-3′ | of putative |
| | (SEQ ID NO: 131) | invertase inhibitor |
| P676 | 5′-GATCTGTTCAG | |
| | CGAGTCCTTT-3′ | |
| | (SEQ ID NO: 132) | |
| P677 | 5′-TAGTCCTAGCA | 0.269 kb fragment |
| | TCGACGAAGA-3′ | of putative |
| | (SEQ ID NO: 133) | invertase inhibitor |
| | 5′-AGCGAAGGGAG | |
| P678 | AAATCCAATAA-3′ | |
| | (SEQ ID NO: 134) | |
| P602 | 5′-CGGCCGATTCT | 0.77 kb fragment |
| | GTTTATCTC-3′ | of actin with genomic |
| | (SEQ ID NO: 135) | DNA and 0.32 kb |
| P603 | 5′-TCCTTCTGGTT | fragment actin with |
| | CATCCCAAC-3′ | RNA/cDNA as template |
| | (SEQ ID NO: 136) | |

Since Camelina is an allohexaploid, there are typically three homeologs of each gene. BLAST searches were performed with the 168 amino acid protein encoded by the Csa15g017550.1 against the Camelina genome database (website://www.camelinadb.ca/prairiegold/cgi-bin/blast.cgi) in efforts to identify the two other homeologs to Csa15g017550.1. The results of the protein BLAST search showing gene IDs and E values are shown in TABLE 5 (homeologs 1-38, 41, 57-66). Apparent candidates for two other ISY gene Csa15g017550 homeologs could not be found. An additional BLAST search was performed using the 507 base pair coding sequence (SEQ ID NO: 6) for the 168 amino acid protein (SEQ ID NO: 5) of Csa15g017550. Homeologs 39, 40, and 42-56 were identified and are indicated with a “*” next to the homeolog number. No apparent candidates for the two other ISY gene Csa15g017550 homeologs could be found. The percent homology of the 168 amino acid protein (SEQ ID NO: 5) to the first twenty best match homeologs, as indicated by E value, were calculated using the ALIGNX alignment function of the Vector NTI software package (ThermoFisher) and are shown in TABLE 5. Their protein sequences are provided as SEQ ID NOS: 21-40. Percent homologies were also calculated for Csa11g101740 (SEQ ID NO: 85) and Csa18g038260 (SEQ ID NO: 87) in TABLE 5, two homeologs of the cell wall invertase inhibitor 2 (CWII2) protein described in Sederoff et al., U.S. Pub. No. 2016/0138038. These proteins are also described in TABLE 6.

TABLE 5
Camelina homeologs to novel Camelina ISY gene Csa15g017550.1

| | | | | % homology to |
|---|---|---|---|---|
| Homeolog | | | | protein in SEQ |
| No. ¹ | Gene ID | E Value | Encoded Protein | ID NO: 5² |
| | Csa15g017550 | | SEQ ID NO: 5 | 100 |
| | | | (168 amino acid protein) | |
| | Csa15g017550 | 6.00E−74 | SEQ ID NO: 2 | 79.8 |
| | | | (134 amino acid protein) | |
| 1 | Csa18g003060 | .00E−53 | SEQ ID NO: 21 | 64.8 |
| | | | (176 amino acid protein) | |
| 2 | Csa11g064960 | 1.00E−51 | SEQ ID NO: 22 | 63.1 |
| | | | (174 amino acid protein) | |
| 3 | Csa11g064980 | 3.00E−51 | SEQ ID NO: 23 | 63.0 |
| | | | (168 amino acid protein)23 | |
| 4 | Csa20g081200 | 1.00E−50 | SEQ ID NO: 24 | 63.1 |
| | | | (175 amino acid protein) | |
| 5 | Csa18g003050 | 1.00E−49 | SEQ ID NO: 25 | 62.5 |
| | | | (175 amino acid protein) | |
| 6 | Csa20g077790 | 2.00E−49 | SEQ ID NO: 26 | 60.5 |
| | | | (176 amino acid protein) | |
| 7 | Csa11g064950 | 2.00E−46 | SEQ ID NO: 27 | 59.1 |
| | | | (176 amino acid protein) | |
| 8 | Csa02g070240 | 7.00E−46 | SEQ ID NO: 28 | 57.4 |
| | | | (175 amino acid protein) | |
| 9 | Csa20g081210 | 7.00E−46 | SEQ ID NO: 29 | 59.1 |
| | | | (176 amino acid protein) | |
| 10 | Csa18g003030 | 8.00E−45 | SEQ ID NO: 30 | 56.8 |
| | | | (175 amino acid protein) | |
| 11 | Csa18g005290 | 9.00E−44 | SEQ ID NO: 31 | 56.5 |
| | | | (176 amino acid protein) | |
| 12 | Csa07g013600 | 3.00E−43 | SEQ ID NO: 32 | 55.4 |
| | | | (176 amino acid protein) | |
| 13 | Csa17g022200 | 4.00E−43 | SEQ ID NO: 33 | 55.9 |
| | | | (176 amino acid protein) | |
| 14 | Csa15g021010 | 1.00E−42 | SEQ ID NO: 34 | 55.9 |
| | | | (176 amino acid protein) | |
| 15 | Csa15g020370 | 2.00E−39 | SEQ ID NO: 35 | 50.0 |
| | | | (147 amino acid protein) | |
| 16 | Csa20g077190 | 6.00E−39 | SEQ ID NO: 36 | 52.8 |
| | | | (175 amino acid protein) | |
| 17 | Csa18g003080 | 2.00E−34 | SEQ ID NO: 37 | 44.3 |
| | | | (183 amino acid sequence) | |
| 18 | Csa20g081180 | 3.00E−34 | SEQ ID NO: 38 | 42.9 |
| | | | (175 amino acid sequence) | |
| 19 | Csa20g081190 | 4.00E−34 | SEQ ID NO: 39 | 45.8 |
| | | | (179 amino acid sequence) | |
| 20 | Csa18g003070 | 2.00E−32 | SEQ ID NO: 40 | 44.9 |
| | | | (178 amino acid sequence) | |
| 21 | Csa11g064990 | 3.00E−32 | | |
| 22 | Csa11g065000 | 3.00E−29 | | |
| 23 | Csa20g081220 | 6.00E−29 | | |
| 24 | Csa18g003020 | 8.00E−16 | | |
| 25 | Csa20g081240 | 3.00E−13 | | |
| 26 | Csa18g003010 | 4.00E−13 | | |
| 27 | Csa20g081230 | 1.00E−10 | | |
| 28 | Csa11g066240 | 3.00E−10 | | |
| 29 | Csa11g064940 | 2.00E−09 | | |
| 30 | Csa10g044780 | 7.00E−06 | | |
| 31 | Csa11g053620 | 2.00E−05 | | |
| 32 | Csa12g083870 | 2.00E−05 | | |
| 33 | Csa13g054980 | 0.013 | | |
| 34 | Csa08g052220 | 0.013 | | |
| 35 | Csa02g005450 | 0.013 | | |
| 36 | Csa11g101740 | 0.017 | SEQ ID NO: 85 | 19.2 |
| | | | (181 amino acid sequence) | |
| 37 | Csa02g074170 | 0.017 | | |
| 38 | Csa11g064930 | 0.037 | | |
| 39* | Csa15g002570 | 0.72 | | |
| 40* | Csa19g004990 | 0.72 | | |
| 41 | Csa18g038260 | 0.083 | SEQ ID NO: 87 | 18.7 |
| | | | (181 amino acid sequence) | |
| 42* | Csa01g001500 | 2.8 | | |
| 43* | Csa01g025990 | 2.8 | | |
| 44* | Csa03g032110 | 2.8 | | |
| 45* | Csa03g059400 | 2.8 | | |
| 46* | Csa03g062690 | 2.8 | | |
| 47* | Csa07g030720 | 2.8 | | |
| 48* | Csa08g002670 | 2.8 | | |
| 49* | Csa08g058620 | 2.8 | | |
| 50* | Csa13g007860 | 2.8 | | |
| 51* | Csa14g036540 | 2.8 | | |
| 52* | Csa15g001530 | 2.8 | | |
| 53* | Csa17g040740 | 2.8 | | |
| 54* | Csa19g002590 | 2.8 | | |
| 55* | Csa19g031990 | 2.8 | | |
| 56* | Csa20g008110 | 2.8 | | |
| 57 | Csa08g034720 | 0.24 | | |
| 58 | Csa01g019830 | 0.31 | | |
| 59 | Csa05g029780 | 0.41 | | |
| 60 | Csa19g024110 | 0.41 | | |
| 61 | Csa12g032200 | 0.7 | | |
| 62 | Csa15g023200 | 0.7 | | |
| 63 | Csa16g012150 | 3.5 | | |
| 64 | Csa07g011430 | 5.9 | | |
| 65 | Csa02g040660 | 5.9 | | |
| 66 | Csa04g024120 | 5.9 | | |

Csa gene IDs are from the Camelina genome database found at (website://www.camelinadb.ca/prairiegold/cgi-bin/blast.cgi).
¹ Entries with a “*” were found with BLAST searches using the 507-bp coding sequence. All other entries were found using BLAST searches with the 168 amino acid protein found in SEQ ID NO: 5,
²Percent homology over the whole protein of SEQ ID NO: 5 was determined using the ALIGNX alignment function of the Vector NTI software package (ThermoFisher).

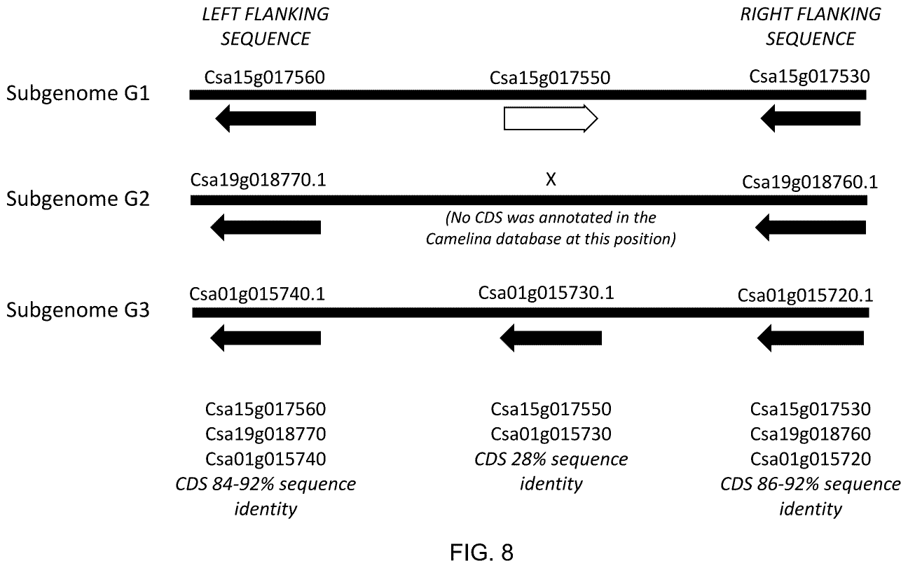

In other efforts to identify two homeologs to the ISY gene Csa15g017550, a syntenic analysis was performed (FIG. 8). Csa15g017550 was found to be on subgenome G1 and is flanked by coding sequences for Csa15g017560 on the left and by Csa15g017530 on the right. The two homeologs of the left flanking gene Csa15g017560 on subgenomes G2 (Csa19g018770) and G3 (Csa01g015740) have been previously identified by Kagale et al. (2014), Nat Commun, 5:3706). The coding sequence identity of Csa15g017560, Csa19g018770, and Csa01g015740 is between 84-92%. The sequence homeolog of the right flanking gene Csa15g017530 on subgenome 2 (Csa19g018760) was also identified by Kagale et al. (2014). Our work has identified Csa01g015720 as the likely other homeolog to Csa15g017530 on subgenome 3 based on its position in the genome and high codon sequence identity (86-92%) to the other two genes. However, no homeologs were identified for Csa15g017550. On subgenome 2, there was no CDS annotated at the expected genomic position in the Camelina genome database. Repeated sequences in the genome were observed in this region suggesting possible gene rearrangement or deletion may have occurred. On subgenome G3, the CDS Csa01g015730.1 is present with only 28% sequence identity to Csa15g017550. This was not annotated as a homeolog of Csa15g017550 by Kagale et al. (2014). It thus appears that ISY gene Csa15g017550 is present as a single copy within the allohexaploid Camelina.

Csa15g017550 is distinct from the prior two CWII genes in Camelina that have been studied (Sederoff et al., U.S. Pub. No. 2016/0138038, at North Carolina State University). Sederoff et al. used RNAi gene silencing to reduce expression of these genes in Camelina and demonstrated an increase in seed yield. Three copies of each of these genes is present (Camelina is an allohexaploid). These homeologs include homeologs of cell wall invertase inhibitor 1 (CWII1) (SEQ ID NO: 76 (Csa03g051630), SEQ ID NO: 78 (Csa14g051860), and SEQ ID NO: 80 (Csa17g075360)) and homeologs of cell wall invertase inhibitor 2(CWII2) (SEQ ID NO: 82 (Csa02g074171), SEQ ID NO: 84 (Csa11g101740), and SEQ ID NO: 86 (Csa18g038260)) found in the Camelina genome. A CLUSTAL alignment of the proteins encoded by these genes to the 134 and 168 amino acid ISY protein is shown in FIG. 2 and the percent of each homeolog to the ISY protein is shown in TABLE 6. The percent homology to the 168 amino acid ISY protein (SEQ ID NO: 5) ranges from 12.3 to 19.2% over the entire length of the sequence. Because of the low homology, only SEQ ID NO: 84 (Csa11g101740) and SEQ ID NO: 86 (Csa18g038260) showed up in our BLAST searches of Csa15g017550 shown in TABLE 5.

TABLE 6
Homology of novel Camelina ISY gene Csa15g017550.1 to previously
described Camelina cell wall invertase inhibitor proteins

| | | Size of | | |
|---|---|---|---|---|
| | | encoded | % homology | % homology |
| | | protein | to protein | to protein |
| | SEQ ID NO | (amino | in SEQ ID | in SEQ ID |
| Gene ID¹ | of Protein | acids) | NO: 2² | NO: 5³ |
| Csa15g017550 | SEQ ID | 168 | 79.8 | 100 |
| | NO: 5 | | | |
| Csa15g017550 | SEQ ID | 134 | 100 | 79.8 |
| | NO: 2 | | | |
| CWII1 homeolog | SEQ ID | 159 | 11.0 | 12.3 |
| SEQ ID NO: 76 | NO: 77 | | | |
| (Csa03g051630) | | | | |
| CWII1 homeolog | SEQ ID | 155 | 11.3 | 12.5 |
| SEQ ID NO: 78 | NO: 79 | | | |
| (Csa14g051860) | | | | |
| CWII1 homeolog | SEQ ID | 159 | 11.0 | 12.9 |
| SEQ ID NO: 80 | NO: 81 | | | |
| (Csa17g075360) | | | | |
| CWII2 homeolog | SEQ ID | 180 | 14.4 | 18.2 |
| SEQ ID NO: 82 | NO: 83 | | | |
| (Csa02g074171) | | | | |
| CWII2 homeolog | SEQ ID | 181 | 14.8 | 19.2 |
| SEQ ID NO: 84 | NO: 85 | | | |
| (Csa11g101740) | | | | |
| CWII2 homeolog | SEQ ID | 181 | 14.3 | 18.7 |
| SEQ ID NO: 86 | NO: 87 | | | |
| (Csa18g038260) | | | | |

¹Csa gene IDs are from the Camelina sativa genome browser (website://www.camelinadb.ca/prairiegold/cgi-bin/gbrowse/camelina/); CWII1 and CWII2 homeologs are the three copies of the CWII genes that were studied in U.S. Pub No. 2016/0138038.
²Percent homology over the whole protein of SEQ ID NO: 2 was determined using the alignment function of the Vector NTI software package (ThermoFisher).
³Percent homology over the whole protein of SEQ ID NO: 5 was determined using the ALIGNX alignment function of the Vector NTI software package (ThermoFisher).

### Example 4. Overexpressina the Csa15a017550.1 Gene Identified in Example 1 in Camelina Alone or in Combination with Expression of CCP1

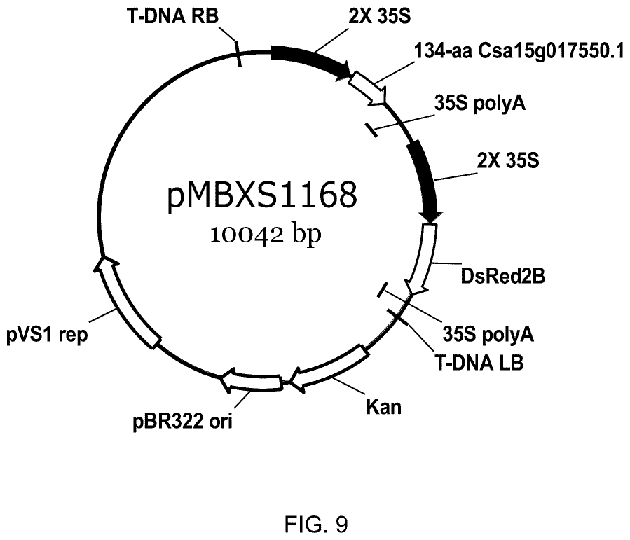

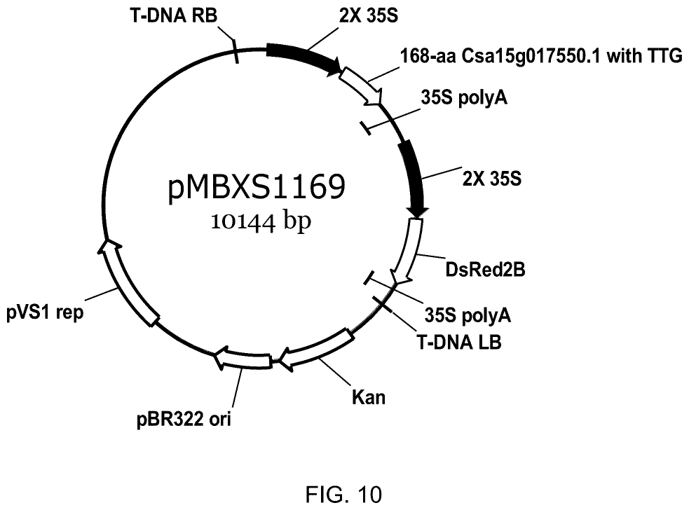

We have previously observed in Camelina, canola, and soybean lines engineered to constitutively express CCP1 that, in addition to increased seed yield, there was a reduction in individual seed weight. This together with the work disclosed by Sederoff et al., U.S. Pub. No. 2016/0138038, on reducing expression of CWII genes to increase seed yield led to the hypothesis that expression of CCP1 results in increased availability of carbon from photosynthesis, increased seed numbers, and increased expression of the Csa15g017550 gene, which may be a CWII or other plant invertase inhibitor that is responsible for reducing the flow of carbon to seed. To test this hypothesis, we overexpressed the Csa15g017550 gene in two different Camelina backgrounds. Based on the fact that the Csa15g017550 gene has two possible translation initiation sites (FIG. 5), we made expression constructs for both for transformation of Camelina (TABLE 7). The Csa15g017550 gene expression cassette in construct pMBXS1168 (FIG. 9, SEQ ID NO: 17) starts at an ATG codon and encodes a 134 amino acid protein (SEQ ID NO: 2) whereas the Csa15g017550 gene expression cassette in construct pMBXS1169 (FIG. 10, SEQ ID NO: 18) starts upstream at a TTG codon and encodes a 168 amino acid protein (SEQ ID NO: 5). Both constructs were transformed into the Camelina Suneson wild-type background and into Suneson lines which had previously been engineered with vector pMBXO58 (FIG. 3, SEQ ID NO: 19) to express CCP1.

TABLE 7
Summary of constructs for transformation into Camelina

| | Size of Csa15g017550.1 | |
|---|---|---|
| Construct | encoded protein | Promoter |
| pMBXS1168 | 134 amino acids | CaMV 35S |
| (SEQ ID NO: 17; FIG. 9) | (SEQ ID NO: 2) | (constitutive) |
| pMBXS1169 | 168 amino acids | CaMV 35S |
| (SEQ ID NO: 18; FIG. 10) | (SEQ ID NO: 5) | (constitutive) |

These constructs were transformed into Camelina as follows.

In preparation for plant transformation experiments, seeds of Camelina sativa germplasm 10CS0043 (abbreviated WT43, obtained from Agriculture and Agri-Food Canada) were sown directly into 4 inch (10 cm) pots filled with soil in the greenhouse. Growth conditions were maintained at 24° C. during the day and 18° C. during the night. Plants were grown until flowering. Plants with a number of unopened flower buds were used in “floral dip” transformations.

Agrobacterium strain GV3101 (pMP90) was transformed with either plasmid pMBXS1168 or pMBXS1169 using electroporation. A single colony of GV3101 (pMP90) containing the construct of interest was obtained from a freshly streaked plate and was inoculated into 5 mL LB medium. After overnight growth at 28° C., 2 mL of culture was transferred to a 500-mL flask containing 300 mL of LB and incubated overnight at 28° C. Cells were pelleted by centrifugation (4,000 rpm, 20 min), and diluted to an OD600 of −0.8-1.0 with infiltration medium containing 5% sucrose and 0.05% (v/v) Silwet-L77 (Lehle Seeds, Round Rock, TX, USA). Plants of Camelina sativa germplasm 10CS0043 (abbreviated WT43; germplasm obtained from Kevin Falk at Agriculture and Agri-Food Canada) were transformed by “floral dip” using pMBXS1168 or pMBXS1169 transformation constructs as follows. Pots containing plants at the flowering stage were placed inside a 460 mm height vacuum desiccator (Bel-Art, Pequannock, NJ, USA). Inflorescences were immersed into the Agrobacterium inoculum contained in a 500-ml beaker. A vacuum (85 kPa) was applied and held for 5 min. Plants were removed from the desiccator and were covered with plastic bags in the dark for 24 h at room temperature. Plants were removed from the bags and returned to normal growth conditions within the greenhouse for seed formation (T1 generation of seed).

T1 seeds were screened by monitoring the expression of DsRed, a marker on the T-DNA in plasmid vectors pMBXS1168 or pMBXS1169 (FIGS. 9 and 10) allowing the identification of transgenic seeds. DsRed expression in the seed was visualized by fluorescent microscopy using a Nikon AZ100 microscope with a TRITC-HQ(RHOD)2 filter module (HQ545/30X, Q570LP, HQ610/75M) as previously described (Malik et al., 2015, Plant Biotechnology Journal, 13, 675). Between 20 and 32 T1 plants were produced from each transformation and progressed through to the T2 generation. T1 seeds were planted in soil and transgenic plants were obtained. Transgenic plant lines were further confirmed using PCR with primers specific to the gene of interest.

Overexpression of the Csa15g017550 gene in 3 independent transgenic lines each of pMBXO58/pMBXS1168 and pMBXO58/pMBXS1169, and two independent transgenic T2 lines each of pMBXS1168 and pMBXS1169 in the wild type Suneson background was determined by semi-quantitative RT-PCR. Overexpression of Csa15g017550 was observed (more transcript of the gene in transgenic lines) in 8 out of 10 lines. Expression of the CCP1 gene was also confirmed in the lines containing pMBXO58 (FIG. 3; SEQ ID NO: 19) along with the overexpression of the Csa15g017550 transcript. Lines were progressed to the T3 generation to generate homozygous Csa15g017550 lines. TABLE 8 shows the constructs and number of lines for each construct used in the subsequent yield testing experiments performed in a plant growth chamber.

TABLE 8
Homozygous T3 line grow-out in a plant growth chamber.

| | Vector | | | # of lines |
|---|---|---|---|---|
| Background | construct | Promoter/ | | selected for |
| Germplasm | transformed¹ | gene overexpressed² | Generation | advancement |
| Suneson/pMBXO58 | pMBXS1168 | 35S/CCP1 | T3 | 4 |
| (called NJ02) | | 35S/Csa15g017550 134 aa | | |
| Suneson/pMBXO58 | pMBXS1169 | 35S/CCP1 | T3 | 6 |
| (called NJ02) | | 35S/Csa15g017550 168 aa | | |
| Suneson | pMBXS1168 | 35S/Csa15g017550 134 aa | T3 | 6 |
| (called WTSU) | | | | |
| Suneson | pMBXS1169 | 35S/Csa15g017550 168 aa | T3 | 7 |
| (called WTSU) | | | | |

¹Sequence ID numbers for constructs are as follows: pMBXO58 (SEQ ID NO: 19); pMBXS1168 (SEQ ID NO: 17); pMBXS1169 (SEQ ID NO: 18).
²Csa15g017550 134 aa, Csa15g017550 with an ATG start codon producing a 134 aa peptide (SEQ ID NO: 2); Csa15g017550 168 aa, Csa15g017550 with a TTG start codon producing a 168 aa peptide (SEQ ID NO: 5).

### Growth Experiment 1

A yield experiment was carried out to evaluate the T3 homozygous Csa15g017550 lines shown in TABLE 8 under equal watering and an equalized fertilizer (100 ppm NPK 20-20-20) feed applied three times a week in Sunshine 4 potting mix. The plant growth chamber was set at a 16 hour photoperiod, a 22° C./18° C. day/night temperature, and ˜350-450 μmol/m2/see light intensity.

No differences were observed in Csa15g017550 and control plants during the first 10 days of plant development. However, as this experiment progressed the Csa15g017550 lines in all backgrounds showed improved vigor and also produced higher numbers of tertiary branches in the majority of Csa15g017550 plants as compared to the controls. As the study progressed further we also noted that Csa15g017550 expressing lines, in addition to having increased vigor and branching, had prolonged flowering. There was evidence of some sterility in several flowers of tertiary branches. The limited sterility on tertiary branches may be due to a limitation of resources available to the plant in a 6 inch (15 cm) pot. Overall the phenotype on the side branches of the Csa15g017550 plants looked healthier and more productive for seed yield. On completion of the growth experiment the following parameters were measured: plant heights were noted, the number of tertiary branches were determined, seed weight and 1000 seed weight was determined, and seed samples were prepared for oil content and oil composition analysis.

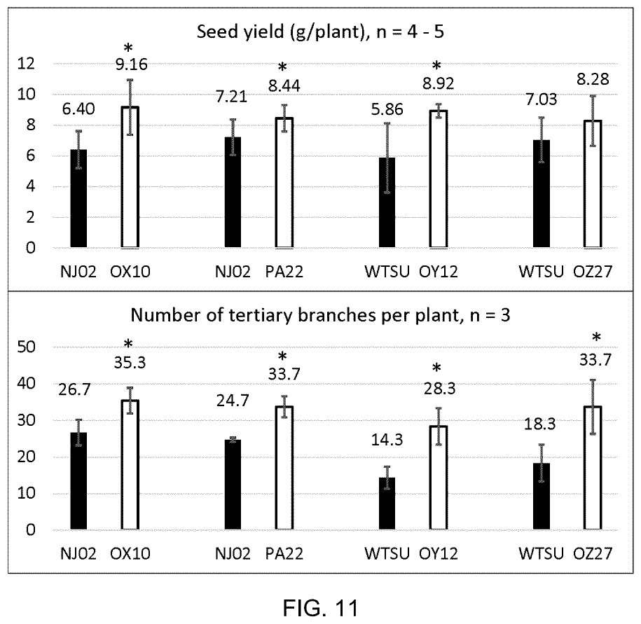

Total seed yield (g/plant) was significantly higher in several Csa15g017550 lines, with or without expression of CCP1, as compared to their background controls (TABLE 9). The highest yielding line was OY15, which expressed pMBXS1168 in the wild-type Suneson background, which produced 9.65±1.46 g of seed, a 65% increase over the 5.86±2.25 g of seed produced by the wild-type Suneson control. This increase in seed production was statistically significant (Student's t-test, p<0.05). Other seed yield related parameters (1000 seed weight, estimated total number of seeds per plant, and plant height at maturity) were also found to be generally increased in the Csa15g017550 lines. Analysis of total seed yield per line revealed that lines expressing Csa15g017550 with a significantly higher number of tertiary branches (40-100% more branching) compared to the controls also produced significantly higher seed yield (FIG. 11).

TABLE 9
T4 seed yield in lines of Camelina transformed
with Csa15g017550 expression constructs.

| Genetic | | | Number | Seed | | | |
|---|---|---|---|---|---|---|---|
| Construct for | Line | Line | of plants | Yield | Standard | % of | |
| Csa15g017550 | transformed¹ | Produced | analyzed | (g) | Deviation | control² | t-test |
| None | — | Wild-type | 4 | 5.86 | 2.25 | 100% | |
| | | Suneson | | | | | |
| pMBXS1168 | Wild-type | OY03 | 4 | 7.40 | 1.36 | 126% | 0.14740 |
| | Suneson | OY04 | 4 | 8.23 | 1.32 | 141% | 0.06517 |
| | | OY12 | 4 | 8.92 | 0.44 | 152%* | 0.03503 |
| | | OY15 | 4 | 9.65 | 1.46 | 165%* | 0.01793 |
| | | OY16 | 3 | 8.57 | 2.97 | 146% | 0.13152 |
| | | OY17 | 4 | 8.95 | 1.14 | 153%* | 0.03209 |
| None | — | NJ02 | 3 | 6.40 | 1.20 | 100% | |
| pMBXS1168 | NJ02 | OX10 | 4 | 9.16 | 1.78 | 143%* | 0.02385 |
| | | OX13 | 4 | 8.66 | 0.94 | 135%* | 0.01348 |
| | | OX20 | 4 | 7.83 | 0.80 | 122% | 0.05060 |
| | | OX21 | 3 | 6.61 | 1.67 | 103% | 0.43094 |
| None | — | Wild-type | 5 | 7.03 | 1.45 | 100% | |
| | | Suneson | | | | | |
| MBXS1169 | Wild-type | OZ05 | 4 | 8.31 | 1.47 | 118% | 0.11822 |
| | Suneson | OZ12 | 4 | 9.56 | 0.90 | 136%** | 0.00790 |
| | | OZ15 | 4 | 10.16 | 1.69 | 144%* | 0.01314 |
| | | OZ19 | 4 | 8.30 | 2.52 | 118% | 0.20825 |
| | | OZ21 | 4 | 9.72 | 1.55 | 138%* | 0.01787 |
| | | OZ26 | 4 | 9.20 | 3.13 | 131% | 0.13398 |
| | | OZ27 | 4 | 8.28 | 1.61 | 118% | 0.13666 |
| None | — | NJ02 | 5 | 7.21 | 1.14 | 100% | |
| pMBXS1169 | NJ02 | PA03 | 4 | 6.74 | 1.05 | 93% | 0.27115 |
| | | PA11 | 4 | 7.09 | 1.81 | 98% | 0.45568 |
| | | PA12 | 3 | 6.75 | 0.76 | 94% | 0.25838 |
| | | PA15 | 4 | 6.57 | 0.63 | 91% | 0.16023 |
| | | PA16 | 4 | 6.99 | 1.27 | 97% | 0.39467 |
| | | PA22 | 4 | 8.44 | 0.85 | 117%* | 0.05358 |

¹Line NJ02 contains pMBXO58 expressing CCP1 from the constitutive 35S promoter transformed into the wild-type Suneson background.
²Statistically significant results according to the student's t-test with a p < 0.05 are indicated with*, and P < 0.01 are indicated with**.

### Growth Experiment 2

A second growth experiment was performed with the same T3 generation seed lot of select lines ISY lines in the wild-type Suneson background. In the second experiment, growth conditions were different. The plants were grown in Sunshine 4 potting mix in 6″ pots with equalized fertilizer (100 ppm NPK 20-20-20) feed three times a week until the end of flowering in a Conviron BDR16 chamber set at 16 hrs photoperiod, 22° C./18° C. day/night temperature and ˜450-650 μmol/m²/see light intensity. The plants received additional water as needed in each pot. Most of the pMBXS1168 lines (OY04, OY12, OY15 and OY17) showed increased seedling vigor and improved overall plant growth compared to the wild type Suneson controls. pMBXS1169 lines (OZ lines) were generally comparable to the wild type at early vegetative stage.

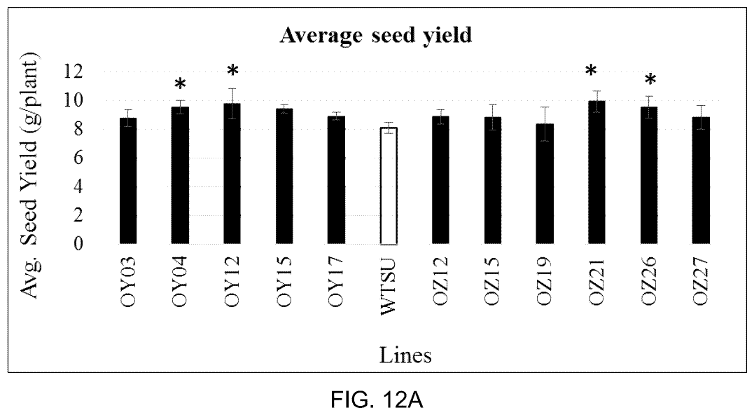

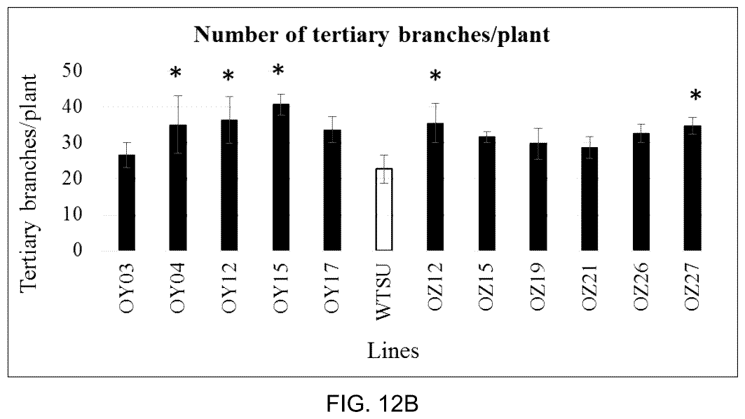

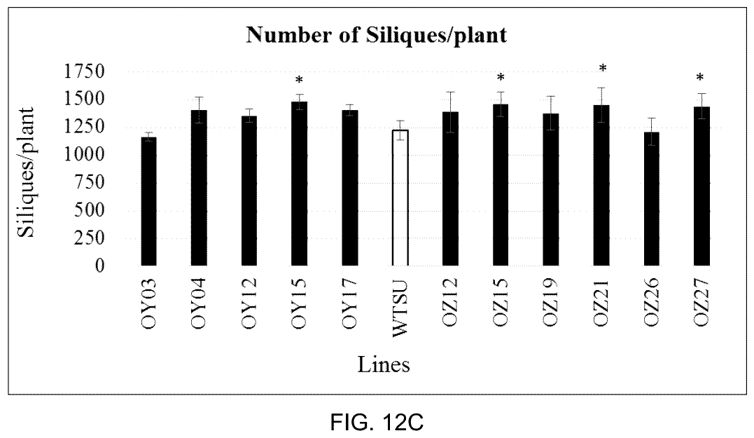

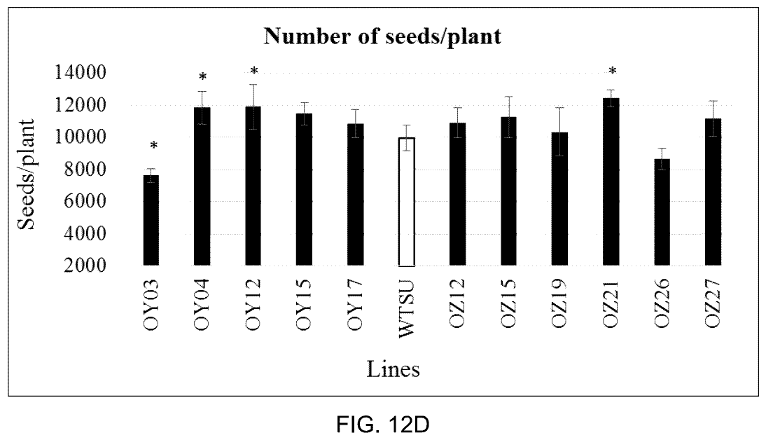

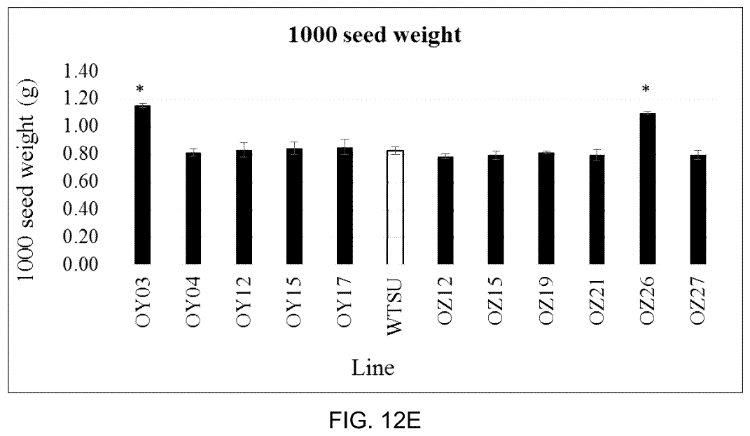

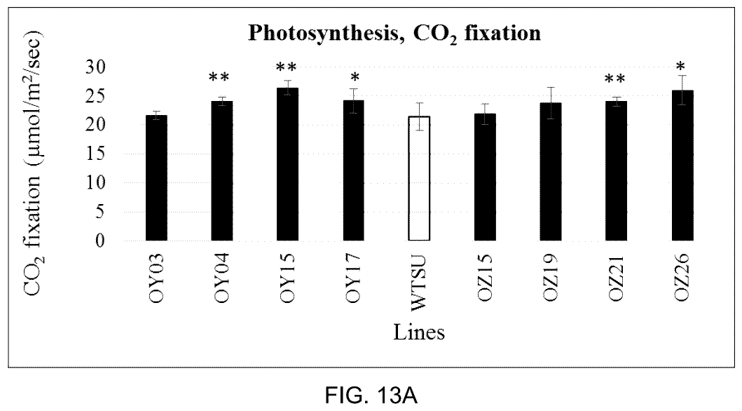

The seed yield of each plant was measured and average seed yield for each line (n=4 plants) was calculated (FIG. 12A). The best performing Suneson line transformed with construct pMBXS1168 (line OY12) produced 9.77±1.08 g of seed per plant, a 20.7% increase over the seed yield of the wild-type Suneson control. This increase was statistically significant (ANOVA & DMRT significant (p=0.05)). Line OY04 also had a statistically significant increase in seed yield producing 9.54±0.48 g of seed per plant, a 17.9% increase above the wild-type control. The best performing lines transformed with pMBXS1169 were OZ21 and OZ26 producing statistically significant increases in seed yield of 22.6% and 17.5%, respectively, compared to the wild-type control (ANOVA & DMRT significant (p=0.05)). Line OZ21 produced an average of 9.93±0.74 g of seed per plant whereas line OZ26 produced an average of 9.51±0.76 g of seed per plant.

The average number of tertiary branches per plant was determined for each line and increases were observed (FIG. 12B). Of the lines transformed with pMBXS1168, the lines with the most tertiary branches were lines OY04, OY12, and OY15 producing statistically significant increases in the number of tertiary branches of 53.9%, 59.7%, and 78.8% respectively, compared to the wild-type control (ANOVA & DMRT significant (p=0.05)). Line OY04 produced 35.00±8.00 branches, line OY12 produced 36.33±6.43 branches, and the Suneson control line produced 22.80±3.94 branches. The lines of pMBXS1169 with the highest number of tertiary branches were OZ12 and OZ27 which were statistically significant with 55.7% and 52.0% more tertiary branches, respectively, compared to the wild-type control (ANOVA & DMRT significant (p=0.05)). Line OZ12 produced 35.50±5.45 branches whereas line OZ27 produced 34.67±2.31 branches.

The average number of siliques per plant was determined for each line and increases were observed (FIG. 12C). Of the lines transformed with pMBXS1168, the line with the most siliques was line OY15 producing 1480.0±68.4 siliques, a statistically significant increase of 21.3% compared to the wild-type control which had 1220.0±86.8 siliques (ANOVA & DMRT significant (p=0.05)). Lines transformed with pMBXS1169 also had statistically significant numbers of siliques (FIG. 12C).

The average number of seeds per plant was also determined (FIG. 12D). The best producing lines transformed with pMBXS1168 were lines OY04 and OY12 which produced statistically significant increases in seed per plant of 19.0% and 19.4%, respectively. Line OY04 had 11,832±1,007 seed per plant whereas line OY12 had 11,879±1,374 seed per plant. The wild-type Suneson control plant had 9,946±779 seeds per plant.

The individual seed weight, as determined by measuring the mass of 1000 seeds which was sampled using a seed counter, was also determined (FIG. 12E). Lines with a 1000 seed weight close to the wild-type control (FIG. 12E) in general had an increase in the number of seeds per plant compared to wild-type (FIG. 12D). There were some lines where there was a tradeoff between the number of seeds per plant and the individual seed weight. Lines OY03 (transformed with pMBXS1168) and OZ26 (transformed with pMBXS1169) with the highest average 1000 seed weight also possessed the least number of seeds per plant (FIG. 12D, E).

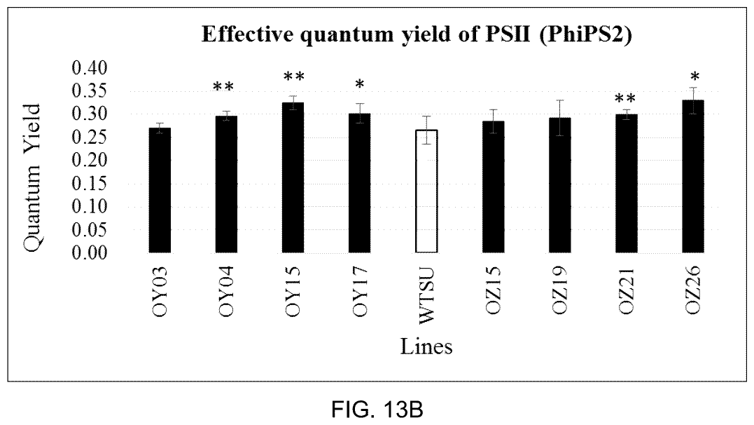

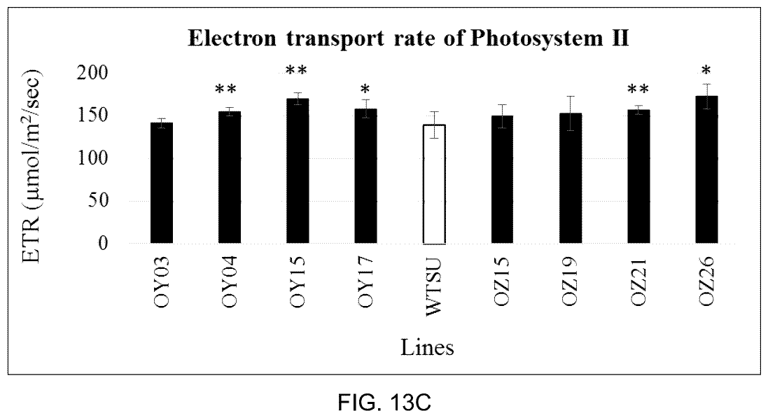

T4 generation seeds of select C3004 plants were planted in a field trial in Saskatoon, Canada, during the 2019 growth season. Photosynthesis measurements were performed on T4 generation plants at a pre-bolting stage about 3.5 weeks after germination using a LI-6400XT Portable Photosynthesis System (LI-COR Inc., Lincoln, NE, USA). Combined photosynthesis and light adapted chlorophyll fluorescence measurements were recorded from fully developed young leaves of each line and the corresponding wild type. This allowed the determination of the net photosynthetic assimilation, electron transfer rate (actual flux of photons driving the PSII) and the quantum yield efficiency (PhiPS2) of the photosystem II (PSII). Camelina sativa cv. Suneson lines transformed with pMBXS1168 that had statistically significant increases in photosynthetic parameters for CO₂ fixation (FIG. 13A), effective quantum yield of Photosystem II (PSII) (FIG. 13B), and electron transport rate (ETR) of PSII (FIG. 13C) included lines OY04, OY15, and OY17 (TABLE 10). Suneson lines transformed with pMBXS1169 that had statistically significant increases in photosynthetic parameters for CO₂ fixation (FIG. 13A), effective quantum yield of Photosystem II (PSII) (FIG. 13B), and electron transport rate (ETR) around PSII (FIG. 13C) included lines OZ21 and OZ26 (TABLE 10). These results indicate a higher proportion of absorbed light used in photochemistry and improved overall photosynthesis. The C3004 lines showing higher photosynthetic efficiencies under field growth condition had higher yields during growth experiments 1 and 2 described above.

TABLE 10
Percent increases in photosynthetic parameters for Camelina sativa
cv. Suneson transformed with either pMBXS1168 or pMBXS1169.

| | | | % Increase | % Increase |
|---|---|---|---|---|
| | | % Increase | in Effective | in Electron |
| | | in Photosynthesis | Quantum | Transport |
| Line | Vector | (CO₂ fixation) | Yield of PSII | Rate of PSII |
| OY04 | pMBXS1168 | 12** | 12** | 12** |
| OY15 | pMBXS1168 | 23** | 23** | 22** |
| OY17 | pMBXS1168 | 12* | 14* | 14* |
| WTSU | — | — | — | — |
| OZ21 | pMBXS1169 | 12** | 13** | 13** |
| OZ26 | pMBXS1169 | 21* | 24* | 24* |

¹Only lines with statistically significant increases are shown. Data analyzed by Student's t-tests.
**, *= t-test significant at p ≤ 0.01 and p ≤ 0.05, respectively

Overall, increased expression of Csa15g017550 in Camelina results in a significant increase in plant growth vigor, number of branches, number of siliques, seed yield, and some photosynthetic parameters. These results are the opposite of what would be predicted based on the hypothesis that Csa15g017550 may be a plant invertase inhibitor gene. In addition with the limited number of plant lines tested for each vector in each genetic background, there is some evidence that the gene encoding the 134 aa ISY protein may be more effective plus or minus the CCP1 gene and hence may be a preferred embodiment. These results also support the theory that Csa15g017550 does not encode a plant invertase inhibitor.

### Example 5. Overexpressing the Camelina ISY Gene in Other Crops: Canola, Soybean, Corn, Wheat, Rice, Potato, Tomato Etc.

For dicot plants, vectors pMBXS1168 and pMBXS1169 can be modified to include a selectable marker appropriate for selection of transformants for the crop of interest.

### Canola Transformation.

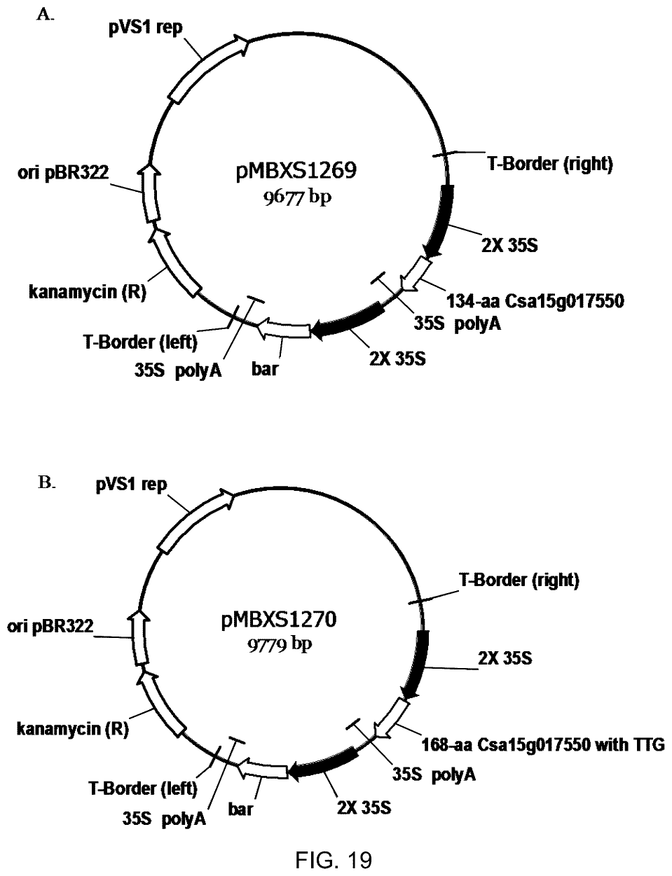

For transformation of canola, vectors pMBXS1168 and pMBXS1169 can be modified to include a cassette for expression of the bar gene, providing resistance to the herbicide bialaphos. Construct pMBXS1269 (FIG. 19A, SEQ ID NO: 197) was prepared that contains the Camelina ISY gene (SEQ ID NO: 1) encoding the 134 amino acid ISY protein (SEQ ID NO: 2) expressed from the double enhanced CaMV35S constitutive promoter (2× 35S). Construct pMBXS1270 (FIG. 19B, SEQ ID NO: 198) was prepared that contains the Camelina ISY gene (SEQ ID NO: 6) encoding the 168 amino acid Camelina ISY protein (SEQ ID NO: 5) expressed from the double enhanced CaMV35S constitutive promoter (2× 35S). An expression cassette for the bar gene, driven by the double enhanced CaMV35S constitutive promoter (2× 35S), was included for selection. Both constructs were transformed into canola.

In preparation for plant transformation experiments, seeds of Brassica napus cv DH12075 (obtained from Agriculture and Agri-Food Canada) were surface sterilized with sufficient 95% ethanol for 15 seconds, followed by 15 minutes incubation with occasional agitation in full strength Javex (or other commercial bleach, 7.4% sodium hypochlorite) and a drop of wetting agent such as Tween 20. The Javex solution was decanted and 0.025% mercuric chloride with a drop of Tween 20 was added and the seeds were sterilized for another 10 minutes. The seeds were then rinsed three times with sterile distilled water. The sterilized seeds were plated on half strength hormone-free Murashige and Skoog (MS) media (Murashige T, Skoog F (1962). Physiol Plant 15:473-498) with 1% sucrose in 15×60 mm petri dishes that were then placed, with the lid removed, into a larger sterile vessel (Majenta GA7 jars). The cultures were kept at 25° C., with 16 h light/8h dark, under approximately 70-80 μE of light intensity in a tissue culture cabinet. 4-5 days old seedlings were used to excise fully unfolded cotyledons along with a small segment of the hypocotyl. Excisions were made so as to ensure that no part of the apical meristem was included.

Separate Agrobacterium strains of GV3101 (pMP90) carrying either pMBXS1269 (FIG. 19A, SEQ ID NO: 197) or pMBXS1270 (FIG. 19B, SEQ ID NO: 198) were prepared and were grown overnight in 5 ml of LB media with 50 mg/L kanamycin, gentamycin, and rifampicin. The culture was centrifuged at 2000 g for 10 min., the supernatant was discarded and the pellet was suspended in 5 ml of inoculation medium (Murashige and Skoog with B5 vitamins (MS/B5; Gamborg OL, Miller RA, Ojima K. Exp Cell Res 50:151-158), 3% sucrose, 0.5 mg/L benzyl aminopurine (BA), pH 5.8). Cotyledons were collected in Petri dishes with ˜1 ml of sterile water to keep them from wilting. The water was removed prior to inoculation and explants were inoculated in a mixture of 1 part Agrobacterium suspension and 9 parts inoculation medium in a final volume sufficient to bathe the explants. After explants were well exposed to the Agrobacterium solution and inoculated, a pipet was used to remove any extra liquid from the petri dishes.

The Petri plates containing the explants incubated in the inoculation media were sealed and kept in the dark in a tissue culture cabinet set at 25° C. After 2 days the cultures were transferred to 4° C. and incubated in the dark for 3 days. The cotyledons, in batches of 10, were then transferred to selection medium consisting of Murashige Minimal Organics (Sigma), 3% sucrose, 4.5 mg/L BA, 500 mg/L MES, 27.8 mg/L Iron (II) sulfate heptahydrate, pH 5.8, 0.7% Phytagel with 300 mg/L timentin, and 2 mg/L L-phosphinothricin (L-PPT) added after autoclaving. The cultures were kept in a tissue culture cabinet set at 25° C., 16 h/8 h, with a light intensity of about 125 μmol m⁻² s⁻¹. The cotyledons were transferred to fresh selection every 3 weeks until shoots were obtained. The shoots were excised and transferred to shoot elongation media containing MS/B5 media, 2% sucrose, 0.5 mg/L BA, 0.03 mg/L gibberellic acid (GA₃), 500 mg/L 4-morpholineethanesulfonic acid (MES), 150 mg/L phloroglucinol, pH 5.8, 0.9% Phytagar and 300 mg/L timentin and 3 mg/L L-phosphinothricin added after autoclaving. After 3-4 weeks any callus that was formed at the base of shoots with normal morphology was cut off and shoots were transferred to rooting media containing half strength MS/B5 media with 1% sucrose and 0.5 mg/L indole butyric acid, 500 mg/L MES, pH 5.8, 0.8% agar, with 1.5 mg/L L-PPT and 300 mg/L timentin added after autoclaving. The plantlets with healthy shoots were hardened and transferred to 6 inch (15 cm) pots in the greenhouse and T1 transgenic seeds were collected.

For genetic construct pMBXS1269, 22 TO lines were isolated and grown to maturity to harvest T1 seeds. T1 seeds of 6 single copy lines, 2 double copy lines and 1 multi-copy lines are grown in a greenhouse to identify homozygous transgenic plants of pMBXS1269.

For genetic construct pMBXS1270, 32 TO lines were isolated and grown to maturity to harvest T1 seeds. T1 seeds of 11 single copy lines are grown in a greenhouse to identify homozygous transgenic plants of pMBXS1270.

Screening of transgenic plants of canola expressing the ISY gene to identify plants with higher yield is performed as follows. T2 generation of seed from each line is harvested. Seed yield from each plant is determined by harvesting all of the mature seeds from a plant and drying them in an oven with mechanical convection set at 22° C. for two days. The weight of the entire harvested seed is recorded. The 100 seed weight is measured to obtain an indication of seed size. The oil content of seeds is measured using published procedures for preparation of fatty acid methyl esters (Malik et al. 2015, Plant Biotechnology Journal, 13, 675-688).

### Soybean Transformation.

For transformation of soybean, the expression cassettes for the ISY gene can be excised from either vector pMBXS1168 or pMBXS1169. Transformation of soybean can be performed with the expression cassette for the ISY gene of choice and an expression cassette for the selectable marker hygromycin resistance as follows. The purified DNA cassettes are co-bombarded using biolistics into embryogenic cultures of soybean Glycine max cultivars X5 and Westag97, to obtain transgenic plants. The hygromycin resistance gene is expressed from a plant promoter, such as the soybean actin promoter (TABLE 1; SEQ ID NO: 44) and is flanked by a suitable 3′ UTR, such as the 3′ UTR from the soybean actin gene (soybean actin Gene ID Glyma.19G147900). The ISY gene can also be expressed from the soybean actin promoter and flanked by the 3′ UTR of the soybean actin gene in cases where it is useful to use only DNA sequences from soybean for regulatory approval purposes under USDA-APHIS in the United States.

The transformation, selection, and plant regeneration protocol is adapted from Simmonds (2003) (Simmonds, 2003, Genetic Transformation of Soybean with Biolistics. In: Jackson J F, Linskens HF (eds) Genetic Transformation of Plants. Springer Verlag, Berlin, pp 159-174) and is performed as follows.

Induction and Maintenance of Proliferative Embryogenic Cultures: Immature pods, containing 3-5 mm long embryos, are harvested from host plants grown at 28/24° C. (day/night), 15-h photoperiod at a light intensity of 300-400 μmol m⁻² s⁻¹. Pods are sterilized for 30 s in 70% ethanol followed by 15 min in 1% sodium hypochlorite (with 1-2 drops of Tween 20 (Sigma, Oakville, ON, Canada)) and three rinses in sterile water. The embryonic axis is excised and explants are cultured with the abaxial surface in contact with the induction medium (MS salts, B5 vitamins (Gamborg OL, Miller RA, Ojima K. Exp Cell Res 50:151-158), 3% sucrose, 0.5 mg/L BA, pH 5.8), 1.25-3.5% glucose (concentration varies with genotype), 20 mg/l 2,4-D, pH 5.7). The explants, maintained at 20° C. at a 20-h photoperiod under cool white fluorescent lights at 35-75 μmol m⁻² s⁻¹, are sub-cultured four times at 2-week intervals. Embryogenic clusters, observed after 3-8 weeks of culture depending on the genotype, are transferred to 125-ml Erlenmeyer flasks containing 30 ml of embryo proliferation medium containing 5 mM asparagine, 1-2.4% sucrose (concentration is genotype dependent), 10 mg/l 2,4-D, pH 5.0 and cultured as above at 35-60 μmol m⁻² s⁻¹ of light on a rotary shaker at 125 rpm. Embryogenic tissue (30-60 mg) is selected, using an inverted microscope, for subculture every 4-5 weeks.

Transformation: Cultures are bombarded 3 days after subculture. The embryogenic clusters are blotted on sterile Whatman filter paper to remove the liquid medium, placed inside a 10×30-mm Petri dish on a 2×2 cm² tissue holder (PeCap, 1005 μm pore size, Band SH Thompson and Co. Ltd. Scarborough, ON, Canada) and covered with a second tissue holder that is then gently pressed down to hold the clusters in place. Immediately before the first bombardment, the tissue is air dried in the laminar air flow hood with the Petri dish cover off for no longer than 5 min. The tissue is turned over, dried as before, bombarded on the second side and returned to the culture flask. The bombardment conditions used for the Biolistic PDS-I000/He Particle Delivery System are as follows: 737 mm Hg chamber vacuum pressure, 13 mm distance between rupture disc (Bio-Rad Laboratories Ltd., Mississauga, ON, Canada) and macrocarrier. The first bombardment uses 900 psi rupture discs and a microcarrier flight distance of 8.2 cm, and the second bombardment uses 1100 psi rupture discs and 11.4 cm microcarrier flight distance. DNA precipitation onto 1.0 μm diameter gold particles is carried out as follows: 2.5 μl of 100 ng/μl of DNA containing the expression cassette for the ISY gene from either pMBXS1168 or pMBXS1169, and 2.5 μl of 100 ng/μl selectable marker DNA (cassette for hygromycin selection) are added to 3 mg gold particles suspended in 50 μl sterile dH₂O and vortexed for 10 sec; 50 μl of 2.5 M CaCl₂ is added, vortexed for 5 sec, followed by the addition of 20 μl of 0.1 M spermidine which is also vortexed for 5 sec. The gold is then allowed to settle to the bottom of the microfuge tube (5-10 min) and the supernatant fluid is removed. The gold/DNA is resuspended in 200 μl of 100% ethanol, allowed to settle and the supernatant fluid is removed. The ethanol wash is repeated and the supernatant fluid is removed. The sediment is resuspended in 120 μl of 100% ethanol and aliquots of 8 μl are added to each macrocarrier. The gold is resuspended before each aliquot is removed. The macrocarriers are placed under vacuum to ensure complete evaporation of ethanol (about 5 min).

Selection: The bombarded tissue is cultured on embryo proliferation medium described above for 12 days prior to subculture to selection medium (embryo proliferation medium contains 55 mg/l hygromycin added to autoclaved media). The tissue is sub-cultured 5 days later and weekly for the following 9 weeks. Green colonies (putative transgenic events) are transferred to a well containing 1 ml of selection media in a 24-well multi-well plate that is maintained on a flask shaker as above. The media in multi-well dishes is replaced with fresh media every 2 weeks until the colonies are approximately 2-4 mm in diameter with proliferative embryos, at which time they are transferred to 125 ml Erlenmeyer flasks containing 30 ml of selection medium. A portion of the proembryos from transgenic events is harvested to examine gene expression by RT-PCR.

Plant regeneration: Maturation of embryos is carried out, without selection, at conditions described for embryo induction. Embryogenic clusters are cultured on Petri dishes containing maturation medium (MS salts, B5 vitamins, 6% maltose, 0.2% gelrite gellan gum (Sigma), 750 mg/l MgCl₂, pH 5.7) with 0.5% activated charcoal for 5-7 days and without activated charcoal for the following 3 weeks. Embryos (10-15 per event) with apical meristems are selected under a dissection microscope and cultured on a similar medium containing 0.6% phytagar (Gibco, Burlington, ON, Canada) as the solidifying agent, without the additional MgCl₂, for another 2-3 weeks or until the embryos become pale yellow in color. A portion of the embryos from transgenic events after varying times on gelrite are harvested to examine gene expression by RT-PCR.

Mature embryos are desiccated by transferring embryos from each event to empty Petri dish bottoms that are placed inside Magenta boxes (Sigma) containing several layers of sterile Whatman filter paper flooded with sterile water, for 100% relative humidity. The Magenta boxes are covered and maintained in darkness at 20° C. for 5-7 days. The embryos are germinated on solid B5 medium containing 2% sucrose, 0.2% gelrite and 0.075% MgCl₂ in Petri plates, in a chamber at 20° C., 20-h photoperiod under cool white fluorescent lights at 35-75 μmol m⁻² s⁻¹. Germinated embryos with unifoliate or trifoliate leaves are planted in artificial soil (Sunshine Mix No. 3, SunGro Horticulture Inc., Bellevue, WA, USA), and covered with a transparent plastic lid to maintain high humidity. The flats are placed in a controlled growth cabinet at 26/24° C. (day/night), 18 h photoperiod at a light intensity of 150 μmol m⁻² s⁻¹ At the 2-3 trifoliate stage (2-3 weeks), the plantlets with strong roots are transplanted to pots containing a 3:1:1:1 mix of ASB Original Grower Mix (a peat-based mix from Greenworld, ON, Canada):soil: sand: perlite and grown at 18-h photoperiod at a light intensity of 300-400 μmolm⁻² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

The selectable marker can be removed by segregation if desired by identifying co-transformed plants that have not integrated the selectable marker expression cassette and the ISY gene cassette into the same locus. Plants are grown, allowed to set seed and germinated. Leaf tissue is harvested from soil grown plants and screened for the presence of the selectable marker cassette. Plants containing only the ISY gene expression cassette are advanced.

### Corn Transformation

An expression cassette for the ISY gene can be constructed using a variety of different promoters for expression. Candidate constitutive and seed-specific promoters for use in monocots including corn are listed in TABLE 2, however those skilled in the art will understand that other promoters can be selected for expression.

In some instances, it may be advantageous to create a hybrid promoter containing a promoter sequence and an intron. These promoters can deliver higher levels of stable expression. Examples of such hybrid promoters include the hybrid maize Cab-m5 promoter/maize hsp70 intron (SEQ ID NO: 64, TABLE 2) and the maize ubiquitin promoter/maize ubiquitin intron (SEQ ID NO: 109 and 110, TABLE 2).

An example expression cassette for the ISY gene for maize includes the genetic elements in TABLE 11, in which the promoter is operably linked to the ISY gene which is operably linked to the termination sequence.

TABLE 11
Example transformation cassette for
the Camelina ISY gene for maize

| Promoter | Gene | Terminator |
|---|---|---|
| hybrid maize Cab-m5 | ISY gene encoding the | Hsp70 3′ UTR |
| promoter/maize hsp70 | 134 amino acid protein | Glyma.02G093200* |
| intron (SEQ ID NO: 64) | (SEQ ID NO: 1) | |

*GENE ID Glyma.02G093200 includes sequence information for coding regions as well as associated promoters, 5′ UTRs, and 3′ UTRs (available at Phytozome, see JGI website phytozome.jgi.doe.gov/pz/portal.html).

Methods to transform the expression cassette described in TABLE 11 into maize are routine and well known in the art and have recently been reviewed by Que et al., (2014), Frontiers in Plant Science 5, article 379, pp 1-19.

Protoplast transformation methods useful for practicing the invention are well known to those skilled in the art. Such procedures include for example the transformation of maize protoplasts as described by Rhodes and Gray (Rhodes, C. A. and D. W. Gray, Transformation and regeneration of maize protoplasts, in Plant Tissue Culture Manual: Supplement 7, K. Lindsey, Editor. 1997, Springer Netherlands: Dordrecht. p. 353-365). For protoplast transformation of maize, the expression cassette described in TABLE 11 can be co-bombarded with an expression cassette for a selectable marker, such as the bar gene imparting transgenic plants resistance to bialaphos.

For Agrobacterium-mediated transformation of maize, the expression cassette described in TABLE 11 can be inserted into a binary vector that also contains an expression cassette for a selectable marker, such as the bar gene. The binary vector is transformed into an Agrobacterium tumefaciens strain, such as A. tumefaciens strain EHA101.

Agrobacterium-mediated transformation of maize can be performed following a previously described procedure (Frame et al. (2006), Agrobacterium Protocols, Wang K., ed., Vol. 1, pp 185-199, Humana Press) as follows.

Plant Material: Plants grown in a greenhouse are used as an explant source. Ears are harvested 9-13 days after pollination and surface sterilized with 80% ethanol.

Explant Isolation, Infection and Co-Cultivation: Immature zygotic embryos (1.2-2.0 mm) are aseptically dissected from individual kernels and incubated in an A. tumefaciens strain EHA101 culture containing the transformation vector of interest for genome editing (grown in 5 ml N6 medium supplemented with 100 μM acetosyringone for stimulation of the bacterial vir genes for 2-5 h prior to transformation) at room temperature for 5 min. The infected embryos are transferred scutellum side up on to a co-cultivation medium (N6 agar-solidified medium containing 300 mg/l cysteine, 5 μM silver nitrate and 100 μM acetosyringone) and incubated at 20° C., in the dark for 3 d. Embryos are transferred to N6 resting medium containing 100 mg/l cefotaxime, 100 mg/l vancomycin and 5 μM silver nitrate and incubated at 28° C., in the dark for 7 d.

Callus Selection: All embryos are transferred on to the first selection medium (the resting medium described above supplemented with 1.5 mg/l bialaphos) and incubated at 28° C. in the dark for 2 weeks followed by subculture on a selection medium containing 3 mg/l bialaphos. Proliferating pieces of callus are propagated and maintained by subculture on the same medium every 2 weeks.

Plant Regeneration and Selection: Bialaphos-resistant embryogenic callus lines are transferred on to regeneration medium I (MS basal medium supplemented with 60 g/l sucrose, 1.5 mg/l bialaphos and 100 mg/l cefotaxime and solidified with 3 g/l Gelrite) and incubated at 25° C. in the dark for 2 to 3 weeks. Mature embryos formed during this period are transferred on to regeneration medium II (the same as regeneration medium I with 3 mg/l bialaphos) for germination in the light (25° C., 80-100 μmol/m²/s light intensity, 16/8-h photoperiod). Regenerated plants are ready for transfer to soil within 10-14 days. Plants are grown in the greenhouse to maturity and T1 seeds are isolated.

The copy number of the transgene insert is determined, through methods such as Southern blotting or digital PCR, and lines are selected to bring forward for further analysis. Overexpression of the ISY gene is determined by RT-PCR and/or Western blotting techniques and plants with the desired level of expression are selected. Homozygous lines are generated. The yield seed of homozygous lines is compared to control lines.

### Rice Transformation

Candidate constitutive and seed-specific promoters for use in monocots can be used in the expression cassettes for the ISY gene in rice and are listed in TABLE 2. Those skilled in the art will understand that other promoters can be selected for expression.

The example expression cassette in TABLE 11 can be used to transform the ISY gene into rice. The expression cassette can be inserted into a binary vector that also contains an expression cassette for a selectable marker, such as the hygromycin selectable marker. The binary vector is transformed into an Agrobacterium tumefaciens AGL1 strain.

In preparation for rice transformation, callus of the rice cultivar Nipponbare is initiated from mature, dehusked, surface sterilized seeds on N6-basal salt callus induction media (N6-CI; contains per liter 3.9 g CHU (N6) basal salt mix (Sigma Catalog #C1416); 10 ml of 100× N6-vitamins (contains in final volume of 500 mL, 100 mg glycine, 25 mg nicotinic acid, 25 mg pyridoxine hydrochloride and 50 mg thiamin hydrochloride); 0.1 g myo-inositol; 0.3 g casamino acid (casein hydrolysate); 2.88 g proline; 10 ml of 100× 2,4-D, 30 g sucrose, pH 5.8 with 4 g gelrite or phytagel). Approximately 100 seeds are used for each transformation. The frequency of callus induction is scored after 21 days of culture in the dark at 27±1° C. Callus induction from the scutellum with a high frequency (of about 96% total callus induction) is observed.

The rice transformation vector is transformed into Agrobacterium strain AGL1. The resulting Agrobacterium strain is resuspended in 10 mL of MG/L medium (5 g tryptone, 2.4 g yeast extract, 5 g mannitol, 5 g Mg₂SO₄, 0.25 g K₂HPO₄, 1 g glutamic acid and 1 g NaCl) to a final OD600 of 0.3. Approximately twenty-one day old scutellar embryogenic callus are cut to about 2-3 mm in size and are infected with Agrobacterium containing the transformation vector for 5 min. After infection, the calli are blotted dry on sterile filter papers and transferred onto co-cultivation media (N6-CC; contains per liter 3.9 g CHU (N6) basal salt mix; 10 ml of 100× N6-vitamins; 0.1 g myo-inositol; 0.3 g casamino acid; 10 ml of 100× 2,4-D, 30 g sucrose, 10 g glucose, pH 5.2 with 4 g gelrite or phytagel and 1 mL of acetosyringone (19.6 mg/mL stock)). Co-cultivated calli are incubated in the dark for 3 days at 25° C. After three days of co-cultivation, the calli are washed thoroughly in sterile distilled water to remove the bacteria. A final wash with a timentin solution (250 mg/L) is performed and calli are blotted dry on sterile filter paper. Callus are transferred to selection media (N6-SH; contains per liter 3.9 g CHU (N6) basal salt mix, 10 ml of 100× N6-vitamins, 0.1 g myo-inositol, 0.3 g casamino acid, 2.88 g proline, 10 ml of 100×, 2,4-D, 30 g sucrose, pH 5.8 with 4 g phytagel and 500 μL of hygromycin (stock concentration: 100 mg/ml) and incubated in the dark for two-weeks at 27±1° C. The transformed calli that survive the selection pressure and that proliferate on N6-SH medium are sub-cultured on the same media for a second round of selection. These calli are maintained under the same growth conditions for another two weeks. The number of plants regenerated after 30 days on N6-SH medium is scored and the frequency calculated. After 30 days, the proliferating calli are transferred to regeneration media (N6-RH medium; contains per liter 4.6 g MS salt mixture, 10 ml of 100× MS-vitamins (MS-vitamins contains in 500 mL final volume 250 mg nicotinic acid, 500 mg pyridoxine hydrochloride, 500 mg thiamine hydrochloride, 100 mg glycine), 0.1 g myo-inositol, 2 g casein hydrolysate, 1 ml of 1,000×1-naphtylacetic acid solution (NAA; contains in 200 mL final volume 40 mg NAA and 3 mL of 0.1 N NaOH), 20 ml of 50× kinetin (contains in 500 mL final volume 50 mg kinetin and 20 mL 0.1 N HCl), 30 g sucrose, 30 g sorbitol, pH 5.8 with 4 g phytagel and 500 μl of a 100 mg/mL hygromycin stock). The regeneration of plantlets from these calli occurs after about 4-6 weeks. Rooted plants are transferred into peat-pellets for one week to allow for hardening of the roots. The plants are then kept in sealable bags for acclimatization. Plants are transferred into pots and grown in a greenhouse to maturity.

Seed is harvested from each panicle (T1 generation) and the seed yield per plant is calculated. T1 seed is grown in a greenhouse to produce T2 seed. The mass of the total seed per plant is collected to compare seed yield of transgenics to wild-type control plants.

### Example 6. Identification of Homologs and/or Orthologs of the Camelina ISY in Other Crop Species

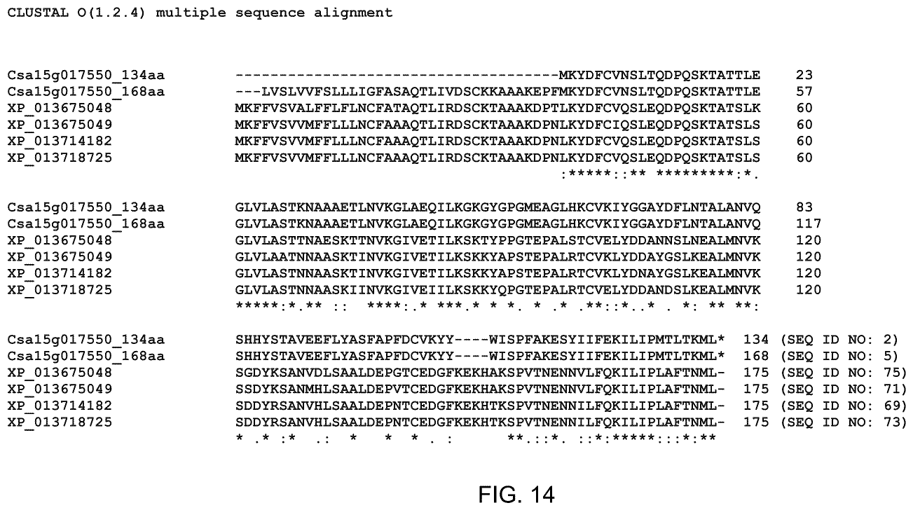

The Brassica napus genome of the cultivar ZS11 was searched for orthologs of the Camelina ISY gene using BLAST searches. The best four orthologs are listed in TABLE 12 with calculations of their percent homology to the Csa15g017550.1 gene encoding both 134 (SEQ ID NO: 2) and 168 (SEQ ID NO: 5) amino acid proteins. A CLUSTAL alignment is shown in FIG. 14.

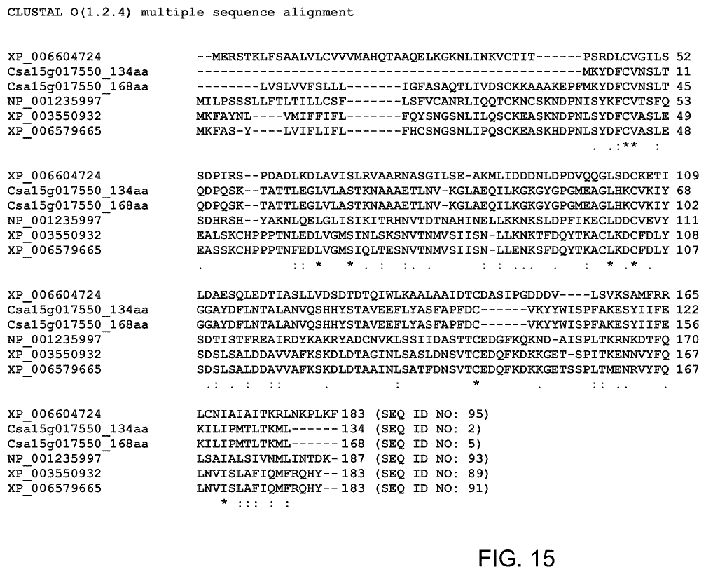

The soybean reference genome (Glycine max Williams 82) was searched for orthologs of the Camelina ISY gene using BLAST searches. The best four orthologs are listed in TABLE 12 with calculations of their percent homology to the Csa15g017550.1 gene encoding both 134 (SEQ ID NO: 2) and 168 (SEQ ID NO: 5) amino acid proteins. A CLUSTAL alignment is shown in FIG. 15.

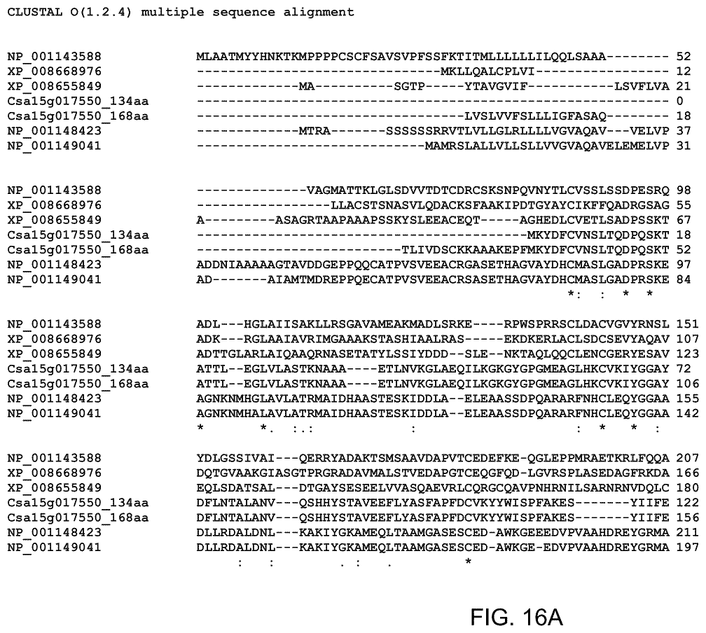

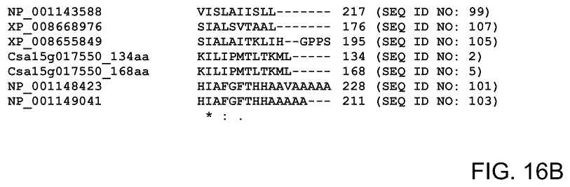

The corn reference genome (Zea mays B73) was searched for orthologs of the Camelina ISY gene using BLAST searches. The best 5 orthologs are listed in TABLE 12 with calculations of their percent homology to the Csa15g017550.1 gene encoding both 134 (SEQ ID NO: 2) and 168 (SEQ ID NO: 5) amino acid proteins. A CLUSTAL alignment is shown in FIG. 16.

A CLUSTAL alignment of the 134 (SEQ ID NO: 2) and 168 (SEQ ID NO: 5) amino acid proteins of Csa15g017550 with the canola, soybean, and corn orthologs listed in TABLE 12 is shown in FIG. 17. Conserved residues in all proteins include a cysteine at residue 40, a leucine at residue 59, a cysteine at residue 98, and a cysteine at residue 138, all based on the numbering of the Csa15g017550 168 amino acid protein (SEQ ID NO: 5). In addition, a cysteine is conserved at residue 25 (based on numbering of Csa15g017550 168 amino acid protein, SEQ ID NO: 5) for all proteins except the truncated Csa15g017550 134 amino acid protein (SEQ ID NO: 2).

TABLE 12
Orthologs to Camelina Csa15g017550 gene in Canola, Soybean and Corn.

| | | | Size of | % homology | % homology |
|---|---|---|---|---|---|
| | | Protein ID and/or | encoded | to protein | to protein |
| | | SEQ ID NO of | protein | in SEQ ID | in SEQ ID |
| Crop | Gene ID¹ | Protein | (amino acids) | NO: 2² | NO: 5³ |
| Camelina | Csa15g017550.1 | SEQ ID NO: 2 | 134 | 100 | 79.8 |
| | (SEQ ID NO: 1) | | | | |
| Camelina | Csa15g017550.1 | SEQ ID NO: 5 | 168 | 79.8 | 100 |
| | (SEQ ID NO: 6) | | | | |
| canola | XM_013858728.2 | XP_013714182 | 175 | 41.1 | 54.3 |
| | (SEQ ID NO: 68) | (SEQ ID NO: 69) | | | |
| canola | XM_013819595.2 | XP_013675049 | 175 | 39.4 | 52.6 |
| | (SEQ ID NO: 70) | (SEQ ID NO: 71) | | | |
| canola | XM_013863271 | XP_013718725 | 175 | 40.6 | 53.7 |
| | (SEQ ID NO: 72) | (SEQ ID NO: 73) | | | |
| canola | XM_013819594.2 | XP_013675048 | 188 | 40.0 | 52.0 |
| | (SEQ ID NO: 74) | (SEQ ID NO: 75) | | | |
| soybean | XM_003550884 | XP_003550932.1 | 183 | 18.0 | 23.0 |
| | (SEQ ID NO: 88) | (SEQ ID NO: 89) | | | |
| soybean | XM_006579602 | XP_006579665.1 | 183 | 16.4 | 22.4 |
| | (SEQ ID NO: 90) | (SEQ ID NO: 91) | | | |
| soybean | NM_001249068.2 | NP_001235997 | 187 | 17.6 | 22.5 |
| | (SEQ ID NO: 92) | (SEQ ID NO: 93) | | | |
| soybean | XM_006604661 | XP_006604724 | 183 | 15.3 | 16.4 |
| | (SEQ ID NO: 94) | (SEQ ID NO: 95) | | | |
| corn | NM_001150116 | NP_001143588 | 217 | 13.8 | 15.6 |
| | (SEQ ID NO: 98) | (SEQ ID NO: 99) | | | |
| corn | NM_001154951 | NP_001148423 | 228 | 15.4 | 21.1 |
| | (SEQ ID NO: 100) | (SEQ ID NO: 101) | | | |
| corn | NM_001155569 | NP_001149041 | 211 | 16.1 | 22.3 |
| | (SEQ ID NO: 102) | (SEQ ID NO: 103) | | | |
| corn | XM_008657627 | XP_008655849 | 195 | 15.4 | 19.0 |
| | (SEQ ID NO: 104) | (SEQ ID NO: 105) | | | |
| corn | XM_008670754 | XP_008668976 | 176 | 16.5 | 23.0 |
| | (SEQ ID NO: 106) | (SEQ ID NO: 107) | | | |

¹Csa gene IDs are from the Camelina sativa genome browser (website://www.camelinadb.ca/prairiegold/cgi-bin/gbrowse/camelina/);
²Percent homology over the whole protein of SEQ ID NO: 2 was determined using the ALIGNX alignment function of the Vector NTI software package (ThermoFisher).
³Percent homology over the whole protein of SEQ ID NO: 5 was determined using the ALIGNX alignment function of the Vector NTI software package (ThermoFisher).

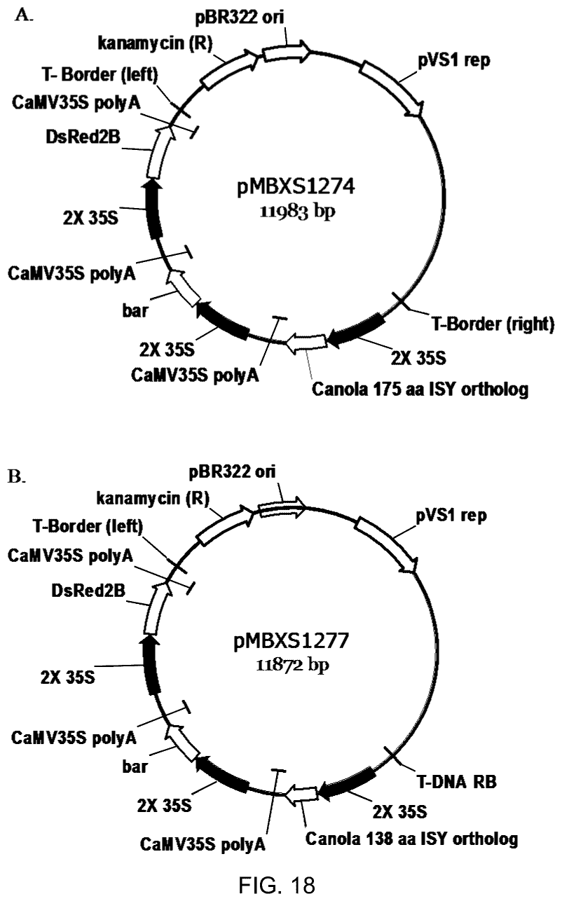

A transformation construct was prepared to express one of the canola orthologs to the Camelina ISY gene (gene sequence XM_013819595.2, SEQ ID NO: 70 encoding protein sequence XP_013675049, SEQ ID NO: 71) in canola. Transformation construct pMBXS1274 (FIG. 18A, SEQ ID NO: 193) expresses a gene fragment that encodes the 175 amino acid protein in SEQ ID NO: 71 expressed from the double enhanced CaMV35S constitutive promoter (2× 35S). Construct pMBXS1274 was transformed into canola as described above and 93 TO lines were isolated.

A Clustal Omega multiple sequence alignment was examined to identify a smaller gene fragment for expression that would yield a canola protein of similar length to 134 amino acid Camelina ISY sequence (SEQ ID NO: 2). FIG. 17 shows alignments of multiple orthologs to the Camelina ISY proteins, including 175 amino acid SEQ ID NO: 71, the chosen Canola ortholog for expression. A 138 amino acid protein fragment of SEQ ID NO: 71 was identified that was similar in length to, and aligned with, the Camelina ISY 134 amino acid protein in SEQ ID NO: 2 (FIG. 15). The first amino acid of this protein fragment was a leucine (L). The first codon of the gene sequence encoding the protein was thus changed so that it would encode a methionine instead of a leucine resulting in SEQ ID NO: 194 (gene sequence) and SEQ ID NO: 195 (protein sequence). Construct pMBXS1277 (FIG. 18B, SEQ ID NO: 196) was prepared that contains the SEQ ID NO: 194 DNA fragment expressed from the double enhanced CaMV35S constitutive promoter (2× 35S). Construct pMBXS1277 was transformed into canola as described above and TO lines were isolated.

### Example 7. Expression of CCP1 in Canola Using Constitutive and Seed-Specific Promoters and Identification of Up-Regulated Genes Having Homology to Genes from the Plant Invertase Inhibitor/Pectin Methylesterase Inhibitor Family Having Higher Seed Yield and Optionally Reduced Individual Seed Weight

Vector pMBXO58 (FIG. 3), engineered for constitutive expression of CCP1, was transformed into Agrobacterium strain GV3101 (pMP90). The resulting strain was used to transform Brassica napus cv DH12075 as described above. Transgenic lines that are resistant to the herbicide bialaphos were obtained.

Screening of transgenic plants of canola expressing the CCP1 gene to identify plants with higher yield was performed as follows. The T1 seeds of several independent lines were grown in a randomized complete block design in a greenhouse maintained at 24° C. during the day and 18° C. during the night. The T2 generation of seed from each line was harvested. Seed yield from each plant was determined by harvesting all of the mature seeds from a plant and drying them in an oven with mechanical convection set at 22° C. for two days. The weight of the entire harvested seed was recorded. The 1000 seed weight was measured to determine individual seed weight. The oil content of seeds can be measured using published procedures for preparation of fatty acid methyl esters (Malik et al. 2015, Plant Biotechnology Journal, 13, 675-688). A number of T1 lines were progressed to the T3 generation to generate homozygous lines followed by seed bulk up in the greenhouse to produce sufficient seed for field tests. These field tests were conducted in Saskatchewan in the 2017 growing season. The best canola line developed in these experiments was line MW82 which had showed a statistically significant increase in seed yield of 13% as compared to a control plant. The 1000 seed weight from this line (an indicator of seed size), in common with the other CCP1 lines tested was approximately 20% lower than the 1000 seed weight from the control plants. This reduced seed size was consistent among the CCP1 lines tested in the field trials. These data indicate that the constitutive expression of CCP1 in canola results in similar phenotypes to that observed in Camelina, i.e. increased seed yield and smaller seed size.

Tissue from plants with higher seed yield and optionally reduced individual seed weight were harvested as follows. Leaf tissue from plants in the vegetative stage and developing seed tissue from higher yield lines were harvested for RNA Seq analysis to identify up-regulated canola genes that have some sequence homology to genes from the plant invertase inhibitor/pectin methylesterase inhibitor family of genes or the gene ontology categories listed in TABLE 16. RNA Seq analysis was performed through a contract vendor and differential gene expression data was obtained and analyzed for up-regulated and down-regulated genes. Results of the RNA-SEQ experiments are described in Example 18.

Similar transformations were performed with construct pMBXO84 (FIG. 4, SEQ ID NO: 96) engineered for seed-specific expression of CCP1. Transgenic lines were obtained and advanced as described above for lines transformed with pMBXO58. Homozygous lines were grown in a field trial during the 2018 growth season in Elm Creek, Manitoba, Canada. The best lines contained a statistically significant 11% increase in seed yield compared to control plants with no significant difference in seed size. RNA SEQ analysis on tissue harvested from these lines can be performed to identify ISY related genes or genes in the gene ontology categories listed in TABLE 16.

### Example 8. Expression of CCP1 in Soybean Using Constitutive and Seed-Specific Promoters and Identification of Up-Regulated Genes Having Homology to Genes from the Plant Invertase Inhibitor/Pectin Methylesterase Inhibitor Family in Lines Having Higher Seed Yield and Optionally Reduced Seed Size

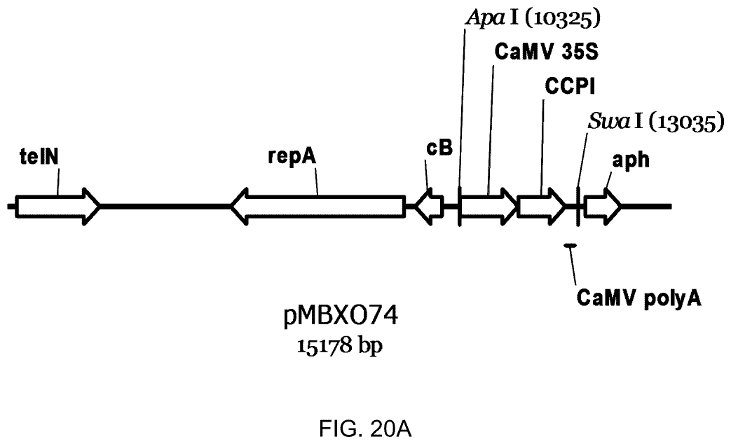

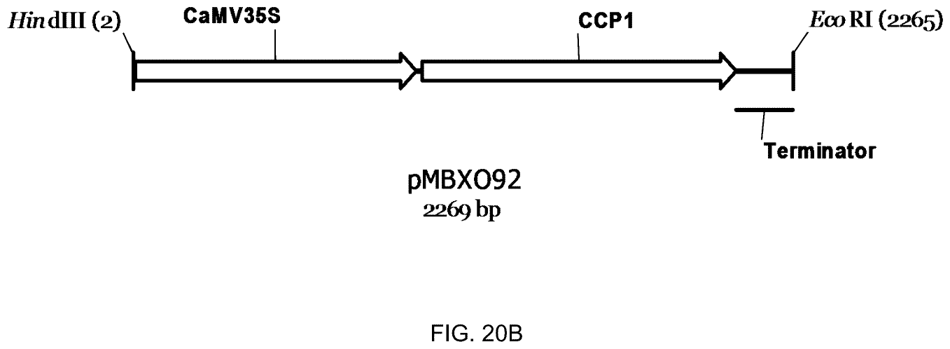

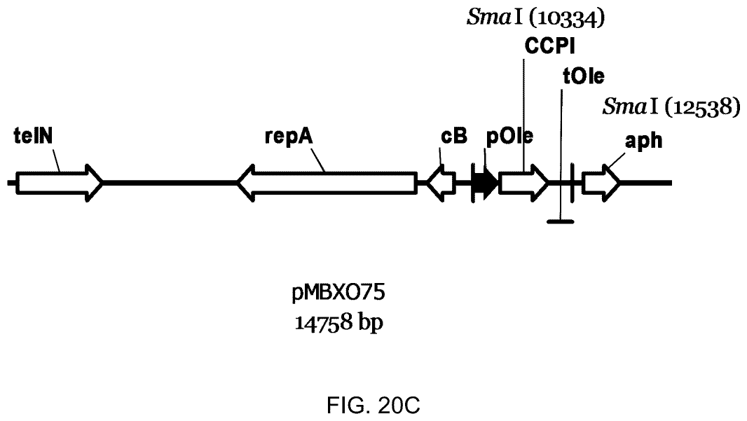

To express CCP1 from a constitutive promoter in soybean, the pJAZZ (Lucigen) based linear vector pMBXO74 (FIG. 20A, SEQ ID NO: 128) was used. A DNA fragment containing the CaMV 35S promoter, the CCP1 gene, and the CaMV polyA termination sequence was excised from pMBXO74 by digestion with the Apa I and Swa I restriction enzymes (FIG. 20A) and a 2.7 kb DNA fragment was isolated and co-bombarded with a hygromycin cassette into soybean embryogenic cultures as described above.

Another constitutive expression cassette for CCP1 was prepared and the Eco RI/Hind III excised fragment used for bombardment is shown in FIG. 20B (pMBXO92, SEQ ID NO: 129). This expression cassette contains a CCP1 gene codon optimized for expression in soybean and was co-bombarded with a hygromycin cassette into soybean embryogenic cultures as described above.

To express CCP1 from a seed-specific promoter in soybean, the pJAZZ (Lucigen) based linear vector pMBXO75 (FIG. 20C, SEQ ID NO: 130) was used. A DNA fragment containing the promoter from the soya bean oleosin isoform A gene, the CCP1 gene, and the soya bean oleosin isoform A termination sequence was excised from pMBXO75 by digestion with the Sma I restriction enzyme (FIG. 20C) and a 2.2 kb, Sma I Oleosin promoter-CCP1-oleosin terminator fragment was excised and co-bombarded with a hygromycin cassette into soybean embryogenic cultures.

Soybean plants from the pMBXO74, pMBXO92, and pMBXO75 transformations were generated as described above and grown for several generations.

Tissue from plants with higher seed yield and/or increased branching and optionally reduced individual seed weight was harvested as follows.

Leaf tissue from plants in the vegetative stage and developing seed tissue from higher yield lines was harvested, RNA was extracted, and RNASeq analysis was performed through a contract vendor. Differential gene expression data was obtained and analyzed for up-regulated and down-regulated genes. Results of these RNA SEQ experiments are described in Example 15.

### Example 9. Expression of CCP1 in Rice Using Constitutive and Seed-Specific Promoters and Identification of Up-Regulated Genes Having Homology to Genes from the Plant Invertase Inhibitor/Pectin Methylesterase Inhibitor Family in Lines Having Higher Seed Yield and Optionally Reduced Seed Size

Several promoters were chosen for expression of the CCP1 gene in rice based on their experimental or in silico predicted expression profiles in rice seed.

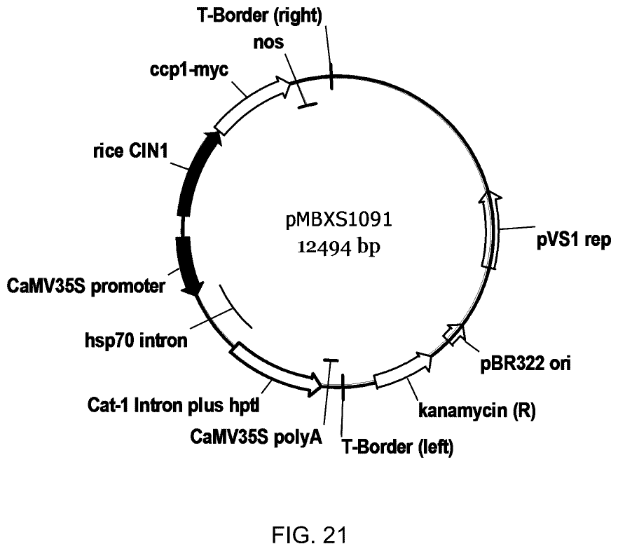

A transformation construct pMBXS1091 (FIG. 21) containing the promoter from the rice beta-fructofuranosidase insoluble isoenzyme 1 (CIN1) gene driving the expression of CCP1 fused to a myc tag was prepared. The CIN1 promoter was chosen based on in silico expression data showing expression throughout various developmental stages but with highest expression in the inflorescence and seeds (Rice Genome Annotation Project; website://rice.plantbiology.msu.edu/cgi-bin/ORF_infopage.cgi?orf=LOC_Os02g33110.1).

Rice was transformed with construct pMBXS1091 as described above using callus of the rice cultivar Nipponbare. Regeneration of plantlets from transformed calli was performed and rooted plants were transferred into peat-pellets to allow for hardening of the roots. The plants were then kept in sealable bags for acclimatization. Plants were transferred into pots and grown in a greenhouse to maturity. The number of tillers and panicles in these primary transformants compared to the wild-type control lines are shown in TABLE 13.

TABLE 13
Comparison of number of tillers and panicles produced
in primary transformants of transgenic rice transformed
with pMBXS1091 compared with wild-type controls.

| | Tillers | | Panicles | |
|---|---|---|---|---|
| | | % of highest | | % of highest |
| | | wild-type | | wild-type |
| Line | Number | control¹ | Number | control² |
| NB-E | 29 | | 26 | 100 |
| (wild-type) | | | | |
| NB-D | 36 | 100 | 22 | |
| (wild-type) | | | | |
| NB-C | 24 | | 0 | |
| (wild-type) | | | | |
| P1091-8B | 81 | 225 | 48 | 185 |
| P1091-9B | 51 | 142 | 43 | 165 |
| P1091-2C | 61 | 169 | 38 | 146 |
| P1091-8A | 48 | 133 | 35 | 135 |
| P1091-1A | 53 | 147 | 32 | 123 |
| P1091-8C | 51 | 142 | 30 | 115 |
| P1091-11C | 43 | 119 | 29 | 112 |
| P1091-2A | 32 | | 29 | 112 |
| P1091-2B | 35 | | 29 | 112 |
| P1091-6A | 48 | 133 | 28 | 108 |
| P1091-9D | 36 | 100 | 28 | 108 |
| P1091-10F | 57 | 158 | 27 | 104 |
| P1091-11B | 42 | 117 | 27 | 104 |
| P1091-4A | 48 | 133 | 27 | 104 |
| P1091-4B | 34 | | 27 | 104 |
| P1091-3B | 29 | | 26 | 100 |
| P1091-7A | 71 | 197 | 26 | 100 |
| P1091-10E | 30 | | 24 | |
| P1091-12B | 31 | | 24 | |
| P1091-11A | 32 | | 23 | |
| P1091-9A | 43 | 119 | 21 | |
| P1091-10A | 34 | | 20 | |
| P1091-1D | 24 | | 17 | |
| P1091-2E | 39 | 108 | 16 | |
| P1091-2D | 36 | 100 | 10 | |
| P1091-4C | 58 | 161 | 9 | |
| P1091-1B | 23 | | 7 | |
| P1091-1E | 19 | | 7 | |
| P1091-5A | 28 | | 6 | |
| P1091-9C | 45 | 125 | 4 | |
| P1091-10C | 63 | 175 | 0 | |
| P1091-10D | 46 | 128 | 0 | |
| P1091-4D | 33 | | 0 | |
| P1091-5C | 31 | | 0 | |

¹The % to wild-type control was calculated using the best wild-type plant that produced the most tillers. Only % to control values equal or greater than 100% are shown.
²The % to wild-type control was calculated using the best wild-type plant that produced the most panicles. Only % to control values equal or greater than 100% are shown.

Seed is harvested from each particle (T1 generation) and the seed yield per plant is calculated. Ti seed is grown in a greenhouse to produce T2 seed. The mass of the total seed per plant is collected to compare seed yield of transgenics to wild-type control plants. Samples of plant tissue were collected for RNA extraction, RNA was extracted, and RNASeq analysis was performed through a contract vendor. Differential gene expression data can be analyzed for up-regulated and down-regulated genes, with a focus on identifying genes from the plant invertase inhibitor/pectin methylesterase inhibitor gene family or the gene ontology categories listed in TABLE 16.

### Example 10. Increased Expression of CCP1 in Maize Using Constitutive and Seed-Specific Promoters and Identification of Up-Regulated Genes Having Homology to Genes from the Plant Invertase Inhibitor/Pectin Methylesterase Inhibitor Family in Lines Having Higher Seed Yield and Optionally Reduced Seed Size

For transformation of maize, a binary vector containing a promoter, the CCP1 gene, and a terminator is constructed. Appropriate constitutive promoters for maize include monocot promoters listed in TABLE 2. For example, an expression cassette containing the hybrid cab5/hsp70 intron promoter (SEQ ID NO: 64) operably linked to the CCP1 gene from Chlamydomonas reinhardtii, operably linked to the nos terminator of the nopaline synthase gene can be used. An expression cassette for a selectable marker, such as the bar gene imparting resistance to the herbicide bialaphos, is included.

For seed specific expression, a binary vector containing a promoter, the CCP1 gene, and a terminator is constructed. Appropriate seed-specific promoters include monocot promoters listed in TABLE 2. For example, an expression cassette containing the maize β-fructofuranosidase insoluble isoenzyme 1 (CIN1) promoter (SEQ ID NO: 66) operably linked to the CCP1 gene from Chlamydomonas reinhardtii, operably linked to the nos terminator of the nopaline synthase gene can be used. An expression cassette for a selectable marker, such as the bar gene imparting resistance to the herbicide bialaphos, is included.

In preparation for transformation, the binary vector is transformed into an Agrobacterium tumefaciens strain, such as A. tumefaciens strain EHA101. Agrobacterium-mediated transformation of maize can be performed following a previously described procedure (Frame et al. (2006), Agrobacterium Protocols, Wang K., ed., Vol. 1, pp 185-199, Humana Press) as described above.

Bialaphos-resistant embryogenic callus lines are regenerated as described above and transferred to soil. Plants are grown in a greenhouse to produce T1 seed. T1 seed is grown in soil in a greenhouse to produce T2 seed. The mass of the total seed per plant is collected to compare seed yield of transgenics to wild-type control plants.

RNA is extracted and RNASeq analysis is performed through a contract vendor. Differential gene expression data can be analyzed for up-regulated and down-regulated genes, with a focus on identifying genes from the plant invertase inhibitor/pectin methylesterase inhibitor gene family or the gene ontology categories listed in TABLE 16.

### Example 11. Use of Genome Editing to Alter the Expression of the ISY Gene in Camelina, or Orthologs of the Camelina ISY Gene in Other Crops

The expression of ISY gene can be modified by replacing the native promoter sequences upstream of the coding sequence with a promoter containing a stronger or more optimal tissue-specific expression profile. Example promoters useful for this purpose are listed in TABLE 1 and TABLE 2. To increase the concentration of the gene product, a stronger promoter than the native one is used. The tissue specificity of expression of the promoter can also be modified, to increase or reduce the types of tissues where the gene is expressed.

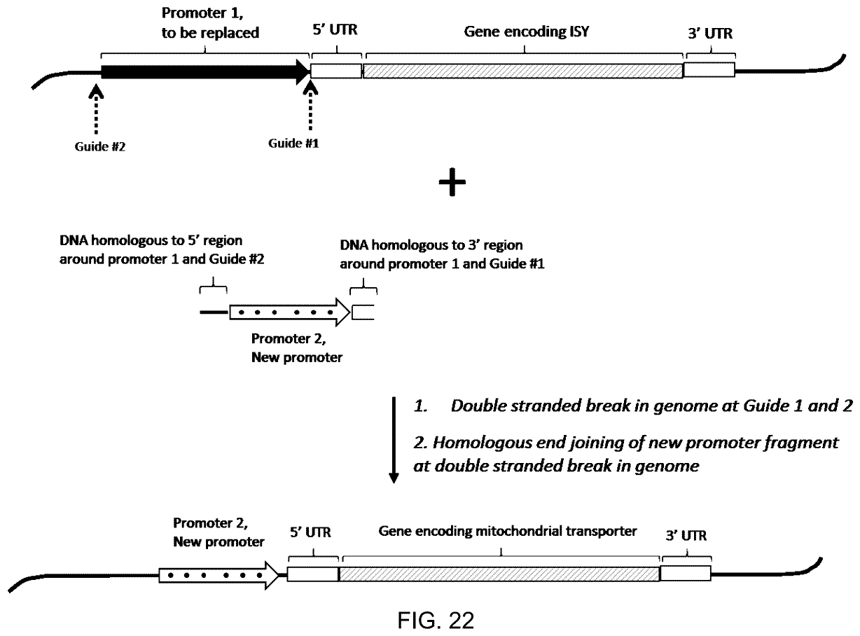

Replacement of the native promoter can be achieved using a genome editing enzyme to make the targeted double stranded cuts to remove the native promoter (Promoter 1) (FIG. 22). A new promoter (Promoter 2) is then inserted via a homology-directed repair (HDR) repair mechanism, in which the new promoter is flanked by DNA sequences with homology to regions upstream and downstream of the original native promoter (Promoter 1). In one embodiment, these promoter flanking regions can additionally be flanked by guide target sequences and adjacent PAM sites to allow release of the promoter by Cas9 from a construct or other DNA.

There are multiple methods to achieve double stranded breaks in genomic DNA, including the use of zinc finger nucleases (ZFN), transcription activator-like effector nucleases (TALENs), engineered meganucleases, and the CRISPR/Cas system (CRISPR is an acronym for clustered, regularly interspaced, short, palindromic repeats and Cas an abbreviation for CRISPR-associated protein) (for review see Khandagale & Nadaf (2016), Plant Biotechnol Rep, 10:327-343). CRISPR/Cas mediated genome editing is the easiest of these methods to implement since all that is needed is the Cas9 enzyme and a single guide RNA (sgRNA) with homology to the modification target to direct the Cas9 enzyme to desired cut site for cleavage. The ZFN, TALENs, and engineered meganucleases methods require more complex design and protein engineering to bind the DNA sequence to enable editing. For this reason, the CRISPR/Cas mediated system has become the method of choice for genome editing.

The sgRNA used in the CRISPR/Cas system is a synthetic RNA chimera created by fusing crRNA with tracrRNA. The guide sequence, located at the 5′ end of the sgRNA, confers DNA target specificity. Therefore, by modifying the guide sequence, it is possible to create sgRNAs with different target specificities. The canonical length of the guide sequence is ˜20 bp. In plants, sgRNAs have been expressed using plant RNA polymerase III promoters, such as U6 and U3. Cas9 expression plasmids for use in the methods of the invention can be constructed as described in the art.

It will be apparent to those skilled in the art that any of these systems can be used for generating the double stranded breaks necessary for promoter excision in this example.

In this example the CRISPR/Cas system is used. There are many variations of the CRISPR/Cas system that can be used for this technology including the use of wild-type Cas9 from Streptococcus pyogenes (Type II Cas) (Barakate & Stephens (2016), Frontiers in Plant Science, 7:765; Bortesi & Fischer (2015), Biotechnology Advances 5:33, 41; Cong et al. (2013), Science, 339:819; Rani et al. (2016), Biotechnology Letters, 1-16; Tsai et al. (2015), Nature Biotechnology, 33:187), the use of a Tru-gRNA/Cas9 in which off-target mutations were significantly decreased (Fu et al. (2014), Nature Biotechnology, 32:279; Osakabe et al. (2016), Scientific Reports, 6:26685; Smith et al. (2016), Genome Biology, 17:1; Zhang et al. (2016), Scientific Reports, 6:28566), a high specificity Cas9 (mutated S. pyogenes Cas9) with little to no off target activity (Kleinstiver et al. (2016), Nature 529:490; Slaymaker et al. (2016), Science, 351:84), the Type I and Type III Cas Systems in which multiple Cas protein need to be expressed to achieve editing (Li et al. (2016), Nucleic Acids Research, 44:e34; Luo et al. (2015), Nucleic Acids Research, 43:674), the Type V Cas system using the Cpf1 enzyme (Kim et al. (2016), Nature Biotechnology, 34:863; Toth et al. (2016), Biology Direct, 11:46; Zetsche et al. (2015), Cell, 163:759), DNA-guided editing using the NgAgo Agronaute enzyme from Natronobacterium gregoryi that employs guide DNA (Xu et al. (2016), Genome Biology, 17:186), and the use of a two vector system in which Cas9 and gRNA expression cassettes are carried on separate vectors (Cong et al. (2013), Science, 339:819).

It will be apparent to those skilled in the art that any of the CRISPR enzymes can be used for generating the double stranded breaks necessary for promoter excision in this example. There is ongoing work to discover new variants of CRISPR enzymes which, when discovered, can also be used to generate the double stranded breaks around the native promoters of the ISY genes.

In this example, the CRISPR/Cas9 system is used. FIG. 22 details a strategy for promoter replacement in front of a native ISY sequence using CRISPR/Cas9 and a homologous directed repair mechanism. An sgRNA containing Guide #1 and Guide #2 are used to excise the promoter to be replaced (Promoter 1). A new promoter cassette (Promoter 2), flanked by sequences with homology to the upstream and downstream region of Promoter 1, is introduced and is inserted into the site previously occupied by Promoter 1 using the homologous directed repair mechanism.

It will be apparent to those skilled in the art that many different promoters are available for expression in plants. TABLE 1 and TABLE 2 list some of the additional options for use in dicots and monocots that can be used as replacement promoters for the genome editing strategy.

The promoter replacement strategy shown in FIG. 22 can also be performed in other crops containing orthologs of the Camelina ISY gene to modify the expression of the ortholog, including soybean, corn, and canola. The native promoter in front of orthologs of the Camelina ISY gene in soybean, corn, and canola listed in TABLE 12 can be replaced with a different promoter using genome editing methods such as the CRISPR-Cas method.

### Example 12. Expression of Orthologs to the Camelina ISY Gene, or Genes with Homology to Plant Invertase Inhibitors/Pectin Methylesterase Inhibitors Identified in CCP1 Lines, in Soybean

For transformation of soybean with orthologs to the Camelina Csa15g017550 ISY gene described in TABLE 12, or for transformation of genes with homology to plant invertase inhibitors/pectin methylesterase inhibitors or genes within the GO categories of TABLE 16 that were identified using the procedures described in Example 8 and Example 15 for CCP1 overexpressing soybean lines, an expression cassette containing a promoter, the gene of interest, and a polyadenylation sequence is prepared. Suitable promoters for dicots are listed in TABLE 1. For example, an expression cassette can contain the constitutive CaMV 35S promoter (SEQ ID NO: 108), the ISY gene, and the CaMV polyadenylation sequence. A DNA fragment containing the cassette is purified and co-bombarded with a hygromycin cassette into soybean embryogenic cultures.

The purified DNA fragment(s) are introduced into embryogenic cultures of soybean Glycine max cultivars X5 and Westag97 via biolistics, to obtain transgenic plants. The transformation, selection, and plant regeneration of soybean is performed as described above.

Germinated transformed embryos are transferred to soil as described above and grown at 18-h photoperiod at a light intensity of 300-400 μmolm² s⁻¹.

T1 seeds are harvested and planted in soil and grown in a controlled growth cabinet at 26/24° C. (day/night), 18 h photoperiod at a light intensity of 300-400 μmol m⁻² s⁻¹. Plants are grown to maturity and T2 seed is harvested. Seed yield per plant and oil content of the seeds is measured.

There are also Agrobacterium-mediated transformation methods for soybean that can be used to generate similar transgenic plants expressing the ISY gene.

### Example 13. Expression of Orthologs to the Camelina ISY Gene, or Genes with Homology to Plant Invertase Inhibitors/Pectin Methylesterase Inhibitors Identified in CCP1 Lines, in Rice and Maize

For transformation of rice with genes with homology to plant invertase inhibitors/pectin methylesterase inhibitors identified using the procedures described in Example 9 for CCP1-overexpressing lines, a binary vector containing an expression cassette with a promoter, the gene of interest, and a polyadenylation sequence, as well as an expression cassette for a selectable marker, such as the hygromycin resistance marker, is prepared. The ISY and hygromycin resistance cassette can be co-localized on one binary vector or, alternatively, positioned on two separate binary vectors that can be co-bombarded.

Several promoters are chosen for expression of genes with homology to plant invertase inhibitors/pectin methylesterase inhibitors in rice based on their experimental or in silico predicted expression profiles in rice seed and are shown in TABLE 2, above. It will be apparent to those skilled in the art that TABLE 2 is not an exhaustive list of promoters that can be used for expression in rice and that there are many additional promoters that would work to practice the invention, depending on the tissue specificity desired for expression of the transgene. The promoter from the rice ADP-glucose pyrophosphorylase (AGPase) gene (TABLE 2, SEQ ID NO: 55) was chosen since it has been shown to be expressed in the seed as well as the phloem of vegetative tissues in rice (Qu and Takaiwa (2004), Plant Biotechnology Journal, 2:113-125). The promoter from the rice glutelin C (GluC) gene (TABLE 2, promoter with SEQ ID NO: 56) has been shown to be expressed in the whole endosperm of rice seed (Qu et al. (2008), Journal of Experimental Biology, 59:2417-2424). The promoter from the rice beta-fructofuranosidase insoluble isoenzyme 1 (CIN1) gene (TABLE 2, SEQ ID NO: 57) was chosen based on in silico expression data showing expression throughout various developmental stages but with highest expression in the inflorescence and seeds (Rice Genome Annotation Project; website://rice.plantbiology.msu.edu/cgi-bin/ORF_infopage.cgi?orf=LOC_Os02g33110.1). The prepared transformation construct is transformed into callus of the rice cultivar Nipponbare using Agrobacterium mediated transformation as described above.

Plantlets are regenerated from calli and transferred to peat-pellets and then soil as described above. Plants are grown in a greenhouse to maturity prior to seed harvest (T1 generation). T1 seed is grown in a greenhouse to produce T2 seed. The mass of the total seed per plant is collected to compare seed yield of transgenics to wild-type control plants.

For transformation of maize with orthologs to the Camelina Csa15g017550 ISY gene described in TABLE 12, or for transformation of genes with homology to plant invertase inhibitors/pectin methylesterase inhibitors identified using the procedures described in Example 10 for maize CCP1-overexpressing lines, a binary vector containing a promoter, the gene of interest, and a terminator is constructed. Appropriate promoters include the monocot promoters listed in TABLE 2. For example, an expression cassette containing the hybrid cab5/hsp70 intron promoter (SEQ ID NO: 64) operably linked to the ISY gene, operably linked to the nos terminator of the nopaline synthase gene can be used. An expression cassette for a selectable marker, such as the bar gene imparting resistance to the herbicide bialaphos, is included. In preparation for transformation, the binary vector is transformed into an Agrobacterium tumefaciens strain, such as A. tumefaciens strain EHA101. Agrobacterium-mediated transformation of maize can be performed following a previously described procedure (Frame et al. (2006), Agrobacterium Protocols, Wang K., ed., Vol. 1, pp 185-199, Humana Press) as described above. Plants are regenerated as described above and are transferred to soil. Plants are grown in a greenhouse to produce T1 seed. T1 seed is grown in soil in a greenhouse to produce T2 seed. The mass of the total seed per plant is collected to compare seed yield of transgenics to wild-type control plants.

### Example 14. Expression of the Camelina ISY Gene, its Orthologs, or Genes with Homology to Plant Invertase Inhibitors/Pectin Methylesterase Inhibitors, in Plants in Combination with CCP1 Genes

The Camelina ISY gene, ortholog genes, or genes with homology to plant invertase inhibitors/pectin methylesterase inhibitors can be co-expressed in a plant with a CCP1 gene by placing expression cassettes for the Camelina ISY gene, an ortholog gene, or a gene with homology to a plant invertase inhibitor/pectin methylesterase inhibitor and the CCP1 gene on the same transformation vector and transforming the plant. The gene cassettes can contain a variety of different promoters to control expression, including seed-specific promoters, constitutive promoters, leaf-specific promoters, or other tissue-specific promoters which will be apparent to those skilled in the art. An expression cassette for a selectable marker, such as the bar gene, providing resistance to the herbicide bialaphos, is also included. The selectable marker is chosen based on the plant to be transformed.

Four examples given here are constructs in which the expression of both genes is controlled by constitutive CaMV35S promoter and terminators, the expression of both genes is controlled by seed-specific oleosin promoters and matching oleosin terminators, the Camelina ISY, ortholog gene, or gene with homology to a plant invertase inhibitor/pectin methylesterase inhibitor expression is controlled by the 35S promoter and the CCP1 gene is controlled by the oleosin promoter, and the Camelina ISY gene, ortholog gene, or gene with homology to a plant invertase inhibitor/pectin methylesterase inhibitor expression is controlled by the oleosin promoter and the CCP1 gene is controlled by the constitutive promoter. These vectors (constructs A, B, C, and D in TABLE 14) are designed for transformation into dicots, including Camelina and canola, whose transformation procedures are described above. Co-transformation of constructs A and B into the same plant may provide enhanced yield benefits.

TABLE 14
Example expression cassettes for co-expression
of an ISY or CWII gene with CCP1.

| | Expression Cassette 1 | | | Expression Cassette 2 | | |
|---|---|---|---|---|---|---|
| Construct | promoter | gene | terminator | promoter | gene | terminator |
| A | constitutive | Camelina ISY | CaMV35S | constitutive | CCP1 | CaMV35S |
| | CaMV35S | gene or gene | | CaMV35S | | |
| | | with homology | | | | |
| | | to plant | | | | |
| | | invertase | | | | |
| | | inhibitor/pectin | | | | |
| | | methylesterase | | | | |
| | | inhibitor | | | | |
| B | seed-specific | Camelina ISY | oleosin | seed-specific | CCP1 | oleosin |
| | oleosin | gene or gene | | oleosin | | |
| | | with homology | | | | |
| | | to plant | | | | |
| | | invertase | | | | |
| | | inhibitor/pectin | | | | |
| | | methylesterase | | | | |
| | | inhibitor | | | | |
| C | constitutive | Camelina ISY | CaMV35S | seed-specific | CCP1 | oleosin |
| | CaMV35S | gene or gene | | oleosin | | |
| | | with homology | | | | |
| | | to plant | | | | |
| | | invertase | | | | |
| | | inhibitor/pectin | | | | |
| | | methylesterase | | | | |
| | | inhibitor | | | | |
| D | seed-specific | Camelina ISY | oleosin | constitutive | CCP1 | CaMV35S |
| | oleosin | gene or gene | | CaMV35S | | |
| | | with homology | | | | |
| | | to plant | | | | |
| | | invertase | | | | |
| | | inhibitor/pectin | | | | |
| | | methylesterase | | | | |
| | | inhibitor | | | | |

It will be apparent to those skilled in the art that co-expression of ISY or CWII and CCP1 genes can also be achieved by co-transformation of separate vectors that contain an ISY or CWII expression cassette on one plasmid and a CCP1 expression cassette on another plasmid and screening the transformants for the presence of both expression cassettes. It will also be apparent to those skilled in the art that co-expression of ISY or CWII and CCP1 genes can be achieved by crossing plants expressing the individual genes to obtain a plant expressing both genes.

Vectors selected from A, B, C and/or D (TABLE 14) can be optimized for transformation into soybean by replacing the bar expression cassette with an expression cassette encoding the hygromycin gene. A DNA fragment(s) containing the CCP1 gene, ISY or CWII gene, and hygromycin resistance gene expression cassettes can be excised and introduced into soybean using the biolistics method described above. In some cases, it may be desirable to optimize the promoter's expression for CCP1 and ISY or the gene with homology to a plant invertase inhibitor/pectin methylesterase inhibitor. Promoters for expression of these transgenes can be selected from those listed in TABLE 1, depending on the desired tissue specificity for expression, or can be selected from any other promoter that provides good expression in dicots.

Vectors selected from A, B, C and/or D (TABLE 14) can be optimized for transformation into rice by replacing the bar expression cassette with an expression cassette encoding the hygromycin gene. In some instances, it may be desirable to optimize the promoters driving the expression of the ISY or the gene with homology to a plant invertase inhibitor/pectin methylesterase inhibitor and CCP1 genes using a monocot specific promoter, such as the ones described in TABLE 2, above, or any other promoter that provides good expression in monocots. The choice of the promoter may be dictated by the desired tissue specificity for expression. The modified binary vectors are introduced into an Agrobacterium strain, such as Agrobacterium strain AGL1, and the rice transformation procedure described above is followed.

Vectors selected from A, B, C and/or D (TABLE 14) can be optimized for transformation into maize by using a monocot specific promoter, such as the ones described in TABLE 2, or any other promoter that provides good expression in monocots, to drive the expression of the ISY or the gene with homology to a plant invertase inhibitor/pectin methylesterase inhibitor and CCP1 genes. The choice of the promoter may be dictated by the desired tissue specificity for expression. The modified binary vectors are introduced into an Agrobacterium strain, such as A. tumefaciens strain EHA101, and the maize transformation procedure described above is followed.

### Example 15. Identification of Up- and Down-Regulated Genes in Soybean Lines Expressing the CCP1 Gene Using RNA Sequencing

Select soybean T2 generation lines, produced as described in Examples 5 and 8, were grown in a growth chamber to generate samples for analysis by RNA Sequencing. TABLE 15 shows lines that were selected for analysis, the tissue type collected, and classifies the lines into groups for analysis purpose. Samples were harvested at the indicated stage and quickly frozen in liquid nitrogen. Samples were stored at −80° C. RNA was extracted using the Omega Bio-Tek E.Z.N.A Plant RNA kit and shipped to a contract service provider that performed RNA Sequencing and data processing.

TABLE 15
Glycine max samples used in the RNA-seq analysis.

| Group | Tissue* | Line** | CCP1 |
|---|---|---|---|
| A | Leaf | Westag wild-type control | − |
| B | Leaf | Null line | − |
| C | Leaf | pMBXO74 (35S-CCP1, heterozygous) | + |
| C-1 | Leaf | pMBXO74 (35S-CCP1, homozygous) | + |
| D | Maturing seed | Westag control | − |
| E | Maturing seed | Null line | − |
| F | Maturing seed | pMBXO74 (35S-CCP1, heterozygous) | + |
| F-1 | Maturing seed | pMBXO74 (35S-CCP1, homozygous) | + |
| L | Maturing seed | Westag control | − |
| M | Maturing seed | Null line | − |
| P | Maturing seed | pMBXO75 (pOle-CCP1, homozygous) | + |
| H | Developing | Westag control | − |
| | seed | | |
| J | Developing | Null line | − |
| | seed | | |
| K | Developing | pMBXO75 (pOle-CCPl, homozygous) | + |
| | seed | | |

*Harvested tissue stages are as follows: Leaf, expanded leaf at V6 stage collected from 5.5 week old plants; Maturing yellow green seed, 1.2-1.3 cm in size collected from 8 week old plants; developing green seed, 1 cm in size collected from 7 week old plants
**Null line generated through segregation.

Due to their possible relationship to the function of the Camelina ISY gene, the Gene Ontology (GO) terms listed in TABLE 16 were investigated. The objective of this work was to search the Glycine max genome for genes that were differentially expressed in CCP1+ lines that could be associated with one or more of these GO terms. Nearly all genes within the RNA-seq data set had a corresponding best BLAST hit in the Arabidopsis thaliana genome, which is very well-annotated with respect to associated GO terms. Therefore, for each of the GO terms in TABLE 16, a list of Arabidopsis thaliana genes associated with the GO term was made. All genes within the Glycine max genome that had one of those Arabidopsis thaliana genes as its best BLAST hit were then identified. Among these Glycine max genes, some were up- or down-regulated in CCP1 lines in a statistically significant way; that is, a Tukey test suggested that the probability was greater than 90% that the means of the transcript levels in transgenic vs. wild-type lines were different.

### Differentially Expressed Genes in Leaves:

There were a large number of very significant changes in harvested leaf tissue, especially in the homozygous line (C-1) compared to the wild-type control (A) (For line information, see TABLE 15). The up- and down-regulated genes along with the comparison corresponding to the fold changes are shown in TABLE 17. Only comparisons in which the fold difference between the two lines C-1 and A exceeds 2 are shown. It is important to note that genes whose expression was also up- or down-regulated in the null line (B) compared to the control line (A) were excluded from consideration because they may not have been a result of addition of the CCP1 gene to the plant.

TABLE 16
Gene Ontology categories used to
investigate RNA Sequencing data.

| GO category | Description |
|---|---|
| GO: 0030570 | pectate lyase activity |
| GO: 0030599 | pectinesterase activity |
| GO: 0046910 | pectinesterase inhibitor activity |
| GO: 0052793 | pectin acetylesterase activity |

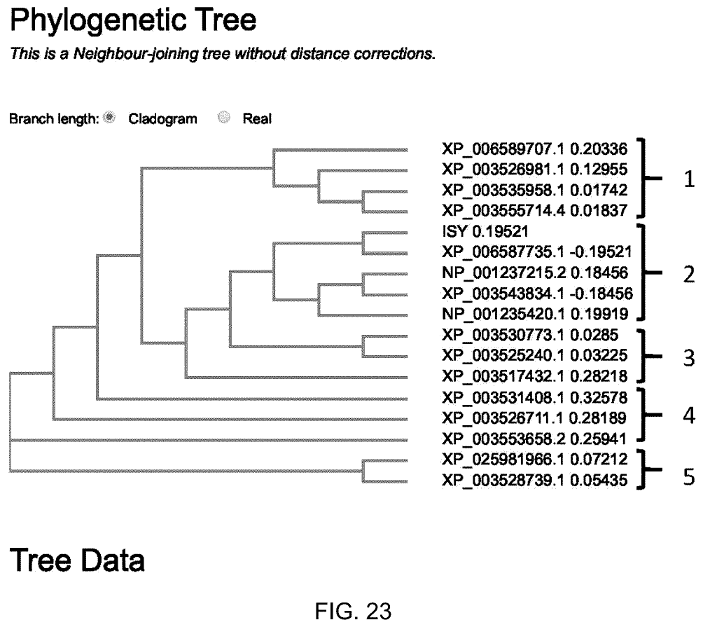

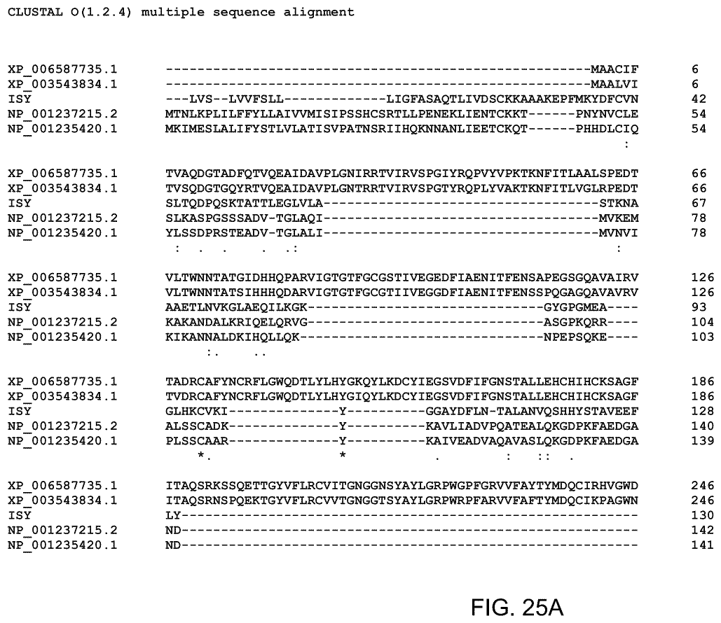

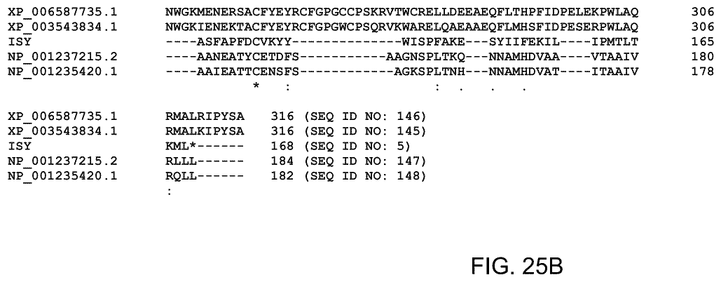

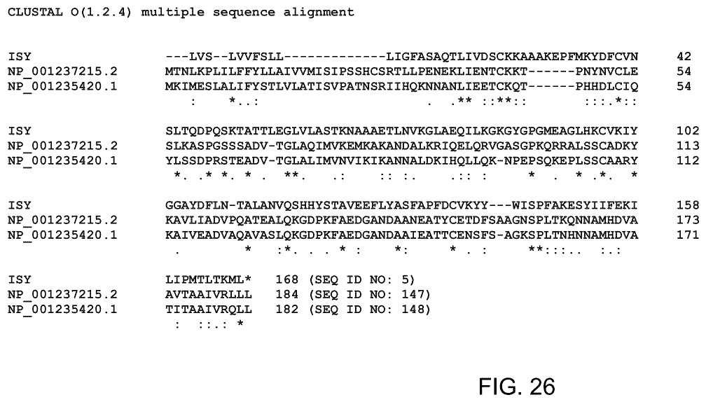

To further sort and examine the relationship of the differentially expressed genes in CCP1+ soybean lines in these Gene Ontology (GO) categories, a phylogenetic tree (cladogram) (FIG. 23) was created using the CLUSTAL Omega program (website://www.ebi.ac.uk/Tools/msa/clustalo/). The Camelina ISY gene was also included to determine if any of the differentially expressed soybean genes were similar to the Camelina ISY gene. Using the phylogenetic tree (FIG. 23) as well as the CLUSTAL Omega multiple sequence alignments, the proteins encoded by the differentially expressed genes were grouped into five groups (FIG. 23 and TABLE 17). In the phylogenetic tree (FIG. 23), the protein encoded by the Camelina ISY gene grouped with the soybean proteins in group 2 which are classified with the GO terms of pectinesterases or cell wall/vacuolar inhibitors of fructosidase 1. The group 2 proteins were investigated more closely with sequence alignments and the Camelina ISY gene appeared to be more closely matched to the cell wall/vacuolar inhibitors of fructosidase 1 (Genes NP_001237215.2 and NP_001235420.1; FIGS. 25 and 26).

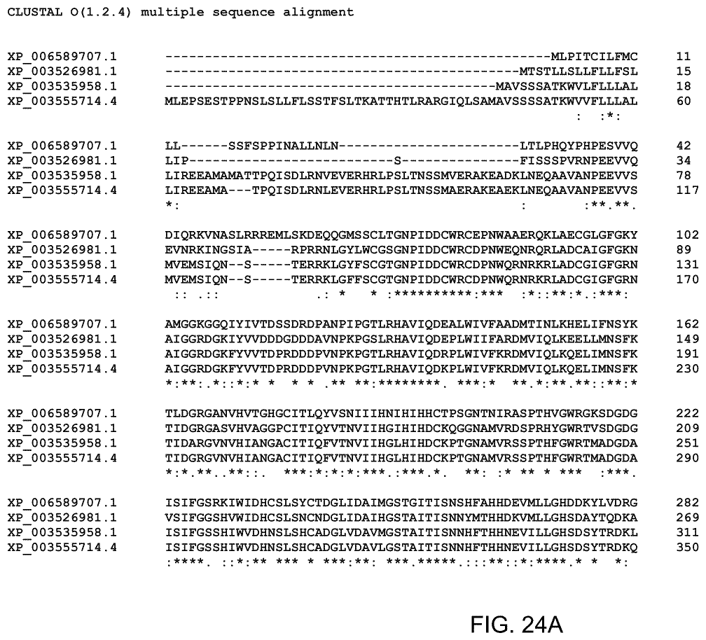

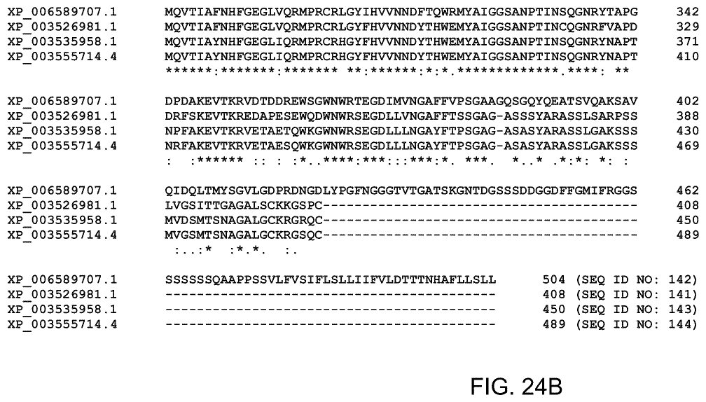

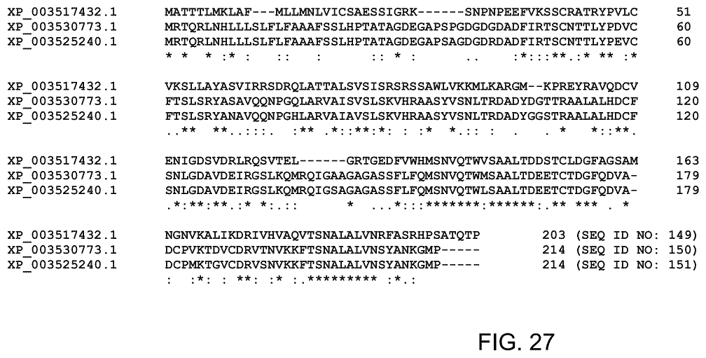

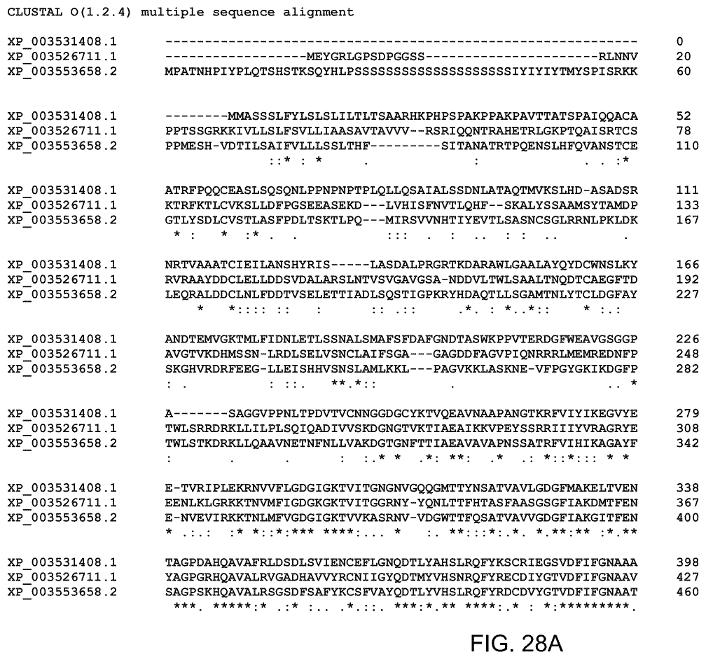

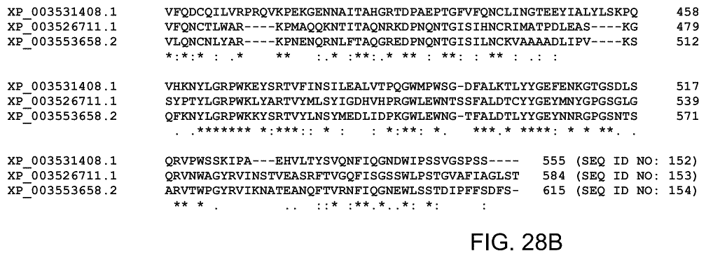

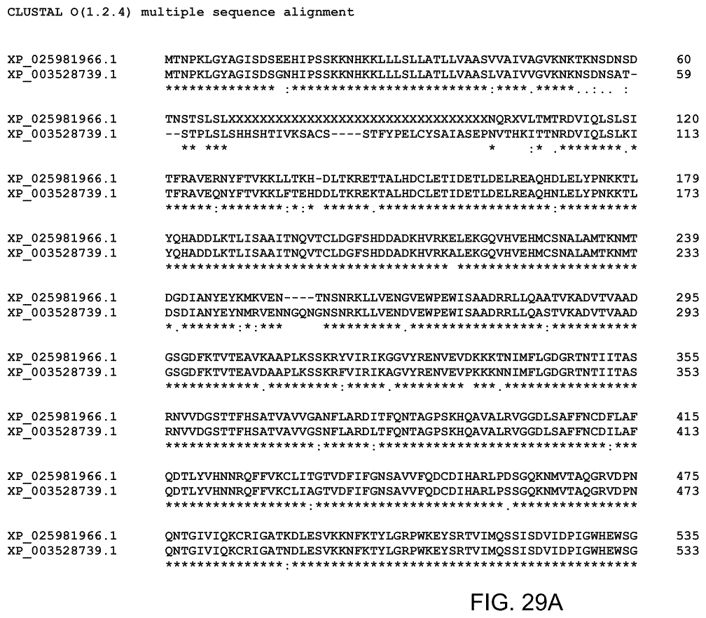

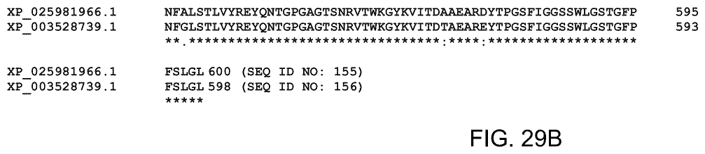

Alignments of proteins in each of the other phylogenetic groupings in TABLE 17 were performed and show a high degree of similarity (FIGS. 24, 27, 28, and 29).

TABLE 17
Differentially expressed Glycine max genes from the plant invertase inhibitor/pectin methylesterase
inhibitor superfamily in CCP1+ homozygous line (C-1) and null line (B) vs. wild type (A).

| | | | | | | Assigned |
|---|---|---|---|---|---|---|
| | | | | | | group in |
| Glycine max | | A. thaliana | A. thaliana | Fold | Fold | phylogenetic |
| locus | Protein ID | locus | description | (C-1)/A | B/A* | tree |
| LOC100787082 | XP_003526981.1 | AT1G67750 | Probable pectate | 3.937 | 0.133 | 1 |
| | (SEQ ID NO: 141) | | lyase 5 | | | |
| LOC100789303 | XP_006589707.1 | AT5G04310 | Pectate lyase | 4.172 | DNP | |
| | (SEQ ID NO: 142) | | | | | |
| LOC100781845 | XP_003535958.1 | AT4G13710 | Probable pectate | 106.093 | DNP | |
| | (SEQ ID NO: 143) | | lyase 15 | | | |
| LOC100808253 | XP_003555714.4 | AT1G04680 | Probable pectate | 16.906 | DNP | |
| | (SEQ ID NO: 144) | | lyase 1 | | | |
| LOC100810259 | XP_003543834.1 | AT3G29090 | Pectinesterase 31 | 0.079 | DNP | 2 |
| | (SEQ ID NO: 145) | | | | | |
| LOC100786786 | XP_006587735.1 | AT3G29090 | Pectinesterase 31 | 4.297 | DNP | |
| | (SEQ ID NO: 146) | | | | | |
| LOC100500640 | NP_001237215.2 | AT1G47960 | Cell | 0.003 | DNP | |
| | (SEQ ID NO: 147) | | wall/vacuolar | | | |
| | | | inhibitor of | | | |
| | | | fructosidase 1 | | | |
| LOC100306719 | NP_001235420.1 | AT1G47960 | Cell | 0.016 | DNP | |
| | (SEQ ID NO: 148) | | wall/vacuolar | | | |
| | | | inhibitor of | | | |
| | | | fructosidase 1 | | | |
| LOC100776636 | XP_003517432.1 | AT1G62770 | Pectinesterase | 0.191 | DNP | 3 |
| | (SEQ ID NO: 149) | | inhibitor 9 | | | |
| LOC100812251 | XP_003530773.1 | AT1G14890 | Plant | 2.328 | DNP | |
| | (SEQ ID NO: 150) | | invertase/pectin | | | |
| | | | methylesterase | | | |
| | | | inhibitor | | | |
| | | | superfamily | | | |
| | | | protein | | | |
| LOC100803842 | XP_003525240.1 | AT1G14890 | Plant | 4.499 | DNP | |
| | (SEQ ID NO: 151) | | invertase/pectin | | | |
| | | | methylesterase | | | |
| | | | inhibitor | | | |
| | | | superfamily | | | |
| | | | protein | | | |
| LOC100801231 | XP_003531408.1 | AT5G09760 | Probable | 44.942 | DNP | 4 |
| | (SEQ ID NO: 152) | | pectinesterase/ | | | |
| | | | pectinesterase | | | |
| | | | inhibitor 51 | | | |
| LOC100776752 | XP_003526711.1 | AT5G53370 | Probable | 25.882 | DNP | |
| | (SEQ ID NO: 153) | | pectinesterase/ | | | |
| | | | pectinesterase | | | |
| | | | inhibitor 61 | | | |
| LOC100813592 | XP_003553658.2 | AT2G45220 | Probable | 192.005 | DNP | |
| | (SEQ ID NO: 154) | | pectinesterase/ | | | |
| | | | pectinesterase | | | |
| | | | inhibitor 17 | | | |
| LOC100776623 | XP_025981966.1 | AT3G14310 | Pectinesterase/ | 14.818 | DNP | 5 |
| | (SEQ ID NO: 155) | | pectinesterase | | | |
| | | | inhibitor 3 | | | |
| LOC100777646 | XP_003528739.1 | AT3G14310 | Pectinesterase/ | 23.049 | DNP | |
| | (SEQ ID NO: 156) | | pectinesterase | | | |
| | | | inhibitor 3 | | | |

*DNP = did not pass Tukey test at P < 0.1 (fold change not significantly different from 1.00). See TABLE 15 for explanation of samples.

An additional analysis was performed with the Glycine max RNA-seq data in which the Bioconductor R package DESeq2 was used to determine differential expression. In this analysis, filtering used a different statistical treatment and additional genes of interest were identified. In the alternative filtering method, the Tukey test was not used to filter the data, but rather all comparisons for which σ/μ<0.75 for both lines were retained (is the mean value of the normalized reads for a particular line and a is their standard deviation). Only comparisons where the two mean read counts from RNA-Seq analysis added up to ≥2.00 were retained.

The genes that fulfilled these criteria and that fall within the GO categories listed in TABLE 16 are shown in TABLE 18. In TABLE 18, only comparisons in which the fold difference between the two compared lines exceeds 1.5 are included.

TABLE 18
Differentially expressed Glycine max genes from the plant invertase inhibitor/pectin methylesterase
inhibitor superfamily in harvested leaves of CCP1+ and null lines vs. wild type control line.*

| Glycine max | | A. thaliana | A. thaliana | Fold | Fold |
|---|---|---|---|---|---|
| locus | Protein ID | locus | description | C1/A | B/A |
| LOC100808438 | XP_003555884.1 | AT3G53190 | Probable pectate lyase 12 | 4.25 | 0.24 |
| | (SEQ ID NO: 159) | | | | |
| LOC100815062 | XP_003541430.1 | AT3G53190 | Probable pectate lyase 12 | 49.89 | |
| | (SEQ ID NO: 160) | | | | |
| LOC100782388 | XP_003551943.1 | AT1G67750 | Probable pectate lyase 5 | 35.65 | |
| | (SEQ ID NO: 157) | | | | |
| LOC100816256 | XP_006591479.1 | AT1G67750 | Probable pectate lyase 5 | 34.37 | |
| | (SEQ ID NO: 158) | | | | |
| LOC100789303 | XP_006589707.1 | AT5G04310 | Pectate lyase | 4.17 | |
| | (SEQ ID NO: 142) | | | | |
| LOC100779940 | NP_001242543.1 | AT5G63180 | Probable pectate lyase 22 | 2.14 | |
| | (SEQ ID NO: 170) | | | | |
| LOC100802068 | XP_003556544.1 | AT3G55140 | Pectate lyase | 0.61 | |
| | (SEQ ID NO: 171) | | | | |
| LOC100777128 | XP_003528738.1 | AT1G53840 | Pectinesterase 1 | 1.51 | 0.58 |
| | (SEQ ID NO: 172) | | | | |
| LOC100814987 | XP_003551027.1 | AT3G43270 | Probable | 2.17 | |
| | (SEQ ID NO: 173) | | pectinesterase/pectinesterase | | |
| | | | inhibitor 32 | | |
| LOC100801231 | XP_003531408.1 | AT5G09760 | Probable | 44.94 | |
| | (SEQ ID NO: 152) | | pectinesterase/pectinesterase | | |
| | | | inhibitor 51 | | |
| LOC100777500 | XP_003546532.1 | AT4G33220 | Probable | 0.47 | |
| | (SEQ ID NO: 174) | | pectinesterase/pectinesterase | | |
| | | | inhibitor 44 | | |
| LOC100816190 | XP_003517421.1 | AT5G47500 | Probable pectinesterase 68 | 870.46 | |
| | (SEQ ID NO: 168) | | | | |
| LOC100810031 | XP_003538735.1 | AT5G47500 | Probable pectinesterase 68 | 262.40 | |
| | (SEQ ID NO: 167) | | | | |
| LOC100796327 | XP_003550907.1 | AT5G47500 | Probable pectinesterase 68 | 245.06 | |
| | (SEQ ID NO: 166) | | | | |
| LOC100793773 | XP_003524409.1 | AT5G19730 | Probable pectinesterase 53 | 64.24 | |
| | (SEQ ID NO: 162) | | | | |
| LOC100819675 | XP_003524299.2 | AT5G09760 | Probable | 48.82 | |
| | (SEQ ID NO: 164) | | pectinesterase/pectinesterase | | |
| | | | inhibitor 51 | | |
| LOC100802832 | XP_006584808.1 | AT5G19730 | Probable pectinesterase 53 | 47.04 | |
| | (SEQ ID NO: 163) | | | | |
| LOC100808236 | XP_006578909.1 | AT5G53370 | Probable | 38.78 | |
| | (SEQ ID NO: 165) | | pectinesterase/pectinesterase | | |
| | | | inhibitor 61 | | |
| LOC100793314 | XP_003516527.2 | AT5G19730 | Probable pectinesterase 53 | 28.80 | |
| | (SEQ ID NO: 161) | | | | |
| LOC100777646 | XP_003528739.1 | AT3G14310 | Pectinesterase/pectinesterase | 23.05 | |
| | (SEQ ID NO: 156) | | inhibitor 3 | | |
| LOC100776623 | XP_025981966.1 | AT3G14310 | Pectinesterase/pectinesterase | 14.82 | |
| | (SEQ ID NO: 155) | | inhibitor 3 | | |
| LOC100786786 | XP_006587735.1 | AT3G29090 | Pectinesterase 31 | 4.30 | |
| | (SEQ ID NO: 146) | | | | |
| LOC100776222 | XP_003527461.1 | AT1G41830 | SKU-similar 6 | 2.55 | |
| | (SEQ ID NO: 175) | | | | |
| LOC100789567 | XP_003527069.1 | AT1G53840 | Pectinesterase 1 | 0.51 | |
| | (SEQ ID NO: 176) | | | | |
| LOC100816686 | XP_003546521.1 | AT5G20740 | Pectinesterase inhibitor 3 | 173.35 | |
| | (SEQ ID NO: 169) | | | | |
| LOC100305900 | NP_001238450.1 | AT5G62350 | Replication protein A 32 kDa | 4.23 | |
| | (SEQ ID NO: 177) | | subunit A | | |
| LOC100812251 | XP_003530773.1 | AT1G14890 | Plant invertase/pectin | 2.33 | |
| | (SEQ ID NO: 150) | | methylesterase inhibitor | | |
| | | | superfamily protein | | |
| LOC100776636 | XP_003517432.1 | AT1G62770 | Pectinesterase inhibitor 9 | 0.19 | |
| | (SEQ ID NO: 149) | | | | |
| LOC100306719 | NP_001235420.1 | AT1G47960 | Cell wall/vacuolar inhibitor | 0.02 | |
| | (SEQ ID NO: 148) | | of fructosidase 1 | | |
| LOC100500640 | NP_001237215.2 | AT1G47960 | Cell wall/vacuolar inhibitor | 0.00 | 0.53 |
| | (SEQ ID NO: 147) | | of fructosidase 1 | | |
| LOC100776478 | XM_006587593.3 | AT5G45280 | Pectin acetylesterase 11 | 0.63 | |
| | (SEQ ID NO: 178) | | | | |
| LOC100781246 | XP_014624577.1 | AT4G19420 | Pectin acetylesterase 8 | 0.61 | |
| | (SEQ ID NO: 179) | | | | |

*Lines are identified in TABLE 15.

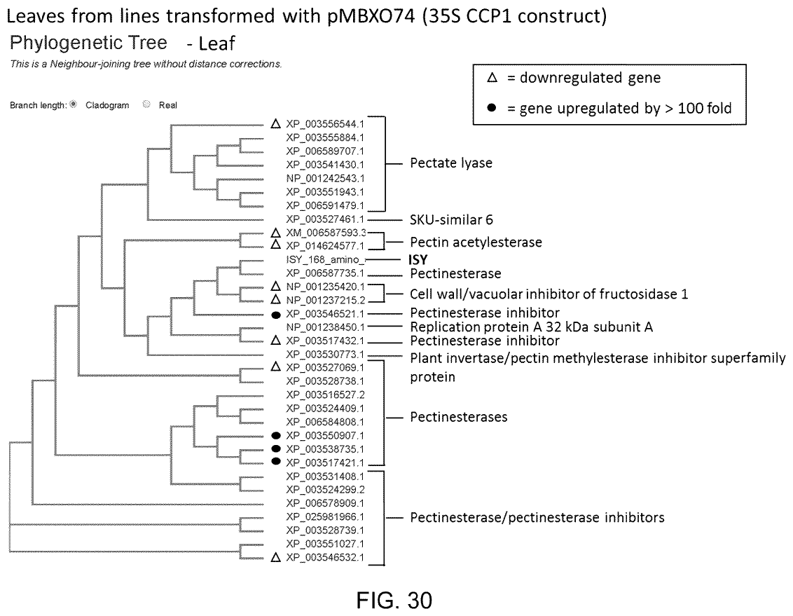

The Clustal Omega multiple sequence alignment tool was used to create a phylogenetic tree (cladogram) (FIG. 30) with all the soybean protein sequences listed in TABLE 18 and the 168 amino acid ISY gene (SEQ ID NO: 5). Differentially expressed genes in the CCP1+ soybean lines with upregulated expression greater than 100 fold, as well as all downregulated genes are labeled in FIG. 30. The phylogenetic tree (FIG. 30) and sequence alignments of the Camelina ISY protein with the proteins in TABLE 18 showed the closest matches to the ISY protein to be two cell wall/vacuolar inhibitors of fructosidase 1 [NP_001237215.2 (SEQ ID NO: 147) and NP_001235420.1 (SEQ ID NO: 148)] and a pectinesterase [XP_006587735.1 (SEQ ID NO: 146)].

### Differentially Expressed Genes in Seeds:

Three different kinds of seed samples were analyzed in the RNA SEQ experiment focusing on seeds: maturing seeds harvested from plants transformed with the 35S constitutive promoter construct (pMBXO74, FIG. 20A, SEQ ID NO: 128) and appropriate controls, as well as developing and maturing seeds from an oleosin seed specific promoter construct (pMBXO75, FIG. 20C, SEQ ID NO: 130) and appropriate controls. Additional information about the samples is described in TABLE 15.

For seed samples, RNA-seq data analysis was performed with the Bioconductor R package DESeq2 to determine differential expression. In this analysis, the Tukey test was not used to filter the data, but rather all comparisons for which σ/μ <0.75 for both lines were retained (is the mean value of the normalized reads for a particular line and a is their standard deviation). Only comparisons where the two mean read counts from RNA-Seq analysis added up to ≥2.00 were retained.

The genes that fulfilled these criteria and that fall within the GO categories listed in TABLE 16 are shown in TABLE 19. In TABLE 19, only comparisons in which the fold difference between the two compared lines exceeds 1.5 are included.

TABLE 19
Differentially expressed Glycine max genes from the plant invertase
inhibitor/pectin methylesterase inhibitor superfamily in maturing and
developing seeds of CCP1+ and null lines vs. the wild type control line.*

| Glycine max | | A. thaliana | A. thaliana | Fold | Fold | Fold |
|---|---|---|---|---|---|---|
| locus | Protein ID | locus | description | F1/D | P/L | K/H |
| LOC100802068 | XP_003556544.1 | AT3G55140 | Pectate lyase | 0.53 | | |
| | (SEQ ID NO: 171) | | | | | |
| LOC100814679 | XP_003523121.1 | AT5G63180 | Probable pectate | 0.35 | 0.63 | |
| | (SEQ ID NO: 180) | | lyase 22 | | | |
| LOC100101868 | XP_003549832.1 | AT5G63180 | Probable pectate | | | |
| | (SEQ ID NO: 181) | | lyase 22 | | | |
| LOC100799009 | XP_014618078.1 | AT3G55140 | Pectate lyase | | | 1.97 |
| | (SEQ ID NO: 182) | | | | | |
| LOC100801813 | XP_003534983.1 | AT4G33220 | Probable | 3.65 | 1.63 | |
| | (SEQ ID NO: 183) | | pectinesterase/pectinesterase | | | |
| | | | inhibitor 44 | | | |
| LOC100784642 | XP_003547912.1 | AT1G53840 | Pectinesterase 1 | 3.25 | | |
| | (SEQ ID NO: 184) | | | | | |
| LOC100808236 | XP_006578909.1 | AT5G53370 | Probable | 2.83 | | |
| | (SEQ ID NO: 165) | | pectinesterase/pectinesterase | | | |
| | | | inhibitor 61 | | | |
| LOC106796002 | XP_014622597.1 | AT5G53370 | Probable | 2.74 | | 1.99 |
| | (SEQ ID NO: 185) | | pectinesterase/pectinesterase | | | |
| | | | inhibitor 61 | | | |
| LOC100776222 | XP_003527461.1 | AT1G41830 | SKU-similar 6 | 2.33 | | |
| | (SEQ ID NO: 175) | | | | | |
| LOC100776752 | XP_003526711.1 | AT5G53370 | Probable | 2.23 | | 1.66 |
| | (SEQ ID NO: 153) | | pectinesterase/pectinesterase | | | |
| | | | inhibitor 61 | | | |
| LOC100777500 | XP_003546532.1 | AT4G33220 | Probable | 1.92 | | |
| | (SEQ ID NO: 174) | | pectinesterase/pectinesterase | | | |
| | | | inhibitor 44 | | | |
| LOC100789567 | XP_003527069.1 | AT1G53840 | Pectinesterase 1 | 1.87 | | 1.88 |
| | (SEQ ID NO: 176) | | | | | |
| LOC102666380 | XP_006593974.1 | AT2G26440 | Probable | 0.41 | | |
| | (SEQ ID NO: 186) | | pectinesterase/pectinesterase | | | |
| | | | inhibitor 12 | | | |
| LOC100819675 | XP_003524299.2 | AT5G09760 | Probable | | 1.52 | |
| | (SEQ ID NO: 164) | | pectinesterase/pectinesterase | | | |
| | | | inhibitor 51 | | | |
| LOC100815836 | XP_006573891.1 | AT1G11580 | Pectinesterase/pectinesterase | | 0.43 | |
| | (SEQ ID NO: 187) | | inhibitor 18 | | | |
| LOC100777646 | XP_003528739.1 | AT3G14310 | Pectinesterase/pectinesterase | | | 1.89 |
| | (SEQ ID NO: 156) | | inhibitor 3 | | | |
| LOC100791264 | XP_003521571.1 | AT3G62820 | Plant invertase/pectin | | 0.51 | |
| | (SEQ ID NO: 188) | | methylesterase | | | |
| | | | inhibitor superfamily protein | | | |
| LOC100797734 | XP_003533952.1 | AT2G01610 | Pectinesterase inhibitor 7 | | | 1.70 |
| | (SEQ ID NO: 189) | | | | | |
| LOC100776636 | XP_003517432.1 | AT1G62770 | Pectinesterase inhibitor 9 | | | 0.55 |
| | (SEQ ID NO: 149) | | | | | |
| LOC100795394 | XP_003529510.2 | AT1G47960 | Cell wall/vacuolar inhibitor | | | 1.64 |
| | (SEQ ID NO: 190) | | of fructosidase 1 | | | |
| LOC100776478 | XM_006587593.3 | AT5G45280 | Pectin acetylesterase 11 | 6.49 | | |
| | (SEQ ID NO: 178) | | | | | |
| LOC100788448 | XP_006604776.1 | AT3G62060 | Pectin acetylesterase 6 | 1.59 | | |
| | (SEQ ID NO: 191) | | | | | |
| LOC100780171 | XP_014623997.1 | AT4G19420 | Pectin acetylesterase 8 | 0.12 | | |
| | (SEQ ID NO: 192) | | | | | |

*Lines are identified in TABLE 15. There were not significant fold changes in the null lines for the genes in TABLE 19 and thus null lines are not shown in the TABLE 19.

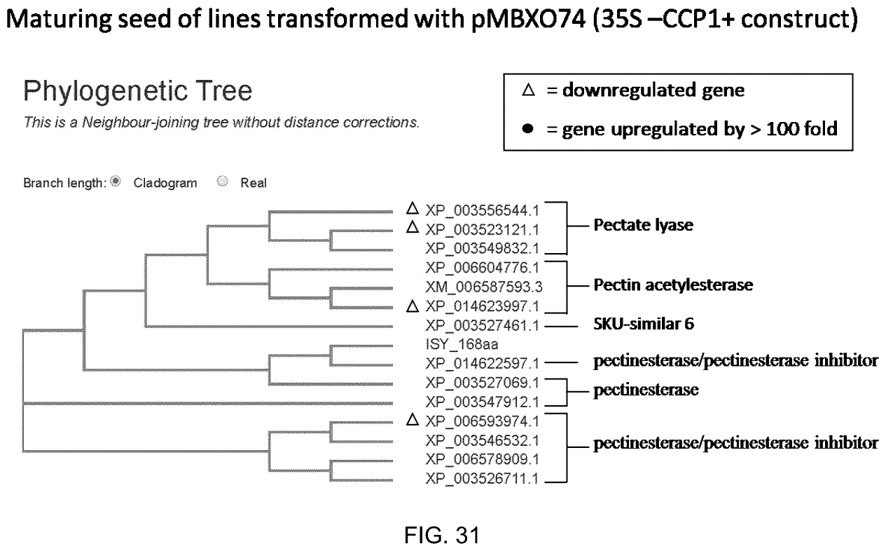

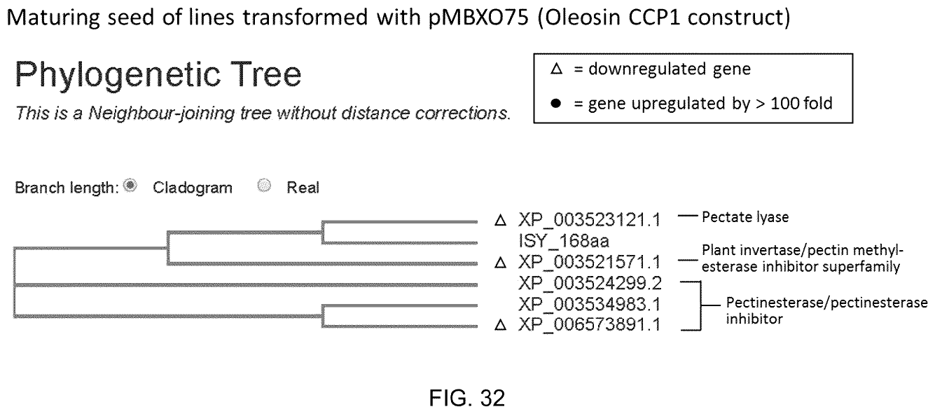

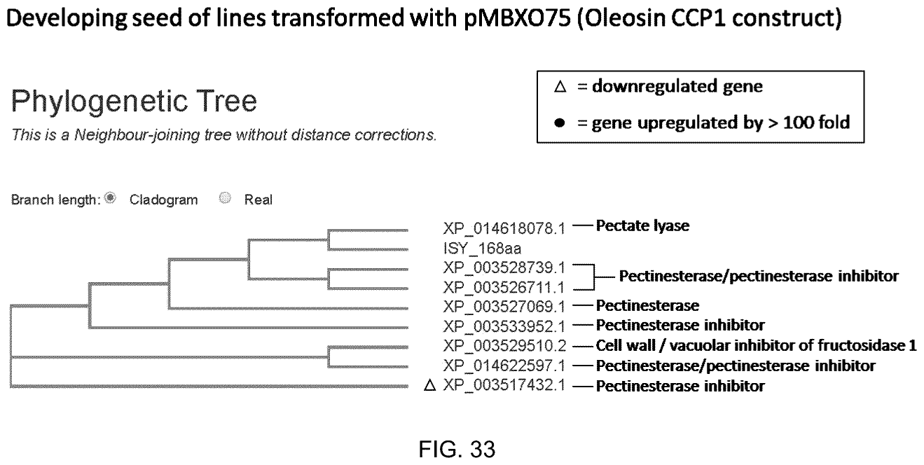

The Clustal Omega multiple sequence alignment tool was used to create phylogenetic trees (cladograms) with the soybean sequences listed in TABLE 19 and the 168 amino acid ISY gene (SEQ ID NO: 5) for the maturing seed of lines transformed with pMBXO74 (35S-CCP1 homozygous line) compared to Westag wild-type control (F1/D comparison in TABLE 19, FIG. 31), maturing seed of lines transformed with pMBXO75 (oleosin promoter—CCP1 homozygous line) compared to Westag (P/L comparison in TABLE 19, FIG. 32), and developing seed of lines transformed with pMBXO75 (oleosin promoter—CCP1 homozygous line) compared to Westag (K/H comparison in TABLE 19, FIG. 33). In FIGS. 31-33, differentially expressed genes in the CCP1+ soybean lines with upregulated expression greater than 100 fold, as well as all downregulated genes are labeled.

For the maturing seed of lines transformed with pMBXO74 (F1/D comparison in TABLE 19, FIG. 31), the phylogenetic tree and subsequent sequence alignments of proteins encoded by the differentially expressed genes with the Camelina ISY protein were used to find the genes most similar to ISY. XP_014622597.1 (SEQ ID NO: 185, a probable pectinesterase/pectinesterase inhibitor 61) and XP_003527069.1 (SEQ ID NO: 176, a pectinesterase 1) were the closest matching proteins in the phylogenetic tree. Interestingly, the gene encoding XP_003527069.1 (SEQ ID NO: 176) was found to be differentially expressed in both leaf (TABLE 18, C1/A comparison, downregulated) and maturing seed tissue (TABLE 19, F1/D comparison, upregulated) of lines transformed with pMBXO74.

For the maturing seed of lines transformed with pMBXO75 (P/L comparison in TABLE 19, FIG. 32), the phylogenetic tree and subsequent sequence alignments of proteins encoded by the differentially expressed genes with the Camelina ISY protein were used to find the genes most similar to ISY. The proteins XP_003523121.1 (SEQ ID NO: 180, a probable pectate lyase) and XP_003521571.1 (SEQ ID NO: 188, a plant invertase/pectin methylesterase inhibitor superfamily protein) were identified, both of which are encoded by downregulated genes in the pMBXO75 transformed lines. Interestingly, the gene encoding XP_003523121.1 (SEQ ID NO: 180) is also downregulated in the maturing seed of lines transformed with pMBXO74 (F1/D comparison in TABLE 19).

For the developing seed of lines transformed with pMBXO75 (K/H comparison in TABLE 19, FIG. 33), the phylogenetic tree and subsequent sequence alignments of proteins encoded by the differentially expressed genes with the Camelina ISY protein were used to find the genes most similar to ISY. The proteins XP_014618078.1 (SEQ ID NO: 182, a pectate lyase), XP 003528739.1 (SEQ ID NO: 156, a pectinesterase/pectinesterase inhibitor 3), and XP_003526711.1 (SEQ ID NO: 153, a probable pectinesterase/pectinesterase inhibitor 61) were identified.

Interestingly, the gene encoding XP_003528739.1 (SEQ ID NO: 156), was found to also be upregulated in the harvested leaves of lines transformed with pMBXO74 compared to wild-type controls (C1/A comparison, TABLE 18). The gene encoding XP_003526711.1 (SEQ ID NO: 153) was found to be upregulated in both maturing seed of lines transformed with pMBXO74 (F1/D comparison) and developing seed of lines transformed with pMBXO75 (K/H comparison).

Differentially expressed genes listed in TABLES 18 and 19 that are present in multiple samples and/or tissue types may be particularly interesting as targets for genetic manipulation through genome editing or other methods. TABLE 20 summarizes the Glycine max genes and their encoded proteins that are differentially expressed in RNA SEQ samples harvested from more than one sample or tissue type. For example, SEQ ID NO: 174, 176, and 178 are proteins encoded by differentially expressed genes that are downregulated in leaf tissue of lines transformed with pMBXO74 compared to wild-type controls (C1/A comparison) but are upregulated in mature seed tissue (F1/D comparison). This may be indicative of some change in carbon partitioning between leaves and seed tissue. Thus targets listed in TABLE 20 may represent preferred targets for engineering to increase seed yield and/or plant productivity.

TABLE 20
Differentially expressed Glycine max genes appearing
in multiple samples from TABLES 18 and 19.

| | | | | | | | |
|---|---|---|---|---|---|---|---|
| Glycine max | | A. thaliana | A. thaliana | FOLD | Fold | Fold | Fold |
| locus | Protein ID | locus | description | C1/A | F1/D | P/L | K/H |

Differentially expressed genes in both leaves and mature seeds of lines
transformed with pMBXO74 (C1/A and F1/D comparisons)

| | | | | | | |
|---|---|---|---|---|---|---|
| LOC100802068 | XP_003556544.1 | AT3G55140 | Pectate lyase | 0.61 | 0.53 | |
| | (SEQ ID NO: 171) | | | | | |
| LOC100808236 | XP_006578909.1 | AT5G53370 | Probable | 38.78 | 2.83 | |
| | (SEQ ID NO: 165) | | pectinesterase/ | | | |
| | | | pectinesterase | | | |
| | | | inhibitor 61 | | | |
| LOC100776222 | XP_003527461.1 | AT1G41830 | SKU-similar 6 | 2.55 | 2.33 | |
| | (SEQ ID NO: 175) | | | | | |
| LOC100777500 | XP_003546532.1 | AT4G33220 | Probable | 0.47 | 1.92 | |
| | (SEQ ID NO: 174) | | pectinesterase/ | | | |
| | | | pectinesterase | | | |
| | | | inhibitor 44 | | | |
| LOC100789567 | XP_003527069.1 | AT1G53840 | Pectinesterase 1 | 0.51 | 1.87 | 1.88 |
| | (SEQ ID NO: 176) | | | | | |
| LOC100776478 | XM_006587593.3 | AT5G45280 | Pectin | 0.63 | 6.49 | |
| | (SEQ ID NO: 178) | | acetylesterase 11 | | | |

Differentially expressed genes in mature seeds of lines transformed with pMBXO74 and pMBXO75
(F1/D and P/L comparisons)

| | | | | | |
|---|---|---|---|---|---|
| LOC100814679 | XP_003523121.1 | AT5G63180 | Probable | 0.35 | 0.63 |
| | (SEQ ID NO: 180) | | pectate lyase 22 | | |
| LOC100801813 | XP_003534983.1 | AT4G33220 | Probable | 3.65 | 1.63 |
| | (SEQ ID NO: 183) | | pectinesterase/ | | |
| | | | pectinesterase | | |
| | | | inhibitor 44 | | |

Differentially expressed genes in both leaves of lines transformed with pMBXO74 (C1/A comparison)
and developing seeds of lines transformed with pMBXO75 (K/H comparison)

| | | | | | | |
|---|---|---|---|---|---|---|
| LOC100789567 | XP_003527069.1 | AT1G53840 | Pectinesterase | 0.51 | 1.87 | 1.88 |
| | (SEQ ID NO: 176) | | 1 | | | |
| LOC100777646 | XP_003528739.1 | AT3G14310 | Pectinesterase/ | 23.05 | | 1.89 |
| | (SEQ ID NO: 156) | | pectinesterase | | | |
| | | | inhibitor 3 | | | |
| LOC100776636 | XP_003517432.1 | AT1G62770 | Pectinesterase | 0.19 | | 0.55 |
| | (SEQ ID NO: 149) | | inhibitor 9 | | | |

Differentially expressed genes in both mature seeds of lines transformed with pMBXO74 (F1/D
comparison) and developing seeds of lines transformed with pMBXO75 (K/H comparison)

| | | | | | | |
|---|---|---|---|---|---|---|
| LOC106796002 | XP_014622597.1 | AT5G53370 | Probable | | 2.74 | 1.99 |
| | (SEQ ID NO: 185) | | pectinesterase/ | | | |
| | | | pectinesterase | | | |
| | | | inhibitor 61 | | | |
| LOC100776752 | XP_003526711.1 | AT5G53370 | Probable | | 2.23 | 1.66 |
| | (SEQ ID NO: 153) | | pectinesterase/ | | | |
| | | | pectinesterase | | | |
| | | | inhibitor 61 | | | |
| LOC100789567 | XP_003527069.1 | AT1G53840 | Pectinesterase 1 | 0.51 | 1.87 | 1.88 |
| | (SEQ ID NO: 176) | | | | | |

### Example 16. Creation of Soybean Lines with Overexpressed Genes Identified in TABLES 17, 18, 19, and 20

Soybean lines can be engineered to overexpress the genes, or combinations of the genes, identified in TABLES 17, 18, 19, and 20 by creating an expression cassette with a promoter operably linked to the gene of interest operably linked to a termination sequence. For constitutive expression, a promoter such as the cauliflower mosaic virus promoter (SEQ ID NO: 108) can be used. For expression primarily in green tissue, a promoter such as the soybean cab5 promoter (TABLE 1, SEQ ID NO: 42) can be used. For seed specific expression, a promoter such as the soybean oleosin promoter (TABLE 1, SEQ ID NO: 50) can be used. Soybean can be transformed with expression cassettes overexpressing the gene sequences identified in TABLES 17, 18, 19, and 20 using the procedures described in Example 5. Gene targets in TABLE 20, that were found to be differentially expressed in more than one harvested sample or tissue type, may be preferred targets for this exercise.

Soybean lines can also be engineered to overexpress the genes identified in TABLES 17, 18, 19, and 20 using a promoter replacement strategy with the CRISPR/Cas system. In this system, the native promoter in front of a gene is replaced with a stronger promoter, or a promoter with a different expression pattern, using a strategy similar to the one described in FIG. 22 and Example 11.

The CRISPR/Cas system can also be used to insert a new expression cassette into the plant genome containing a promoter operably linked to a gene identified in TABLES 17, 18, and 19, operably linked to a termination sequence. For this procedure, one or more sgRNAs are used with the Cas enzyme to generate a double stranded break. The new expression cassette, flanked by sequences with homology to the insertion site of the plant genome, is inserted using the plant's homology directed repair mechanism.

### Example 17. Creation of Soybean Lines with Reduced Activity of Genes Identified in TABLES 17, 18, 19, and 20

Soybean lines can be engineered for reduced expression of one or more genes listed in TABLES 17, 18, 19, and 20 using antisense technology or RNAi technology. A preferred method is to use genome editing, such as the CRISPR/CAS genome editing technology.

To reduce expression of a native plant gene, the CRISPR/Cas system can be used to replace the native promoter in front of a gene with a weaker promoter using a strategy similar to the one described in FIG. 22 and Example 11. The expression of the pectinesterase (XP_003543834.1, SEQ ID NO: 145), the cell wall/vacuolar inhibitor of fructosidase 1 (NP_001237215.2, SEQ ID NO: 147 and NP_001235420.1, SEQ ID NO: 148), and/or the pectinesterase inhibitor (XP_003517432.1, SEQ ID NO: 149) genes can be reduced using this strategy. Other useful targets for downregulation or deletion include XP_003556544.1 (SEQ ID NO: 171), XP_003523121.1 (SEQ ID NO: 180), XP_003546532.1 (SEQ ID NO: 174), XP_003527069.1 (SEQ ID NO: 176), XP_006593974.1 (SEQ ID NO: 186), XP_006573891.1 (SEQ ID NO: 187), XP_003521571.1 (SEQ ID NO: 188), XM_006587593.3 (SEQ ID NO: 178), and XP_014623997.1 (SEQ ID NO: 192).

Alternatively, the CRISPR/Cas system can be used to weaken the strength of a promoter by inducing a mutation in an important region of the promoter and allowing non-homologous end joining (NHEJ) to occur.

Alternatively, the activity of a protein can be reduced or eliminated by removing, inserting, or changing one or more base pairs in the sequence of a gene. This can be achieved by designing a sgRNA with its ˜20 bp guide sequence targeting the region of the gene to be modified. With the CRISPR/Cas enzyme, the sgRNA can be used to create INDELs (insertion or deletion of a small number of bases). The Cas nuclease makes a double strand break near the binding of the sgRNA and the error prone DNA repair pathway, with non-homologous end joining, corrects the break creating a mutation. Out of frame mutations can lead to elimination of the enzyme's activity. In frame insertion or deletion mutations can reduce the activity of the protein. The activities of the pectinesterase (XP_003543834.1, SEQ ID NO: 145), the cell wall/vacuolar inhibitor of fructosidase 1 (NP_001237215.2, SEQ ID NO: 147 and NP_001235420.1, SEQ ID NO: 148), and/or the pectinesterase inhibitor (XP_003517432.1, SEQ ID NO: 149) genes in TABLE 17 can be reduced or eliminated using this strategy.

Other useful targets for downregulation or deletion include XP_003556544.1 (SEQ ID NO: 171), XP_003523121.1 (SEQ ID NO: 180), XP_003546532.1 (SEQ ID NO: 174), XP_003527069.1 (SEQ ID NO: 176), XP_006593974.1 (SEQ ID NO: 186), XP_006573891.1 (SEQ ID NO: 187), XP_003521571.1 (SEQ ID NO: 188), XM_006587593.3 (SEQ ID NO: 178), and XP_014623997.1 (SEQ ID NO: 192).

Gene targets in TABLE 20, expressed in more than one sample or tissue type, may be preferred targets for this exercise.

### Example 18. Identification of Up- and Down-Regulated Genes in Canola Lines Expressing the CCP1 Gene Using RNA Sequencing

Select canola T4 generation lines expressing the CCP1 gene (TABLE 21), produced as described in Example 5, were chosen for analysis by RNA sequencing. These lines were grown in the field at a site in Canada during the spring/summer of 2017. TABLE 21 shows lines that were selected for analysis, the tissue type collected, and classifies the lines into groups for analysis purposes. Samples were harvested at the indicated stage and quickly frozen in liquid nitrogen. Samples were stored at −80° C. RNA was extracted using the Agilent Plant RNA isolation kit and shipped to a contract service provider that performed RNA Sequencing and data processing. Differentially expressed genes from this analysis are shown in TABLE 22.

TABLE 21
Brassica napus samples used in the RNA-seq analysis.

| | | | Ni- | | Increased |
|---|---|---|---|---|---|
| | | | trogen | | seed yield |
| | | | addi- | | relative to |
| Group | Tissue¹ | Line | tion² | CCP1 | control³ |
| Q | Leaf | BN00 wild-type control | 150% | − | — |
| R | Leaf | MW82 (pMBXO58, 35S- | 150% | + | 13.1%* |
| | | CCP1 homozygous) | | | |
| S | Silique | BN00 wild-type control | 150% | − | — |
| T | Silique | MW82 (pMBXO58, 35S- | 150% | + | 13.1%* |
| | | CCP1 homozygous) | | | |
| U | Leaf | BN00 wild-type control | 50% | − | — |
| W | Leaf | MW82 (pMBXO58, 35S- | 50% | + | 8.7%* |
| | | CCP1 homozygous) | | | |
| X | Silique | BN00 wild-type control | 50% | − | — |
| Y | Silique | MW82 (pMBXO58, 35S- | 50% | + | 8.7%* |
| | | CCP1 homozygous) | | | |

¹Harvested tissue stages are as follows: Leaf, newly expanded leaf from 39 day old canola plants; silique, developing silique collected 15 days after flowering;
²50% or 150% of the nitrogen required to provide a 40 bushel per acre canola yield. AGROTAIN(R) fertilizer used.
³Statistically significant, P < 0.1. The map of plasmid pMBXO58 is shown in FIG. 3.

In order to analyze genes in the same family as the ISY gene (Csa15g017550.1; SEQ ID NO: 6), the GO terms listed in TABLE 16 were investigated. The resulting up- and down-regulated genes, along with the comparison corresponding to the fold change, are given in TABLE 22. Only comparisons in which the fold difference between the two lines exceeds 1.5 are included.

TABLE 22
Differentially expressed Brassica napus genes from the plant invertase inhibitor/pectin
methylesterase inhibitor superfamily in CCP1+ lines vs. wild type.
GO:0030570 pectate lyase activity

| | | Fold change* | | | |
|---|---|---|---|---|---|
| | | Leaf | Leaf | Silique | Silique |
| B. napus locus | Description | R/Q | W/U | T/S | Y/X |
| LOC106367786 | Probable pectate lyase 12 | 3.2 | | | |
| LOC106361318 | Probable pectate lyase 18 | 0.49 | | | |
| LOC106363861 | Probable pectate lyase 1 | 2.9 | | | |
| LOC106452978 | Probable pectate lyase 8 | | | 2.1 | |
| LOC106430113 | Probable pectate lyase 9 | | | | 1.7 |

| | | Fold change | | | |
|---|---|---|---|---|---|
| | | Leaf | Leaf | Silique | Silique |
| B. napus locus | Description | R/Q | W/U | T/S | Y/X |

GO:0030599 pectinesterase activity

| | | |
|---|---|---|
| LOC111205093 | Probable pectinesterase 29 | 0.41 |

GO:0046910 pectinesterase inhibitor activity

| | | | | |
|---|---|---|---|---|
| LOC106447685 | Probable pectinesterase/ | 2.0 | | |
| | pectinesterase inhibitor 34 | | | |
| LOC106371735 | Probable pectinesterase/ | 1.8 | | |
| | pectinesterase inhibitor 64 | | | |
| LOC106416267 | Pectinesterase/pectinesterase | 1.8 | | |
| | inhibitor 18 | | | |
| LOC106412069 | Pectinesterase/pectinesterase | 1.6 | | |
| | inhibitor 18 | | | |
| LOC106434483 | Probable pectinesterase/ | | | 0.66 |
| | pectinesterase inhibitor 16 | | | |
| LOC106347212 | Plant invertase/pectin | 1.6 | | |
| | methylesterase inhibitor | | | |
| | superfamily protein | | | |
| LOC106392321 | Pectinesterase inhibitor 6 | 2.9 | | |
| LOC106436652 | Pectin methylesterase inhibitor 1 | | 1.7 | |
| LOC106450421 | Pectinesterase inhibitor 6 | | 0.51 | |
| LOC111205564 | Pectinesterase inhibitor 6 | | 0.48 | |
| LOC106397464 | Pectinesterase inhibitor 10 | | | 1.5 |

GO:0052793 pectin acetylesterase activity

| | | | |
|---|---|---|---|
| LOC106368499 | Pectin acetylesterase 6 | | 0.67 |
| LOC106450464 | Pectin acetylesterase 3 | | 1.8 |
| LOC106450462 | Pectin acetylesterase 3 | | 1.6 |
| LOC106377080 | Pectin acetylesterase 7 | 1.6 | |

*Groups described in TABLE 21.

An alternative filtering of the genes was performed to see if any new genes appeared. In this case, the Tukey test was not used to filter the data, but rather all comparisons for which ab/0.75 for both lines were retained, where is the mean value of the normalized reads for a particular line and a is their standard deviation. The up- and downregulated genes obtained with this filtering method, along with the comparison corresponding to the fold change, are given in TABLE 23. Only comparisons in which the fold difference between the two lines exceeds 2.0 are included.

TABLE 23
Differentially expressed Brassica napus genes from the
GO categories in TABLE 16 in CCP1+ lines vs. wild type.
GO:0030570 pectate lyase activity

| | | Fold change* | | | |
|---|---|---|---|---|---|
| B. napus locus | Description | RQ | W/U | T/S | Y/X |
| LOC106367786 | probable pectate lyase 12 | 3.2 | — | — | — |
| LOC106422643 | probable pectate lyase 22 | 3.2 | — | — | — |
| LOC106363861 | probable pectate lyase 1 | 2.9 | — | — | — |
| LOC106409544 | probable pectate lyase 5 | 2.5 | — | — | — |
| LOC106419852 | probable pectate lyase 1, transcript | 2.3 | — | — | — |
| | variant X1 | | | | |
| LOC106436935 | probable pectate lyase 18 | 2.2 | — | — | — |
| LOC106381139 | probable pectate lyase 5 | 2.2 | — | — | — |
| LOC106370994 | probable pectate lyase 1 | 2.1 | — | — | — |
| LOC106361318 | probable pectate lyase 18 | 0.50 | — | — | — |
| LOC106352880 | probable pectate lyase 13 | — | 2.4 | — | — |
| LOC106452978 | probable pectate lyase 8 | — | — | 2.1 | — |
| LOC111208330 | probable pectate lyase 18 | — | — | 2.1 | — |

| | | Fold change | | | |
|---|---|---|---|---|---|
| B. napus locus | Description | R/Q | W/U | T/S | Y/X |

GO:0030599 pectinesterase activity

| | | | | | |
|---|---|---|---|---|---|
| LOC106447685 | probable pectinesterase/ | 2.0 | — | — | — |
| | pectinesterase inhibitor 34 | | | | |
| LOC106365512 | probable pectinesterase/ | 0.43 | — | — | — |
| | pectinesterase inhibitor 41 | | | | |
| LOC111205093 | probable pectinesterase 29 | — | — | — | 0.41 |

GO:0046910 pectinesterase inhibitor activity

| | | | | | |
|---|---|---|---|---|---|
| LOC106392321 | pectinesterase inhibitor 6-like | 2.9 | — | — | — |
| LOC106365512 | probable pectinesterase/ | 0.43 | — | — | — |
| | pectinesterase inhibitor 41 | | | | |

GO:0052793 pectin acetylesterase activity

| | | | | | |
|---|---|---|---|---|---|
| LOC106377080 | pectin acetylesterase 7 | 2.1 | — | — | — |
| LOC106416258 | pectin acetylesterase 6-like | 2.1 | — | — | — |
| LOC106450464 | pectin acetylesterase 3-like | — | 0.44 | — | — |

*Groups described in TABLE 21.

### Example 19. Identification of Up- and Down-Regulated Genes in Camelina sativa Lines Expressing the CCP1 Gene Using RNA Sequencing

Select homozygous Camelina sativa lines expressing the CCP1 gene (TABLE 24) were chosen for analysis by RNA sequencing. These lines were grown in the field at a site in Canada during the spring/summer of 2016. TABLE 24 shows lines that were selected for analysis, the tissue type collected, and classifies the lines into groups for analysis purposes. Samples were harvested at the indicated stage and quickly frozen in liquid nitrogen. Samples were stored at −80° C. RNA was extracted from newly expanded leaves (33 days after planting) and developing siliques (15 days after flowering) using the Agilent Plant RNA isolation kit and shipped to a contract service provider that performed RNA Sequencing and data processing.

TABLE 24
Camelina sativa samples used in the RNA-seq analysis.

| Group | Tissue* | Line | CCP1 |
|---|---|---|---|
| A | Leaf | MI116 | + |
| B | Leaf | WT43 | − |
| C | Leaf | NJ01 | + |
| D | Leaf | NJ02 | + |
| E | Leaf | WTSU | − |
| F | Silique | MI116 | + |
| G | Silique | WT43 | − |
| H | Silique | NJ01 | + |
| I | Silique | NJ02 | + |
| J | Silique | WTSU | − |

*Leaf, newly expanded leaves 33 days after planting; Silique, siliques harvested 15 days after flowering. WT43, abbreviation for Camelina sativa cultivar 10CS0043 that was isolated in a breeding program at Agriculture and Agri-Food Canada (Malik et al., 2018, Plant Cell Reports, 37, 1367). MI116, line obtained upon transformation of Camelina cultivar WT43 with plasmid pMBXO58 (FIG. 3). WTSU, abbreviation for Camelina sativa cultivar Suneson (Malik et al., 2018, Plant Cell Reports, 37, 1367). NJ01 and NJ02, lines obtained upon transformation of Camelina cultivar Suneson with plasmid pMBXO58.

In order to analyze genes in the same family as the ISY gene (Csa15g017550.1; SEQ ID NO: 6), the GO terms listed in TABLE 16 were investigated. The objective was to search the Camelina sativa genome for differentially regulated genes that could be associated with one or more of these GO terms. Nearly all genes within the RNA-seq data had a corresponding best BLAST hit in the Arabidopsis thaliana genome, which is very well-annotated with respect to associated GO terms. Therefore, for each of the above GO terms, a list of Arabidopsis thaliana genes associated with each GO term was made, then all genes within the Camelina sativa genome that had one of those Arabidopsis thaliana genes as its best BLAST hit were identified. Among these Camelina sativa genes, some were up- or down-regulated in CCP1 lines in a statistically significant way; that is, a Tukey test suggested that the probability was greater than 90% that the means of the transcript levels in transgenic vs. wild-type lines were different.

The up- and down-regulated genes, along with the comparison corresponding to the fold change, are given in TABLE 25. Only comparisons in which the fold difference between the two lines exceeds 1.5 are included.

TABLE 25
Differentially expressed Camelina sativa genes from the
GO categories in TABLE 16 in CCP1+ lines vs. wild type.
GO: 0030570 pectate lyase activity

| | | Fold change* | | | | | |
|---|---|---|---|---|---|---|---|
| | | Leaf | Leaf | Leaf | Silique | Silique | Silique |
| C. sativa locus | Description | A/B | C/E | D/E | F/G | H/J | I/J |
| LOC104734443 | Pectate lyase | 0.55 | | | | | |
| LOC104747027 | Probable pectate | | | 1.7 | | | |
| | lyase 10 | | | | | | |
| LOC104701382 | Probable pectate | | | | 2.0 | | |
| | lyase 5 | | | | | | |
| LOC104717795 | probable pectate | | | | | 2.4 | 2.3 |
| | lyase 18 | | | | | | |
| LOC104722501 | probable pectate | | | | | 2.2 | 2.5 |
| | lyase 18 | | | | | | |
| LOC104726924 | Probable pectate | | | | | 0.57 | 0.60 |
| | lyase 22 | | | | | | |
| LOC104726926 | Probable pectate | | | | | 0.54 | 0.59 |
| | lyase 22 | | | | | | |
| LOC104762495 | probable pectate | | | | | 2.1 | — |
| | lyase 1 | | | | | | |
| LOC104754610 | probable pectate | | | | | 2.0 | 1.8 |
| | lyase 1 | | | | | | |
| LOC104738994 | probable pectate | | | | | 1.9 | |
| | lyase 1 | | | | | | |
| LOC104762495 | Probable pectate | | | | | | 1.7 |
| | lyase 1 | | | | | | |

| | | Fold change | | | | | |
|---|---|---|---|---|---|---|---|
| | | Leaf | Leaf | Leaf | Silique | Silique | Silique |
| C. sativa locus | Description | A/B | C/E | D/E | F/G | H/J | I/J |

GO: 0030599 pectinesterase activity

| | | | | | |
|---|---|---|---|---|---|
| LOC104758734 | Pectinesterase 2 | 0.31 | | | |
| LOC104735956 | Probable | | 0.66 | | |
| | pectinesterase 29 | | | | |
| LOC104736702 | Putative pectinesterase 52 | | 1.7 | | |
| LOC104726108 | Pectinesterase QRT1 | | | 0.56 | |
| LOC104732089 | Pectinesterase QRT1 | | | 0.53 | 0.66 |
| LOC104742103 | Putative pectinesterase | | | 1.8 | 1.9 |
| LOC104777520 | Putative pectinesterase | | | 1.7 | 1.7 |
| LOC104757890 | Putative pectinesterase | | | 1.5 | 1.7 |
| LOC104736040 | Probable | | | | 1.6 |
| | pectinesterase 53 | | | | |
| LOC104770344 | Probable | | | | 1.6 |
| | pectinesterase 53 | | | | |

GO: 0046910 pectinesterase inhibitor activity

| | | | | | |
|---|---|---|---|---|---|
| LOC104745251 | Probable pectinesterase/ | | 1.5 | | |
| | pectinesterase inhibitor 25 | | | | |
| LOC104707868 | Probable pectinesterase/ | | | 2.7 | |
| | pectinesterase inhibitor 41 | | | | |
| LOC104708142 | Probable pectinesterase/ | | | 1.5 | |
| | pectinesterase inhibitor 51 | | | | |
| LOC104791148 | Probable pectinesterase/ | | | 1.7 | |
| | pectinesterase inhibitor 34 | | | | |
| LOC104760099 | Probable pectinesterase/ | | | 2.7 | 2.8 |
| | pectinesterase inhibitor 7 | | | | |
| LOC104754377 | Probable pectinesterase/ | | | 2.5 | 2.1 |
| | pectinesterase inhibitor 7 | | | | |
| LOC104778832 | Probable pectinesterase/ | | | 0.66 | |
| | pectinesterase inhibitor 6 | | | | |
| LOC104769166 | Probable pectinesterase/ | | | | 1.7 |
| | pectinesterase inhibitor 51 | | | | |
| LOC104708141 | Probable pectinesterase/ | | | | 1.5 |
| | pectinesterase inhibitor 51 | | | | |
| LOC104770309 | Pectinesterase/pectinesterase | | | | 1.8 |
| | inhibitor 18 | | | | |
| LOC104728508 | Plant invertase/pectin | 2.0 | | | |
| | methylesterase inhibitor | | | | |
| | superfamily protein | | | | |
| LOC109132214 | Plant invertase/pectin | 0.37 | | | |
| | methylesterase inhibitor | | | | |
| | superfamily protein | | | | |
| LOC104765570 | Plant invertase/pectin | 0.36 | | | |
| | methylesterase inhibitor | | | | |
| | superfamily protein | | | | |
| LOC104749039 | Plant invertase/pectin | | 2.0 | | |
| | methylesterase inhibitor | | | | |
| | superfamily protein | | | | |
| LOC104699600 | Plant invertase/pectin | | 1.6 | | |
| | methylesterase inhibitor | | | | |
| | superfamily protein | | | | |
| LOC104788614 | Plant invertase/pectin | | 1.6 | | |
| | methylesterase inhibitor | | | | |
| | superfamily protein | | | | |
| LOC104715795 | Invertase/pectin | | 2.1 | | |
| | methylesterase inhibitor | | | | |
| | family protein | | | | |
| LOC104779478 | Plant invertase/pectin | | 1.7 | | |
| | methylesterase inhibitor | | | | |
| LOC104714617 | Pectinesterase inhibitor 10 | | | 0.11 | 0.080 |
| LOC104707529 | Pectin methylesterase | | | 2.1 | 1.7 |
| | inhibitor 1 | | | | |
| LOC104737245 | Pectin methylesterase | | | 1.8 | 1.5 |
| | inhibitor 1 | | | | |
| LOC104717621 | Pectin methylesterase | | | 1.6 | |
| | inhibitor 1 | | | | |
| LOC104790946 | Pectinesterase inhibitor 11 | | | 2.1 | |
| LOC104711196 | Pectinesterase inhibitor 11 | | | 2.1 | |
| LOC104730912 | Pectinesterase inhibitor 4 | | | 2.7 | 2.0 |
| LOC104778832 | Probable pectinesterase/ | | | 0.66 | |
| | pectinesterase inhibitor 6 | | | | |
| LOC104756693 | Plant invertase/pectin | | | 2.1 | 2.1 |
| | methylesterase inhibitor | | | | |
| | superfamily protein | | | | |
| LOC104776449 | Plant invertase/pectin | | | 1.7 | 1.6 |
| | methylesterase inhibitor | | | | |
| | superfamily protein | | | | |
| LOC104756686 | Plant invertase/pectin | | | 1.7 | |
| | methylesterase inhibitor | | | | |
| | superfamily protein | | | | |
| LOC104741040 | Plant invertase/pectin | | | 1.9 | |
| | methylesterase inhibitor | | | | |
| | superfamily protein | | | | |
| LOC104745508 | Plant invertase/pectin | | | | 0.65 |
| | methylesterase inhibitor | | | | |
| | superfamily protein | | | | |
| LOC104765054 | Plant invertase/pectin | | | | 0.62 |
| | methylesterase inhibitor | | | | |
| | superfamily protein | | | | |
| LOC104777086 | Plant invertase/pectin | | | | 0.50 |
| | methylesterase inhibitor | | | | |
| | superfamily protein | | | | |
| LOC104765554 | CASP-like protein | | | | 1.7 |

GO: 0052793 pectin acetylesterase activity

| | | | | |
|---|---|---|---|---|
| LOC104704143 | Pectin acetylesterase 6 | 0.64 | | |
| LOC104714970 | Pectin acetylesterase 3 | | 2.0 | |
| LOC104723107 | Pectin acetylesterase 7 | | 1.7 | 1.7 |
| LOC109124411 | Pectin acetylesterase 11 | | 1.7 | |

*Groups described in TABLE 24.

An alternative filtering of the genes was performed to see if any new genes appeared. In this case, the Tukey test was not used to filter the data, but rather all comparisons for which σ/μ<0.75 for both lines were retained, where i is the mean value of the normalized reads for a particular line and a is their standard deviation. The up- and down-regulated genes obtained with the altered filtering method, along with the comparison corresponding to the fold change, are shown in TABLE 26. Only comparisons in which the fold difference between the two lines exceeds 2.0 are included.

TABLE 26
Differentially expressed Camelina sativa genes from the
GO categories in TABLE 16 in CCP1+ lines vs. wild type.
GO: 0030570 pectate lyase activity

| | | Fold change* | | | | | |
|---|---|---|---|---|---|---|---|
| | | Leaf | Leaf | Leaf | Silique | Silique | Silique |
| C. sativa locus | Description | A/B | C/E | D/E | F/G | H/J | I/J |
| LOC104717795 | probable pectate | — | — | — | — | 2.4 | 2.3 |
| | lyase 18 | | | | | | |
| LOC104722501 | probable pectate | — | — | — | — | 2.2 | 2.5 |
| | lyase 18 | | | | | | |
| LOC104762495 | probable pectate | — | — | — | — | 2.1 | — |
| | lyase 1 | | | | | | |
| LOC104754610 | probable pectate | — | — | — | — | 2.0 | — |
| | lyase 1 | | | | | | |
| LOC104730966 | probable pectate | — | — | — | — | — | 2.3 |
| | lyase 18 | | | | | | |

| | | Fold change | | | | | |
|---|---|---|---|---|---|---|---|
| | | Leaf | Leaf | Leaf | Silique | Silique | Silique |
| C. sativa locus | Description | A/B | C/E | D/E | F/G | H/J | I/J |

GO: 0030599 pectinesterase activity

| | | | | | | | |
|---|---|---|---|---|---|---|---|
| LOC104781810 | probable pectinesterase/ | — | — | — | — | 2.4 | — |
| | pectinesterase inhibitor 35 | | | | | | |
| LOC104769165 | probable pectinesterase/ | — | — | — | — | 2.3 | 2.1 |
| | pectinesterase inhibitor 51 | | | | | | |
| LOC104782819 | probable pectinesterase/ | — | — | — | — | 2.3 | — |
| | pectinesterase inhibitor 16 | | | | | | |
| LOC104779154 | pectinesterase/ | — | — | — | — | 2.2 | — |
| | pectinesterase inhibitor 3 | | | | | | |
| LOC104780230 | probable pectinesterase/ | — | — | — | — | 2.1 | — |
| | pectinesterase inhibitor 32 | | | | | | |
| LOC104788138 | probable pectinesterase/ | — | — | — | — | 2.1 | — |
| | pectinesterase inhibitor 35 | | | | | | |
| LOC104728462 | probable pectinesterase/ | — | — | — | — | 2.1 | — |
| | pectinesterase inhibitor 61 | | | | | | |
| LOC104699225 | probable pectinesterase/ | — | — | — | — | 2.1 | — |
| | pectinesterase inhibitor 35 | | | | | | |
| LOC104709803 | probable pectinesterase/ | — | — | — | — | 2.1 | 3.4 |
| | pectinesterase inhibitor 39 | | | | | | |

GO: 0046910 pectinesterase inhibitor activity

| | | | | | | | |
|---|---|---|---|---|---|---|---|
| LOC104783356 | 21 kDa protein-like | — | — | — | 3.0 | — | — |
| LOC104730912 | 21 kDa protein-like | — | — | — | — | 2.7 | — |
| LOC104780536 | 21 kDa protein-like | — | — | — | — | 2.5 | — |
| LOC104781810 | probable pectinesterase/ | — | — | — | | 2.4 | — |
| | pectinesterase inhibitor 35 | | | | | | |
| LOC104782819 | probable pectinesterase/ | — | — | — | | 2.3 | — |
| | pectinesterase inhibitor 16 | | | | | | |
| LOC104703885 | 21 kDa protein-like | — | — | — | — | 2.3 | — |
| LOC104779154 | pectinesterase/ | — | — | — | — | 2.2 | — |
| | pectinesterase inhibitor 3 | | | | | | |
| LOC104790946 | 21 kDa protein-like | — | — | — | — | 2.1 | — |
| LOC104756693 | 21 kDa protein-like | — | — | — | — | 2.1 | — |
| LOC104707529 | 21 kDa protein | — | — | — | — | 2.1 | — |
| LOC104788138 | probable pectinesterase/ | | | | | 2.1 | |
| | pectinesterase inhibitor 35 | | | | | | |
| LOC104711196 | 21 kDa protein-like | — | — | — | — | 2.1 | — |
| LOC104728462 | probable pectinesterase/ | — | — | — | — | 2.1 | — |
| | pectinesterase inhibitor 61 | | | | | | |
| LOC104699225 | probable pectinesterase/ | — | — | — | — | 2.1 | — |
| | pectinesterase inhibitor 35 | | | | | | |
| LOC104709803 | probable pectinesterase/ | — | — | — | — | — | 3.4 |
| | pectinesterase inhibitor 39 | | | | | | |
| LOC104777086 | uncharacterized | — | — | — | — | — | 0.50 |
| | LOC104777086 | | | | | | |
| LOC104714617 | 21 kDa protein-like | — | — | — | — | — | 0.08 |

*Groups described in TABLE 24.

### Example 20. Genetically Engineered Land Plants that Express the Genes Identified in TABLES 17, 18, 19, 20, 22, 23, 25, and 26

The results described in Examples 15, 18, and 19 and the genes identified in TABLES 17, 18, 19, 20, 22, 23, 25, and 26 validate the use of constitutive and seed specific expression of the mitochondrial transporter encoded by the algal gene CCP1 in soybean, canola, and Camelina sativa for identification of gene targets that regulate sugar metabolism in plants. The results reveal new genes that, following expression of CCP1, are upregulated or downregulated. By analogy with the ISY gene, it is believed that the upregulation or downregulation of the new genes, with or without the expression of the algal CCP1 gene, potentially may result in an increase in branching, seed yield, and, in some plants, an increase in the average size of individual seeds too. Upregulation can be achieved by insertion of an expression cassette with a promoter operably linked to the gene of interest followed by an appropriate termination sequence. Upregulation of gene expression or the pattern of gene expression can also be achieved using the promoter replacement strategy with the CRISPR system similar to the one described in FIG. 22 and Example 11. Downregulation can be achieved using the promoter replacement strategy by choosing a weaker promoter or by using CRISPR to delete nucleotides to remove amino acids, or to delete or insert nucleotides to achieve a frameshift mutation.

Given the results obtained with the truncated version of the Camelina ISY gene, it is also possible that the expression of N-terminal truncated versions of the genes in TABLES 17, 18, 19, 20, 22, 23, 25, and 26 may result in an increase in branching, seed yield, and, in some plants, an increase in the average size of individual seeds too.

### Additional Genetically Engineered Land Plants Modified to Express the Gene Targets Identified in Soybean

Considering the gene targets identified in soybean in particular, a genetically engineered land plant that comprises a modified gene for a protein identified in TABLES 17, 18, 19, and 20 is disclosed. The protein comprises one or more of SEQ ID NOS: 141-192. The modified gene comprises (i) a promoter and (ii) a nucleic acid sequence encoding the protein. The promoter is non-cognate with respect to the nucleic acid sequence encoding the protein. The modified gene is configured such that transcription of the nucleic acid sequence is initiated from the promoter and results in expression of the protein.

In some examples, the protein is heterologous with respect to the genetically engineered land plant. In some examples, the protein is homologous with respect to the genetically engineered land plant.

In some examples, the genetically engineered land plant exhibits increased expression of the protein in comparison to a reference land plant that does not include the modified gene. In some of these examples, the increased expression of the protein can be in one or more tissues of the genetically engineered land plant, e.g. in leaf tissue and/or mature seed tissue. Also in some of these examples, the protein comprises one or more of SEQ ID NOS: 141, 142, 143, 144, 146, 150, 151, 152, 153, 154, 155, 156, 157, 158, 159, 160, 161, 162, 163, 164, 165, 166, 167, 168, 169, 170, 172, 173, 175, 177, 182, 183, 184, 185, 189, 190, or 191.

In some examples, the genetically engineered land plant exhibits decreased expression of the protein in comparison to a reference land plant that does not include the modified gene. In some of these examples, the decreased expression of the protein can be in one or more tissues of the genetically engineered land plant, e.g. in leaf tissue and/or mature seed tissue. Also in some of these examples, the protein comprises one or more of SEQ ID NOS: 145, 147, 148, 149, 171, 179, 180, 186, 187, 188, or 192.

In some examples, the genetically engineered land plant exhibits both increased expression and decreased expression of the protein in comparison to a reference land plant that does not include the modified gene. In some of these examples, the increased expression of the protein can be in one or more tissues of the genetically engineered land plant, e.g. in mature seed tissue, and the decreased expression of the protein can be in one or more other tissues of the genetically engineered land plant, e.g. in leaf tissue. Also in some of these examples, the protein comprises one or more of SEQ ID NOS: 174, 176, or 178.

### Additional Genetically Engineered Soybean Plants

Considering the gene targets identified in soybean further, a genetically engineered soybean plant that comprises a modified gene for a protein identified in TABLES 17, 18, 19, and 20 also is disclosed. The protein comprises one or more of SEQ ID NOS: 141-192. The modified gene comprises (i) a promoter and (ii) a nucleic acid sequence encoding the protein. The modified gene is configured such that transcription of the nucleic acid sequence is initiated from the promoter and results in a difference in expression of the protein in comparison to a reference soybean plant that does not include the modified gene.

In some examples, the difference in expression is an increase in expression. In some of these examples, the increase in expression can be in one or more tissues of the genetically engineered soybean plant, e.g. in leaf tissue and/or mature seed tissue. Also in some of these examples, the protein comprises one or more of SEQ ID NOS: 141, 142, 143, 144, 146, 150, 151, 152, 153, 154, 155, 156, 157, 158, 159, 160, 161, 162, 163, 164, 165, 166, 167, 168, 169, 170, 172, 173, 175, 177, 182, 183, 184, 185, 189, 190, or 191.

In some examples, the difference in expression is a decrease in expression. In some of these examples, the decrease in expression can be in one or more tissues of the genetically engineered soybean plant, e.g. in leaf tissue and/or mature seed tissue. Also in some of these examples, the protein comprises one or more of SEQ ID NOS: 145, 147, 148, 149, 171, 179, 180, 186, 187, 188, or 192.

In some examples, the difference in expression is both an increase in expression and a decrease in expression. In some of these examples, the increase in expression can be in one or more tissues of the genetically engineered soybean plant, e.g. in mature seed tissue, and the decrease in expression can be in one or more other tissues of the genetically engineered soybean plant, e.g. in leaf tissue. Also in some of these examples, the protein comprises one or more of SEQ ID NOS: 174, 176, or 178.

In some examples, the protein comprises two or more of SEQ ID NOS: 141-192, e.g. two, three, four, five, or more of SEQ ID NOS: 141-192. In some of these examples, the protein comprises at least one of SEQ ID NOS: 141, 142, 143, 144, 146, 150, 151, 152, 153, 154, 155, 156, 157, 158, 159, 160, 161, 162, 163, 164, 165, 166, 167, 168, 169, 170, 172, 173, 175, 177, 182, 183, 184, 185, 189, 190, or 191 with increased expression. In some of these examples, the protein comprises at least one of SEQ ID NOS: 145, 147, 148, 149, 171, 179, 180, 186, 187, 188, or 192 with decreased expression. In some of these examples, the protein comprises at least one of SEQ ID NOS: 174, 176, or 178 with both increased expression and decreased expression. In some of these examples, the protein comprises a combination of at least one of SEQ ID NOS: 141, 142, 143, 144, 146, 150, 151, 152, 153, 154, 155, 156, 157, 158, 159, 160, 161, 162, 163, 164, 165, 166, 167, 168, 169, 170, 172, 173, 175, 177, 182, 183, 184, 185, 189, 190, or 191 with increased expression, and at least one of SEQ ID NOS: 145, 147, 148, 149, 171, 179, 180, 186, 187, 188, or 192 with decreased expression, and optionally also at least one of SEQ ID NOS: 174, 176, or 178 with both increased expression and decreased expression.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims. REFERENCE TO A “SEQUENCE LISTING,” A TABLE, OR A COMPUTER PROGRAM LISTING APPENDIX SUBMITTED AS AN ASCII TEXT FILE

The material in the XML file, named “YTEN-59704US4-Sequence-Listing.xml”, created Aug. 28, 2024, file size of 421,888 bytes, is hereby incorporated by reference.

What is claimed is:

1. A genetically engineered land plant that expresses a protein that increases seed yield with increased expression (“an ISY protein”), the genetically engineered land plant comprising a modified gene for the ISY protein, wherein:
the ISY protein comprises one or more of (i) an ISY protein of Camelina sativa comprising SEQ ID NO: 2; (ii) a fragment of an ISY protein of Camelina sativa comprising SEQ ID NO: 2; (iii) a Camelina sativa homolog of an ISY protein of Camelina sativa comprising SEQ ID NO: 2; or (iv) an ortholog of an ISY protein of Camelina sativa comprising SEQ ID NO: 2;
the ISY protein has homology to a plant invertase inhibitor and/or a pectin methylesterase inhibitor;
the modified gene comprises (i) a promoter and (ii) a nucleic acid sequence encoding the ISY protein;
the promoter is non-cognate with respect to the nucleic acid sequence encoding the ISY protein; and
the modified gene is configured such that transcription of the nucleic acid sequence is initiated from the promoter and results in expression of the ISY protein.

2. The genetically engineered land plant of claim 1, wherein the ISY protein comprises the ISY protein of Camelina sativa comprising SEQ ID NO: 2.

3. The genetically engineered land plant of claim 2, wherein the ISY protein of Camelina sativa comprising SEQ ID NO: 2 corresponds to one or more of ISY protein of Camelina sativa of SEQ ID NO: 2, ISY protein of Camelina sativa of SEQ ID NO: 5, modified ISY protein of Camelina sativa of SEQ ID NO: 138, or modified ISY protein of Camelina sativa of SEQ ID NO: 140.

4. The genetically engineered land plant of claim 1, wherein the ISY protein comprises the fragment of an ISY protein of Camelina sativa comprising SEQ ID NO: 2.

5. The genetically engineered land plant of claim 4, wherein the fragment corresponds to 114 to 133 contiguous amino acids of SEQ ID NO: 2.

6. The genetically engineered land plant of claim 1, wherein the ISY protein comprises the Camelina sativa homolog of an ISY protein of Camelina sativa comprising SEQ ID NO: 2.

7. The genetically engineered land plant of claim 6, wherein the Camelina sativa homolog corresponds to one or more Camelina sativa proteins of SEQ ID NOS: 21-40.

8. The genetically engineered land plant of claim 1, wherein the ISY protein comprises the ortholog of an ISY protein of Camelina sativa comprising SEQ ID NO: 2.

9. The genetically engineered land plant of claim 8, wherein the ortholog corresponds to one or more (i) canola proteins of SEQ ID NOS: 69, 71, 73, and/or 75; (ii) soybean proteins of SEQ ID NOS: 89, 91, 93, and/or 95; or (iii) corn proteins of SEQ ID NOS: 99, 101, 103, 105, and/or 107.

10. The genetically engineered land plant of claim 1, wherein the ISY protein comprises (i) (a) a cysteine residue at position 40, (b) a leucine residue at position 59, (c) a cysteine residue at position 98, and (d) a cysteine residue at position 138, with numbering of positions relative to ISY protein of Camelina sativa of SEQ ID NO: 5, and (ii) an overall identity of at least 10% with ISY protein of Camelina sativa of SEQ ID NO: 2.

11. The genetically engineered land plant of claim 10, wherein the ISY protein further comprises only between 1 to 14 amino acid residues N-terminal to the cysteine residue at position 40, with numbering of positions relative to ISY protein of Camelina sativa of SEQ ID NO: 5.

12. The genetically engineered land plant of claim 1, wherein the promoter is a constitutive promoter.

13. The genetically engineered land plant of claim 1, wherein the promoter is a seed-specific promoter.

14. The genetically engineered land plant of claim 1, wherein the genetically engineered land plant exhibits increased expression of the ISY protein in comparison to a reference land plant that does not include the modified gene.

15. The genetically engineered land plant of claim 1, wherein the genetically engineered land plant exhibits increased seed yield, fruit yield, and/or tuber yield in comparison to a reference land plant that does not include the modified gene.

16. The genetically engineered land plant of claim 1, wherein the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.

17. A genetically engineered land plant that expresses an RNA that increases seed yield with increased expression (“an ISY RNA”), the genetically engineered land plant comprising a modified gene for the ISY RNA, wherein:
the ISY RNA comprises a contiguous sequence of codons encoding a protein that has homology to a plant invertase inhibitor and/or a pectin methylesterase inhibitor (“an encoded protein”);
the encoded protein comprises one or more of (i) a protein of Camelina sativa comprising SEQ ID NO: 2; (ii) a fragment of a protein of Camelina sativa comprising SEQ ID NO: 2; (iii) a Camelina sativa homolog of a protein of Camelina sativa comprising SEQ ID NO: 2; or (iv) an ortholog of a protein of Camelina sativa comprising SEQ ID NO: 2;
the modified gene comprises (i) a promoter and (ii) a nucleic acid sequence encoding the ISY RNA;
the promoter is non-cognate with respect to the nucleic acid sequence encoding the ISY RNA; and
the modified gene is configured such that transcription of the nucleic acid sequence is initiated from the promoter and results in expression of the ISY RNA.

18. The genetically engineered land plant of claim 17, wherein the encoded protein comprises the protein of Camelina sativa comprising SEQ ID NO: 2.

19. The genetically engineered land plant of claim 17, wherein the encoded protein comprises the fragment of a protein of Camelina sativa comprising SEQ ID NO: 2, and further wherein the fragment corresponds to 114 to 133 contiguous amino acids of SEQ ID NO: 2.

20. The genetically engineered land plant of claim 17, wherein the genetically engineered land plant is an oilseed crop plant selected from the group consisting of camelina, Brassica species (e.g. B. napus (canola), B. rapa, B. juncea, and B. carinata), crambe, soybean, sunflower, safflower, oil palm, flax, and cotton.


SEQUENCE LISTING (198 sequences)

SEQ ID NO: 1 - DNA, 405 bp, Camelina sativa
  source 1..405: mol_type = genomic DNA
atgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtga

SEQ ID NO: 2 - protein, 134 aa, Camelina sativa
MKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 3 - DNA, 3376 bp, Camelina sativa
  source 1..3376: mol_type = genomic DNA
tttaaaaaaatttgatgtatttagaaattaggtgttattcaattgtatattttaaataatctattaaaatatgtgttatttaatatttactggattttgtgtgattttggatttcaacgtactttgcttctaatatacgaacaaacacaatatcacactttctcaaacatttcatttttctctcctctctctctctagatagatctctagcctttaacatattattgtttgattttttttctaaatccttaaatataaccatgcttattcataagatctgtgttatttttgagaatttttttttttgtgttctttaatttttttttctgggttcaagatctgtaacttttttttttctgggtttataatataatctcacttttttttcttctaggttcaatgtctgttttttcttccacaaagtctatgtctataatcacacatattcatcagatctttgttttttttttagatttttttttgtgttctttaacttttttttctgggttcttgatttaaaactttttttttctgggttcatgatataatcacaccattttcttcttgattcaatgtaaaagtctggatgttgttaatttaatatggttttggtgtataatacagtttatgaatattttttttcttttttttcttctataaataactttaaaaaaatccataacaatcaccaaaaacagattataaatgactttcaaatccaccttatatgcattataaaatccacataaatacattagacttttaaatccactaatgtcctgcgtcaattaaaatccattaaaatccttaatgcaataacacctctttggatttatgtatttttaactaaacaaatcctctagaatccctaaaacttaataaaatctaaatccacaaacttttttgaataacaataaattttaaagtagatttttaaatgatcaattcaataacagtggattttaatggactttttataatccatgattgaataacataggatttgtaaattttacacaaataatttaaaatgttatgttgaatacatccccctaagttttaataatttacactttagggtttatgcctttatccctaaaatatacaaaacaagtctcaaattataaagcatgaaaataataaagtttgatgtaaagtaaaatatttttctttttgattagtatgaaataattgatgtcaaatagttagaatgtgattaaaaaaaagaacttttccaaccaataataatgcaccatgtatcttagtggtcccaggtttaatccacattcaagatggtttaatccacataaatttttaatattattcaactcccttattgtataaattcaactgttgcattaatgagtcaacacctctattgtagatagactaagagtcttaccataaaattctatgttctcttatactataaattgaaaaacaaagtgaacatgttcttcatcaatcaattcttattttatatactttccccaaaagaaaaacaaaataaagttattggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtgatgaatttgtcaaagtattttactaatcaaaacaacaattaatgtttaccatgtaaaattgagagcaaagagactgtatcttaatggaaataaaaattttctttacatgtctaggtttttttttgccagcaacattgacatattggctttctattaaaatttataattctttttttttggaaagataaataccaaggtattagagtttgaagaggctatactacaactgtggatttgaaacaatcatcaaatatcttagtccaaaaaaacactgatagaaatggttcacatacagctaaacaaaagaatatggttaaaggcttacgatgattttctgtataagaagaacaatcaatatattttactaaagaactaatgaaattacaaatggtaaagtttagaaattttttttgatatgcatgtgttctcctactttattatataagattgattaaaagaggcaatttttttcaagatattattctttagtatttaaaaatcaatatagtttaatttagtaattattagatatggacctgcctgatgtgcagggttaaagttttgtatatcaaataaaatttaataaaaatatattaaatttattgtaagttatttataaatttaattttgctataatacctgattttatatctattcacaaattttttatgtatgttaccattaaaagtgtattttttacacctaaatatattgtatgttacaaatatagtatttacctagaaaatgtctatataaagacattaagccaactattatgtttctttattttcacttttgtatagtttttttaattactaaaattgtttgctcaaaaaacatttcgaataaaaaatatattacataagactactaatcaatgatgtattatcacaatagtgtatggccaaccatggagagaatctcgactctgccctcttcctttatagagagaaccttgtatccaacctcctccacgatggagagaaccttggctccgtcttcctcccttgaggagataaccttgcatccaacctcctccacctttctcctttggaaagataaacttgtgtccaacctcctccattatagagagaacatcggctctgcccttctcctgtgtggagagaatatgttcacgtattttagctatctcaaattggcttgctctggcaaccaatgaaactaattaaagaccaatttctaatgtcacaaaatcttttgataataaccaatgttaaaatttttaatgtattcgtagaagtgtctttgacacaccatgatgtagcctctgtctctgtaacgccctgaccgccaccttgcttagttaccccatgttcactcccagtccatggtcc

SEQ ID NO: 4 - protein, 190 aa, Camelina sativa
TCSSSINSYFIYFPQKKNKIKLLVSLVVFSLLLIGFASAQTLIVDSCKKAAAKEPFMKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 5 - protein, 168 aa, Camelina sativa
LVSLVVFSLLLIGFASAQTLIVDSCKKAAAKEPFMKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 6 - DNA, 507 bp, Camelina sativa
  source 1..507: mol_type = genomic DNA
ttggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtga

SEQ ID NO: 7 - DNA, 576 bp, Camelina sativa
  source 1..576: mol_type = genomic DNA
tgaacatgttcttcatcaatcaattcttattttatatactttccccaaaagaaaaacaaaataaagttattggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtga

SEQ ID NO: 8 - protein, 176 aa, Zea mays
MKLLQALCPLVILLACSTSNASVLQDACKSFAAKIPDTGYAYCIKFFQADRGSAGADKRGLAAIAVRIMGAAAKSTASHIAALRASEKDKERLACLSDCSEVYAQAVDQTGVAAKGIASGTPRGRADAVMALSTVEDAPGTCEQGFQDLGVRSPLASEDAGFRKDASIALSVTAAL

SEQ ID NO: 9 - protein, 180 aa, Arabidopsis thaliana
MASSLIFLLLVTLTFSASTLISAKSNTTTIIESTCKTTNYYKFCVSALKSDPRSPTADTKGLASIMVGVGMTNATSTANYIAGNLSATVKDTVLKKVLQDCSEKYALAADSLRLTIQDLDDEAYDYASMHVLAAQDYPNVCRNIFRRVKGLAYPVEIRRREASLRRICGVVSGILDRLVE

SEQ ID NO: 10 - protein, 205 aa, Arabidopsis thaliana
MKMMKVMMLIVMMMMVMVMVSEGSIIEPTCKETPDFNLCVSLLNSDPRGSSADTSGLALILIDKIKGLATKTLNEINGLYKKRPELKRALDECSRRYKTILNADVPEAIEAISKGVPKFGEDGVIDAGVEASVCQGGFNGSSPLTSLTKSMQKISNVTRAIFYSNSIVKEEACGSSWPSLALNIDSKACVVSLQNIQFNRGRTCW

SEQ ID NO: 11 - protein, 175 aa, Solanum lycopersicum
MRNLFPILMLLTNLSLNIDNNNNNNIIRATCRETPYYSLCLSVLESDPRSYKAEGSDDITTLGLIMVDAVKSKSIEIMKKLKELEKSNPEWRVPLNQCYMVYNTVLRADVTVAVEALKRGVPKFAEDGMDDVVVEAQTCEFSFNYYNKSDFPISNMSKDIVELSKVAKSIIRMLL

SEQ ID NO: 12 - protein, 171 aa, Solanum lycopersicum
MKILIFLIMFLAMLLVTSGNNNLVETTCKNTPNYNLCVKTLSLDKRSEKAGDITTLALIMVDAIKSKANQAANTISKLRHSNPPQAWKDPLKNCAFSYKVILTASMPEAIEALTKGDPKFAEDGMVGSSGDAQECEEYFKATTIKYSPLSKLNIDVHELSDVGRAIVRNLL

SEQ ID NO: 13 - protein, 184 aa, Beta vulgaris
MTTLNTSLPHLHLLFITLLTLFTTSTLAYSRKTTNDLVTTTCKQTPDPILCEASLRSDSRSSKAADSEGLILIMIDVVKTRFSDSFRYVEDLTRKTHDPDVIRALQECKQLYRVVLDVSVGLAVRAVKQGDPKFGEQAMVDAGNEAEGCRMAFPEGKVPGRIVGRTRMLHGVSNVAASMIKSLE

SEQ ID NO: 14 - protein, 172 aa, Nicotiana tabacum
MRNLFPIFMLITNLAFNDNNNSNNIINTTCRATTNYPLCLTTLHSDPRTSEAEGADLTTLGLVMVDAVKLKSIEIMKSIKKLEKSNPELRLPLSQCYIVYYAVLHADVTVAVEALKRGVPKFAENGMVDVAVEAETCEFSFKYNGLVSPVSDMNKEIIELSSVAKSIIRMLL

SEQ ID NO: 15 - protein, 166 aa, Nicotiana tabacum
MKNLIFLTMFLTILLQTNANNLVETTCKNTPNYQLCLKTLLSDKRSATGDITTLALIMVDAIKAKANQAAVTISKLRHSNPPAAWKGPLKNCAFSYKVILTASLPEAIEALTKGDPKFAEDGMVGSSGDAQECEEYFKGSKSPFSALNIAVHELSDVGRAIVRNLL

SEQ ID NO: 16 - protein, 181 aa, Solanum tuberosum
MRNLFPILMLITNLALNNDNNNNNNNNNNYNLIHATCRETPYYSLCLTTLQSDPRSNEVEGDDAITTLGLIMVDVVKSKSIEIMEKIKELEKSNPEWRAPLSQCYVAYNAVLRADVTVAVEALKKGVPKFAEDGMDDVVVEAQTCEYSFNYYNKLDFPISNLSREIIELSKVAKSIIRMLL

SEQ ID NO: 17 - DNA, 10042 bp, synthetic construct
  misc_feature 1..10042: note = Synthetic construct vector pMBXS1168
  source 1..10042: mol_type = other DNA
ggccagtgccaagcttggcgcgcccctaggcctcagcttaattaagcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccagatctaaaatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtgacctcaggctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcggaccgctcgagcaattgtacgtagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgac

SEQ ID NO: 18 - DNA, 10144 bp, synthetic construct
  misc_feature 1..10144: note = Synthetic construct vector pMBXS1169
  source 1..10144: mol_type = other DNA
ggccagtgccaagcttggcgcgcccctaggcctcagcttaattaagcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccagatctaaattggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtgacctcaggctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcggaccgctcgagcaattgtacgtagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgac

SEQ ID NO: 19 - DNA, 11136 bp, synthetic construct
  misc_feature 1..11136: note = Synthetic construct vector pMBXO58
  source 1..11136: mol_type = other DNA
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaagaacagaaactgatctctgaagaagatctgtgaaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 20 - protein, 179 aa, Camelina sativa
YFPQKKNKIKLLVSLVVFSLLLIGFASAQTLIVDSCKKAAAKEPFMKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 21 - protein, 176 aa, Camelina sativa
MKFLVSLVMCSLLLNGLASAQTLIQDSCKKAAAKDPQMKYDFCVNSLTQDPQSKTATTLEGLVIASTKNAAAKTTNVKGIVEQILKGKRYGPGIETVLHDCIEHYDDANGSLNTALASVQSHDYSTATVNLGAALDAPGNCEDGFKERKQQKSPVTNENNILFQKILIPLAFTNML

SEQ ID NO: 22 - protein, 174 aa, Camelina sativa
MKFLVSLVMCSLLLNGFASAQTLIQDSCKKAAAKDRQMKYDFCVDSLTQDPQSKTATTLEGLVLASTKNAAVKTMNVKGIVEQILKGKGYGGIEAELRQCAEFYDDANDNLNTALASVKSHDYETANVDFSAALDVPVNCEDGIKERKRQKSPVTNENNILFQKILIPLAFTNM

SEQ ID NO: 23 - protein, 168 aa, Camelina sativa
MCSLLLNGFASAQTLIQDSCKKAAAKDPQMKYDFCVNSLTQDPQSKTATTLEGLVIASTKNAAAKTTNVKGIVEQILKGKRYGPGIETVLHDCVELYDYANGSLNTALASVQSHDYSSATVNLGAALDAPGNCEDGFKERKQQKSPVTNENNILFQIILIPLAFTNML

SEQ ID NO: 24 - protein, 175 aa, Camelina sativa
MKFLVSLVMCSLLLNGFASAQTLIQDSCKKAAAKDRQMKYDFCVNSLTQDPQSKTATTLEGLVLASTKNAAVKTMNVKGIVEQIIKGKGYEGIEAELRDCAEFYDDANDNLNTALASVKSHDYETANVDFSAALDVPVNCEDGIKERKKQKSPVTNENNILFQKILIPLAFTTML

SEQ ID NO: 25 - protein, 175 aa, Camelina sativa
MKFLVSLVMCSLLLNGFASAQTLIQDSCKKAAAKDRQMKYHFCVDSLTQDPQSKTATTLEGLVLASTKNAAVKTVNVKGIVEQILKGKRYGGIEAELRECAEFYDDANDNLNTALASVKSHDYETANVDFSAALDVPVNCEDGIKERKKEKSPVTNENNILFQKILIPLAFTNML

SEQ ID NO: 26 - protein, 176 aa, Camelina sativa
MKFLVSLVMFSLLVNGFAFAQTLIQDSCKKAAVKEPALIYNFCVDSLTQDPQSKTATTLEGLILASTKNAAVKTMNVKGIAEQIFKGKGYGGIEAELHDCVEFYDDANDLLNTALASVQSQDYKTANEDFSIALDVLGNCKDGIQEINKQKSPVSNENNILLQKILIPFVFNNMLR

SEQ ID NO: 27 - protein, 176 aa, Camelina sativa
MNFLVSLVMFSLLLNDFTSAQTLIQDSCKKAAATNMQLKYDFCVNSLTQDPESKTATTLEGLVFASTKNAAAKIMDVKRFVEQILKAKKYGPGMEAVLRTCVELYDEANGALNTAFSSVQSHDYNTANVFMSAALDAPDNCEDGFKEGKLEKSPVTNENNILLQKILIPLAFTNML

SEQ ID NO: 28 - protein, 175 aa, Camelina sativa
MKFLVSLIMFSLLLNGFAFAQTLIQDSCKKATVIEPGLTYNFCVDSLTQDPQSKTATTLEGLIIASTKNAEAEAMNVKGIVEHILKGKGYASIEAELRLCVEFYGDAKDSLHTALASVRSHDYKEANVNFSIAEDVPRNCEDAIKDRIPKSPISNENYILFQKIAIPSVFDYMLI

SEQ ID NO: 29 - protein, 176 aa, Camelina sativa
MKFLVSLVMFSLLLNDFTSAQTLIQDSCKKAAATNLQLKYDFCVNSLTQDPESKTATTLEGLVFASTKNAAAKIMKVKRFVEQILKAKKYGPGMEAVLSTCVELYDEANGSLNTAFSSVQSHDYNTANLYMSAALDAPDNCEDGFKEGKLKKSPVTKENNILLQTILIPLAFTNML

SEQ ID NO: 30 - protein, 175 aa, Camelina sativa
MNFLVSLVMYSLLLNDFTSAQTLIQDSCKKAAAINLQLKYDFCVNSLTQDPESKTATTLEGLVFASTKNAAAKIMDVKRFVEQILKAKKYGPGMEAALSTCVELYDEANGSLNTAFSSVQSHDYNTANVYISAALDAPDNCEDGFKEGKLEKSPVTNKNNILLQTILIPLAFTNM

SEQ ID NO: 31 - protein, 176 aa, Camelina sativa
MKFLVSLVMFSLLLNGFTFAQTLIQDSCKKAAVKVPDLDYNFCVDSLTQDPQSKTATTLEGLILSSTKNAEAKAMNVKGIVEQILKGKKYESIEAELRDCVEFYDDANDSLNTALASVRSQDYITANENFSIALDVPGNCEEEIKERNKQHSPVSDENNILLQKISIPCALNYMLI

SEQ ID NO: 32 - protein, 176 aa, Camelina sativa
MKFLVSLVMFSLLLNGFAFAQTLLQDSCKKATVIEPGLIYNFCVDSLTQDPQSKTATTLEGLILPSTKNAEAKAMNVKEIVEHILKGKKYESIEAELRDCVEFYDDANDSLNTALASVRSHDYKTANENFSIALDVPGNCEDGIKERNKQHSPVSNENNDLFQKILIPLVFNYMLI

SEQ ID NO: 33 - protein, 176 aa, Camelina sativa
MKFLVSLVMFSLLLNGFAFAQTLIQDSCKKAAMKDSNLIYKFCVDSLTQDPQSKTATTLEGLILASTKNAEAQAINVKRIVEHILKGKGYRSIEAELRDCVEFYGDANDSLHTALASFRSKDYESANSDFGIALDVPGNCEEGIKERNKQNSPVSNENNILFQKILIPFAFNNMLI

SEQ ID NO: 34 - protein, 176 aa, Camelina sativa
MKFLVSLVMFSLLLNGFAFAQTLIQDSCKKAAMKDSAMIYNFCVDSLTQDPQSKTATTLEDLILASTKNAEAKAINVKRIVEQILKGKGYRSIEAELRLCVEFYGDANDSLHTALASFRSKDYASANSDFGIALDVPGNCEDAIKERNKQKSPVSNENNDLFQKILIPFAFNNMLS

SEQ ID NO: 35 - protein, 147 aa, Camelina sativa
MFSLLLNGFAFSQTLIQDSCKKATVKEPALIYNLCVDSLTQDPQSRTATTLEGLIIASTKNAEAKAMNVKGIIEHILKGKGYASIEAELRDCVGFYDDANDLLNTALASVQSHDYKTANGDFSIALDVPENCEDGIKERNKQNSPLL

SEQ ID NO: 36 - protein, 175 aa, Camelina sativa
MKFLVSLVMFSLLLNCFTFAQTLIQDSCKKAAMKIPDFYYNFCVDSLTQDPQSKNATTLKGLILSSTKNAEANAMKVKGIVEHILKGKKYESIEAELRDCVEFYDDANDSLNTALASVRSQDYKTANENFGIALDVPRDCEDGIKERTQHSPITDENNTLLQKISVPCGLNYILI

SEQ ID NO: 37 - protein, 183 aa, Camelina sativa
MKFLLYLVMFILLLNVFATAQSLIRDSCKKAAAKYPKLKYEFCVKSLEEHPQSKTAKSLEGLVFASTKNAVSKTTSLKGLANKILKENKYDVERPLRDCLELYTGAIDSLNQSLDTIKSRDYKIATMLMSAAMDATGNCETGFTKRKKPVKSPFTKENDVLFHMVLIPLAFTNMLDMNLPKVL

SEQ ID NO: 38 - protein, 175 aa, Camelina sativa
MFILLLNVFATAQSLIRDSCKKAAAKDPKLKYDFCIKSLEEHPQSKTAKSLEGLVFASTKNAVSKTTSLKGMANKILKENKYDVERPLRDCIELYTGAIDSLNQSLDTIKSRDYNIATMLMSAAMDATGNCETGFTKRKKPVKSPFTKENDVLFHMVLIPLAFTNMLDMKLPKML

SEQ ID NO: 39 - protein, 179 aa, Camelina sativa
MKYLLYLIVTFFLLLNGFSTAQTLIRDSCKKAAAKNPKFKNDICVKSLETNPQSKAAKDLAGLVVASTKNAASKATSLKGTVDKILKGKKMNKMSEMALRDCLQLYTDAIDSLNQALASVKSRDYNTVATVLSAAMDAPSTCETGFKEIKTPQKSPVTKDNDTLYQMILIPLSFTNMLK

SEQ ID NO: 40 - protein, 178 aa, Camelina sativa
MKFLLYLIVTFFLLLNAFSTAQTLIRDSCKKAAAKNPKFKNDICVKSLETNPQSKAAKDLAGLVVASTKNAASKATSLKGTVNKILKGKKMNKISEMPLRDCLQLYTDAIDSLNQALASMKSRDYNTVVTVLSAAMDAPSTCETGFKERKTPQKSPVTKDNDTLYQMILIPLSFTNML

SEQ ID NO: 41 - DNA, 1148 bp, synthetic construct
  misc_feature 1..1148: note = Synthetic construct Glycine max Hsp70 promoter
  source 1..1148: mol_type = other DNA
gcaacagaagacccaaaactcaaaaaagttagtttcgggccaacatttcctcttgagggatgacacgtgacctgctactctggcccttatctggcatgtccatccttcttggcgcgacatttaattcgtcgtcagaaataactgaaggacaccttgcttgtttctcttttggccgccaccggtcttgtcatcgtcgaaggcgcccttgcgcttgtcggcagaacctttttcggcgacctccttgccttttcctttggccttgttcgtcatttctacagagaatgcaatgagaccaacgccaattgcatggttagagttagagaaatggagagaggaagaagtgcgtgactagagtgtgtgtaactgtgaagaacgacgagtccaaaatgaattttactgtaaataatttgaggaaaaaagtgatcaatacatatcatgcggtgcatacaagaatcggccattggtcaacttgtgagaggaaaaaatcatttaactaataccaaataatcttaaaattaataaaataatttaactaattaacccacggaagaaccttcttccgttgactctggcggaagaagttcttccgcatagttccatggaagatggttcttccgcagttcttctttcgttgacactcgcggaagaaatgttccacgggcgtccgcggaagaactttcttccgcaaagctaaagagcatttttgccatgtcgaaatcatcgccaatgaccagggtaacagaaccacgccctcttatgttggtttcaccgattcagagcgtttgatcggtgatgccgccaagaatcaggtcgccatgaaccccgtcaacaccgtcttcggtaagatccctagccgacacttcgccttttcaggatttgcattgttcctagatttttggatctgttgtttgaaactccacttttctattttggtaatttttagttttattttgtaatcctgctgtttatatgtcttattgttattattaatcgttgcatggtctgaactggtttagaactctacttgtattgtttgttaaaatcttatttgaaatcgaatagtaatataattttaatcgaatggtgatatgcataaacatcgtatttgttcgtcgaattctggttttgaattgaataatattgttatg

SEQ ID NO: 42 - DNA, 1378 bp, synthetic construct
  misc_feature 1..1378: note = Synthetic construct Glycine max Cab5 promoter
  source 1..1378: mol_type = other DNA
ctagaaattaaatgtttttaacaggtaatttgagaaaaatgtacttcaaaataattagttttaccagtttatgtcttctttttctcttttttatctttattctatgtttcaaattctaataatacatcatttaaatatttttaatttaaaagtgcttactaaattttaaaaaaatcatatttatcaaataacttctactttaaatttaaacttcattatttttaacttaaaaataacttttaaattaaaaaaatgaaaacaaacactacctaaaccctaaacactatctatctaagtcacattacttaatgattcttaatttatgttctttgtaaactttcatttcttcctccttttggctatacatgttcatttctgtgtactttactatattattagtaaaagccttttatataggtatatcaaatcaaataattaatataatatataattctcttaatttcatttcttcatataaatgtatttcaaaagtatttcttctagaataaactaaagctattacagatgaaaaattcttaaaaaattatttgaccttcatatatgggtccttttctaattaataattaactatataggtgcattctaaatgctcctatattatctgctttctcctcttctttccttttttcctagtcgctcacgaaaatctcctataatcctctgcagttttcgaaatcaataaccgactcctagaacctgtccatgtctaacttaataaatcgtgagggtgtgattgtgattactttgaatctttaatttttgacattaaaacaagaccaaacaaaaaccttcaggttacgtgagactccaacctacccaagttatgtattagtttttcctggtccagaagaaaagagccatgcattagtttattacaactaactatatttcaatttcatgtaagtgtgccccctcattaaaatcgacctgtgtaaccatcaacctgtagttcgctcttttcaccatttgtctctctgtctttatcttccctcccccattgccaatatttgttgcaatacaacatctctccgttgcaatcactcatttcaaattttgtggttctcatttgccctagtacaacattagatgtggacccaaaaatatctcacattgaaagcatatcagtcacacaattcaatcaattttttccacatcacctcctaaattgaataacatgagaaaaaaatagctaagtgcacatacatatctactggaatcccatagtcctacgtggaagacccacattggccacaaaaccatacgaagaatctaacccatttagtggattatgggggtgccaagtgtaccaaacaaaatctcaaacccccaatgagattgtagcaatagatagcccaag

SEQ ID NO: 43 - DNA, 1500 bp, synthetic construct
  misc_feature 1..1500: note = Synthetic construct Glycine max PPDK promoter
  source 1..1500: mol_type = other DNA
gatcctcacaaacctcacttggagacataggtgtgagggtaacctttttccctttatgtacaaatgaaaatttgtttgtgacaccattatggacaacatccttacactactaaaaaagcttttttttacgacatcatatttacgacagtcatacaaaaacgtcttagtatgtataaggatggcaatttcgtaaatatttcaaacatttcaaaggcagtttcagaaaaccgtctttgaatgcggccattttaatttttaacgcgcccctcgcatccgttcctcttctttccgcaaatgtggtgctcgttccttttctttcccagctggcatctgttcctctccccactcgctagctatcttctgcttctcctcttctctcctcttcccattacatttctccaccttctccctggtaccaccaccgccccccactccacattcgtcctccgcccccattcccctatcctccagtaaaattacaaaaaaccctaacaccaaaaaaacccaaacccctgtcgcaatgaaatctccacccccaaatagctctttggaatagaatcaaggaacttaccaaatccattatatgctattggggttttggcatgtttccggtgtgaaagaaggaaaaagaaatgcgtatgcgatggtgatgtacgtaggtacgccgaaggactacgaattctacatagccatactcgtgcttctcaaatcgctggctacgctcgacgttgaaattgatcttgctgtgattgcttcccttgatgttcctcctcgatggattcgagctctgtaagtctcactccttcaccatcatttgccactttatttttatgtacttttactttattattatttgtaacctgtatttttatttggtttcggatatctgttgctttattattcaccctggaatttggttgattttattatttttgaaaaataaggaaagagatttatttgttagcttaattgttttaattggcgaatatgtttttcttttcccttttttgcacagagtgaagctttgttcttagggtaatggattcccttttttgtgatgctagtggatgatttgactgattagtgtttagtggaatgaagaaccagaactagtagtaggtagagggaatcacttttggttttggatgtaaacttagaaatgtgcagcactgcacagaattgatatttgatcgtgggtcaaattgtcaaaatgtgcaaagaatacaaaggcacaggtgatatcattccattttacgttttttaacgaagctgttagtttcaattcaattatttacatatataataaatatattgatacttgctttagtttcatgaattaaaagaatttgattttgtaaatttcatttgaatttgtttttgtacaagctctcaacttttattatatgaacgagaagtttcttttttcctttttgagtttatttgaacttgtggtgttctaattgtatatatttttgtgcaggtgtcaatcggtactactac

SEQ ID NO: 44 - DNA, 1261 bp, synthetic construct
  misc_feature 1..1261: note = Synthetic construct Glycine max actin promoter
  source 1..1261: mol_type = other DNA
atctctcgacagttgcgaactgaacgctgagttggtaatgctatgccctatcgctttttgcaccgtcccatgatcatttcccccacaccaccccatcaacctctaaaaagttaagagtgaaaattacacacacccgaggagaagaaaagctgcttcttctaagcatcacaacctagttactttacttgtagggccttttccatttcccctaaattacccctcttttcatcatatgataataatatccagctcagactatagtatgatattatgatgtcagcataataggttggcactaaagtcttaaagggcattgtacatgttgcacctggcattcaaattcataaatactaacactgtgaaatagattataaatcctcaaataaatgtcacacggttggggttcgaatccactcaaaaaggctaatgggatgggatttaagtgccaaggaatataccatggactttaacagcaacacaatttacaatctaaaatgtattacttttttttttcaaaaaagatatacaaaataaggtaccaagaataaaaggagtatttagaaacagtggcaccaatttaataaattatttatataaaatgacacttatttaatttatcaatgataaaagtaatattgatttattctctgattaactgttcaattaatagtgttattatcataatctgtcgcaaaagttatttttatcaacaacaataattgatacaagtagtataaaattaagcctcttagttaatatagactacttgatactaaaaccatgttacaccaaaaagtaatttttatgtcacttgtctatataataattacgactaaattaataatttttaaaaatattactgaatccattaaccgaacttttataatgaaagtatttttatgctttaaaatcacaaacattgaataaactaaaaatgataccacggaattggaacaagagacgttccacacaaaagaaaaaaatatgttgaataattgaaacggtgacaagaaaagtggaataataatacaaagatggcagatggggttattgttattggaggagatgagtgaaataatgagtgaggggggtgtaactggaaagcaagaaaaagcgcaagagtgccagctatttccaacaacaaacgtggcccgtgggatgcgatattcgtaacgaacggcgaggatggaaggacgtgcaatttgcgcttcatttgaggcgaatttcatttggccagaccttccttttttaaaccacaggg

SEQ ID NO: 45 - DNA, 1094 bp, synthetic construct
  misc_feature 1..1094: note = Synthetic construct Glycine max AGPase promoter
  source 1..1094: mol_type = other DNA
tgtgtcaatgttgtttctggtgaattgacataatgaattctacctgtacggagtagagaataactatttacccaacaagaatgattatctcattaatttttgaagtagacgcaataacgaatatattatacattcagaaaaatttcaccatattattctcaaatcacaacaataatttgttttttttttgcttgatataaaaccaatactctatactttttaaggttaatttaaacttaaagagtatttttaagatgcatgtactttaaggaataatagaaacatgacaacatcataaaagaatgaagaaactgaatcataacgtagtttgttacgccttccatttggtggttgatttggatacaatctagattggtttgctaaatggtttataagttatgtagacgtttttattactactattttagacaaatcaaatacacaccttcactttattctattcaaataacatgatttttcctaacattttttaaaaaaattactttttaaatataaactaattattttagaaatagttttataaaaatccacgccaaaaaaattaagttgtttttataaatataaacatcgggcttcaatcttaaatttataaatgtacgaaataatttgacagttaaatggaaattgctagcatggaagtgtttttatcatttatcaaactcaaccaaactgaacatcagaataattattagtgacaaattttgcagcatatgaagtggcttgcatagctccaaggctggcgatcatatgtcagattagagcaggctctctttggtactatgatacatttcaagcaaataacaaccgtaaaaattcacgccaaaatttttggaacgaatctatatattattattttatttcttttgatttcatgtacgtacagtgcccgtaattgacatgtctttgttccttaatgcctttcccacgtggaacaggcacctagaaacttggactaagtagggaattgagggccatggactatagtgccaaaccaacatcattttatatatatatatatatatatatatatatatgctattgttttctatagtttttggaaattaatacttatc

SEQ ID NO: 46 - DNA, 1449 bp, synthetic construct
  misc_feature 1..1449: note = Synthetic construct Glycine max GluC promoter
  source 1..1449: mol_type = other DNA
atttgtactaaaaaaaaatatgtagattaaattaaactccaattttaattggagaacaatacaaacaacacttaaaacctgtaattaatttttcttctttttaaaagtggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 47 - DNA, 1321 bp, synthetic construct
  misc_feature 1..1321: note = Synthetic construct Glycine max CIN1 promoter
  source 1..1321: mol_type = other DNA
aaaaacacaaaaaaaaattatacaaaaatgtttctcacaacatgagaagtaaaatccctcaaagaatttcacatcatcatatcagaatcaaaggaatcaaaatcataggtcaaaaatacaaaaacaccaagaacactcaatttattaactaatttgcatcatgacatcaattggtccatcaaacacaacaatcttgtaattataatcgtaacgaaagaattacaatgcaataaacatcccaaaataaacctcaatttaatcctctaaggatccctatacatgttcattctaaccccaattgtgataaattcatcccttacctctaagcaggctcacgtgtgtagtctggcagtgatagaggcatctctagtggttttctaatagtcctcaagcttgtttttcctctagttgttctgttaggattttcaagcgttagagagaagaagaagagattggagcctctatttcactgttaccgtacaagggatatttttctcaccataaacattattttgcaaatcccaacgaaggagatgtccgtacataagttcgaaacctggtgctcgaatttcacgacgattcaatggttaacaagtccaagattgtatttttactgtgacagatttgagtgtatacaagaaaaagagagctccatgcgaggaatatttctctcacagtagacattatttcataaatcccaatggtaaaaatatgcaaaaatgagtttcaaacctgcttttaaaatttcatgacgactcaacggttaacgtgtccgggattatattttcactggaacaagtttgagtgcatgcgggaaaagagagggttttgggagaggaaaaaaggaaaacaaatttaagaggaagagagagcgtaaaaatttatcgtaaatgtaaaaaatgacctaatatatctctatttataactagggtactctcaatctattatttactcatttttttattttattattttataaaaaagaattttattttacttcctatcaaattaataaataaaacattcttcttattttctaagatcacatatttattttatttaccttaaaatcatcattttaattaataaaattatttcttcttatttatttaattacaaaaatcttattatttttttaaaattttatttatttttaaataaaatattttttaatttattttataaaaaatgagatgttacattgaattataaaataaatagccaacaataaatagccgacttgcttttgcattgactaaggaagtcaagtcatcaataaatataatttccagttggcaatattctcaaagttggtctatat

SEQ ID NO: 48 - DNA, 514 bp, synthetic construct
  misc_feature 1..514: note = Synthetic construct Glycine max MADS-Box promoter
  source 1..514: mol_type = other DNA
agatttgatcgatacttcattaaattgacattttattttaacacataatacattattaaaaatataaataaacatttacagcgaagttatataattaaaagcctggtctatgtaatggtaggaaatttgaaaatctaaaagcaaacaaaaattgttgtttatggtgctaagttgcacctggaaagatgcattgtttagctaaaacattcacgtcgagtacttggtttgggaaaaaaagccattcaagcttagctggtcctctctcctgtctctctctctctgtctgtctctctctgtctgtctctctctcaagcacatacacaaacaaagtaagggctataaataggagggatggaagtggaagaaagtctatagcgaagtttcatttctttggattagaaatttttcccaaagctgatcgagaagccagccaggccaggtctgtagttttctttttttctttttaatattaattcattattgtgttcttcatcatataatataattaagcctt

SEQ ID NO: 49 - DNA, 702 bp, synthetic construct
  misc_feature 1..702: note = Synthetic construct Glycine max Glycinin subunit G1 promoter
  source 1..702: mol_type = other DNA
cgcgccgtacgtaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcaccgcggccgc

SEQ ID NO: 50 - DNA, 579 bp, synthetic construct
  misc_feature 1..579: note = Synthetic construct Glycine max oleosin isoform A promoter
  source 1..579: mol_type = other DNA
acgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgc

SEQ ID NO: 51 - DNA, 1500 bp, synthetic construct
  misc_feature 1..1500: note = Synthetic construct Oryza sativa Hsp70 promoter
  source 1..1500: mol_type = other DNA
gcagctgttttcgcggtacagggtgcaacaaaagcccatgacggcccacacctgcctctctccgctccaaacaccgaaacaagggggtgggtgcaatgggccggcgctcgaagaccgcgaactctttccaacagcccagcgcattagcccctcctcctactctctctaccttctttttaacatgcgactttctttctgtggacgacggcatcaacgacgggagcaggagcgggggctgaagcacggtgcgtgggctcctggagtggcgacggcctctccggcgagcttcctctggcgaactccctccgctcctcctatggcgaaatccaaacaagggtcagtttcgactccaaccttctcccaccaccacctcctgaccgtgccaccacccggccttgtcggcactgaaaggcgtcaacttgtcagcgcgggcctgctcggtcggtctcctcctcccctatttcgtttagctttgcccccgccaccaacaccggcccacggcccatggccgaccccgcggctttggcgccgccatcgctatctcgccgctgtccttttttcatgaccttcggtgccatccctctaaattcgatgcacctccctggctctatctccctttacctccgaaatcctaaccctacccataatctctagtgagtcttgtctttatttatggcctctttgaatcgcaggattgataaaacgtaggattttgataggaatgtaagtgtaaaacacatgattgtaaaatagaggaaaaacataggaatggccgtttgattgaaccgcagaaaaaacacaggaattagatgagagagatagactcaaagttactaagagattgaagcttttgctaaatttcctccaaaatctctataggattggccattccatagaaatttcaaaagatttaataggattcaatcctttgtttcaaaaaacttcatagaaaatttttctatagaattaaaatcctctaaaattcctatgttttttctccaattcaaaggggcccttaggttggaatttggaaagtgttcgcgagaaatcaagcggtcgcacgttagcgaattaggatttccggaaacaaaggaccgactccgcctatccatcgtcacgagcacagtgtagaacctcccagacctcaagagaccgttcaaaaagcgcgcgcccaagcggggcccaccaacgcgtccccaccgtgtcgcctcctgattggttgtcccctcttcctttcacgcgaaccggcaccctcccgacccttccagaacccccaatccgacggccaggatcgcccgcgcgcgaacgttctagacccccgccacctccgccacaaaacctctgcccctcccctctccccccgcttcgtctcgttcgagaaatcagaaagagagagaaattcccacgcagcagcaagcaatccaatccgagagcgcgcgtttgcgattattcgctttcgattccgcgaggtttttggagagggaggagaaggaggaggag

SEQ ID NO: 52 - DNA, 1500 bp, synthetic construct
  misc_feature 1..1500: note = Synthetic construct Oryza sativa Cab5 promoter
  source 1..1500: mol_type = other DNA
acagcatttattgtagtctggtcaagcgtgtcacgctgcatgcaacgcagtacagcgcgttcctttacccggtctgtgaccagtcacagaccggtcagatcacgggttaggtggcgactggcggtctgacgcacgccttgccccatcccgtcaagacgaaagcctctaggcactcgtctcaagccggagctagcgtgttatctcttagagatggcacgttagccctggttagatttataccaggcttcatcctaaccattacaggcaaggtgttacacgaagaagggcaaaacatgcacgttgttaaactgacgcgtgggggacaagaatgaccggtctgacactggtcgcatcagcaacgggcagccacgatcccgcgtcatctccgtctccgccgggagtggaggtaggtgtgggctgtcccatcagaagggctcccggatggaaaccgtaccgatctccgcccattaaagagaaaaagaacagtccagtttggaaagagaagggtgcatgtggtatccccttgaagtataaaaggaggaccttgcccatagagaagggggttgattctttccagattcagagcctagaacgagggagaggtgggctcacactttgtaacttgtccatacacaaatccacaaaaacacaggagtagggtattacgcttccgagcggcccgaacctgtatagatcgtccgtgtctcgcgtttcttgctggctgacgatccttccacatacagagagagagagagcttgggatctcaccctaagcccccggccgaaccggcaaaggggggcctgcgcggtctcccggtgaggagcctcgagctccgtcagacatgttcagtttcattatattatgaaatgtcacgtactgtttgttctagttagtgaattgtcatatggtaagaatatataaaaattaggttttctggactctatcttccaatgtatttttggatcctataacaaaatattttcataaatatattttttaagaatctaaacttttttgaaataaaagagcaacaaagaaaataaaaacgctctctcgtaagtaactcgtgaagatccatcgagagccactcgtttgaatcgtcgacacaaaagaacacttcattgattgcttttcgtcaattagccgcacagcacagtactctccaatctgctaaaccaaaaccaatctcatccatccatacccttcttgacaccaagtggcaactcctgattggacgcgccctatcctacatggcacccccaagattctctcgataggctacaggggccacaccgaccctccacgtcatcgtccacgtcaccctcatcccggcccatccagccaatcccagcccagcaaaaaatcttcccaagtggccaccagataagcctctccacgtattaatacgccaagtgttcgtcgccatgacacagcacgcacacacaccccaccagcagcagcagcagtagctgagcttgaagcagcagagcgaggtagac

SEQ ID NO: 53 - DNA, 1500 bp, synthetic construct
  misc_feature 1..1500: note = Synthetic construct Oryza sativa PPDK promoter
  source 1..1500: mol_type = other DNA
tccacctctgttggttgcatcgacgtcgcttccctagctcccgtctctagtccggatcctattcctccttggagaccgaagctaccgcaaccattgctcggtggttagcgagcgtggagctgtcctccccactttcgcgtcctcgttcgccaccacagccatacttcgcatggtgatgtcttctccttcactcaccgctaaactcagtgcaaccgtttctaccctagccccggccgccgctctcatagaggtgaaagttcatttacatgtaggtcccacatgttttatgttttttatttttcttttactgattagcatgccacgtaaatcaaaacaacaatccatagtgttttaagtatttttatttaatacgtgagatggagtacaaaaacgagagatgcaaagtgaacttgctaaaacacattttctggttgattacagtcgcttgttgagccattggatcggtcataggattcgtgctagcatacttaattacgcgtaactagttgtgctttataggttacaggtcgctaattagcggtctactggagaactttgctactatttttttcttcactgcatgcactcgatcaagtatgagtatttgtaccgaccagcgaaacacatatgtaattaaagtataaatatgtaattagtatatattagtagtatatttagacagtagttacaccctacatacacaccacttacatatataattagtatgtaattttgtaacttacatatgtaattttagtacttacatatgtaattttgagacttacattgtaaatacactaaaattacatatgtaatttagtaacctacaatgtaaatacatgccgactaacttttgatgaaaaatatggtgttataaatatagctactcccgaactttattccttctctgtgagatatcagtggaaacgctcggtggaatcgggggagtatttgggagcacgcgccgacgcgcgcgtcgtgcgtgccgtcgtctttgtcgcggtggagcggagcgcgcccacttgcgcgcctgggccggaggcgggcgcgccgggggttcgggaatcccctggagccacacgtaaaggcgcgggcgggagggagggaggggccagctaggataaggcacgcgcggccgctgcgattggggcgcttgtgaacaccggggcgccacgtggagaggacgttacactccagccgccaaatttccactcccacacccgcgctcccctcccctctcttttccgtgatcgcacctcgcccacgcgccccccgccacacacaatctctgcagctctccagcttcgttggaactcgcgaatctctctccgatcccaggtaaagcagcgaacgacgtcacgcacgacgctgctcggtggatttcgttccttgctggggaaaaccatgcagagacgaaggtgaatgatctgcttttgtgtacttgcgtttaccaggtgaagcgcgagcttggagttggaggggagatcgatcagggccagg

SEQ ID NO: 54 - DNA, 1500 bp, synthetic construct
  misc_feature 1..1500: note = Synthetic construct Oryza sativa actin promoter
  source 1..1500: mol_type = other DNA
ataattaattaattaatcaatcacttttcgtgctgtaaaaaatctcacccgatttgctgaaacgaactgagccgggcgactgtgatattctttcacgatttctgtttgtggcagtgggacattgctgtttattcgaaacaattttcaagtaaaaaaaaatactcaatggtaaggttgctagtaatagtttaacagtttgtttgcagctcagcaaatttcgtttcctcacagatgacacataactgaaagcactcaatgtaatgttgtgcttagctgctaaagcatgtcacgtcttagaaaacaactactccaccatggagaatttttcctcctacttactcctcacatacttaccatctccatataagttcccttgtcgtatcatatgtcttattcttcttgagcacagttattacagcagattttgtagaatagttatcgcatcaaaattttcctatgtcacctttgatcatgtgttatgtgtgcctcttgagtcttagggttaatgtggttgtaatgtgtttaaaaaactatatgaaagctcgtgtgttgctacgggagagagatacctcgaatgaatgtgagagatctccatttgagttgtgtaccttgagagagtgaaagatcacactatttatagacggttaataatggttactgaggtcgattcaccacatcgtcttaaacatttaatgagcatcctccacgtgaaaagtagagatgatagcgtgtaagagtggttcggccgatatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcataag

SEQ ID NO: 55 - DNA, 1945 bp, synthetic construct
  misc_feature 1..1945: note = Synthetic construct Oryza sativa AGPase promoter
  source 1..1945: mol_type = other DNA
aaggtttcatgcgtatcgtgacagatgttacataatgacaaattccccagctggagcacctttatccctgctgtttgcatgaaattagcttgtcttgtagttccctccagcaaaaagaagtctgaaacaaaacaacatttcgaaaaaaaggcatccatgagttagcatttctacagttgtctatagaggggaaggctgcacgacaaagtttccaggcttggaaacaacctcttatgtaaaatttttcgtatgtatcagatgatttgtttgcgttacggcatctccacctaacatcaccttcatcatgcgcctatggtctttctcttgcctgttttatacgtaaaattggaaacgacagaaacttttgccatctttattaaaggaaggcaaatatgcaaatataggcatcaagatcacagttagtggattatcatctttgtaggttaacatgtcctaccccaggggagcttatactcaagtactccatgcattttcatgaaatgagaaaaaacgatttttaagagaaatgtactttcttgtatttatgccaaatggcaaggactgaaagggaaaaactaagaaagggaacgttacagtaaggctctgtggggactggggacttcagagaaacgtgaaccctgcttccttcctctgcatgaacataacaccagaggtttccagcctttcacacagttgttgatggcttcacacaattcatctctacctcctgactctttataaggacccccagcatcaccacaattgcacaagtacaggcattagatccacaagaacacttgggcaggcaagcacctctttgatctttaagccgttgttatgttctatttctgagcatatggtttctagttatattctttttcttcattcgtttcatatctttgaagtgttgatgcaaatgcggtgaacaactatcaactgtgtactctccaagtgaatgcgaataatcatttcctgtgagaattgtgggctagataaacgaatgaaatgctgttttatctatgtcatgtgtggaaatttagttaattttccggtctttttatgcattgagatgggtatgctgtttttttagttgggtcccatcatcttgagaattctttcaaatttccttttctttatcctatataaaggatagagaaggcgtatgcctaggtgcaccaaccctgaaagttttattctaattgcgggaatggtttgtaatttttgcttgttcaggttctttttcgtggcctttcttttttttccccttattttgcttagtctttcacagtccaatttttgggaagtagtatatcttagtttggtcctaaggcaccatgttgtactgcaggaaaaaaaagagtaattgtattctgttttttccttgattactatatccctgttttaattaattttgtgcctttgttgtttgatgttggaacttcaatgcccataattagtcatttgacttgttttgggttttgacgctatcttgagtgccataggaaactggtagaatttagtaataattttatatagactgaatgttgagcccaccacaaatggtttccttctgtacaagtatttaataactcaagcacaggaaacatcagatctctaatctaaaggttaacaatgggctcaagcaggagcagtagttcagctctatctgtatatttagaagggctggatctacctgtccaccagcttttaattttaccctggcagctggataacttcttgtctgttaatttcatttagtgctgtgttattttcttcttgttgttcaggatggatgcttttgaatttctggaatttcgtattttgttctatctctttatgaaatgacgttatggcacactttttctgcatattcttgatgaaaataattacctagtcatttttttagttgcaggtttgtctgggactttgagtacccatgcaattc

SEQ ID NO: 56 - DNA, 2315 bp, synthetic construct
  misc_feature 1..2315: note = Synthetic construct Oryza sativa GluC promoter
  source 1..2315: mol_type = other DNA
gttcaagatttatttttggtatttaatttacttgcttaagtcagatatattcccatcgttgcaggtttgtcacttagtattattattaagcgctctagcactaggactctggataaataagaaagtttattcacgaggctagagtagtaatcaataacataagcgtggtgtctaggtcagcggttatcttcatatgtagtgtgctccatggaaagtgaggtaggaggaaggtggtgacagtcccgtccgtcctttgtatccctccatgttcgggtatatcatagagctacaggctagacttagcttggcagactaggggagagccggtgctcgaagcaatccatgaggctttacatttaacataagttagtaaattaacccataggaatcatctctagactgaacctaccagtagttgtgcttggatataattatattcctacatatacatacacgttccctgcgattagatacccttggaatactctaaggtgaagtgctacagcggtatccgtgcgcttgcggatttatctgtgaccgtatcaaataccaacaggtagatacaaggaatcatctctcctatccattggtttatcatcttttaaaattatctcttgctctcctattgcctctgcaactgcggataggtgtttctcaacaatgaaggttgtgaagaatgctttgtgcaacaagatggatgacaagtatctcagccatagcctcatttgctttgtagaaaaggatatgtcggacacaatcactaagtatcaccgtggaaaggatgcactgtatgccctatctatatttaccatttagtaatatttatatggcttgtgctaactttatgttgtctttacaggcaataacattatttggaaggcatatctatatattactatttaagataatgtaatatctcaaagtttttataagctgcaatgaggtgagtttcacttagctttctaacttgttatgagttatagatgcatgccaccagtcattttttatcttgcatcagcccctgcctgttagaatatgtttctttgtctgggagtccatgtcaactagccaatttccaaatatatgaacaaaactatgtggcctttgtaacccaaatgagataaagactactctccatagaaatttagcaaacatggcactcaaagaaaatgtgttggatagtttcatcatgcatacaaaagcaacacttttgaactaccattccaaatcctttttgtaaattatctttgcttaacactacccctttgagcaaatgtggctttgtgcggaaaaaactcaaacttggtagggtagacatccatttatataattggatccatgtacataagttgttgagtacttcaagtacttacccttgtgatatacatctcaaatatattgaagaagagaagttctttttttgagagaggttgaagaagagaagtttgtccatagctgaagaggagttttatagtgtctagcttaccttgctgctgattgcatgtctaaaatgtcgtttaatttgggctataatgaaatattcaccaatatttctgctggtctattaaagtttaatagttactcgtaactcatttattttgggctataatttaatattcacctatgtttttgttagtctattttatttccctagtgtgcactagcttaaccccaaattagttttgaacacttaacctaaatgtgtctattatggtcagacactctctcacggcactctaacaaaaagtgaattttgttgttatgtttttgtcatgatctcacaagcaatgtacatgtacgtttctagagtgcaatcttatgctagcctgattgtgaatttagtgtagtttgttttctctttttgtagctacactaccaataacctattgtcctctagtcataccacgtaatcacaaggcaaatccctaactctcacctttaaaagcatgtctttattttcttgggtggcactaatacaaaatctttttcagcattcctatgtgcgatagcaagaaaacatggcataactcttgcttcactctaacaaaaaaaacacttttccaactttaaaacaatggtatctatgtgtttaatgatcaatcaagcatataatgacttacaagtttttacctatgccctttttgcatcatcttgtttgcaacagacaaactagatattcctttaggctataaacacatcagcatgataaagagattaggtaagtttgttatccctttttgcatatattctcgtctactccgtgtatataagcccctctcctccaactcgtccatccatcaccaagagcagtggga

SEQ ID NO: 57 - DNA, 1194 bp, synthetic construct
  misc_feature 1..1194: note = Synthetic construct Oryza sativa CIN1 promoter
  source 1..1194: mol_type = other DNA
ttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttct

SEQ ID NO: 58 - DNA, 1500 bp, synthetic construct
  misc_feature 1..1500: note = Synthetic construct Oryza sativa MADS-Box promoter
  source 1..1500: mol_type = other DNA
gcaaagaaggccagtggcctttgcagctaagctagctagctagcccttcttcctctctttcctgctttccctttgccttctcctattaatcctctgcacctcacacagcagcagaaaacccaccaactggagctctcctttcctactccaagaaacgaaggtagagaaagaaagatcagatcagcttcaggaccaattttagctaggttatatatctctttgcgtgctaatgtgttttagttatctgggtgtgtgtagagttctttgttaaggcactgattcagctgcagtttagattcaagtttgtatgttctctctttgaggaaaagaaacccttttcctgtgcttcgagttcttgcaaagagaaactgtgatgcttggcttccagtttgatgcttctttgttcagattggaaattcttcctagcttctttctctatttatgtagcaaggattctttccggcccagtgatcctggtttcttttggaaggtttcagttttttcgttctttcttgaaatttctcttcttgccttaggcagatctttgatcttgtgaggagacaggagaaaaggaagaagctagtttcctgcggccgacctcttgcttctcactttgtgatgagttttctttggtcaattcttagctagatatgttaagatagttagttaagcaaatcgaaattgctagcttttccatgctttcttaaacatgattcttcagatttggttggttcttttttttcctttttgtggagacgtgctgttcttgcatcttatccttcttgattcatctacccatctggttctttgagctttctttttcgcttcttcccttcattatttcgagcaatctctgcacatctgaaagttttgtttcttgagactacttttgctagatcttgtttactcgatcactctatacttgcatctaggctcctttctaaataggcgatgattgagctttgcttatgtcaaatgatgggatagatattgtcccagtctccaaatttgatccatatccgccaagtctttcatcatctttttctttcttttttatgagcaaaaatcatctttttctttcaaagttcagcttttttctcttgttttacccctctttagctatagctggtttcttattccttttggatttacatgtataaaacatgcttgaatttgttagatcgatcactttatacacatactatgtgaatcacgatctcagatctctcagtatagttgaattcattaatttcttagatcgatcagcgtgtgatgtagtactgtaaatcactactagatctttcatcagtctcttttctgcatctatcaatttctcatgcaagttttagttgtttctttaatccggtctctctctcttttttaatcagctgagagtttgtgctgttctttaatcattaccagatctttcatcagtactctctcttctgcatctatcaaacttctcatgcaatgtttttgctgttctttgatctgatctct

SEQ ID NO: 59 - DNA, 1500 bp, synthetic construct
  misc_feature 1..1500: note = Synthetic construct Zea mays Hsp70 promoter
  source 1..1500: mol_type = other DNA
agttttcgcttgtctattcaccctctataggcaactttcaattatgtaatcactttttttttcttttttctgtttaaaatctcagtttcaaacttccaattgattttgaatacgaggtttgggtttaaattcatattggaggcaaaaatcgaaagttccacgtgatgctaggttttatttcggttttctatctcctattgtttttcacgtttcaacttgattcaaattctagttttttttaacttaagcacaattaaatacaacataaaaacaacatggattcaagttctatttcaatttttattaactattatgttgtctagtctgttcaagcacataatacttataaatataaaattaaacgaaatcacatatttccacaaatcttgggtactacactcggagacgacgatggattccatctcaatttggatgttgattatagctctatttcagttgtcactgttgtcctaacacgccctattgtgcatgatagtgcacgtgctcaacgtaaaagaaaagagatcagtaacaagtagcagcactgtacaaggtaagccgtgattcaattaaaactgtttgagcaattcagttgctagatcgttccaccatcgataattcgatatgtacgatgatataaaaagagcccataagtttgtcttgaaaaggttgatcaaataatttaaattagatgataaaaaacatggaagatgtgggagtggacgacggctatgaagaatagtactatatcaggtttatacgtaaaatttatttttgaaatgtttttataatctgtttgaattgtattttttgcttaattatgtgattggatgttttttcatgaaatgtcgagttttattttaaataaaattctgtaaagagaagttgctgcgctgagaaaactataaatcgatagtaaaggctgtacgcaacgtttaagtccttgtttgaatgcgtatgaatctgagaaagttcagaatgattaaatcttttttatttaattttaatttgagagagattaagttctctccaattctctttaatttagacgtaatcgaacaagctggttgccaaactagatgagtacattttgtccactgccatagagccatcgactacaaaagtctagaacacagtggaaagcaccagacaacgcgcgaccaaaagggcccaggccccagcgccccagtccgggggttgtgttcgccgacctgtgcgtgcctgctcgtcacgtcacgtccctatttgcccgtcttcctcccctccagacccttctcgaacgccccttcgttctggatccaacggtcggtctctgccgggctcgaacgttctcgaaaccacgtcacccccgataaaaccccacgcacagcctcctcccttcctcaaccatcattgcaaaagcgaagcaagcaatccgaattctctgcgatttctctagatctcgaccacccctactagttttggttcctcctttcgttcgagagagcgtttctagtggca

SEQ ID NO: 60 - DNA, 1500 bp, synthetic construct
  misc_feature 1..1500: note = Synthetic construct Zea mays Cab5 promoter
  source 1..1500: mol_type = other DNA
caacttacaagcgatgaggccaagacgattagacgaatagctacagaacaagacaatgagagttcagcactcactttttgccagttccttctccttggcagcagccaggcgcttgagtttagcagcttgtgcaaatgtggacggcctacagcagacatacaggcaaagaagcgaggagtaatttgcagttggaaatcattcttcgatcaatagggaaactctgagtcacagcgaaaggaaggttaattgcctacgttgacaactgatcagcctccttgagaagttgcttgatttcaagccgcactttgatctgctcatcactaagtcctccgctctggatgacaaaagcacagaacgcatgagtggcaagtggaaacactagagcgaaataaatacaaaaccgcagactacaggctaacagatagggagaccgggaagacaaagactcgagcctgcattcaacagttacagtcgcctcggccaaaggttgagaaatttgcatcaaaatccaaactgtctagggccatgggaaatagttcctcggaatcagagttcaattcatggacgaaatagatggaactgatggtaggctactcttccgcccaatcagaattcacggaagatccaggtctcgagactaggagacggatgggaggcgcaacgcgcgatggggaggggggcggcgctgacctttctggcgaggtcgaggtagcggtagagcagctgcagcgcggacacgatgaggaagacgaagatagccgccagggacatggtcgccggcggcggcggagcgaggctgagccggtctctccggcctccgatcggcgttaagttggggatcgtaacgtgacgtgtctcctctccacagatcgacacaaccggcctactcgggtgcacgacgccgcgacaagggtgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 61 - DNA, 1500 bp, synthetic construct
  misc_feature 1..1500: note = Synthetic construct Zea mays Cab5 promoter
  source 1..1500: mol_type = other DNA
tctcataaaagcaataaaacaatatctcacaaaatacaagtggcaaacattatacaaacatacacatagtcagaaagtcacaactcaggaccttaaaaaatgaaactatccgattgaaaatacattgataacaattgaacactagaaaataatatcacaaatcaaactatggagcatataactagccatataactcttataatacaataataaaatcatcatatatttaaataaaacactagcaagtctaataacatatgactatagaatcaagatgtgtatgatgacatgacacttgcaattttatcatctcctactactcgacatagtcaatataattgatgtcctccttatctttaaagtttccatgcgaattataaatatatgtatgaagagtaatgattgataagaaactataaataagagtcacaatagttcaaacaactctaaactatatatcattagatagatcttgattttagaaaaataacgaaatcagtttcataattttctaagttaagatgaatttacaaagattagtttagatttaatattttttctgaaaaaataccgatttcggaaacgggcaaaagagatccaaactatttctgtttttttttaccgatttcatttccgtattttcggtaacggtttccggtttcgtatgaccctaaattttggtaaagtttcgaaaaaaaatattttaagaactgaaaattaacgttcctgttttcatccatactaatggctctttaccgctaaaatgttgcccacaatcattgagtaggtttagacgtgagagcaaacagtacaacattacgattcgcccttgcccaaatttacatgccttttccctacggaaacaacatagaatcaagttgacggggttacttacattgaagtggccaaactgatggtagctgtagatttggatgtatgttttctataaattagtcaaaattgagacaaaataaactgcaatttaaaactgaggaaatagtaaaaaaaaggtgaagaagggaggaagaggaaatcagaagcaaaaaatgggcaactttaggcccattatctcgatggtctcgtcggagtccagatatgtgattgacggattggattgggccgtacatcttgcatgagagttcgccaagatttcattgtttaacaagaagcgcgtgacaacaaaaccaagcctatctcatccactctttttttcccttcccacaatggcaagtggcagctcctgattcgctctggccattcctacgtggcacacaccaggattcttgtgtgataggccactgggtcccacccaccaggtgccacatcagacgccaagccatcccggcagaaccaatcccagcccagcaacagatggtctgctatccagttccaactgtataaaagcagctgctgtgttctgttaatggcacagccatcacacgcacgcatacacagcacagagtgaggtaagcatccgaaaaaagctgtgatctgatcgac

SEQ ID NO: 62 - DNA, 1500 bp, synthetic construct
  misc_feature 1..1500: note = Synthetic construct Zea mays PPDK promoter
  source 1..1500: mol_type = other DNA
cgagaatatatgttatcttcgtcgttagagaaatctagacagtatacaacaagatccacgtactacaggtaaacttttaggggtattgtgaacaagaggatgagtaaactctaaaagaacaaagctccaatgaaaatttaggtttttatgtggttagtcatagggcaagttgcaaacaggtgttgatctaaaaaggaagtagtagggaaatgtgaagtgtctttgcgaggaattggaaaatgaagatcacattttctttgggtgcatcatgggaagaaccatttgggactcttttaaggaggcctaagaatgccataaagtttgcaagatctttttgaagagtgtctacctataaacaatagtaaatatcatgtcaaaattttcatcttcgccattattctttaggagaatttagaatgttccgaataaaatatggatagaaaagaagttcccaaagtcatccaattttctacaaaatcttcaactttaagattgagagtgggtgttgtaaagttcttggaagatgagttgaaccccatggaggcgttggctaaagtactgaaagcaatctaaagacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagaggccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggattcacccgttcgcctctcaccttttcgctgtactcactcgccacacacaccccctctccagctccgttggagctccggacagcagcaggcgcggggcggtcacgtagtaagcagctctcggctccctctccccttgctccat

SEQ ID NO: 63 - DNA, 1500 bp, synthetic construct
  misc_feature 1..1500: note = Synthetic construct Zea mays actin promoter
  source 1..1500: mol_type = other DNA
cgataagaacaatgttggacacaacttaagtctgttttacaacaatgtctctcaaaactatagttttacaatattatactttgcaattatcatgacaataatgtagtttcggtagctccaaaaatacagtagttttgagaaacattgtttagatacaatattataaatcatgtattagacaaaagatagccatgccattaaaactttgaattggactgtagttttttcaatactccaaaaatattatggtacctagaatacgatgtctagaaaacatattttttaaaatgcaaccaaacatcatatgacataaataatatagtatttttttgaaaaccatggtattacctaaaaactacagaatacttcattctgaaataggtcctaacaagttgcagcagctaggtcgtacatcagcaaatagctacttcatcaatctcagaataaacatattttatagatgagttaaactaaaaatatagaagaacaacgtacacgcgttgaatcacaacgtagcgcgatatccattcaactttttggaagtttttactgagcacaaattcgaaaatgggaagcgccacgtaacacgagcgctgggccaatttctgccagtgccagttatcccggcccacatccaatcctggggaagacgcgaacccggctccgcggcacgagttgtccgcacgtacggcacgtcggggctggctcgtccgcccgcgagtgggaggccactgtttcctctgcctcaccgggtcgtgtggcggaggggcgtggggccatggttcgcagcgcggggcgacgagcgcgctcctcctctcgcgcagcgccagcgccaccccgcaccgtggctttatatacacccctcctcccaaccctaccgaatcatcactaccaccgctctctcttcctctcctccatctctcaacgcctgaagctcaccgcacctcccctcctcgccgcggatcccccactactccggtaaccgtctctccattcaccctgcctgctgtctcgctagaatcgcctgcctctgccagcgccgtgacgcgggggcgcggtatggctctcccagatccgcctggcattgctcgctcgggtcgtgccaggccgatctgatctcgcatttgctgcgcgctcctcctgctgcggatcccaccggatctcgctggaatcggagcgcgcgtctctttgaaatgccgcagatctgcgtgcttgcgcgcgtgatctaagtccgggcctttcgttaacgaaatggtccgatctgtggtttggtggaggcaatgccatggtttttccccgtgaattttttttgctgattttaggagcttttttctactgtcctatgttagtaggacaaaaaaaaagaaacatagattagcttcaataggcgccttttagaacagattctgtacagcaactcgtggaaacaaatctgcttccttaatgatgttgcttgttttaacaaatgcggcatcgggcgagcttttctgtaggtagaaa

SEQ ID NO: 64 - DNA, 1694 bp, synthetic construct
  misc_feature 1..1694: note = Synthetic construct Zea mays hybrid cab5/hsp70 intron promoter
  source 1..1694: mol_type = other DNA
cacggaagatccaggtctcgagactaggagacggatgggaggcgcaacgcgcgatggggaggggggcggcgctgacctttctggcgaggtcgaggtagcgatcgagcagctgcagcgcggacacgatgaggaagacgaagatagccgccatggacatgttcgccagcggcggcggagcgaggctgagccggtctctccggcctccggtcggcgttaagttggggatcgtaacgtgacgtgtctcgtctccacggatcgacacaaccggcctactcgggtgcacgacgccgcgataagggcgagatgtccgtgcacgcagcccgtttggagtcctcgttgcccacgaaccgaccccttacagaacaaggcctagcccaaaactattctgagttgagcttttgagcctagcccacctaagccgagcgtcatgaactgatgaacccactaccactagtcaaggcaaaccacaaccacaaatggatcaattgatctagaacaatccgaaggaggggaggccacgtcacactcacaccaaccgaaatatctgccagaatcagatcaaccggccaataggacgccagcgagcccaacacctggcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccagcactttcacgctcagcttcagcaagatctaccgtcttcggtacgcgctcactccgccctctgcctttgttactgccacgtttctctgaatgctctcttgtatggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcatacggtatttatttaagcacctgttgctgctatagggcacttgtattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgacgataagtcaagatcagatgcacttgttttaaatattgttgtctgaagaaataagtactgacagttttttgatgcattgatctgcttgtttgttgtaacaaaattttaaaataaagagttccctttttgttgctctccttacctcctgatggtatctagtatctaccaactgatactatattgcttctctttacatacgtatcttgctcgatgccttctcctagtgttgaccagtgttactcacatagtctttgctcatttcattgtaatgcagataccaagcgg

SEQ ID NO: 65 - DNA, 1500 bp, synthetic construct
  misc_feature 1..1500: note = Synthetic construct Zea mays AGPase promoter
  source 1..1500: mol_type = other DNA
tttaaatttggaacgtcgatccaacatctaacagaagcaccaattttacaaagaacccctttcaccttcctcacttggtgggacggttcttaatcaaattaactgcagccgctggtatacatgtacatgtgggcccgcctagcccggcacggcacaggcccacaaaaacacggtccacaaaagcacgacccacaaaagcacatatctaattatgggccgtgccgtgccagcacgtgtgcccagtcatcggcccacaattagttatgtgtgccaggccgacccaaatagcccaaaataccttaatatgccagaccggctcatatacatacaacagtaatacatcaacaaaacgtataaaatatatatatgaccaaaataaaactaagatgttttgtggatgcacattataaacctttggtcagaaagaaaaaaatattacaactagctcacaaaaaatatccagttctctgtttagtgtttaattgagtactatacatccatacagaataaatatacaatgatcatcatcactattcactatccatatctaggtattggttctcgatggcttattaaagctctagattctccaagttatgctagtcatgtgggctttgacagaccttagttaaatactgagtctatattttgtgggccttagttaaatgggtcgtggcaggccggcccgtgggcttgacttgaggcccaggcacggcccacaatgtgggccgtgccggcccatgcccacaattaggttgggcagtgccagatatgggccgtgccagaaattgtgtgctttgggccggcctattaggcacaacataaatgtacacctatagccgcatagccgctggatgtgagatgaatgtctcagatttaaaatgtgcacttgagcaccgtacctctttgaacaacagatatgttcctttaagattgatggtggaaaaaaattagtcagtacctcactgtatggcggcattgtttgattatttcagttcgcacccgttggaccttgctcattaaaaaagtttataccatggagtctttgcatgtagttgtgtagtaggggaagagtggcataggaggaatcacaacttcagctagcttctctagccttagggtatttttgtctttttgcagttcggtcttttcgcagccctgcgctgccccccctgtccgcctgtccctagacctgttttgcgtcggcggggaagacagttgacaggaaggacacgatcttcgtgtccgatgccgatcttcatgcgagcagcgagccactacgttgcgctgccagtgtcggctatggtatccaggcattcgttgtgcacgttgacgatgagctcgaagccggtccgggtgaacgcgagcagcacggtgaggtcaacgtcgtacatccgcacgtcgatgctgaggccagccagcagcggcatgacagattgcggcgtcaggagattgtgccagtaggtggcggggctgggggcagaccggcaggcgaggcct

SEQ ID NO: 66 - DNA, 1500 bp, synthetic construct
  misc_feature 1..1500: note = Synthetic construct Zea mays CIN1 promoter
  source 1..1500: mol_type = other DNA
caaaattttctattttttaaaaaatatgaattctagatttgggattgaacacatctaggctacaacgttgaattgatgaacaatagtgcttgttaataaattgctcacattcacattgtcgctcttacttcaaccatcatacatccatctacagtggtcacccatatttaatcctatggactaaagatgacagatgaacttctctcgttatatatatcactgtcctacatatatgagaaatgatatgtcctaaactcacctaaaaacaacaacatagtttaaatttaatcatagatgagcctacagaggtcgaacgtgatttggaaacatagctctattgttctctatctcatgcataaatatggtgcaatgaagaatattagggttatgatgtcgaaatctcactcgaactcgtgcctcatcataaatagcacactatcaattgttctatggctgttcaaatagggacaatcttgaaacaacatttctcacatgtaaaacgttgtgaagtatgccaactgaaacggatgacacatacacttcgtgaaccaatcgatattttacttgcttctatgttaaataatgttataatacaatattttattcaaatgctaaaacttattactagataaaaataaaatttaattatcttcaaaaactaaccaatagatattccatcataactacatttaccaaactaatatactaaaaaatataggataattactaaattaatcgtgcaataatcagtatttatgagattgataattttaaattttgtgggctacaaacaaaaattaaaacttacttttcaagttggagataagaacaatggtagacgtagctcgggatggtatggcgtcggtgcagacggttaccctttgtgcgaagtggcgcgggcacgagggtggggacttggtacatgcatgagagagaggaagaacgaaacaacttctcaaattaaagcatatgaaaatcacctaatttttgtctgtcggtggaaactaataactagtttttattatcttttttaataaggatccacgaaaattatttttgaccgatgaaaatcctggatcttcgtattatgtttcgccttttcccgactctttgcatgctagatttccatgcttggactaaaacgaagataataaaaccaatctatcattttcacacgatgtattcatacttgcaatagataaaccactactccgacgggatttgctttctgacctctgaaatcttggaaggattatgtgtctacacttctcgatcgaggggaaaaagtcgtagtaccaagttgtagttaaatttgtttcttcgatgacaaaacaaaggagaggggcccgcgcggcgcagcgcagcgcagttggctggttccggaacacgaaaaccaagcacactccaccagctgccatccaccgggttggatggagattacaatactcgaatagtcagccagccagccggcttgaacgtgcagttttcccctataaaacg

SEQ ID NO: 67 - DNA, 1500 bp, synthetic construct
  misc_feature 1..1500: note = Synthetic construct Zea mays MADS-Box promoter
  source 1..1500: mol_type = other DNA
acacttgctctcttcgcgtggtcatttagcccccgaacattccaagaaaaaatagcacatttttgattcataaggtaaagactgccactccacttaacacagcacgctgccaccacacatggattagcaggagagcctgctgtaaaatcctaacaggagggagaacctccaaacaagggttcgccgagcaaaaacacagcccgaccacaaccgacaacctgaaagaacaacagagatacacaggcatgctgggggacctagaccagcgcccagaagtaataacgccagcggagatacaaccgctccgagagagcctgaccatctgagaacacattggtcaccaaaagcaccaccaaccggcctagacaaagcagctcagttgacccccgcctcgacatcttcgatggccggcatcacctttctccccttctttttattcttcgctgtcttcaccttgtcttgatttaacagctccatgattgcatccatttgcttcttggagagaggctttgtgagaaggcttgtcatctgctcaaatgactcatcaaagttagtacattttgaagaactaattattattatatagaatgcactgcacatatattactattaccagttttcttgggcacagcagaaaacatgcacacgcagatagaaaaaggagaggccataaaccaaaaggctttaagaatatatgtaaagatatgtctaaatatatggctatatctggttaagcaagataacagggctctggtcatcagtagtagtggccttttgcccttgcccctctctctcacctctcttttctcagccttgcttccgatggatcccatcccactgccatcctttctttcccttgcgcgcattgcctagccggccggccggcctgctattaaaccactttacccgccccctctcgctcacgctcgacgcagctcccttttccttgtttgcttattgcaagtctctgcaagaacctgctagagaggaacaaggtagagtagtatcgcttttttccatctaggttatctctttttacatgaaaaatttcagccgtatttcgttctccatcagtcctgcgataatatatacgcgcgtcttgtgtgatccggcatatgtatagttcctgctaactgatcgagatcgctctcgtttgtactttctccctttgaggaaagagtttccccttttctgtgcttcaagttcttgtaaggaaaaccatgcctgccagcttcttctgctacttgtatgatgattcttatttgcttattacttgatttccgttttttttcttgctttctatatgtatgtatctgggctgtcttcccctgcgtctcgttactgctaagctttggaaggtttcaactctttgtatacgatgaggtttctgctcctagtagcagatccgcgcatatgactagatgtttgaggaaaagaaaagggcaagacgctatatatatatgcagcacgcagtcgcacatatattcagttttccaatct

SEQ ID NO: 68 - DNA, 528 bp, Brassica napus
  source 1..528: mol_type = genomic DNA
atgaagttcttcgtttcagttgtaatgttttttctcctcttaaactgtttcgcagccgcgcaaactttgattcgagattcttgcaaaacagctgcagcaaaagatcctaatctcaaatatgatttttgcgtccaatcacttgaacaagatccgcaaagcaaaactgcaactagtctatcaggattagtcctagcgtcaacgaataacgctgcgtccaaaacaattaacgtgaaagggatagttgagactattctcaaaagcaaaaagtatgcaccgagtactgaacccgcgttacgcacttgcgtaaagctttatgacaatgcatatggttctttaaaagaagctttgatgaacgttaaatccgatgattacagaagtgctaatgtgcatctgagtgctgctttggatgaaccgaacacttgtgaggatggtttcaaagagaagcacacgaaatctcccgttacaaacgagaacaatattttgtttcagaagattttgattcctttagcttttacaaatatgctctga

SEQ ID NO: 69 - protein, 175 aa, Brassica napus
MKFFVSVVMFFLLLNCFAAAQTLIRDSCKTAAAKDPNLKYDFCVQSLEQDPQSKTATSLSGLVLASTNNAASKTINVKGIVETILKSKKYAPSTEPALRTCVKLYDNAYGSLKEALMNVKSDDYRSANVHLSAALDEPNTCEDGFKEKHTKSPVTNENNILFQKILIPLAFTNML

SEQ ID NO: 70 - DNA, 528 bp, Brassica napus
  source 1..528: mol_type = genomic DNA
atgaagttcttcgtttcagttgtaatgttctttctcctcctaaactgtttcgcagccgcgcaaaccttgattcgagattcctgcaaaacagctgcagcaaaagaccctaatctcaagtatgatttttgcatccaatcacttgaacaagatccgcaaagcaaaactgcaactagtctatcaggattggtcctagcggcaacgaataatgctgcatccaaaacaattaacgtgaaagggatagttgagactattctcaagagcaaaaagtatgcaccgagtactgaacccgcgttacgcacttgcgtaaagctttatgacgatgcttatggttctttaaaagaagctttgatgaacgttaaatccagtgattacaaaagtgctaatatgcatctgagtgctgctttggatgaacctgtcacttgtgaagatggtttcaaagagaagcacgctaaatctcccgttacaaacgagaacaatgttttgtttcagaagattttgattcctttagcttttaccaatatgctctga

SEQ ID NO: 71 - protein, 175 aa, Brassica napus
MKFFVSVVMFFLLLNCFAAAQTLIRDSCKTAAAKDPNLKYDFCIQSLEQDPQSKTATSLSGLVLAATNNAASKTINVKGIVETILKSKKYAPSTEPALRTCVKLYDDAYGSLKEALMNVKSSDYKSANMHLSAALDEPVTCEDGFKEKHAKSPVTNENNVLFQKILIPLAFTNML

SEQ ID NO: 72 - DNA, 528 bp, Brassica napus
  source 1..528: mol_type = genomic DNA
atgaagttcttcgtttcagttgtaatgttttttctcctcttaaactgtttcgcagccgcgcaaactttgattcgagattcttgcaaaacagctgcagcaaaagatcctaatctcaaatatgatttttgcgtccaatcacttgaacaagatccgcaaagcaaaactgcaactagtctatcaggattagtcctagcgtcaacgaataacgctgcgtccaaaataattaacgtgaaagggatagttgagattattctcaagagcaaaaagtatcaaccgggtactgaacccgcgttacgcacttgcgtagaactttatgacgatgctaatgattctttaaaagaagctttgatgaacgttaaatccgatgattacagaagtgctaatgtgcatctgagtgctgctttggatgaaccgaacacttgtgaggatggtttcaaagagaagcacacgaaatctcccgttacaaacgagaacaatattttgtttcagaagattttgattcctttagcttttacaaatatgctctga

SEQ ID NO: 73 - protein, 175 aa, Brassica napus
MKFFVSVVMFFLLLNCFAAAQTLIRDSCKTAAAKDPNLKYDFCVQSLEQDPQSKTATSLSGLVLASTNNAASKIINVKGIVEIILKSKKYQPGTEPALRTCVELYDDANDSLKEALMNVKSDDYRSANVHLSAALDEPNTCEDGFKEKHTKSPVTNENNILFQKILIPLAFTNML

SEQ ID NO: 74 - DNA, 528 bp, Brassica napus
  source 1..528: mol_type = genomic DNA
atgaagttcttcgtttcagttgcattgttctttctcttcttaaactgtttcgctaccgcgcaaactcttattcgagattcttgcaaaacagctgcagcaaaagatcctactctcaaatatgatttttgcgtccaatctcttgaacaagatccacagagcaaaaccgcaactagtctaaaaggattggtcttagcatcaactactaacgctgagtccaaaacaactaacgtgaaagggatagttgagactattctcaagagcaaaacgtatccaccgggtactgaacctgcgttaagcacttgcgtagagctttatgacgatgctaataattctttaaatgaagctttgatgaacgttaaatccggcgattacaaaagtgctaatgtggatctgagtgctgctttggatgaaccgggcacttgtgaagatggtttcaaagagaagcacgcgaaatctcccgttacaaacgagaacaatgttttgtttcagaagattttgattcctttagcttttacaaatatgctctga

SEQ ID NO: 75 - protein, 175 aa, Brassica napus
MKFFVSVALFFLFLNCFATAQTLIRDSCKTAAAKDPTLKYDFCVQSLEQDPQSKTATSLKGLVLASTTNAESKTTNVKGIVETILKSKTYPPGTEPALSTCVELYDDANNSLNEALMNVKSGDYKSANVDLSAALDEPGTCEDGFKEKHAKSPVTNENNVLFQKILIPLAFTNML

SEQ ID NO: 76 - DNA, 480 bp, Camelina sativa
  source 1..480: mol_type = genomic DNA
atgaagctgatcgtgatggtgatgacgatgatgatgataagtgaaggaagtatgatagatcagacatgtaaacagacaccagacttcaaactttgtgtctctctactcaactccgacccacgtggctcctctgccgacatctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaatgctgatgttcccgaagccatcgaagctatctctaaaggagtccctaaattcggcgaagacggcgtgattgacgccggggtagaagcttctgtttgtgaagaagggtttcaagggaaatctccgttgactagtttaaccaaatcaatgcaaaacatctctagtgtgactagagccgttgtgagaatgttgctttga

SEQ ID NO: 77 - protein, 159 aa, Camelina sativa
MKLIVMVMTMMMISEGSMIDQTCKQTPDFKLCVSLLNSDPRGSSADISGLALILIDKIKVLATKTLTEINGLYKKRPELKQALDQCSRRYKTILNADVPEAIEAISKGVPKFGEDGVIDAGVEASVCEEGFQGKSPLTSLTKSMQNISSVTRAVVRMLL

SEQ ID NO: 78 - DNA, 465 bp, Camelina sativa
  source 1..465: mol_type = genomic DNA
atgaagctgatcgtgatggtgatgacgatgatgatgataagtgaaggaagtatgatagatcagacatgtaaacagacaccagacttcaaactttgtgtctctctactcaactccgacccacgtggctcctctgccgacacctctggcctcgctctcatcctcatcgataaaatcaaggtgctggcgacaaagaccttaaccgagatcaacggtctatataaaaagagaccggaactaaaacaggctttggaccaatgtagtcgaagatacaaaacgattttaaacgctgatgttcccgaagccattgaagctatctctaaaggagtccctaaattcggcgaagatggtgtgatggacgccggggtagaagcttctgcttgtgaagaagggtttcaagggaaatctccgttgactagtttaaccaaatcaatgcaaaacatctctagtgtgactagagccgttgtg

SEQ ID NO: 79 - protein, 155 aa, Camelina sativa
MKLIVMVMTMMMISEGSMIDQTCKQTPDFKLCVSLLNSDPRGSSADTSGLALILIDKIKVLATKTLTEINGLYKKRPELKQALDQCSRRYKTILNADVPEAIEAISKGVPKFGEDGVMDAGVEASACEEGFQGKSPLTSLTKSMQNISSVTRAVV

SEQ ID NO: 80 - DNA, 480 bp, Camelina sativa
  source 1..480: mol_type = genomic DNA
atgaagatgatcgtgatggttatgatgatgatgatgatgagtgaaggaagtatgatagatcaaacatgtaaacagacaccagacttcaatctctgtgtctctctactcaactccgacccacgtggctcttctgccgacatctctggcctcgctctcatcctcatcgataaaatcaaggttctggcgacaaagaccttaaacgaaatcaacggtctatataaaaagagaccggaactaaaacaggctttagaccaatgtagtcgaagatacaaaacgatcttaaatgctgatgttcccgaagccatcgaagctatctctaaaggagtccctaaatttggcgaagatggtgtgatcgacgccggggtagaagcttctgtttgtgaagaagggtttcaagggaaatctccgttgactagtttaaccaaatcaatgcaaaacatctctagtgtgactagagccgttgtgagaatgttgctttga

SEQ ID NO: 81 - protein, 159 aa, Camelina sativa
MKMIVMVMMMMMMSEGSMIDQTCKQTPDFNLCVSLLNSDPRGSSADISGLALILIDKIKVLATKTLNEINGLYKKRPELKQALDQCSRRYKTILNADVPEAIEAISKGVPKFGEDGVIDAGVEASVCEEGFQGKSPLTSLTKSMQNISSVTRAVVRMLL

SEQ ID NO: 82 - DNA, 543 bp, Camelina sativa
  source 1..543: mol_type = genomic DNA
atggcttcttctcttatcttcctcctcctcatctttaccctatcctttccatcctcaaccctaatctcagccaaatccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacttccaccgcaacttacatcgccggaaacctaacatccgctgcaaacgacgtcgtccttaaaaaggtgttacaagattgctccgagaagtatgctctcgccgctgattctctccgtcaaacaattcaagatcttgatgatgaagcttatgactatgcttccatgcatgtgctggcggcggaggattatcctaatgtttgccgcaatattttccgccgagctaaggggctgtcttatccggtggggattcgtcggcgtgaacagagtctgagacgtatctgtggtgttgtctctgggattcttgatcgtcttgttgaatga

SEQ ID NO: 83 - protein, 180 aa, Camelina sativa
MASSLIFLLLIFTLSFPSSTLISAKSNATIIESTCKTTNNYKFCVSALKSDPRSPTADTKGLAAIMIGVGMTNATSTATYIAGNLTSAANDVVLKKVLQDCSEKYALAADSLRQTIQDLDDEAYDYASMHVLAAEDYPNVCRNIFRRAKGLSYPVGIRRREQSLRRICGVVSGILDRLVE

SEQ ID NO: 84 - DNA, 546 bp, Camelina sativa
  source 1..546: mol_type = genomic DNA
atggcttcttctcttatcttcctcctcttcctcatctttaccctatccttttcatcaccaaccctaatctcagccaaatccaacgcgacaataatcgaatcaacttgcaaaaccacgaacaactacaaattctgtgtctcggctctcaaatccgacccaagaagtcccacagccgacacaaaaggtctcgcagccattatgattggcgttggtatgacaaacgccacctccactgcaacttacatcgccgggaacctaacatccgccgcgaacgacgtcgtccttaaaaaggtgttacaagattgctccgagaagtatgctctcgccgttgattctctccgccaaacaattcaagatcttgatagtgaagcttatgactatgcttccatgcatgtgctggcggcggaggattatcctaatgtttgccgcaatattttccgccgagctaaggggctgtcttatccggtggagattcgtcggcgtgaacagagtctgagacgtatctgtggtgttgtctccgggattcttgatcgccttgttgaataa

SEQ ID NO: 85 - protein, 181 aa, Camelina sativa
MASSLIFLLFLIFTLSFSSPTLISAKSNATIIESTCKTTNNYKFCVSALKSDPRSPTADTKGLAAIMIGVGMTNATSTATYIAGNLTSAANDVVLKKVLQDCSEKYALAVDSLRQTIQDLDSEAYDYASMHVLAAEDYPNVCRNIFRRAKGLSYPVEIRRREQSLRRICGVVSGILDRLVE

SEQ ID NO: 86 - DNA, 546 bp, Camelina sativa
  source 1..546: mol_type = genomic DNA
atggcttcttctcttatcttcctcctgttcctcatcttcaccctatccttttcatcctcaaccctagtctcagccaaatccaacgcgacaataatcgaatcaacttgcaaaaccacgaacaactacaaattctgtgtctcggctctcaaatccgacccaagaagtcccacagccgacacaaaaggtctcgcagccattatgatcggcgttggtatgacaaacgccacctccaccgcaacttacatcgccggaaacctaacatccgccgcgaacgacgtcgtccttaaaaaggtgttaaaagattgctccgagaagtatgctctcgccgccgattctctccgtcagacaattcaagatcttgatgatgaagcttatgactatgcttccatgcatgtgttggcggcggaggattatcctaatgtttgccgcaatattttccgccgagctaaggggctgtcttatccggtggagattcaccggcgtgaacagagtctgagacgtatctgtggtgttgtctccgggattcttgatcgccttgttgaatga

SEQ ID NO: 87 - protein, 181 aa, Camelina sativa
MASSLIFLLFLIFTLSFSSSTLVSAKSNATIIESTCKTTNNYKFCVSALKSDPRSPTADTKGLAAIMIGVGMTNATSTATYIAGNLTSAANDVVLKKVLKDCSEKYALAADSLRQTIQDLDDEAYDYASMHVLAAEDYPNVCRNIFRRAKGLSYPVEIHRREQSLRRICGVVSGILDRLVE

SEQ ID NO: 88 - DNA, 552 bp, Glycine max
  source 1..552: mol_type = genomic DNA
atgaaattcgcttataacctagtgatgattttctttatctttctctttcaatactcaaacggctccaaccttatccttcaatcatgcaaagaggcctcaaagaatgatccaaatttgagctatgatttctgtgttgcatcccttgaagaagccttatccaagtgtcacccaccacccaccaaccttgaagaccttgtgggcatgtcaattaatctatccaaatccaacgtaaccaacatggtttccatcatttcaaacctattgaagaacaaaacttttgatcaatacactaaggcttgcctaaaagattgcttcgatctttactcggattcgctttcggctttagatgatgctgtggttgctttcaagtccaaggatttggacacggctggtataaacttgagtgcttccttggataattctgttacatgtgaagatcaattcaaggataagaaaggtgaaacttctcctataacaaaggagaacaacgtgtacttccaactcaatgtaatatctctagccttcatccaaatgtttcgtcaacattattga

SEQ ID NO: 89 - protein, 183 aa, Glycine max
MKFAYNLVMIFFIFLFQYSNGSNLILQSCKEASKNDPNLSYDFCVASLEEALSKCHPPPTNLEDLVGMSINLSKSNVTNMVSIISNLLKNKTFDQYTKACLKDCFDLYSDSLSALDDAVVAFKSKDLDTAGINLSASLDNSVTCEDQFKDKKGETSPITKENNVYFQLNVISLAFIQMFRQHY

SEQ ID NO: 90 - DNA, 552 bp, Glycine max
  source 1..552: mol_type = genomic DNA
atgaaattcgcttcatacctagtgattttcttgatctttctctttcattgctcaaatggttccaatcttatccctcaatcatgcaaagaggcctccaagcatgacccaaatttgagctatgatttctgtgttgcatcccttgaagaagcctcctccaagtgtcacccaccacccaccaactttgaagaccttgtgggcatgtcaattcagctaacagaatccaacgtaaccaacatggtttccatcatttcaaaccttttggagaacaaaagttttgatcaatacacaaaagcttgcctaaaagattgctttgatctttactcggattcgctttcggctttagatgatgctgtggttgctttcaagtccaaggatttggacacggctgctataaacttgagcgctaccttcgataattctgttacatgtgaagatcaattcaaggataagaaaggtgaaacttcttctcctttaacaatggagaaccgcgtgtacttccaactcaatgtaatatctctggccttcatccaaatgtttcgtcaacattattga

SEQ ID NO: 91 - protein, 183 aa, Glycine max
MKFASYLVIFLIFLFHCSNGSNLIPQSCKEASKHDPNLSYDFCVASLEEASSKCHPPPTNFEDLVGMSIQLTESNVTNMVSIISNLLENKSFDQYTKACLKDCFDLYSDSLSALDDAVVAFKSKDLDTAAINLSATFDNSVTCEDQFKDKKGETSSPLTMENRVYFQLNVISLAFIQMFRQHY

SEQ ID NO: 92 - DNA, 564 bp, Glycine max
  source 1..564: mol_type = genomic DNA
atgatccttccctcttcttcccttttattcactctcacaatcctcttgtgttctttcctttcttttgtttgtgccaaccgtctcatccaacaaacatgcaagaattgctcaaaaaatgaccccaacataagctacaaattttgcgttacatcattccaatcagaccataggagccactacgccaaaaacctccaagaactaggactaatctccatcaagataacaaggcacaacgtgacggatactaatgctcatatcaatgagcttttgaagaaaaacaagagcttggacccatttatcaaagaatgcttagatgattgcgttgaagtctattccgacaccatttccacatttagagaagccattagagattacaaggcaaaacgttatgccgattgtaatgtcaagctaagctcaataattgatgcctctacgacatgtgaagatggattcaagcaaaagaatgatgccatttcgccactaacgaaaagaaacaaggatacctttcagctttccgctatagcactttccattgttaacatgctcatcaatacggataaataa

SEQ ID NO: 93 - protein, 187 aa, Glycine max
MILPSSSLLFTLTILLCSFLSFVCANRLIQQTCKNCSKNDPNISYKFCVTSFQSDHRSHYAKNLQELGLISIKITRHNVTDTNAHINELLKKNKSLDPFIKECLDDCVEVYSDTISTFREAIRDYKAKRYADCNVKLSSIIDASTTCEDGFKQKNDAISPLTKRNKDTFQLSAIALSIVNMLINTDK

SEQ ID NO: 94 - DNA, 552 bp, Glycine max
  source 1..552: mol_type = genomic DNA
atggaacgctctacaaaactcttctcggcggcgttggttctctgcgtggttgtgatggcacaccagacagcagcacaggaactaaaaggaaagaacctaatcaacaaggtttgcacaatcaccccgagcagagacctctgcgttgggatcctctcatcggacccaatcagaagccctgacgcagacctcaaggacttggcagttatctcccttagggttgctgccagaaacgcctccggcatcctcagcgaagccaaaatgttgatcgatgacgacaacctagaccccgacgtccaacaaggtttgtctgattgcaaggagacaatcttggacgccgagagccagctcgaggacaccattgcatccttgttggtggattccgacaccgacacacagatttggctcaaggctgccttggccgccatcgacacctgcgacgcttccattcccggagacgacgacgttctctccgtcaagagtgccatgttccgcaggctgtgcaacattgccattgccatcaccaagcgcttgaacaaacctcttaaattctaa

SEQ ID NO: 95 - protein, 183 aa, Glycine max
MERSTKLFSAALVLCVVVMAHQTAAQELKGKNLINKVCTITPSRDLCVGILSSDPIRSPDADLKDLAVISLRVAARNASGILSEAKMLIDDDNLDPDVQQGLSDCKETILDAESQLEDTIASLLVDSDTDTQIWLKAALAAIDTCDASIPGDDDVLSVKSAMFRRLCNIAIAITKRLNKPLKF

SEQ ID NO: 96 - DNA, 11380 bp, synthetic construct
  misc_feature 1..11380: note = Synthetic construct vector pMBXO84
  source 1..11380: mol_type = other DNA
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 97 - DNA, 12494 bp, synthetic construct
  misc_feature 1..12494: note = Synthetic construct vector pMXS1091
  source 1..12494: mol_type = other DNA
catgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagggatcccggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagttatcatcatcatcatagacacacgaaataaagtaatcagattatcagttaaagctatgtaatatttacaccataaccaatcaattaaaaaatagatcagtttaaagaaagatcaaagctcaaaaaaataaaaagagaaaagggtcctaaccaagaaaatgaaggagaaaaactagaaatttacctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtagaggagctcgccgcttggtatctgcattacaatgaaatgagcaaagactatgtgagtaacactggtcaacactagggagaaggcatcgagcaagatacgtatgtaaagagaagcaatatagtgtcagttggtagatactagataccatcaggaggtaaggagagcaacaaaaaggaaactctttatttttaaattttgttacaacaaacaagcagatcaatgcatcaaaatactgtcagtacttatttcttcagacaacaatatttaaaacaagtgcatctgatcttgacttatggtcacaataaaggagcagagataaacatcaaaatttcgtcatttatatttattccttcaggcgttaacaatttaacagcacacaaacaaaaacagaataggaatatctaattttggcaaataataagctctgcagacgaacaaattattatagtatcgcctataatatgaatccctatactattgacccatgtagtatgaagcctgtgcctaaattaacagcaaacttctgaatccaagtgccctataacaccaacatgtgcttaaataaataccgctaagcaccaaattacacatttctcgtattgctgtgtaggttctatcttcgtttcgtactaccatgtccctatattttgctgctacaaaggacggcaagtaatcagcacaggcagaacacgatttcagagtgtaattctagatccagctaaaccactctcagcaatcaccacacaagagagcattcagagaaacgtggcagtaacaaaggcagagggcggagtgagcgcgtaccgaagacggtagatctctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccttgcatgccgtcgtcttaagcgtccgcgtgtgaaaatcggattttcgcatacggttgaaccggtcgcatgcaaagatcgcgatcttcgcagacgatttggcacatgcggttgcaccaaccgtatgcgaaaacccttctcgcccgtatgcaaaaaccatctttgttgtagtgtacggttcacaatggtttggatgggaaatcattgtgaaccaaaagtgatagactgatttcgacgagtgtttttttttaagtagtgccacaattttggtcatcatacgtcgtgtctaaaattgtaacttttgaaaaccaatttacattaaattaaatttataagactaaataaagacgatggtcattgaacaattgttgagaaaaatctacacacatgtgtgtccaacacaaatgtttacacatatactactatgttcatagtcgaagttagattttttttttccttaaagggaaagtctgttttcaaattttagacctcactccttccgtttcaaatatatcgtgtatttttttttctagggcaagcttttgaccaatgattactctattatgacacaatgttaaagggatagattcatattcaaaattactattataattataattttgtcatataaataatattttaagcaattgttagccaaaatctcgtcctaacgaaacaaaatacgccttatttttaaaaacacggagtatatccttaaatatttctctatccaatataaaaggtcaatcttttaaaattccgatcatcaataatttctcaaataattactttgaaataaaaaaacatatgcaaatttgtgtcgtcataatatccaatgaacttattcaaatttataaacttattttaattcaaaatttgatcattaattttttttttaaaaaaaaaccaaatcttatcataaacgtcaaatatatttttgatagtgggggcgataataccataaaactaacaacagaagagacatgatactactactgtaatcctaatacgtacgtacgtatacttctacgccggatgcataacttcagccttgtgagacacaacagttgctgcctagctcgtggtcgttggttttttcgctcgagaaaccactacgcgtaaaccgtgaagtatattatatatagccaactggtcttctcgcaaatccgcacatccctttctgcccctcgtcttctggtaccatgtcctctgatgcaatgacgattaacgaaagcctgatggaggttgagcacaccccagctgttcacaagaggatcctggatatactgccgggtattagcggcggtgtcgcgcgtgtcatgattgggcagccattcgacacgatcaaagtccgcctccaagtattgggccagggaaccgctctggccgcaaaattacctccgagtgaagtgtacaaggactctatggactgtatcagaaaaatgatcaagtccgagggtcctctttcattctataagggcacagtggctccactcgtgggcaacatggtgctactggggatccattttcccgtgttttccgccgtgcggaagcagctcgagggggatgatcattattcaaacttctcgcacgccaacgtgctcttgtccggcgctgctgccggagcggcgggcagcctgatctcggcaccggttgaactcgtccgcacaaagatgcagatgcagaggagggcagcattggccggcactgtcgccgccggcgcggccgcctcggcgggagcggaggagttctacaagggctctctcgactgcttcaaacaagtcatgagcaaacatggcattaagggcctatacaggggtttcaccagcactatccttcgggacatgcagggttacgcttggttcttcctcggttacgaggccactgtcaatcactttctccaaaatgccggaccaggcgttcatacaaaggcggatttgaactaccttcaagttatggcggctggcgtagttgccgggttcggactatgggggtcaatgtttcctatcgataccattaagtctaaacttcaagctgacagtttcgcgaagccccagtactcatccacgatggactgcctgaagaaggtcctggcttccgagggccaagccggtctttggagagggttcagcgcagcgatgtatcgagctatacctgtaaacgcaggaattttcctggcggtggagggcacccgccagggcataaagtggtacgaagagaatgtcgaacacatctacgggggcgtgatcgggcccgccacgccgacagcagcccaggaacagaagttaatctcggaggaggacctctgacccgggggcgcgccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtg

SEQ ID NO: 98 - DNA, 654 bp, Zea mays
  source 1..654: mol_type = genomic DNA
atgctagctgcaacaatgtactatcacaataagacgaagatgccaccaccaccttgctcttgcttctctgctgtctctgttcccttctcctccttcaaaaccataacaatgctcctcctcctcctcctcatccttcagcaactgtctgcggctgcggttgcagggatggccaccaccaagcttgggttgtctgacgtggtaaccgacacctgcgacagatgcagcaagagcaatccacaggtgaactacaccctctgcgtctcgtctctgtcgtcggaccccgagagcaggcaggcggacctccacggtctcgccatcatctcggccaagctgctgaggtcaggcgcggtggccatggaggccaagatggcggacctcagcaggaaggagcgcccatggtccccccggaggtcctgcctggacgcctgcgtcggggtgtaccgcaactccctctacgacctcggcagctccatcgtagccatccaggagaggaggtacgcggacgccaagacgtccatgagcgccgcggtggatgcgcccgtcacctgcgaggatgagttcaaggagcagggcttggagccacccatgagagcggagaccaaacgcctgtttcaacaggcggtcatctccctcgcaatcatctccctgctatga

SEQ ID NO: 99 - protein, 217 aa, Zea mays
MLAATMYYHNKTKMPPPPCSCFSAVSVPFSSFKTITMLLLLLLILQQLSAAAVAGMATTKLGLSDVVTDTCDRCSKSNPQVNYTLCVSSLSSDPESRQADLHGLAIISAKLLRSGAVAMEAKMADLSRKERPWSPRRSCLDACVGVYRNSLYDLGSSIVAIQERRYADAKTSMSAAVDAPVTCEDEFKEQGLEPPMRAETKRLFQQAVISLAIISLL

SEQ ID NO: 100 - DNA, 687 bp, Zea mays
  source 1..687: mol_type = genomic DNA
atgacccgtgccagcagcagtagcagcagccggcgtgtgacgctggtactgctcggtctccgcctgctgcttctggttggtgttgcgcaggcggtagtggagttggtgcctgctgatgataatatcgccgccgccgctgctggcacggcggtggacgatggcgagccgcctcagcagtgcgcgaccccggtgagcgtggaggaggcgtgccgcggcgcgtccgagacgcacgccggcgtggcctacgaccactgcatggcgtcgctgggcgccgacccgcgcagcaaggaggccggcaacaagaacatgcacgggctggcggtgctggccaccaggatggccatcgaccacgccgccagcaccgagtccaagatcgacgacctcgcggagctggaggcggcgtcgtcggatccgcaggcgcgcgcccgcttcaaccactgcctggagcagtacggcggcgccgccgacctcctccgcgacgcgctggacaacctcaaggcgaagatctacggcaaggccatggagcagctgaccgccgcaatgggcgcctccgagagctgcgaggacgcgtggaagggcgaggaggaggatgtccccgtcgccgcgcacgacagggagtacggtcggatggcgcacatcgccttcggattcacacaccacgccgccgtcgccgccgccgccgcatga

SEQ ID NO: 101 - protein, 228 aa, Zea mays
MTRASSSSSSRRVTLVLLGLRLLLLVGVAQAVVELVPADDNIAAAAAGTAVDDGEPPQQCATPVSVEEACRGASETHAGVAYDHCMASLGADPRSKEAGNKNMHGLAVLATRMAIDHAASTESKIDDLAELEAASSDPQARARFNHCLEQYGGAADLLRDALDNLKAKIYGKAMEQLTAAMGASESCEDAWKGEEEDVPVAAHDREYGRMAHIAFGFTHHAAVAAAAA

SEQ ID NO: 102 - DNA, 636 bp, Zea mays
  source 1..636: mol_type = genomic DNA
atggcgatgaggtcgctagcgctactggtcctcctcagcctgctggttgttggtgtggcgcaggcggtggagttggagatggagttggtgcctgctgatgcaattgcgatgactatggatcgtgagccgccgcaggagtgcgcgaccccggtgagcgtggaggaggcgtgccgcagcgcgtcggagacgcacgccggcgtggcctacgaccactgcatggcgtcgctgggcgccgacccgcgcagcaaggaggccggcaacaagaacatgcacgcgctggcggtgctggccaccaggatggccatcgaccacgccgccagcaccgagtccaagatcgacgacctcgcggagctggaggcggcgtcgtcggatccgcaggcgcgcgcacgcttcaaccactgcctggagcagtacggcggcgccgccgacctcctccgcgacgcgctggacaacctcaaggccaagatctacggcaaggccatggagcagctgaccgcggcgatgggcgcctccgagagctgcgaggacgcgtggaagggcgaggaggatgtccccgtcgccgcgcacgacagggagtacgggcggatggcgcacatcgccttcggattcacacaccacgccgccgccgccgcatga

SEQ ID NO: 103 - protein, 211 aa, Zea mays
MAMRSLALLVLLSLLVVGVAQAVELEMELVPADAIAMTMDREPPQECATPVSVEEACRSASETHAGVAYDHCMASLGADPRSKEAGNKNMHALAVLATRMAIDHAASTESKIDDLAELEAASSDPQARARFNHCLEQYGGAADLLRDALDNLKAKIYGKAMEQLTAAMGASESCEDAWKGEEDVPVAAHDREYGRMAHIAFGFTHHAAAAA

SEQ ID NO: 104 - DNA, 588 bp, Zea mays
  source 1..588: mol_type = genomic DNA
atggcttccggaacgccctacactgccgtcggcgtcatcttcctctccgtcttcctcgtcgccgcggcatccgcaggccgcaccgcggcacctgcggccgcgccgtcgagcaagtactcgctcgaggaagcgtgcgagcagaccgcggggcacgaggacctgtgcgtggagacgctgtccgcggacccgtcgtccaagactgccgacactacggggctcgcacggttggccatccaggcggcacagcggaacgcgtcggagacggcgacctacctctccagcatctacgacgacgacagccttgagaacaagacggcgcagctgcagcagtgccttgaaaactgcggcgagaggtacgagtcggcggtggagcagctgtcggacgcgacgtcggcgctggacacgggcgcgtacagcgagtcggaggagctggtggtggcgagccaggctgaggtgaggctgtgtcagcgtggctgccaagccgtgccgaaccaccgcaacatcctctcggcgcgcaaccgcaacgtcgaccagctctgcagcatcgcgctcgccatcaccaagctcatccacggaccgccatcttga

SEQ ID NO: 105 - protein, 195 aa, Zea mays
MASGTPYTAVGVIFLSVFLVAAASAGRTAAPAAAPSSKYSLEEACEQTAGHEDLCVETLSADPSSKTADTTGLARLAIQAAQRNASETATYLSSIYDDDSLENKTAQLQQCLENCGERYESAVEQLSDATSALDTGAYSESEELVVASQAEVRLCQRGCQAVPNHRNILSARNRNVDQLCSIALAITKLIHGPPS

SEQ ID NO: 106 - DNA, 531 bp, Zea mays
  source 1..531: mol_type = genomic DNA
atgaagcttctgcaagctctgtgccctctcgtcatcctcctcgcctgctccacgtccaacgcttccgtcctacaagacgcgtgcaagtccttcgccgctaagatcccggacaccggctacgcctactgcatcaagttcttccaggccgacaggggaagcgccggcgcggacaagcgtggcctcgccgccatcgccgtgaggatcatgggggcagccgccaagagcaccgccagtcacatcgccgccctgcgggcctccgagaaggacaaggagcggctggcgtgcctcagcgattgctccgaggtgtacgcgcaggccgtggaccagaccggcgtggcggcgaagggcatcgcctcgggcacgccccggggccgcgcggacgcggtgatggcgctcagcacggtggaggatgcccccggcacctgtgagcaggggttccaggacctgggcgtgcgttcgccgctggcctcggaggacgccgggttccggaaggatgcgtccatcgcgctgtctgtaacggccgcgttgtaa

SEQ ID NO: 107 - protein, 176 aa, Zea mays; identical to SEQ ID NO: 8

SEQ ID NO: 108 - DNA, 770 bp, synthetic construct
  misc_feature 1..770: note = Synthetic construct CaMV35S promoter
  source 1..770: mol_type = other DNA
agagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcg

SEQ ID NO: 109 - DNA, 1507 bp, synthetic construct
  misc_feature 1..1507: note = Synthetic construct Zea mays hybrid promoter
  source 1..1507: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 110 - DNA, 1996 bp, synthetic construct
  misc_feature 1..1996: note = Synthetic construct Maize ubiquitin promoter/intron
  source 1..1996: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctataaagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaaatctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagg

SEQ ID NO: 111 - DNA, 1500 bp, synthetic construct
  misc_feature 1..1500: note = Synthetic construct Maize trpA promoter
  source 1..1500: mol_type = other DNA
gattcgcgcgtgctggaactcgggattggattcatgcgtgctggaacttggaagtctggagtggactttggaagcctggattccagaacaagaactcaagaagtctaggagccgccgagcaggtagggaattagggaaataaagagaagaggcggctggcgttcgacgttccatcttcagtagaggcggctggcgtttcaactccctgtagtcgggccgcctgccaaaaaagcccacgaaggcaggaaatcaaaaaatctaggtcctaaacctagtcgcgcagaaccggctaatcgagcgactaatcgaccctaatcgtcgactagtcggacggccagggcgattaggtactctaatcgagtcggttgtgctaatcgagctctgctaaccgactagcccgaccgcgattagtcagatgacttgaaaacaaagatagagacatacttttttatattctttgcattgttttgtttctatccaaaactgctatttagaaattggaaaatctgcacattgaaaaatctaatggattagatatgttgatttgtttttattcacgagcataatcaaataaattagatttagaattggactgcacgcagtgaactactgaactgaactgtgttcaataatttaaatactcacggctgagccgtgagctgtaggctggagcacaagcacgagccagcaccgagcggcggagcactggagcagcaggcgagcagggaggcggccaggcgggagcagccagccagcaagcaggcagcagcggagcagcccacagccgagcgcccaagctggagctgctgcagagcctgcagcgtgccgctgcgcgccggcaggacaggagcggccgagcgggagtgcaggactgtggcctgcgggacgcggggatgggcggacggcgtagcgcttacagtccgcggacagcggactcacggtggcggctaagatagtgagaccgatgacctaatctctatttggaccggttcaaggttttgcccagttaatattggaccatattgggccttccgcccctgctcgcaagacacactgaacaaagaatccacacggctctccaaaagatagagagataattcacatgcttctctctctctgaaaaaaaggaacttgcatggttgacacggaaaacgtcattaaacgcgcacgtggctgcaaatgcaacgtaacagatccatcatctatccatccatagaatcagacggccacagaaggcaacgaccgtgtgcctgtccaccggcgcaggtggcccacagacgcccgtgcgattcatccgtctcggcccaccaaccacgggaggggccccagggccctccttagtccttacaaataccggcagcagcatcacccggccaccaccacccacccgttttatccacgcacggcgtcgaacaccccgcggtcgctcacgtgaggcgccaccccgcgcacccagtcagcgcccgcctccaccacccacccacacgacaaaaatccgcc

SEQ ID NO: 112 - protein, 358 aa, Chlamydomonas reinhardtii
MSSDAMTINESLMEVEHTPAVHKRILDILPGISGGVARVMIGQPFDTIKVRLQVLGQGTALAAKLPPSEVYKDSMDCIRKMIKSEGPLSFYKGTVAPLVGNMVLLGIHFPVFSAVRKQLEGDDHYSNFSHANVLLSGAAAGAAGSLISAPVELVRTKMQMQRRAALAGTVAAGAAASAGAEEFYKGSLDCFKQVMSKHGIKGLYRGFTSTILRDMQGYAWFFLGYEATVNHFLQNAGPGVHTKADLNYLQVMAAGVVAGFGLWGSMFPIDTIKSKLQADSFAKPQYSSTMDCLKKVLASEGQAGLWRGFSAAMYRAIPVNAGIFLAVEGTRQGIKWYEENVEHIYGGVIGPATPTAAQ

SEQ ID NO: 113 - protein, 356 aa, Gonium pectorale
MVSMTMNDTLNQVEHTPVNPPHKKVLELLPGISGGVARVMIGQPFDTIKVRLQVLGAGTALAAKLPPSEVYKDSMDCVRKMIRTEGPLSFYKGTVAPLIGNMILLGIHFPTFSSVRKQLEGDDHYSNFSYTNTLIAGAAAGAAGSLVSTPVELVRTKMQMQRRAALAGSVAGSAASSGAEEFYKGSVDCFKQVLSKHGIKGLYRGFTSTVLRDMQGYAWFFLGYEATVNYFLQNAGPGVHSKADLNYLQVMAAGVVAGFGLWGSMFPIDTIKSKMQADSLAKPQYTTTMDCLRKVLKTEGQVGLWRGFSAAMYRAIPVNAGIFLAVEGSRQGIKWYEENVEHIYGGVVGAAPGAAS

SEQ ID NO: 114 - protein, 354 aa, Gonium pectorale
MSSMTVNDTLNEVEHTPKDPPHKRVLELLPGISGGVARVMIGQPFDTIKTRLQVLGAGTALAAKLPPSEVYKDSMDCVRKMVRSEGPLSFYKGTVAPLFGNMILLGIHFPVFSHVRKQLEGDDHYSNFSYTNALISGAAAGAAGSLVSTPVELVRTKMQMQRRAALAGSAGSAAASSGAEVFYKGSVDCFKQVLSKHGVKGLYRGVTSTVLRDMQGYAWFFLGYEATVNYFLQNAGPGVHSKADLNYLQVMAAGVVAGFGLWGSMFPIDTIKSKMQADSLVKPQYSTTYDCVRKVLKTEGNNGLWRGFSAAMYRAIPVNAGIFLAVEATRQGIKLYEENVEHIYGGVVGTTTAA

SEQ ID NO: 115 - protein, 339 aa, Volvox carteri
MNDTLNQVEHTPPVHKRILDILPGISGGVARVMIGQPFDTIKVRLQVLGQGTALAAQLPPSEVYKDSLDCVRKMVRNEGPLSFYKGTVAPLVGNMVLLGIHFPTFSYVRKQLEGDDHYTNFSYTNTLLSGAAAGAAGSLVSTPVELVRTKMQLQSAASSASDEFYKGSVDCFKQVLSKYGIKGLYRGFTATVLRDMQGYAWFFLGYESTVNYFLQKAGPGLHSKADLNYMQVMSAGVVAGFGLWGSMFPIDTVKSKLQADTLATPQYRSTYDCLSKVLKSEGQAGLWRGFSAAMYRAIPVNAGIFLAVEGTRQGIKWYEENVEHLYGGVVGPATPAATS

SEQ ID NO: 116 - protein, 353 aa, Ettlia oleoabundans
MPATAQVMNDTLMEVEHTPPVHKRILDILPGVSGGVARIMVGQPFDTIKTRLQVLGKGTIGAAGMPPEMVYNSGMDCVRKMMKSEGPMSLYKGTVAPLLGNMVLLGIHFPTFTKTRAYLEAGDAPGSFSPWKILAAGAAAGAAGSVVSSPTELIRTKMQMVRKNNILAQIKGSAAGGLNPEENYKGNWDCAKKIFRNHGLRGMYSGYLSTLLRDMQGYAWFFFGYEATIHYLAGPGKTKADLDYSQVMLAGVMAGFGLWGSMFPIDTIKSKIQADSLSKPEFKGTLDCVRRSVQIEGYGGLWRGVTAALWRAIPVNAAIFLAVEGTRQLIADTEESIDAFVDQVSGKTSEAAL

SEQ ID NO: 117 - protein, 353 aa, Chlorella sorokiniana
MVARTINETLMEVEHTPPVHKRVLDVLPGVSGGVTRVLVGQPFDTIKTRLQVMGQGTALAKMLPPSDVYINSSDCLKKMVRNEGALSLYRGVVAPLLGNMVLLGIHFPTFSNTRKYLESVDATPAGEFPYWKVLAAGGAAGLAGSFISCPSEHIRTKMQLQRRAALAAQMGLKAQGLETYKGSWDCAVQILRNHGIKGLYRGMTSTVLRDIQGYAWFFLCYEATLHALAGPAHTRSELDYKHVLGAGVMAGFGLWGSMFPIDTIKSKMQGDSLSNPQYRNTLDCLRQSVAVEGFGGLFRGFGAAMYRAIPVNAGIFLAVEGTRQLLNKYEGYIDEKLGISVPASAATVPAPAQ

SEQ ID NO: 118 - protein, 352 aa, Erigeron breviscapus
MPATPQLMNETLMEVEHTPAVHKRILDILPGVSGGVARIMVGQPFDTIKTRLQVLGKGTIGAAGMPPEMVYTSGMDCVRKMIKSEGPLSLYKGTIAPLLGNMVLLGIHFPTFHKTRAYLEREDAPGTHTPWKILAAGATAGAAGSIVSTPTELIRTKMQMVRKNNILQQIKGAGAGGLNPEENYKGNWDCAKKIFRNHGVRGLYSGYLSTLLRDMQGYAWFFFGYEATIHYLAGPGKTKADLDYTQVMLAGVIAGFGLWGSMFPIDTIKSKIQADSLSKPEFKGTLDCLKRSLAVEGQRGLWRGVTAALWRAIPVNAAIFLAVEGTRQLIADTEESVDKFVNNLTGKETAAV

SEQ ID NO: 119 - protein, 354 aa, Zea nicaraguensis
MPIATGQVMNDTLMEVEHTPPVHKRILDILPGVSGGVARIMVGQPFDTIKTRLQVLGAGTIGAQGMPADMVYNNGMDCVRKMIKSEGPGSLYKGTVAPLLGNMVLLGIHFPTFTKTRAYLEQGDAPGTFSPWKILAAGAAAGAAGSVVSTPTELIRTKMQMVRKNNLMAQMKGAAATLNPEENYKGNWDCAKKILRNHGLRGIYSGYVSTLLRDMQGYAWFFFGYEATIHMMCTEGKTKADLNFLQVMGAGVIAGFGLWGSMFPIDTIKSKIQADSLSKPEFKGTMDCLKRSLAVEGHAGLWRGVTAALWRAIPVNAAIFVAVEGTRQLIADTEESVDAFVNNLTGSGSTAAAV

SEQ ID NO: 120 - protein, 141 aa, Poa pratensis
YKGNWDCAKKILRNHGLRGIYSGYVSTLLRDMQGYAWFFFGYEATIHYLAGQHGKTKADLEYWQVMGAGVMAGFGLWGSMFPIDTIKSKIQADSLSKPEFKGTIDCLKRSLAVEGYAGMWRGVTAALWRAIPVNAAIFLAV

SEQ ID NO: 121 - protein, 354 aa, Cosmos bipinnatus
MPSATPQVINDTLMEVEHTPAVHKRILDILPGVSGGVARIMVGQPFDTIKTRLQVLGKGTIGAKGMPADMVYNNGMDCVRKMIKSEGAGSLYKGTVAPLLGNMVLLGIHFPTFTKTRAYLEQGDAPGTFSPAKILAAGAAAGAAGSVVSTPTELIRTKMQMVRKNNILAQMKGAAATLNPEENYKGNWDCAKKILRNHGLRGIYSGYVSTLLRDMQGYAWFFFGYEATIHMMCTDGKTKADLNFLQVMGAGVIAGFGLWGSMFPIDTIKSKIQADSLSKPEFKGTMDCLKRSLAVEGHAGLWRGVTAALWRAIPVNAAIFVAVEGTRQLIADTEESVDAFVNNLTGSSSTTAAV

SEQ ID NO: 122 - protein, 297 aa, Glycine max
MGDVAKDLTAGTVGGAAQLIVGHPFDTIKVKLQSQPTPLPGQLPKYSGAIDAVKQTVAAEGPRGLYKGMGAPLATVAAFNAVLFTVRGQMEALLRSHPGATLTINQQVVCGAGAGVAVSFLACPTELIKCRLQAQSVLAGTGTAAVAVKYGGPMDVARQVLRSEGGVKGLFKGLVPTMAREVPGNAAMFGVYEALKRLLAGGTDTSGLGRGSLMLAGGVAGAAFWLMVYPTDVVKSVIQVDDYKNPKFSGSIDAFRRISASEGIKGLYKGFGPAMARSVPANAACFLAYEMTRSALG

SEQ ID NO: 123 - protein, 296 aa, Zea mays
MGDVAKDLTAGTVGGAANLIVGHPFDTIKVKLQSQPTPAPGQLPKYAGAIDAVKQTVAAEGPRGLYKGMGAPLATVAAFNAVLFSVRGQMEAFLRSEPGVPLTVKQQVVAGAGAGIAVSFLACPTELIKCRLQAQSSLAEAATASGVALPKGPIDVAKHVVRDAGAKGLFKGLVPTMGREVPGNALMFGVYEATKQYLAGGPDTSGLGRGSQVLAGGLAGAAFWLSVYPTDVVKSVIQVDDYKKPKYSGSLDALRKIVAADGVKGLYKGFGPAMARSVPANAATFVAYEITRSALG

SEQ ID NO: 124 - protein, 296 aa, Oryza sativa
MGDVVKDLVAGTVGGAANLIVGHPFDTIKVKLQSQPTPAPGQFPKYAGAVDAVKQTIATEGPRGLYKGMGAPLATVAAFNALLFTVRGQMEALLRSEPGQPLTVNQQVVAGAGAGVAVSFLACPTELIKCRLQAQSALAEAAAASGVALPKGPIDVAKHVVREAGMKGLFKGLVPTMGREVPGNAVMFGVYEGTKQYLAGGQDTSNLGRGSLILSGGLAGAVFWLSVYPTDVVKSVIQVDDYKKPRYSGSVDAFKKILAADGVKGLYKGFGPAMARSVPANAATFLAYEITRSALG

SEQ ID NO: 125 - protein, 324 aa, Triticum aestivum
MEFWPEFLASSGGHEFVAGGVGGMAGVLAGHPLDTLRIRLQQPPRPVSPGITAARVTRPPSAVALLRGILRAEGPSALYRGMGAPLASVAFQNAMVFQVYAILSRSLDRRMSTSEPPSYTSVALAGVGTGALQTLILSPVELVKIRLQLEAAGRKRQGPVDMARDIMRREGLRGIYRGLTVTALRDAPSHGVYFWTYEYARERLHPGCRRTGQESLATMLVSGGLAGVASWVCCYPLDVVKSRLQAQTQTHPPSPRYRGVVDCFRKSVREEGLPVLWRGLGTAVARAFVVNGAIFSAYELALRFLVRNNGRQTLVMEEMKCHDH

SEQ ID NO: 126 - protein, 296 aa, Sorghum bicolor
MGDVARDLTAGTVGGVANLVVGHPFDTIKVKLQSQPTPAPGQLPKYAGAIDAVKQTIAAEGPRGLYKGMGAPLATVAAFNALLFSVRGQMEALLRSEPGVPLTVKQQVVAGAGAGIAVSFLACPTELIKCRLQAQSSLAEAAAASGVALPKGPIDVAKHVVRDAGAKGLFKGLVPTMGREVPGNAMMFGVYEATKQYLAGGPDTSNLGRGSQILAGGLAGAAFWLSVYPTDVVKSVIQVDDYKKPRYSGSLDALRKIVAADGVKGLYKGFGPAMARSVPANAATFVAYEITRSALG

SEQ ID NO: 127 - protein, 323 aa, Solanum tuberosum
MCDELSRCLIWCCLRSASISPISVFSQMDIMKDLTAGTVGGAAQLIVGHPFDTIKVKLQSQPTPLPGQPPKYAGAIDAVRKTVASEGPRGLYKGMGAPLATVAAFNALLFTVRGQTEALLRSEPGAPLTVKQQILCGAVAGTAASFLACPTELIKCRLQAHSALASVGSASVAIKYTGPMDVARHVLRSEGGVRGLFKGMCPTLAREVPGNAVMFGVYEALKQYFAGGMDTSGLGRGSLIVAGGLAGGSVWFAVYPTDVIKSVIQVDDYRSPKYSGSFDALKKILASEGVKGLYKGFGPAITRSIPANAACFLAYEMTRSSLG

SEQ ID NO: 128 - DNA, 15178 bp, synthetic construct
  misc_feature 1..15178: note = Synthetic construct vector pMBXO74
  source 1..15178: mol_type = other DNA
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagagaccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcaatggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaaccttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcgggttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtccggtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaggggatcctctagacgcagaaaggcccacccgaaggtgagccagtgtgattacatttgcggcctaactgtggccagtccagttacgctggagtcactagtgcggccgcgacaacttgtctagggcccatggcaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgcccgggaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgagcggccgcgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtcgagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtatgacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 129 - DNA, 2269 bp, synthetic construct
  misc_feature 1..2269: note = Synthetic construct vector pMBXO92
  source 1..2269: mol_type = other DNA
aagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggaccaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcgagctcggtaccaaaatgtcctcagacgctatgaccattaatgagagcttgatggaggttgaacacacacctgccgtgcataaaaggatcctcgatattttgcctggaatatcagggggagttgctagagttatgatagggcaaccttttgatacaattaaggtcagactgcaggttttgggccaaggtacagccttggcagctaagttgcccccatctgaagtgtataaagactctatggactgcatcagaaaaatgattaaatctgaggggccattgtcattctataagggaaccgtggctcccttggttggcaatatggtccttttgggaatccatttccctgtgttttccgcagtcagaaagcaactggaaggagatgatcattatagtaacttttcccatgctaatgttttgctgtccggggctgccgctggggctgctggctcactcatctccgctcccgttgaactggttagaaccaagatgcagatgcagaggcgcgcagccctcgccggaactgttgccgctggtgctgctgcatctgctggcgccgaagagttttacaaagggagtctcgactgtttcaaacaggtaatgtcaaagcatgggattaaaggtctttataggggatttacttccacaatactcagagacatgcaaggttatgcttggttttttttggggtatgaggctactgtcaaccacttccttcaaaatgctggccctggtgttcataccaaagcagacctcaattaccttcaagtcatggctgccggcgtggtcgccggttttgggctttggggtagtatgttcccaatcgataccattaagagcaaactccaagctgattctttcgccaagcctcaatacagttccactatggattgtttgaaaaaagtgcttgcctcagagggacaagcaggtttgtggaggggttttagtgccgctatgtatagggctattcctgttaacgcagggatttttttggcagtggagggaaccaggcaaggaattaagtggtacgaggaaaacgtggagcatatctacggaggcgtgattggacctgctacccctactgccgctcagtaaggcatgcaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattc

SEQ ID NO: 130 - DNA, 14758 bp, synthetic construct
  misc_feature 1..14758: note = Synthetic construct vector pMBXO75
  source 1..14758: mol_type = other DNA
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgagaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagagaccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcaatggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaaccttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcgggttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtccggtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaggggatcctctagacgcagaaaggcccacccgaaggtgagccagtgtgattacatttgcggcctaactgtggccagtccagttacgctggagtcactagtgcggccgcgacaacttgtctagggcccaatggcccgggactggcgcgccgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcggtaccaaaatgtcctcagatgcaatgactatcaatgagagtctgatggaagtcgagcacacacctgccgtacataaaagaatccttgatattctccccggaatttctggcggagtagctagagttatgattggacagccattcgatacaattaaagttaggctccaagtgcttggtcaggggactgcacttgccgctaagcttcctccttctgaggtgtataaggattcaatggattgcattcgcaaaatgattaagtccgaaggaccactttcattttacaagggaacagttgccccccttgttggaaatatggttcttcttggtatccacttcccagttttttctgccgttaggaaacaacttgagggagatgaccattactctaatttcagtcatgcaaatgttcttctcagtggggccgcagctggcgctgcaggtagtttgattagtgcacctgtggagttggtcaggacaaagatgcaaatgcaacgcagagcagctttggccgggactgtggcagccggtgccgctgcttctgcaggagccgaagagttttataagggctcccttgactgcttcaaacaagtaatgtccaaacatggtattaagggtctttaccgtggttttacatccactatcctccgcgatatgcagggttacgcttggttttttcttggctacgaggcaactgtcaatcattttcttcagaatgctggccctggagttcataccaaagcagatcttaactaccttcaagtcatggcagctggagttgttgcaggctttggattgtggggatctatgtttcctatagatacaatcaagagtaagctccaagccgatagcttcgcaaagccacagtacagtagtaccatggactgcctgaaaaaggttttggcaagcgaaggtcaagcagggttgtggaggggcttctctgctgcaatgtatcgtgccatacctgtgaacgctggtatatttctcgcagttgaaggaactagacagggaatcaagtggtacgaagaaaacgtcgaacacatttatggcggtgttattggacctgctactcctaccgctgcacagtaatctagagcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaaggcgcgccagtcccgggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtcgagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtatgacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 131 - DNA, 22 bp, synthetic construct
  misc_feature 1..22: note = Synthetic construct primer P675
  source 1..22: mol_type = other DNA
ccttggttgtgttctctcttct

SEQ ID NO: 132 - DNA, 21 bp, synthetic construct
  misc_feature 1..21: note = Synthetic construct primer P676
  source 1..21: mol_type = other DNA
gatctgttcagcgagtccttt

SEQ ID NO: 133 - DNA, 21 bp, synthetic construct
  misc_feature 1..21: note = Synthetic construct primer P677
  source 1..21: mol_type = other DNA
tagtcctagcatcgacgaaga

SEQ ID NO: 134 - DNA, 22 bp, synthetic construct
  misc_feature 1..22: note = Synthetic construct primer P678
  source 1..22: mol_type = other DNA
agcgaagggagaaatccaataa

SEQ ID NO: 135 - DNA, 20 bp, synthetic construct
  misc_feature 1..20: note = Synthetic construct primer P602
  source 1..20: mol_type = other DNA
cggccgattctgtttatctc

SEQ ID NO: 136 - DNA, 20 bp, synthetic construct
  misc_feature 1..20: note = Synthetic construct primer P603
  source 1..20: mol_type = other DNA
tccttctggttcatcccaac

SEQ ID NO: 137 - DNA, 507 bp, synthetic construct
  misc_feature 1..507: note = Synthetic construct L1M variant gene
  source 1..507: mol_type = other DNA
atggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtga

SEQ ID NO: 138 - protein, 168 aa, synthetic construct
  REGION 1..168: note = Synthetic construct L1M variant protein
MVSLVVFSLLLIGFASAQTLIVDSCKKAAAKEPFMKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 139 - DNA, 510 bp, synthetic construct
  misc_feature 1..510: note = Synthetic construct L1ML variant gene
  source 1..510: mol_type = other DNA
atgttggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtga

SEQ ID NO: 140 - protein, 169 aa, synthetic construct
  REGION 1..169: note = Synthetic construct L1ML variant protein
MLVSLVVFSLLLIGFASAQTLIVDSCKKAAAKEPFMKYDFCVNSLTQDPQSKTATTLEGLVLASTKNAAAETLNVKGLAEQILKGKGYGPGMEAGLHKCVKIYGGAYDFLNTALANVQSHHYSTAVEEFLYASFAPFDCVKYYWISPFAKESYIIFEKILIPMTLTKML

SEQ ID NO: 141 - protein, 408 aa, Glycine max
MTSTLLSLLFLLFSLLIPSFISSSPVRNPEEVVQEVNRKINGSIARPRRNLGYLWCGSGNPIDDCWRCDPNWEQNRQRLADCAIGFGKNAIGGRDGKIYVVDDDGDDDAVNPKPGSLRHAVIQDEPLWIIFARDMVIQLKEELLMNSFKTIDGRGASVHVAGGPCITIQYVTNVIIHGIHIHDCKQGGNAMVRDSPRHYGWRTVSDGDGVSIFGGSHVWIDHCSLSNCNDGLIDAIHGSTAITISNNYMTHHDKVMLLGHSDAYTQDKAMQVTIAFNHFGEGLVQRMPRCRLGYFHVVNNDYTHWEMYAIGGSANPTINCQGNRFVAPDDRFSKEVTKREDAPESEWQDWNWRSEGDLLVNGAFFTSSGAGASSSYARASSLSARPSSLVGSITTGAGALSCKKGSPC

SEQ ID NO: 142 - protein, 504 aa, Glycine max
MLPITCILFMCLLSSFSPPINALLNLNLTLPHQYPHPESVVQDIQRKVNASLRRREMLSKDEQQGMSSCLTGNPIDDCWRCEPNWAAERQKLAECGLGFGKYAMGGKGGQIYIVTDSSDRDPANPIPGTLRHAVIQDEALWIVFAADMTINLKHELIFNSYKTLDGRGANVHVTGHGCITLQYVSNIIIHNIHIHHCTPSGNTNIRASPTHVGWRGKSDGDGISIFGSRKIWIDHCSLSYCTDGLIDAIMGSTGITISNSHFAHHDEVMLLGHDDKYLVDRGMQVTIAFNHFGEGLVQRMPRCRLGYIHVVNNDFTQWRMYAIGGSANPTINSQGNRYTAPGDPDAKEVTKRVDTDDREWSGWNWRTEGDIMVNGAFFVPSGAAGQSGQYQEATSVQAKSAVQIDQLTMYSGVLGDPRDNGDLYPGFNGGGTVTGATSKGNTDGSSSDDGGDFFGMIFRGGSSSSSSSQAAPPSSVLFVSIFLSLLIIFVLDTTTNHAFLLSLL

SEQ ID NO: 143 - protein, 450 aa, Glycine max
MAVSSSATKWVLFLLLALLIREEAMAMATTPQISDLRNVEVERHRLPSLTNSSMVERAKEADKLNEQAAVANPEEVVSMVEMSIQNSTERRKLGYFSCGTGNPIDDCWRCDPNWQRNRKRLADCGIGFGRNAIGGRDGKFYVVTDPRDDDPVNPKPGTLRHAVIQDRPLWIVFKRDMVIQLKQELIMNSFKTIDARGVNVHIANGACITIQFVTNVIIHGLHIHDCKPTGNAMVRSSPTHFGWRTMADGDAISIFGSSHIWVDHNSLSHCADGLVDAVMGSTAITISNNHFTHHNEVILLGHSDSYTRDKLMQVTIAYNHFGEGLIQRMPRCRHGYFHVVNNDYTHWEMYAIGGSANPTINSQGNRYNAPTNPFAKEVTKRVETAETQWKGWNWRSEGDLLLNGAYFTPSGAGASASYARASSLGAKSSSMVDSMTSNAGALGCKRGRQC

SEQ ID NO: 144 - protein, 489 aa, Glycine max
MLEPSESTPPNSLSLLFLSSTFSLTKATTHTLRARGIQLSAMAVSSSSATKWVVFLLLALLIREEAMATPQISDLRNLEVERHRLPSLTNSSMAERAKEAEKLNEQAAVANPEEVVSMVEMSIQNSTERRKLGFFSCGTGNPIDDCWRCDPNWQRNRKRLADCGIGFGRNAIGGRDGKFYVVTDPRDDDPVNPKPGTLRHAVIQDKPLWIVFKRDMVIQLKQELIMNSFKTIDGRGVNVHIANGACITIQFVTNVIIHGLHIHDCKPTGNAMVRSSPTHFGWRTMADGDAISIFGSSHIWVDHNSLSHCADGLVDAVLGSTAITISNNHFTHHNEVILLGHSDSYTRDKQMQVTIAYNHFGEGLIQRMPRCRHGYFHVVNNDYTHWEMYAIGGSANPTINSQGNRYNAPTNRFAKEVTKRVETAESQWKGWNWRSEGDLLLNGAYFTPSGAGASASYARASSLGAKSSSMVGSMTSNAGALGCKRGSQC

SEQ ID NO: 145 - protein, 316 aa, Glycine max
MAALVITVSQDGTGQYRTVQEAIDAVPLGNTRRTVIRVSPGTYRQPLYVAKTKNFITLVGLRPEDTVLTWNNTATSIHHHQDARVIGTGTFGCGTIIVEGGDFIAENITFENSSPQGAGQAVAVRVTVDRCAFYNCRFLGWQDTLYLHYGIQYLKDCYIEGSVDFIFGNSTALLEHCHIHCKSAGFITAQSRNSPQEKTGYVFLRCVVTGNGGTSYAYLGRPWRPFARVVFAFTYMDQCIKPAGWNNWGKIENEKTACFYEYRCFGPGWCPSQRVKWARELQAEAAEQFLMHSFIDPESERPWLAQRMALKIPYSA

SEQ ID NO: 146 - protein, 316 aa, Glycine max
MAACIFTVAQDGTADFQTVQEAIDAVPLGNIRRTVIRVSPGIYRQPVYVPKTKNFITLAALSPEDTVLTWNNTATGIDHHQPARVIGTGTFGCGSTIVEGEDFIAENITFENSAPEGSGQAVAIRVTADRCAFYNCRFLGWQDTLYLHYGKQYLKDCYIEGSVDFIFGNSTALLEHCHIHCKSAGFITAQSRKSSQETTGYVFLRCVITGNGGNSYAYLGRPWGPFGRVVFAYTYMDQCIRHVGWDNWGKMENERSACFYEYRCFGPGCCPSKRVTWCRELLDEEAEQFLTHPFIDPELEKPWLAQRMALRIPYSA

SEQ ID NO: 147 - protein, 184 aa, Glycine max
MTNLKPLILFFYLLAIVVMISIPSSHCSRTLLPENEKLIENTCKKTPNYNVCLESLKASPGSSSADVTGLAQIMVKEMKAKANDALKRIQELQRVGASGPKQRRALSSCADKYKAVLIADVPQATEALQKGDPKFAEDGANDAANEATYCETDFSAAGNSPLTKQNNAMHDVAAVTAAIVRLLL

SEQ ID NO: 148 - protein, 182 aa, Glycine max
MKIMESLALIFYSTLVLATISVPATNSRIIHQKNNANLIEETCKQTPHHDLCIQYLSSDPRSTEADVTGLALIMVNVIKIKANNALDKIHQLLQKNPEPSQKEPLSSCAARYKAIVEADVAQAVASLQKGDPKFAEDGANDAAIEATTCENSFSAGKSPLTNHNNAMHDVATITAAIVRQLL

SEQ ID NO: 149 - protein, 203 aa, Glycine max
MATTTLMKLAFMLLMNLVICSAESSIGRKSNPNPEEFVKSSCRATRYPVLCVKSLLAYASVIRRSDRQLATTALSVSISRSRSSAWLVKKMLKARGMKPREYRAVQDCVENIGDSVDRLRQSVTELGRTGEDFVWHMSNVQTWVSAALTDDSTCLDGFAGSAMNGNVKALIKDRIVHVAQVTSNALALVNRFASRHPSATQTP

SEQ ID NO: 150 - protein, 214 aa, Glycine max
MRTQRLNHLLLSLFLFAAAFSSLHPTATAGDEGAPSPGDGDGDADFIRTSCNTTLYPDVCFTSLSRYASAVQQNPGQLARVAISVSLSKVHRAASYVSNLTRDADYDGTTRAALALHDCFSNLGDAVDEIRGSLKQMRQIGAAGAGASSFLFQMSNVQTWMSAALTDEETCTDGFQDVADCPVKTDVCDRVTNVKKFTSNALALVNSYANKGMP

SEQ ID NO: 151 - protein, 214 aa, Glycine max
MRTQRLNHLLLSLFLFAAAFSSLHPTATAGDEGAPSAGDGDRDADFIRTSCNTTLYPEVCFTSLSRYANAVQQNPGHLARVAIAVSLSKVHRAASYVSNLTRDADYGGSTRAALALHDCFSNLGDAVDEIRGSLKQMRQIGSAGAGASSFLFQMSNVQTWLSAALTDEETCTDGFQDVADCPMKTGVCDRVSNVKKFTSNALALVNSYANKGMP

SEQ ID NO: 152 - protein, 555 aa, Glycine max
MMASSSLFYLSLSLILTLTSAARHKPHPSPAKPPAKPAVTTATSPAIQQACAATRFPQQCEASLSQSQNLPPNPNPTPLQLLQSAIALSSDNLATAQTMVKSLHDASADSRNRTVAAATCIEILANSHYRISLASDALPRGRTKDARAWLGAALAYQYDCWNSLKYANDTEMVGKTMLFIDNLETLSSNALSMAFSFDAFGNDTASWKPPVTERDGFWEAVGSGGPASAGGVPPNLTPDVTVCNNGGDGCYKTVQEAVNAAPANGTKRFVIYIKEGVYEETVRIPLEKRNVVFLGDGIGKTVITGNGNVGQQGMTTYNSATVAVLGDGFMAKELTVENTAGPDAHQAVAFRLDSDLSVIENCEFLGNQDTLYAHSLRQFYKSCRIEGSVDFIFGNAAAVFQDCQILVRPRQVKPEKGENNAITAHGRTDPAEPTGFVFQNCLINGTEEYIALYLSKPQVHKNYLGRPWKEYSRTVFINSILEALVTPQGWMPWSGDFALKTLYYGEFENKGTGSDLSQRVPWSSKIPAEHVLTYSVQNFIQGNDWIPSSVGSPSS

SEQ ID NO: 153 - protein, 584 aa, Glycine max
MEYGRLGPSDPGGSSRLNNVPPTSSGRKKIVLLSLFSVLLIAASAVTAVVVRSRIQQNTRAHETRLGKPTQAISRTCSKTRFKTLCVKSLLDFPGSEEASEKDLVHISFNVTLQHFSKALYSSAAMSYTAMDPRVRAAYDDCLELLDDSVDALARSLNTVSVGAVGSANDDVLTWLSAALTNQDTCAEGFTDAVGTVKDHMSSNLRDLSELVSNCLAIFSGAGAGDDFAGVPIQNRRRLMEMREDNFPTWLSRRDRKLLILPLSQIQADIVVSKDGNGTVKTIAEAIKKVPEYSSRRIIIYVRAGRYEEENLKLGRKKTNVMFIGDGKGKTVITGGRNYYQNLTTFHTASFAASGSGFIAKDMTFENYAGPGRHQAVALRVGADHAVVYRCNIIGYQDTMYVHSNRQFYRECDIYGTVDFIFGNAAVVFQNCTLWARKPMAQQKNTITAQNRKDPNQNTGISIHNCRIMATPDLEASKGSYPTYLGRPWKLYARTVYMLSYIGDHVHPRGWLEWNTSSFALDTCYYGEYMNYGPGSGLGQRVNWAGYRVINSTVEASRFTVGQFISGSSWLPSTGVAFIAGLST

SEQ ID NO: 154 - protein, 615 aa, Glycine max
MPATNHPIYPLQTSHSTKSQYHLPSSSSSSSSSSSSSSSSSSSSIYIYIYTMYSPISRKKPPMESHVDTILSAIFVLLLSSLTHFSITANATRTPQENSLHFQVANSTCEGTLYSDLCVSTLASFPDLTSKTLPQMIRSVVNHTIYEVTLSASNCSGLRRNLPKLDKLEQRALDDCLNLFDDTVSELETTIADLSQSTIGPKRYHDAQTLLSGAMTNLYTCLDGFAYSKGHVRDRFEEGLLEISHHVSNSLAMLKKLPAGVKKLASKNEVFPGYGKIKDGFPTWLSTKDRKLLQAAVNETNFNLLVAKDGTGNFTTIAEAVAVAPNSSATRFVIHIKAGAYFENVEVIRKKTNLMFVGDGIGKTVVKASRNVVDGWTTFQSATVAVVGDGFIAKGITFENSAGPSKHQAVALRSGSDFSAFYKCSFVAYQDTLYVHSLRQFYRDCDVYGTVDFIFGNAATVLQNCNLYARKPNENQRNLFTAQGREDPNQNTGISILNCKVAAAADLIPVKSQFKNYLGRPWKKYSRTVYLNSYMEDLIDPKGWLEWNGTFALDTLYYGEYNNRGPGSNTSARVTWPGYRVIKNATEANQFTVRNFIQGNEWLSSTDIPFFSDFS

SEQ ID NO: 155 - protein, 600 aa, Glycine max
  REGION 69..101: note = misc_feature - X can be any naturally occurring amino acid
  SITE 105: note = misc_feature - X can be any naturally occurring amino acid
MTNPKLGYAGISDSEEHIPSSKKNHKKLLLSLLATLLVAASVVAIVAGVKNKTKNSDNSDTNSTSLSLXXXXXXXXXXXXXXXXXXXXXXXXXXXXXXXXXNQRXVLTMTRDVIQLSLSITFRAVERNYFTVKKLLTKHDLTKRETTALHDCLETIDETLDELREAQHDLELYPNKKTLYQHADDLKTLISAAITNQVTCLDGFSHDDADKHVRKELEKGQVHVEHMCSNALAMTKNMTDGDIANYEYKMKVENTNSNRKLLVENGVEWPEWISAADRRLLQAATVKADVTVAADGSGDFKTVTEAVKAAPLKSSKRYVIRIKGGVYRENVEVDKKKTNIMFLGDGRTNTIITASRNVVDGSTTFHSATVAVVGANFLARDITFQNTAGPSKHQAVALRVGGDLSAFFNCDFLAFQDTLYVHNNRQFFVKCLITGTVDFIFGNSAVVFQDCDIHARLPDSGQKNMVTAQGRVDPNQNTGIVIQKCRIGATKDLESVKKNFKTYLGRPWKEYSRTVIMQSSISDVIDPIGWHEWSGNFALSTLVYREYQNTGPGAGTSNRVTWKGYKVITDAAEARDYTPGSFIGGSSWLGSTGFPFSLGL

SEQ ID NO: 156 - protein, 598 aa, Glycine max
MTNPKLGYAGISDSGNHIPSSKKNHKKLLLSLLATLLVAASLVAIVVGVKNKNSDNSATSTPLSLSHHSHTIVKSACSSTFYPELCYSAIASEPNVTHKITTNRDVIQLSLKITFRAVEQNYFTVKKLFTEHDDLTKREKTALHDCLETIDETLDELREAQHNLELYPNKKTLYQHADDLKTLISAAITNQVTCLDGFSHDDADKHVRKALEKGQVHVEHMCSNALAMTKNMTDSDIANYEYNMRVENNGQNGNSNRKLLVENDVEWPEWISAADRRLLQASTVKADVTVAADGSGDFKTVTEAVDAAPLKSSKRFVIRIKAGVYRENVEVPKKKNNIMFLGDGRTNTIITASRNVVDGSTTFHSATVAVVGSNFLARDLTFQNTAGPSKHQAVALRVGGDLSAFFNCDILAFQDTLYVHNNRQFFVKCLIAGTVDFIFGNSAVVFQDCDIHARLPSSGQKNMVTAQGRVDPNQNTGIVIQKCRIGATNDLESVKKNFKTYLGRPWKEYSRTVIMQSSISDVIDPIGWHEWSGNFGLSTLVYREYQNTGPGAGTSNRVTWKGYKVITDTAEAREYTPGSFIGGSSWLGSTGFPFSLGL

SEQ ID NO: 157 - protein, 405 aa, Glycine max
MEIPIPIPLSSMLLFLLLVPCCICSSPLQDPELVVEDVQKSINASRRNLAFLSCGTGNPIDDCWRCDPNWEKNRKRLADCSIGFGKHAVGGRDGKLYVVTDPGDHPVNPKPGTLRYGVIQEEPLWIIFKRDMVIKLKQELMMNSFKTIDGRGVSVHIAGGPCITIQYVTNIIIHGINIHDCKQGGNAYVRDSPTHYGWRTLSDGDGVSIFGGSHVWVDHCSLSNCRDGLIDAIHGSTAITISNNYMTHHNKVMLLGHSDTFTRDKNMQVTIAFNHFGEGLVQRMPRCRHGYFHVVNNDYTHWRMYAIGGSAAPTINSQGNRFLAPNDNTFKEVTKRENSPQSKWKNWNWRSNGDLMLNGAFFTASGAGASSSYARASSLAAKSSSLVSSITASAGSLRCRKGSRC

SEQ ID NO: 158 - protein, 402 aa, Glycine max
MEIPIPLSFMLLLLLVPSCICSSPLQDPELVVEDVQKSINASRRNLAFLSCGTGNPIDDCWRCDPNWEKNRKRLADCSIGFGKHAVGGRDGKIYVVTDPGDHPVNPKPGTLRYGVIQEEPLWIIFKRDMVIKLKQELMMNSFKTIDGRGASVHIAGGPCITIQYVTNIIIHGINIHDCKQGGNAYVRDSPTHYGWRTLSDGDGVSIFGGSHVWVDHCSLSNCRDGLIDAIHGSTGITISNNYLTHHNKVMLLGHSDTFTRDKNMQVTIAFNHFGEGLVQRMPRCRHGYFHVVNNDYTHWRMYAIGGSAAPTINSQGNRFLAPNDNTFKEVTKRENSAQSKWKNWNWRSSGDLMLNGAFFTASGAGASSSYARASSLAAKSSSLVSSITASAGSLSCRKGSRC

SEQ ID NO: 159 - protein, 502 aa, Glycine max
MLHITCILLMCLLSSFSPPINALLNLTLPHQHPHPESVVHDLQRKVNASLWRREMLSKEDQQEGMSSSSCLTGNPIDDCWRCDPNWAADRQKLAECGLGFGKYAMGGKGGQIYIVTDSSDRDPANPVPGTLRHAVIQDEPLWIVFAADMTINLKHELIFNSYKTLDGRGANVHVTGHGCITLQYVSNIIIHNIHVHHCTPSGNTNIRASPTHVGWRGKSDGDGISIFGSRKIWIDHCSLSYCTDGLIDAIMGSTGITISNSHFAHHDEVMLLGHDDKYLPDRGMQVTIAFNHFGEGLVQRMPRCRLGYIHVVNNDFTQWKMYAIGGSANPTINSQGNRYTAPADPDAKEVTKRVDTDDREWSGWNWRTEGDIMVNGAFFVPSGAGQSAQYAEATSVQAKSAVQIDQLTMYSGVFGDPRDNGDLYPGFNGGGTVTGATSKGNTEGSSSDDGDFFGMIFRGSSSSQAAPPSPSSSIVFVSTFLSLLIIFILDTTTNHAILLSLL

SEQ ID NO: 160 - protein, 477 aa, Glycine max
MLQTTCIILFSVVAIFLPHGTAMLNLTLPGQHPDPEAVAREVHRKVNASMARREMLGVSEKEVASCLTGNPIDDCWKCDPDWANNRQRLADCAIGFGQNAKGGKGGQFYIVTDSSDEDPVNPKPGTLRYAVIQNEPLWIVFPSNMMIKLSQELIFNSYKTIDGRGADVHIVGGGCITLQYISNVIIHNIHIHHCHPSGNANVRSSPEHYGYRTESDGDGISIFGSRDIWIDHCTLSRCKDGLIDAVMGSSAITISNNHFSHHNDVMLLGHSDHYLPDSGMQVTIGFNHFGEGLVQRMPRCRRGYIHVVNNDFTRWEMYAIGGSAGPTINSQGNRYTAPEDPYAKQVTKRLDAGEGEWSGWNWRSEGDVLLNGAFFVASGAVAEPNYQNAYSTQPKNVDRISLLTMSAGVLGVARDNNLGMWIRGPNDGTVYFSDSGPEYTDEMSRSTMPLLPSRILILVSALLCFLGHTTTLTTLPN

SEQ ID NO: 161 - protein, 379 aa, Glycine max
MSNLHYMFILFAVLLVQNPSRIHCHTKGIRPRLSAGKGLSTNITRVQYSEQQFMKWVNFVGSLKHSVFKSAKNKLVASYTLHVDKDPGAGDFTSIQEAIDSLPFINLVRVVIKVHAGVYTEKVNIPPLKSYITIEGAGTDKTIVKWGDTAQTPGPNGRPLGTYGSATFAVNSPYFLAKNITFQNTTPVPAPGAVGKQAVALRISADTAAFVGCKFLGAQDTLYDHLGRHYYKDCYIEGSVDFIFGNSLSLFEGCHVHAIAQNTGAVTAQGRSSMLEDTGFSFVNCKVTGSGALYLGRAWGPFSRVVFAYTFMDNIIIPKGWYNWGDPNREMTVFYGQYKCTGLGASFAGRVPWSRELTDEEAAPFLSLSFIDGTEWIKV

SEQ ID NO: 162 - protein, 375 aa, Glycine max
MSNLHFIFYGLVVILLLQNPSATQCHTKGIQPKPGNGLSTNMTRVEFSEQQFMKWVKFVGGLKHSVFRTANNKLFPSHTLHVSKKHGKGGFSSIQAAIDSLPFINVVRVVIKVHAGVYTEKVNISPFKSFITIQGEGADKTIVQWGDTAQSQPLGTYGSATFAVNSAYFIAKNITFKNTAPIPAPGAVGKQGVALRISADTAVFQGCKFLGAQDTLYDHIGRHYYKDCYIEGSVDFIFGNALSLFEGCHVHAIAQLTGALTAQGRSSLLEDTGFSFVHCKVTGSGALYLGRAWGPFSRVVFAYTYMDNIIIPKGWYNWGDPNREMTVFYGQYKCTGPGASYAGRVSWSRELTDEEAKPFISLSYIDGSEWINLSL

SEQ ID NO: 163 - protein, 375 aa, Glycine max
MSNLHFIFYGLVVILFLQNPSATQCHTKGIRPKPGNGLSTNMTRVEFSEQQFMKWVKFVGGLKHSVFRTAKNKLFPSHTLHVSKKHGKGGFSSIQAAIDSLPFINVVRVVIKVHAGVYTEKVNISPFKSFVTIQGEGADKTIVQWGDTAQSQPLGTYGSATFAVNSPYFIAKNITFKNTAPIPAPGAVGKQGVALRISADTAVFLGCKFLGAQDTLYDHIGRHYYKDCYIEGSVDFIFGNALSLFEGCHVHAIAQLTGALTAQGRNSLLEDTGFSFVHCKVTGSGALYLGRAWGPFSRVVFAYTYMDNIIIPKGWYNWGDPNREMTVFYGQYKCTGPGASYAGRVSWSRELSDEEAKPFISLSYIDGSEWINLSL

SEQ ID NO: 164 - protein, 554 aa, Glycine max
MMVASLFYLSLSLSLILTLSSAAHHKPPHSPSKPPANPAVTAASPAIEQACAATLFPQQCEASLSQSQNLPPNPTPLQLLQSAIALSSDNLATAQTMAKSLLDASADSRNRTVAAATCIEILANSHHRISLASDALPRGRTKDARAWLGAALAYQYDCWNSLKYANDTQMVGKTMSFIDNLEILSSNALSMAFSFDAFGNDIASWKPPATERVGFWGTVGSGGPGPAGGVPLNLTPDVTVCKNGGDGCYKTVQEAVNAAPDNGNRTKRFVIHIKEGVYQETVRVPLAKRNVVFLGDGIGKTVITGDANVGQQGMTTYNSATVAVLGDGFMAKDLTIENTAGPDAHQAVAFRLDSDLSVIENCEFLGNQDTLYAHSLRQFYKSCRIEGNVDFIFGNAAAIFQDCQILVRPRQVKPEKGENNAITAHGRTDPAQPTGFVFQNCLINGTEEYMTLYHSKPQVHKNYLGRPWKEYSRTVFINSFLEVLVTPQGWMPWSGDFALKTLYYGEFESKGPGSYLSQRVPWSSKIPAEHVLTYSVQNFIQGNDWIPSIGSPSS

SEQ ID NO: 165 - protein, 581 aa, Glycine max
MEYGRLGPSDPGGSSRLNPPRSSGRKNIVFLSLFAVLLIAASAVTAVAVRSRTKNTGGDGTSLGKFTQAISRTCSKTRFKMLCMKSLLDFPGSQGASEKDLVHISFNVTLQHFSKALYSSATISYTAMDPRVRAAYHDCLELLDDSVDALARSLNTVSVGAVGSANDDVLTWLSAALTNQDTCAEGFADAAGTVKDQMANNLKDLSELVSNCLAIFSGAGAGDDFAGVPIQNRRRLMAMREDNFPTWLNGRDRRLLSLPLSQIQADIVVSKDGNGTVKTIAEAIKKVPEYSSRRIIIYIRAGRYEEDNLKLGRKKTNVMFIGDGKGKTVITGGRNYYQNLTTFHTASFAASGSGFIAKDMTFENYAGPGRHQAVALRVGADHAVVYRCNIIGYQDTMYVHSNRQFYRECDIYGTVDFIFGNAAVVFQNCTLWARKPMAQQKNTITAQNRKDPNQNTGISIHNCRIMATPDLEASKGSYPTYLGRPWKLYARTVFMLSYIGDHVHPRGWLEWNTSSFALDTCYYGEYMNYGPGSALGQRVNWAGYRAINSTVEASRFTVGQFISGSSWLPSTGVAFIAGLST

SEQ ID NO: 166 - protein, 354 aa, Glycine max
MAPLFISMQMLLLVFTCILLHVTCTTYHDSTPKTVPSTNSSKWVGPIGHRVITVDVNGGAHFRSVKAAVNAVPENNRMNVLIQISAGYYIEKVVVPVTKPYITFQGAGRDVTVIEWHDRASDPGPNGQQLRTYRTASVTVFANYFSARNISFKNTAPAPMPGMEGWQAAAFRISGDKAYFSGCGFYGAQDTLCDDAGRHYFKECYIEGSIDFIFGNGRSMYKDCRLHSIATRFGSIAAQDRQFPYEKTGFSFVRCKVTGTGPIYVGRAMGQYSRIVYAYTYFDGIVAHGGWDDIDWNTSNNNKTVFFGVYKCWGPGAAAIRGVPLAQELDFESAHPFLVKSFVNGRHWIAPSDA

SEQ ID NO: 167 - protein, 358 aa, Glycine max
MASSSLLVSLQVALVVFTCIFHAATVTCDRSFKIFSSMNNSSKSSHHWIGPIGHRKITVDVNGGGHYRSVQDAVNAVPDNNRKNVLVQINAGCYKEKVVVPVTKPYITFQGAGKEVTVIEWHDRASDPGPSGQQLRTYRTASVTVFATYFSARNISFKNTAPAPMPGMQGRQAVAFRISGDKAYFSGCGFYGAQDTLCDDAGRHYFKECYIEGSIDFIFGNGRSMYKDCELHSIATRFGSIAAHDRKEAEEKTGFAFVGCKVTGTGPLYVGRAMGQYSRIVYSYTYFDDIVAHGGWDDWDHADNKNKTVFFGVYKCWGPGAEAVRGVSWARDLNFESAHPFIRKSFVNGRHWIAPSDA

SEQ ID NO: 168 - protein, 363 aa, Glycine max
MTSSSLLVSLQVALAIFACIFHAATVTCKPHHRDRTLKIFSSTSNSSKSSHHWIGPIGHRKITVDINGGGHYRSVQDAVNAVPDNNRRNVLIQINAGCYKEKVVVPVTKPYITFEGAGKEVTVIEWHDRASDPGPSGQQLRTYRTASVTVFASYFSARNISFKNTAPAPMPGMQGWQAVAFRISGDKAYFSGCGFYGAQDTLCDDAGRHYFKECYIEGSIDFIFGNGRSMYKDCELHSIATRFGSIAAHDRKQPEEKTGFAFVRCKVTGTGPLYVGRAMGQYSRIVYSYTYFDDIVAHGGWDDWDHAHNKNKTVFFGVYKCWGPGAEAVRGVSWARDLDFEAAHPFIRKSFVNGRHWIAPSDA

SEQ ID NO: 169 - protein, 216 aa, Glycine max
MQLQPTLSTLYLSLLLTITLTSPTLAAQSKPQDLVRSSCVHARYPRLCLRTLSNYPGPANTPLDVARAALRVSLAHTRRASKFLHALSHGGAAAMSKRQRSALRDCNEQISDSVDQLRRSLDELQHLRSETFKWQMSNALTWVSAALTNGDTCLDGFGGNARPDVKRRVTDVARVTSNALYMINRLGQSRTGKPKPKPKPRSRPQPRSASSTEKLN

SEQ ID NO: 170 - protein, 406 aa, Glycine max
MATTFLSLLFLIFSLLTPALISSSPVQDPEFVAQEVNRKINASVARRNLGYLSCATGNPIDDCWRCDPNWEKNRQRLADCAIGFGKNAIGGKNGKIYVVTDSGDDDPVTPKPGTLRYAVIQDEPLWIIFARDMVIKLKEERIMNSFKTIDGRGASVHIAGGPCITIQYVTNVIIHGINIHDCKQGGNAMVRDSPRHYGWRTVSDGDGVSIFGGSHVWVDHCSLSNCNDGLIDAIHGSTAITISNNYMTHHDKVMLLGHSDSYTQDKNMQVTIAFNHFGEGLVQRMPRCRHGYFHVVNNDYTHWEMYAIGGSANPTINSRGNRFVAPDDRFSKEVTKHEDAAESEWKGWNWRSEGDLLVNGAFFTASGAGASSSYARASSLSARPSSLVGSITTGAGALTCRKGSRC

SEQ ID NO: 171 - protein, 334 aa, Glycine max
MAGMQKLPYGDVDSSLRALAGRAEGFGRLAIGGLHGPLYFVTTLSDDGPGSLREGCRRKEPLWIVFEVSGTIHLSSYLSVSSYKTIDGRGQRVKLTGKGLRLKECEHIIICNLEFEGGRGHDVDGIQIKPNSRHIWIDRCTLRDYDDGLIDITRQSTDITVSRCCFGQHDKTMLIGADPTHIGDRCIRVTIHHCFFDGTRQRQPRVRFGKVHLYNNYTRNWGIYAVCASVESQIYSQCNVYEAGTKKKTFEFYTEKAVDKEEQKSGFIISEGDMFLNGAEPSLLTENREESMFHPSEYYPTWTMEAAGHSLREVLLLCTGWQSICRPVDNMVLQ

SEQ ID NO: 172 - protein, 587 aa, Glycine max
MDTIKSFKGYGKVDELEQQAYQKKTRKRLIIIAVSSIVLFAVIIAAVAGVVIHKRNTSSSPSSDSPPQTELTPAASLKAVCHVTQYPNSCFSAISSLPESNTTDPELLFKLSLRVAIDELSKLSSFPSKLRANAEHDARLQKAIDVCGNVFGDALEQLNDSISALGSGAAEAGKIISPASVGDVETWISAALTDQDTCLDALAELNSTASRGALREIETAMRNSTEFASNSLAIVTKILGLLSKFDSPIHHRRLLGFPEWLGAAERRLLQVNSSETTPDAVVASDGSGQFRTIGEALRLVKKKSEKRFVVHVKEGRYVENIDLDKNTWNVFIFGDGKEKTVVVGSRNFMDGTPTFETATFAVKGKGFIAKDIGFVNNAGASKHQAVALRSGSDRSVFFRCSFDGFQDTLYAHSNRQFYRDCDITGTIDFIFGNAAAVFQNCKIMPRQPLPNQFNTITAQGKKDPNQNTGIIIQKSKFIPLGNNLTAPTYLGRPWKDFSTTVIMQSDIGSFLKPVGWISWVSNVEPVSTIFYAEYQNTGPGADVSQRVKWAGYKPTLTDVEADKFTVQSFIQGPEWLPNAAVEFDSTL

SEQ ID NO: 173 - protein, 518 aa, Glycine max
MAIPSLINQPQRTPSLTFSFLLFLAICTPLDAAHTDFAGSACLKVSPSHFAGSVTEVIAAIRQLASILSRFGSPLANFRLSTAIADCLDLLDLSSDVLSWALSASQNPKGKHNSTGNLSSDLRTWLSAALAHPETCMEGFEGTNSIVKGLVSAGIGQVVSLVEQLLAQVLPAQDQFDAASSKGQFPSWIKPKERKLLQAIAVTPDVTVALDGSGNYAKIMDAVLAAPDYSMKRFVILVKKGVYVENVEIKKKKWNIMILGQGMDATVISGNRSVVDGWTTFRSATFAVSGRGFIARDISFQNTAGPEKHQAVALRSDSDLSVFFRCGIFGYQDSLYTHTMRQFFRDCTISGTVDYIFGDATAVFQNCFLRVKKGLPNQKNTITAHGRKDPNEPTGFSFQFCNITADSDLIPSVGTAQTYLGRPWKSYSRTVFMQSYMSEVIGAEGWLEWNGNFALDTLYYAEYMNTGAGAGVANRVKWPGYHALNDSSQASNFTVSQFIEGNLWLPSTGVTFTAGLTV

SEQ ID NO: 174 - protein, 528 aa, Glycine max
MELSSKKELLSRIVSTLTPTITLIFFFLVLSPSLCTSLGSTNTVGSELLKVAPSEFEGTVRTVVDVLQEVTSILSEFGSGFGDSRLSNAVSDCLDLLDMSSDELDWSVSATQSPKGKHNSTGNTSSDLRTWLSAALANQDTCIDGFDGTNGMVKGLVSTGIGQVMSLLQQLLTQVKPVSDHFSFSSPQGQYPSWVKTGERKLLQANVVSFDAVVAADGTGNYTKVMDAVLAAPNYSMQRYVIHIKRGVYYENVEIKKKKWNLMMVGDGMDATIISGNRSFIDGWTTFRSATFAVSGRGFIARDITFQNTAGPEKHQAVALRSDSDLSVFFRCGIFGYQDSLYTHTMRQFYRECKISGTVDFIFGDATAIFQNCHISAKKGLPNQKNTITAHGRKNPDEPTGFSIQFCNISADYDLVNSVNSFNSTHTYLGRPWKPYSRTIFMQSYISDVLRPEGWLEWNGDFALDTLYYAEYMNYGPGAGVANRVKWQGYHVMNDSSQASNFTVSQFIEGNLWLPSTGVTFTAGLGVV

SEQ ID NO: 175 - protein, 547 aa, Glycine max
MPLKLAGVATVGSLLLCVAISLFHIAGAEDPYRFFNWNITYGDIYPLGVRQTGILINGQFPGPDIHSVTNDNLIINVFNSLDEPFLLSWNGIQQRRNSFEDGVFGTTCPIPAGKNFTYILQVKDQIGTFYYFPSLAFHKAAGGFGGIRILSRPRIPVPFPDPAGDYTVLIGDWYKSNHTTLKARLDRGKKLPFPDGILINGRGPNGVSFNVEQGKTYRLRISNVGLQHSLNFRIQNHKMKLVEVEGTHTLQTMYSSLDVHVGQSYSVLVTADQPAQDYYIVVSTRFSYKVLTTTGVLRYSNSAGPVSGPPPGGPTIQIDWSLNQARSIRTNLTASGPRPNPQGSYHYGMINTTKTIILASSAGQVNGKQRYAINSVSYVVPDTPLKLADYFKISGVFRPGSISDRPTGGGIYLDTSVLQADYRNFVEIVFQNNENIVQSYHLDGYSFFVVGMDGGQWTTASRNQYNLRDAVARCTTQVYPFSWTAIYIALDNVGMWNLRSEFWARQYLGQQLYLRVYTTSTSIRDEFPVPKNAILCGRASGRHTRPL

SEQ ID NO: 176 - protein, 226 aa, Glycine max
MDSLKMLKGYGKVEHHLEDHRNPKPKPKFSKPFIAAISVFAILFLTLTFAFALASMLHHSHHTESQQQLLNSAESIRVVCNVTRFPGACLAAIPPSANATNPQAILSLSLRASLHALQSLNSSLGTKNSRLADCRDQLDDALGRLNDALSAAAALTEAKISDVQTWVSAAITDQQTCLDGLEEVGDVAAMEEMKKMMKRSNEYTSNSLAIVANIRNLLQRFHMALH

SEQ ID NO: 177 - protein, 193 aa, Glycine max
MTTISNCSLLFLFLFLSTLHIASTLSTPTNFIKSSCSTTQYPALCIQSLSVYASTIQQDPHELVQTALSLSLNHTEATKTFVAKCNKFRGLKPREYAALKDCAEEISDSVDRLSRSLKELKLCKVKGEDFTWHISNVETWVSSALTDESTCGDGFAGKALNGKIKEAIRARMVNVAQVTSNALSLINQYAAQH

SEQ ID NO: 178 - protein, 450 aa, Glycine max
MENARISQWLILLVCALLLLISEASYVPITIVQNAVAKGAVCLDGSPPAYHFDRGFGSGINNWLVAFEGGGWCNNVTTCLARKTNRLGSSKQMAKLIAFSGILNNREMFNPDFYNWNRIKVRYCDGSSFTGDVEAVNPVTKLHFRGGRIFNAVMEDLLAKGMKNARNAIISGCSAGGLTSVLHCDRFRALLPRGARVKCLSDAGYFINGKDVLGEQHIEQYFSQVVATHGSARNLPQSCTSRLSPRLCFFPQYLVSRITTPIFFVNAAYDSWQIKNILAPGVADPEGHWHSCKLDINNCSPDQLDLMQGFRTEFLRAITVLGNSSSKGMFIDSCYAHCQTEMQETWLRSDSPELKKTTIAKAVADWFYERRPFHQIDCPYPCNPTCHNRVFDPQDDHPGKNVTAKGTSDASATKPNFPKLTNKAQWIELVCISRGISGLLLTLVLLNKMK

SEQ ID NO: 179 - protein, 399 aa, Glycine max
MEITRIGQWLSLLICVLLLLQTEGVPVGITFVENAVAKGAVCLDGSPPAYHFHKGSGAGINNWIVHFEGGGWCNNVTTCLSRRDTRLGSSKKMDTSLSFSGFFSNSKKFNPDFYDWNRIKVRYCDGSSFTGDVEAVDPKTNLHFRGARVFAVVVEDLLAKGMKNAQNAIISGCSAGGLASILNCDRFKSLLPATTKVKCLADAGFFINVKDVSGAQRIEEFYSQVVQTHGSAKNLPTSCTSRLRPGLCFFPQNVVSQISTPIFFVNAAYDSWQIKNILAPGAADPRGQWRECKLDIKNCSPNQLSVMQGFRTDFLRAFSVVGNAASKGHFIDGCYAHCQTGIQETWLRNDSPVVAKTSIAKAVGDWFYDRRPFREIDCAYPCNPTCHNRIFDQNERPDV

SEQ ID NO: 180 - protein, 408 aa, Glycine max
MTSTLLSLLFLLFSLLTPTLISSSPVLNPQEVVQEVNKKINGSIARPRRNLGYLSCGSGNPIDDCWRCDPNWEQNRQRLADCAIGFGKNAIGGRDGKIYVVEDDGDDDAVNPKPGTLRHAVIQDEPLWIIFARDMVIQLKEELLMNSFKTIDGRGASVHVAGGPCITIQYVTNVIIHGIHIHDCKQGGNAMVRDSPRHYGWRTVSDGDGVSIFGGSHVWVDHCSLSNCNDGLIDAIHGSTAITISNNYMTHHDKVMLLGHSDSYTQDKDMQVTIAFNHFGEGLVQRMPRCRLGYFHVVNNDYTHWEMYAIGGSANPTINCQGNRFVAPDDRFSKEVTKREDTPESEWQDWNWRSEGDLLVNGAFFTASGAGASSSYARASSLSARPSSLVGSITTGAGALSCKKGSPC

SEQ ID NO: 181 - protein, 406 aa, Glycine max
MATTFLSLLFLLFSLLTPTLISSSPVQDPEFVAQEVHRKINASVARRNLGYLSCATGNPIDDCWRCDPNWEKNRQRLADCAIGFGKNAIGGKNGKIYVVTDSGDDDPVTPKPGTLRYAVIQDEPLWIIFARDMVIKLKEELIMNSFKTIDGRGASVHIAGGPCITIQYVTNVIIHGINIHDCKQGGNAMVRDSPRHYGWRTISDGDGVSIFGGSHVWVDHCSLSNCNDGLIDAIHGSTGITISNNYMTHHDKVMLLGHSDSYTQDKSMQVTIAFNHFGEGLVQRMPRCRHGYFHVVNNDYTHWEMYAIGGSANPTINSQGNRFVAPDDRFSKEVTKHEDAPESEWKGWNWRSEGDLLVNGAFFTASGAGASSSYARASSLSARPSSLVGSITTGAGALSCRKGSRC

SEQ ID NO: 182 - protein, 379 aa, Glycine max
MGNAHHSQWHRKHGNSVLPPHKYNPQPPPTTSNSNMLSLPYTHVDTTLRSLAAQAEGFGRFAIGGLHGPLHHVTSLADDGPGSLRNACRRKEPLWIVFEVSGTIQLSSYLNVSSHKTIDGRGQRIKLSGKGLRLKECEHVIICNLEFEGGRGHDVDAIQIKPNSKHIWIDRCTLSDFDDGLIDITRESTDITISRCHFSQHDKAMLIGADPSHVGDRCMRVTIHHCFFNGTRQRQPRVRFAKVHLYNNYIRNWGIYAVCASVESQIFSQHNIYEAGQKKVAFKYLTEQAADKEVGATGTIMSEGDIFLNGAKAGLMAVDVSCNMFHPSEHYPSWTVEAPTDDLKPILLHCTGGHFFVGPAVGPFGALGHNRLLEVRVLL

SEQ ID NO: 183 - protein, 528 aa, Glycine max
MALSSKKELSQIAATPKLTITLIFFFVLFLTALGNTNTNTVGSELLKVAPSEFAGTVRTVVDVLQDITSILSEFGSGFGDSRLSNAVSDCLELLDMSSDELDWSVSATQSPKGKHNSTGNTSSDLRTWLSAALANQDTCMDGFDGTNGIVKGLVSTGLGQVMSLLQQLLTQVNPVSDHYTFSSPQGHFPPWVKPGERKLLQAANGVSFDAVVAADGTGNFTKVMDAVLAAPNYSMQRYVIHIKRGVYNENVEIKKKKWNLMMVGDGMDNTVISGNRSFIDGWTTFRSATFAVSGRGFVARDITFQNTAGPEKHQAVALRSDSDLSVFFRCGIFGYQDSLYTHTMRQFYRECKISGTVDFIFGDATAIFQNCHISAKKGLPNQKNTITAHGRKNPDEPTGFSIQFCNISADYDLVNSINNNSNNSIGTYLGRPWKPYSRTVFMQSYISDVLRPEGWLEWNGDFALDTLYYAEYMNYGPGAGVANRVKWPGYHVMNDSSQASNFTVSQFIEGNLWLPSTGVTFTAGLGGV

SEQ ID NO: 184 - protein, 586 aa, Glycine max
MDTIKSFKGYGKVDELEQQAYQKKTRKRLIIITVSSIVLIAVIIAAIAGVVIHKRNTSSSPSSDSPPQTELTPAASLKAVCDVTQYPNSCFSAISSLPDSNTTDPELLFKLSLRVAIDELSKLSSFPSKLRANAEHDARLQKAIDVCGNIFGDALDRLNDSISALGSSGGAGKIISPASVSDVETWISAALTDQDTCLDALGELNSTAASGALREIETAMRNSTEFASNSLAIVTKILGLLSQFAAPIHHRRLLGFPEWLGAAERRLLQVNSSETTLDAVVAQDGSGQFRTIGEALKLVKKKSEKRFVVHVKEGRYLENIDLDKNTWNVFIFGDGKDKTVVVGSRNFMDGTPTFETATFAVKGKGFIAKDIGFVNNAGASKHQAVAFRSGSDRSVFFRCSFNGFQDTLYAHSNRQFYRDCDITGTIDFIFGNAAAVFQNCKIMPRQPLPNQFNTITAQGKKDRNQNTGIIIQKSKFTPLENNLTAPTYLGRPWKDFSTTVIMQSDIGSFLKPVGWMSWVPNVEPVSTIFYAEYQNTGPGADVSQRVKWAGYKPTLTDGEAGKFTVQSFIQGPEWLPNAAVQFDSTL

SEQ ID NO: 185 - protein, 262 aa, Glycine max
MFIGDGKGKTVITGKKNVIDGMTTFHSASFAASGAGFIARDITFENYAGPAKHQAVALRVGADHAVVYRCNIVGYQDSCYVHSNRQFFRECNIYGTVDFIFGNAAVVFQKCNIYARKPMAQQKNTITAQNRKDPNQNTGISLHNCRILPAPDLAPVKGSFPTYLGRPWKQYSRTVYLVSYMGDHIHPRGWLEWNGDFALNTLYYGEYMNYGPGAAVGQRVQWPGYRVIKSTMEANRFTVAQFISGSAWLPSTGVAFAAGLST

SEQ ID NO: 186 - protein, 520 aa, Glycine max
MAIPSPKIFLLLTFFFSHSIAVKSSNVSTTPTLHATFPFPEVSSFNSSKLSISININPNIINTLLQSLQAAISEATKLSDLLNNAGHNIIENKIGAVQDCRELQQSTLASLKRSLSGIRSQDSRKLVDARTYLSAALTNKDTCLESLDSASGTLKPVLVNSVINSYKDVSDSLSMLPKPERKASKGHKNRRLLWLSTKNRRLLQSNDGGELVVAADGTGNFSTITEAINFAPNNSVGRTVIYVKEGTYEENVEIPSYKTNIVLLGDGKDVTFITGNRSVIDGWTTFRSATLAVSGEGFLARDIAFENKAGPEKHQAVALRVNADFTAFYRCAMYGYQDTLYVHSFRQFYRECEIFGTIDYIFGNAAVVLQASNIITRMPMLGQFTVITAQSRDSPDEDTGISIQNCSILATTDLYSNSGSVKSYLGRPWRVYSRTVFLESYIDQFIDPMGWKEWSGDQGLDTLYYGEYANYGPGSGTDNRVNWAGFHVMDYDSAYNFTVSEFIIGDAWLGSTSFPYDDGI

SEQ ID NO: 187 - protein, 553 aa, Glycine max
MAIQQSLLDRPRKSVSKTICLIFSIAAVMISSAFVGSYLIKSTSFFNQSSPQHLCDHALDRATCLTHVSEVVQGPILTPTKDHKFNLLQSFLMKYTSHIKRVMNTASSIKLRINSPKEEEALHDCVELMDLSISRVRDSMVTLTKQTIESQQDAHTWLSSVLTNHATCLDGLEGSARAFMKDELEDLISRARTSLAMFVAVLPPKVEQIIDEPLSGDFPSWVSSKDRRLLESTVGDIKANVVVAKDGSGKFKTVAEAVASAPDNGKTRYVIYVKKGTYKENVEIGKKKTNVMLVGDGKDATVITGNLNFIDGTTTFKTATVAAVGDGFIAQDIWFQNTAGPQKHQAVALRVGADQSVINRCRIDAFQDTLYAHSNRQFYRDSFITGTVDFIFGNAAVVFQKCDLVARKPMDKQNNMVTAQGREDPNQNTGTSIQQCNLTPSSDLKPVVGSIKTFLGRPWKKYSRTVVMQSTLDSHIDPTGWAEWDAQSKDFLQTLYYGEYMNNGPGAGTSKRVNWPGYHIIKTAAEASKFTVAQLIQGNVWLKNTGVNFIEGL

SEQ ID NO: 188 - protein, 197 aa, Glycine max
MIIIARLMFLLALFLLLVTNTGQMVFAQDDNNVRDACSVTRFQSLCVQTLGHFSRTAGTSPSKWARAGVSVSIGEVKNVEAYLAQVKRQGQLKGRNSVALSDCVETFGYAIDELHKSLGVLRSLSKSKFSTQMGDLNTWISAALTDEVTCLDGFEGSKGTNVKLLQNRVQNASYITSNALALINKLATEGLGSINDP

SEQ ID NO: 189 - protein, 208 aa, Glycine max
MKPTFLLSLLFFTFTLSHLTPPATAGDRYVSGDNSGDADFIRASCNATLYPDLCFSSLSRYAAAVQSSHAALARVAVAVALAKAHGAAAYLSHQTAAASDDDSGAGSALHDCFSNLEDAVDEIRGSLKQMRRLKPAGAGNSDSSSVRFGLSNVLTWMSAALTDEETCTDGFEGVEEGPVKTSVCDRVTRVKKFTSNALALVNGFANNL

SEQ ID NO: 190 - protein, 191 aa, Glycine max
MRLLPSSLISLSTMTNLKPLILLAIIVMISIPSSHCRTLLPENEKLIENTCRKTPNYNVCLESLKASPGSSSADVTGLAQIMVKEMKAKANYALKRIQELQRVGAGPNKQRRALSSCVDKYKTVLIADVPQATEALQKGDPKFAEDGANDAANEATFCEADFSAGNSPLTKQNNAMHDVAAVTAAIVRLLL

SEQ ID NO: 191 - protein, 420 aa, Glycine max
MEMERVLWVGIAIGLVFSNWVVGYEQYHFNETELSLLEAHEASLSYAGTERNNLLLVGLTLIQNAAAKGAVCLDGTLPGYHWHRGYGSGANSWLINLEGGGWCNNIRTCVYRKKTRRGSSDFMEKEIPFTGILSNKAEENPDFFNWNRVKLRYCDGASFTGDSEDETAELQFRGQRIWAAAMEDLMSKGMRFANQALLSGCSAGGLATIIHCDEFRGLFPRTTKVKCLSDAGLFLDVIDVSGGHTLRNLYSGVVGLQGAQKNLPQICTNHLDPISCFFPQNLIASVKTPLFILNAAYDSWQIQSSLAPPSADPHGYWQQCRLNHAKCTGPQIQFLQGFRNHMLNAIKYFSRSKQNGLFINSCFSHCQTERQDTWFADNSPVIRNKAIALAVGDWYFDRAGVKAIDCPYPCDNTCHHLIFR

SEQ ID NO: 192 - protein, 398 aa, Glycine max
MESARISQWLNLLVCVLLLLKAEGSLVPLILVENAESKGAVCLDGSPPAYHFDKGFGEGINSWIVHIEGGGWCNNIESCLDRKDTRLGSSKQMEDIYFSGILSNEQQFNPDFYNWNRVKVRYCDGSSFTGDVEEVDPTTNLHFRGARIFSAVIEELLAKGLERAENAILSGCSAGGLTTILHCDSFKTFLPSRANVKCVPDAGYFVNVEDISGAHFIQQYYSEVVSTHGSAKNLPTSCTSKLSPTLCFFPQYVASHISTPIFVVNSAYDSWQIRYIFVPGSADPSDSWNSCKVNMSNCSPDQLSKLQGFKSEFERALSEVGDSPSKGMFIDSCYAHCQTEPQETWFKTDSPKLANTTIAKAVADWFYGRSSFRHVDCNYPCNPSCQNRVFDLKDLPGI

SEQ ID NO: 193 - DNA, 11983 bp, synthetic construct
  misc_feature 1..11983: note = Synthetic construct vector pMBXS1274
  source 1..11983: mol_type = other DNA
taagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccagatctaaaatgaagttcttcgtttcagttgtaatgttctttctcctcctaaactgtttcgcagccgcgcaaaccttgattcgagattcctgcaaaacagctgcagcaaaagaccctaatctcaagtatgatttttgcatccaatcacttgaacaagatccgcaaagcaaaactgcaactagtctatcaggattggtcctagcggcaacgaataatgctgcatccaaaacaattaacgtgaaagggatagttgagactattctcaagagcaaaaagtatgcaccgagtactgaacccgcgttacgcacttgcgtaaagctttatgacgatgcttatggttctttaaaagaagctttgatgaacgttaaatccagtgattacaaaagtgctaatatgcatctgagtgctgctttggatgaacctgtcacttgtgaagatggtttcaaagagaagcacgctaaatctcccgttacaaacgagaacaatgttttgtttcagaagattttgattcctttagcttttaccaatatgctctgaatttaaatcctcaggctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagactagtcgtcaaagggcgacaccccctaattagcccaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcaggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgaaacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccgg

SEQ ID NO: 194 - DNA, 417 bp, synthetic construct
  misc_feature 1..417: note = Synthetic construct open reading frame encoding shortened canola ortholog to ISY
  source 1..417: mol_type = other DNA
atgaagtatgatttttgcatccaatcacttgaacaagatccgcaaagcaaaactgcaactagtctatcaggattggtcctagcggcaacgaataatgctgcatccaaaacaattaacgtgaaagggatagttgagactattctcaagagcaaaaagtatgcaccgagtactgaacccgcgttacgcacttgcgtaaagctttatgacgatgcttatggttctttaaaagaagctttgatgaacgttaaatccagtgattacaaaagtgctaatatgcatctgagtgctgctttggatgaacctgtcacttgtgaagatggtttcaaagagaagcacgctaaatctcccgttacaaacgagaacaatgttttgtttcagaagattttgattcctttagcttttaccaatatgctctga

SEQ ID NO: 195 - protein, 138 aa, synthetic construct
  REGION 1..138: note = Synthetic construct shortened canola ortholog to ISY
MKYDFCIQSLEQDPQSKTATSLSGLVLAATNNAASKTINVKGIVETILKSKKYAPSTEPALRTCVKLYDDAYGSLKEALMNVKSSDYKSANMHLSAALDEPVTCEDGFKEKHAKSPVTNENNVLFQKILIPLAFTNML

SEQ ID NO: 196 - DNA, 11983 bp, synthetic construct; identical to SEQ ID NO: 193
  misc_feature 1..11983: note = Synthetic construct vector pMBXS1277
  source 1..11983: mol_type = other DNA

SEQ ID NO: 197 - DNA, 9677 bp, synthetic construct
  misc_feature 1..9677: note = Synthetic construct vector pMBXS1269
  source 1..9677: mol_type = other DNA
ctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccagatctaaaatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtgacctcaggctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcggaccgctcgagcaattgtacgtagaattcgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcacgtgtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcag

SEQ ID NO: 198 - DNA, 9779 bp, synthetic construct
  misc_feature 1..9779: note = Synthetic construct vector pMBXS1270
  source 1..9779: mol_type = other DNA
ctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcctcagcttaattaagcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccagatctaaattggtttccttggttgtgttctctcttctcttgatcggttttgcatctgcgcaaactctcatagtagattcttgcaagaaagcagccgcaaaagagccgtttatgaaatatgatttctgcgtcaattctcttacacaagatccacaaagcaaaacggcgaccaccctcgaaggtttagtcctagcatcgacgaagaatgctgcggcggaaacactgaacgtaaaaggactcgctgaacagatcctcaaggggaagggatatgggccaggtatggaggcagggctacacaagtgcgtcaagatttatggaggtgcttatgattttttaaacactgctttagcgaacgttcaatcacaccattatagtactgctgtagaggaatttctttatgcttcatttgcaccgttcgactgcgtgaaatattattggatttctcccttcgctaaggagagctatattatctttgagaagattttgattcctatgactttaactaaaatgttgtgacctcaggctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcggaccgctcgagcaattgtacgtagaattcgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcacgtgtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaacagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcag